SEQUENCE LISTING (92 sequences)

SEQ ID NO: 1 - protein, 630 aa, Artificial Sequence
  Synthetic Peptide
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVV

SEQ ID NO: 2 - protein, 601 aa, Artificial Sequence
  Synthetic Peptide
MSSLRPAQSSSSSSRTRQSSQARILAQTTLDAELNAEYEESGDSFDYSKLVEAQRSTPPEQQGRSGKVIAYLQHIQRGKLIQPFGCLLALDEKSFRVIAFSENAPEMLTTVSHAVPNVDDPPKLGIGTNVRSLFTDPGATALQKALGFADVSLLNPILVQCKTSGKPFYAIVHRATGCLVVDFEPVKPTEFPATAAGALQSYKLAAKAISKIQSLPGGSMQALCNTVVKEVFDLTGYDRVMAYKFHEDEHGEVFAEITKPGIEPYLGLHYPATDIPQAARFLFMKNKVRMICDCRARSVKIIEDEALSIDISLCGSTLRAPHSCHLQYMENMNSIASLVMAVVVNENEDDDEPESEQPPQQQKRKKLWGLIVCHHESPRYVPFPLRYACEFLAQVFAVHVNKEFELEKQIREKSILRMQTMLSDMLFKESSPLSIVSGSPNIMDLVKCDGAALLYGDKVWRLQTAPTESQIRDIAFWLSEVHGDSTGLSTDSLQDAGYPGAASLGDMICGMAVAKITSKDILFWFRSHTAAEIKWGGAKHDPSDEDDSRRMHPRLSFKAFLEVVKMKSLPWSDYEMDAIHSLQLILRGTLNDALKPAQSSG

SEQ ID NO: 3 - protein, 601 aa, Artificial Sequence
  Synthetic Peptide
MSSSRPTQCSSSSSRTRQSSRARILAQTTLDAELNAEYEEYGDSFDYSKLVEAQRTTGPEQQARSEKVIAYLHHIQRAKLIQPFGCLLALDEKTFNVIALSENAPEMLTTVSHAVPSVDDPPKLRIGTNVWSLFTDPGATALQKALGFADVSLLNPILVQCKTSGKPFYAIVHRATGCLVVDFEPVKPTEFPATAAGALQSYKLAAKAISKIQSLPGGSMEVLCNTVVKELFDLTGYDRVMAYKFHEDDHGEVFAEITKPGLEPYLGLHYPATDIPQAARFLFMKNKVRMICDCRARSIKIIEDESLHLDISLCGSTLRAPHSCHLQYMENMNSIASLVMAVVVNENEDDDEVGADQPAQQQKRKKLWGLLVCHHESPRYVPFPLRYACEFLAQVFAVHVNKEFELERQVREKSILRMQTMLSDMLLRESSPLSIVSGTPNIMDLVKCDGAALLYGGKVWRLQNAPTESQIRDIAFWLSDVHRDSTGLSTDSLHDAGYPGAAALGDMICGMAVAKINSKDILFWFRSHTAAEIRWGGAKHDPSDKDDSRRMHPRLSFKAFLEVVKMKSLPWNDYEMDAIHSLQLILRGTLNDDIKPTRAAS

SEQ ID NO: 4 - protein, 599 aa, Artificial Sequence
  Synthetic Peptide
MSSSRPASSSSSRNRQSSRARVLAQTTLDAELNAEYEESGDSFDYSKLVEAQRDGPPVQQGRSEKVIAYLQHIQKGKLIQTFGCMLALDEKSFNVIAFSENAPEMLTTVSHAVPSVDDPPRLGIGTNVRSLFSDQGATALHKALGFADVSLLNPILVQCKTSGKPFYAIVHRATGCLVVDFEPVKPTEFPATAAGALQSYKLAAKAISKIQSLPGGSMEVLCNTVVKEVFDLTGYDRVMAYKFHEDDHGEVFAEITKPGLEPYLGLHYPATDIPQAARFLFMKNKVRMICDCRARSIKVIEAEALPFDISLCGSALRAPHSCHLQYMENMNSIASLVMAVVVNENEEDDEAESEQPAQQQQKKKLWGLLVCHHESPRYVPFPLRYACEFLAQVFAVHVNREFELEKQLREKSILKMQTMLSDMLFREASPLTIVSGAPNIMDLVKCDGAALLYGGKVWRLRNAPTESQIHDIAFWLSDVHRDSTGLSTDSLHDAGYPGASALGDMICGMAVAKINSKDIIFWFRSHTAAEIRWGGAKHDSSDMDDSRRMHPRLSFKAFLEVVKMKSLPWTDYEMDAIHSLQLILRGTLNDASKPKREAS

SEQ ID NO: 5 - protein, 599 aa, Artificial Sequence
  Synthetic Peptide
MSGSRPTQSSEGSRRSRHSARIIAQTTVDAKLHADFEESGSSFDYSTSVRVTGPVVENQPPRSDKVTTTYLHHIQKGKLIQPFGCLLALDEKTFKVIAYSENASELLTMASHAVPSVGEHPVLGIGTDIRSLFTAPSASALQKALGFGDVSLLNPILVHCRTSAKPFYAIIHRVTGSIIIDFEPVKPYEVPMTAAGALQSYKLAAKAITRLQSLPSGSMERLCDTMVQEVFELTGYDRVMAYKFHEDDHGEVVSEVTKPGLEPYLGLHYPATDIPQAARFLFMKNKVRMIVDCNAKHARVLQDEKLSFDLTLCGSTLRAPHSCHLQYMANMDSIASLVMAVVVNEEDGEGDAPDATTQPQKRKRLWGLVVCHNTTPRFVPFPLRYACEFLAQVFAIHVNKEVELDNQMVEKNILRTQTLLCDMLMRDAPLGIVSQSPNIMDLVKCDGAALLYKDKIWKLGTTPSEFHLQEIASWLCEYHMDSTGLSTDSLHDAGFPRALSLGDSVCGMAAVRISSKDMIFWFRSHTAGEVRWGGAKHDPDDRDDARRMHPRSSFKAFLEVVKTRSLPWKDYEMDAIHSLQLILRNAFKDSETTDVNTKV

SEQ ID NO: 6 - protein, 598 aa, Artificial Sequence
  Synthetic Peptide
MSSSRPSQSSTTSARSKHSARIIAQTTIDAKLHADFEESGDSFDYSSSVRVTSVAGDERKPKSDRVTTAYLNQIQKGKFIQPFGCLLALDEKTFKVIAFSENAPEMLTMVSHAVPSVGELPALGIGTDIRTIFTGPSAAALQKALGFGEVSLLNPVLVHCKTSGKPYYAIVHRVTGSLIIDFEPVKPYEVPMTAAGALQSYKLAAKAITRLQALPSGSMERLCDTMVQEVFELTGYDRVMTYKFHDDDHGEVVAEITKPGLDPYLGLHYPATDIPQAARFLFMKNKVRMICDCRAKHVKVVQDEKLPFDLTLCGSTLRAPHYCHLQYMENMSSIASLVMAVVVNDGDEEGESSDSTQSQKRKRLWGLVVCHNTTPRFVPFPLRYACEFLAQVFAIHVNKELELESQILEKNILRTQTLLCDMLMRVAPLGIVSQSPNIMDLVKCDGAALLYKNKIHRLGMTPSDFQLHDIVSWLSEYHTDSTGLSTDSLYDAGFPGALALGDVVCGMAAVRISDKGWLFWYRSHTAAEVRWGGAKHEPGEKDDGRKMHPRSSFKAFLEVVKTRSVPWKDYEMDAIHSLQLILRNASKDADAMDSNTNI

SEQ ID NO: 7 - protein, 584 aa, Artificial Sequence
  Synthetic Peptide
MGFESSSSAASNMKPQPQKSNTAQYSVDAALFADFAQSIYTGKSFNYSKSVISPPNHVPDEHITAYLSNIQRGGLVQPFGCLIAVEEPSFRILGLSDNSSDFLGLLSLPSTSHSGEFDKVKGLIGIDARTLFTPSSGASLSKAASFTEISLLNPVLVHSRTTQKPFYAILHRIDAGIVMDLEPAKSGDPALTLAGAVQSQKLAVRAISRLQSLPGGDIGALCDTVVEDVQRLTGYDRVMVYQFHEDDHGEVVSEIRRSDLEPYLGLHYPATDIPQAARFLFKQNRVRMICDCNATPVKVVQSEELKRPLCLVNSTLRAPHGCHTQYMANMGSVASLALAIVVKGKDSSKLWGLVVGHHCSPRYVPFPLRYACEFLMQAFGLQLQMELQLASQLAEKKAMRTQTLLCDMLLRDTVSAIVTQSPGIMDLVKCDGAALYYKGKCWLVGVTPNESQVKDLVNWLVENHGDDSTGLTTDSLVDAGYPGAISLGDAVCGVAAAEFSSKDYLLWFRSNTASAIKWGGAKHHPKDKDDAGRMHPRSSFTAFLEVAKSRSLPWEISEIDAIHSLRLIMRESFTSSRPVLSGNG

SEQ ID NO: 8 - protein, 586 aa, Artificial Sequence
  Synthetic Peptide
MSSNTSRSCSTRSRQNSRVSSQVLVDAKLHGNFEESERLFDYSASINLNMPSSSCEIPSSAVSTYLQKIQRGMLIQPFGCLIVVDEKNLKVIAFSENTQEMLGLIPHTVPSMEQREALTIGTDVKSLFLSPGCSALEKAVDFGEISILNPITLHCRSSSKPFYAILHRIEEGLVIDLEPVSPDEVPVTAAGALRSYKLAAKSISRLQALPSGNMLLLCDALVKEVSELTGYDRVMVYKFHEDGHGEVIAECCREDMEPYLGLHYSATDIPQASRFLFMRNKVRMICDCSAVPVKVVQDKSLSQPISLSGSTLRAPHGCHAQYMSNMGSVASLVMSVTINGSDSDEMNRDLQTGRHLWGLVVCHHASPRFVPFPLRYACEFLTQVFGVQINKEAESAVLLKEKRILQTQSVLCDMLFRNAPIGIVTQSPNIMDLVKCDGAALYYRDNLWSLGVTPTETQIRDLIDWVLKSHGGNTGFTTESLMESGYPDASVLGESICGMAAVYISEKDFLFWFRSSTAKQIKWGGARHDPNDRDGKRMHPRSSFKAFMEIVRWKSVPWDDMEMDAINSLQLIIKGSLQEEHSKTVV

SEQ ID NO: 9 - protein, 598 aa, Artificial Sequence
  Synthetic Peptide
MSSSRSNNRATCSRSSSARSKHSARVVAQTPMDAQLHAEFEGSQRHFDYSSSVGAANRSGATTSNVSAYLQNMQRGRFVQPFGCLLAVHPETFALLAYSENAAEMLDLTPHAVPTIDQREALAVGTDVRTLFRSHSFVALQKAATFGDVNLLNPILVHARTSGKPFYAIMHRIDVGLVIDLEPVNPVDLPVTATGAIKSYKLAARAIARLQSLPSGNLSLLCDVLVREVSELTGYDRVMAYKFHEDEHGEVIAECKRSDLEPYLGLHYPATDIPQASRFLFMKNKVRMICDCSATPVKIIQDDSLTQPISICGSTLRAPHGCHAQYMASMGSVASLVMSVTINEDEDDDGDTGSDQQPKGRKLWGLMVCHHTSPRFVPFPLRYACEFLLQVFGIQINKEVELAAQAKERHILRTQTLLCDMLLRDAPVGIFTQSPNVMDLVKCDGAALYYQNQLWVLGSTPSEAEIKNIVAWLQEYHDGSTGLSTDSLVEAGYPGAAALGDVVCGMAAIKISSKDFIFWFRSHTAKEIKWGGAKHEPIDADDNGRKMHPRSSFKAFLEVVKWRSVPWEDVEMDAIHSLQLILRGSLQDEDANKNNNAK

SEQ ID NO: 10 - protein, 596 aa, Artificial Sequence
  Synthetic Peptide
MSLPSNNRRTCSRSSSARSKHSARVVAQTPVDAQLHAEFEGSQRHFDYSSSVGAANRPSASTSTVSTYLQNMQRGRYIQPFGCLLAVHPDTFALLAYSENAPEMLDLTPHAVPTIDQRDALGIGVDVRTLFRSQSSVALHKAAAFGEVNLLNPILVHARTSGKPFYAILHRIDVGLVIDLEPVNPADVPVTAAGALKSYKLAAKAISRLQSLPSGNLSLLCDVLVREVSELTGYDRVMAYKFYEDEHGEVISECRRSDLEPYLGLHYPATDIPQASRFLFMKNKVRMICDCCATPVKVIQDDSLAQPLSLCGSTLRASHGCHAQYMANMGSVASLAMSVTINEDEEEDGDTGSDQQPKGRKLWGLVVCHHTSPRFVPFPLRYACEFLLQVFGIQLNKEVELAAQAKERHILRTQTLLCDMLLRDAPVGIFTRSPNVMDLVKCDGAALYYQNQLLVLGSTPSESEIKSIATWLQDNHDGSTGLSTDSLVEAGYPGAVALREVVCGMAAIKISSKDFIFWFRSHTTKEIKWGGAKHEPVDADDDGRRMHPRSSFKAFLEVVKWRSVPWEDVEMDAIHSLQLILRGSLPDEDANRNNVR

SEQ ID NO: 11 - protein, 596 aa, Artificial Sequence
  Synthetic Peptide
MSSPSNNRGTCSRSSSARSKHSARVVAQTPVDAQLHADFEGSQRHFDYSSSVGAANRPSASTSTVSTYLQNMQRGRYIQPFGCLLAVHPDTFALLAYSENAPEMLDLTPHAVPTIDQRDALTIGADVRTLFRSQSSVALHKAATFGEVNLLNPILVHARTSGKPFYAILHRIDVGLVIDLEPFNPADVPVTAAGALKSYKLAAKAISRLQSLPSGNLSLLCDVLVREVSELTGYDRVMAYKFHEDEHGEVISECRRSDLEPYLGLHYPATDIPQASRFLFMKNKMRMICDFSATPVLIIQDGSLAQPVSLCGSTLRASHGCHAQYMANMGSVASLVMSVTINDDEEEDGDTDSDQQPKGRKLWGLVVCHHTSPRFVPFPLRYACEFLLQVFGIQLSKEVELAAQAKERHILRTQTLLCDMLLRDALVGIFTQSPNVMDLVKCDGAALYYQNQVLVLGSTPSESEIKSIATWLQENHDGSTGLSTDSLVEAGYPGAAALREVVCGMVAIKISSKNFIFWFRSHTTKEIKWSGAKHEPFDADDNGRKMHPRSSFKAFLEVVKWRSVPWEDVEMDAIHSLQLILRDSLQGEDANRNNIR

SEQ ID NO: 12 - protein, 592 aa, Artificial Sequence
  Synthetic Peptide
MSTPKKTYSSTSSAKSKAHSVRVAQTTADAALQAVFEKSGDSGDSFDYSKSVSKSTAESLPSGAVTAYLQRMQRGGLTQSFGCMIAVEGTGFRVIAYSENAPEILDLVPQAVPSVGEMDTLRIGTDVRTLFTASSVASLEKAAEAQEMSLLNPITVNCRRSGKQLYAIAHRIDIGIVIDFEAVKTDDHLVSAAGALQSHKLAAKAITRLQALPGGDIGLLCDTVVEEVRELTGYDRVMAYRFHEDEHGEVVAEIRRADLEPYLGLHYPGTDIPQASRFLFMKNKVRIIADCSAPPVKVIQDPTLRQPVSLAGSTLRSPHGCHAQYMGNMGSIASLVMAVIINDNEEDSHGSVQRGRKLWGLVVCHHTSPRTVPFPLRSACGFLMQVFGLQLNMEVELAAQLREKHILRTQTLLCDMLLRDAPIGIVSQIPNIMDLVKCDGAALYYGKRFWLLGTTPTESQIKDIAEWLLEYHKDSTGLSTDSLADANYPAAHLLGDAVCGMAAAKITAKDFLFWFRSHTAKEIKWGGAKHDPGEKDDGRKMHPRSSFKAFLEVVKRRSLPWEDVEMDAIHSLQLILRGSFQDIDDSDTKTMI

SEQ ID NO: 13 - protein, 591 aa, Artificial Sequence
  Synthetic Peptide
MSAPKKTYSSTSSAKSKAHSVRVAQTTADAALHAVFEKSGVSGDNFDYSKSVSKSTAGSLHTGAVTAYLQRMQRGGLTQSFGCMVAVEETGFRVIAYSENAPEFLDLMPQAVPNIGEINTLGIGTDVRTLFTPSSAASLEKAAETQEISLLNPITVYCRSKKPLYAIAHRIDIGIVIDFEAVNMNDVTISADGALQSHKLAAKAITRLQALPGGDIGLLCDTVVEEVRELTGYDRVMAYKFHEDEHGEVVAEIRRTDLEPYLGLHYPATDIPQASRFLFMKNRVRMIGDCSAPPVKIVQDPNLRQPVSLAGSTLRSPHGCHAQYMGNMGSISSIVMAVIINDNEDDSRGSVQRGRKLWGLVVCHHTSPRTVPFPLRSACEFLMQVFGLQLNMEVELAAQLREKHILRTQTLLCDMLLRDAPIGIVSQVPNIMDLVKCDGAALYYGKRFWLLGTTPTESQIKDIAEWLLEYHKDSTGLSTDSLADANYPAAHLLGDAVCGMAAAKITSKDFLFWFRSHTAKEIKWGGAKHDPGEKDDNRKMHPRSSFKAFLEVVKRRSLPWEDVEMDAIHSLQLILRGSFQDIDDSDTKTMI

SEQ ID NO: 14 - protein, 598 aa, Artificial Sequence
  Synthetic Peptide
MSTTKLTYSSGSSAKSKHSVRVAQTTADAKLHAVYEESGESGDSFDYSKSINATKSTGETIPAQAVTAYLQRMQRGGLVQPFGCMLAVEEGSFRVIAFSDNAGEMLDLMPQSVPSLGSGQQDVLTIGTDARTLFTAAASALEKAAGAVDLSMLNPIWVQSKTSAKPFYAIVHRIDVGLVMDLEPVKASDTRVGSAAGALQSHKLAAKAISRLQSLPGGDIGLLCDTVVEEVRDVTGYDLVMAYKFHEDEHGEVVAEIRRSDLEPYLGLHYPATDIPQASRFLFMKNRVRMICDCSAPPVKITQDKELRQPISLAGSTLRAPHGCHAQYMGNMGSVASLVMAMIINDNDEPSGGGGGGGQHKGRRLWGLVVCHHTSPRSVPFLRSACEFLMQVFGLQLNMEAAVAAHVREKHILRTQTLLCDMLLRDAPIGIVSQSPNIMDLVKCDGAALYYGKRFWLLGITPSEAQIKDIAEWLLEHHKDSTGLSTDSLADAGYPGAASLGDEVCGMAAAKITAKDFLFWFRSHTAKEVKWGGAKHDPDDKDDGRKMHPRSSFKAFLEVVKRRSLPWEDVEMDAIHSLQLILRGSFQDIDDSDTKTMI

SEQ ID NO: 15 - protein, 595 aa, Artificial Sequence
  Synthetic Peptide
MSTPKLAYSSGSSVKSKHSVRVAQTTADAKLQAVYEESGDSGDSFDYSKSVHASKSTGENVSAQAVTAYLQRMQRGGLMQTFGCMLCVEESNFRVIAFSENAPEMLDLMPQAVPSVGQQEVLGIGTDARTLFTPSSAAALEKCAGAVDVTMLNPISVHCRSSGKPFYAILHRIDVGLVIDFEPVRSNDAIVSSAGVLQSHKLAAKAISRLQALPGGDIGLLCDIVVQEVRELSGYDRVMAYKFHEDEHGEVLAEIRRSDLEPYLGLHYPATDIPQASRFLFMKNKVRMIGDCFASPVKVIQDKDLRQPISLAGSTLRAPHGCHAQYMGNMNSIASLVMAVIVNDPDEDPNARGGQQRGRKLWGLVVCHHTSPRTVSFPLRSACEFLMQVFGLQLNMEVELAAQLREKHILRTQTLLCDMLLRDAPIGIVSQSPNIMDLVKCDGAALYYGKRFWLLGITPNDAQIKEIADWLLEHHQDSTGLSTDSLADAGYPGAAQLGDAVCGMAAAKITSKDFLFWFRSHTAKEIKWGGAKHDPDEKDDGRKMHPRSSFKAFLVVVKRRSLPWEDIEMDAIHSLQLILRGSFQDIDDSDTKTMI

SEQ ID NO: 16 - protein, 595 aa, Artificial Sequence
  Synthetic Peptide
MSTTKLAYSSGSSVKSKHSVRVAQTTADAKLQAVYEESGDSGDSFDYSKSVHASKSTGENVPALAVTAYLQRMQRGGLVQTFGCMLCVDESSFRVIAYSENAPEMLDLMPQAVPSVGQQEVLGIGTDARTLFTPSSAAALEKCAGAVDVTMLNPISVHCRSSGKPFYAILHRIDVGLVIDFEPVRPNDAVVSSAGALQSHKLAAKAISRLQALPGGDIGLLCDTVVEEVRQLSGYDRVMAYKFHEDEHGEVLAEIRRSDLEPYLGLHYPATDIPQASRFLFMKNRVRMIGDCYAPPVKVVQDKDLRQPISLAGSTLRAPHGCHAQYMGNMNSIASLVMAVIVNDPDEDPNSRGGQQRGRKLWGLVVCHHTSPRTVSFPLRSACEFLMQVFGLQLNMEVELAAQLREKHILRTQTLLCDMLLRDAPIGIVSQSPNIMDLVKCDGAALYYGKRFWLLGITPNEVQIKEIADWLLEHHQDSTGLSTDSLADAGYPGAAQLGDAVCGMAAAKITPRDFLFWFRSHTAKEIKWGGAKHDPDEKDDGRKMHPRSSFKAFLEVVKRRSLPWEDIEMDAIHSLQLILRGSFQDIDDSDTKTMI

SEQ ID NO: 17 - protein, 592 aa, Artificial Sequence
  Synthetic Peptide
MASAKLAYSTGSGIKSKHSVRVQQTTADAKLQAAYEESNDSGDSFDYSKSVGQAAKSTVQQVPAQAVTAYLQRMQRGGLTQTFGCMLAVEENTFRVIAYSENALDMLDLMPQAVPSVGQQDVLGIGTDSRSLFTPSSAAALERATQQSDLSMVNPVSVHSRSSGKPFYAILHRIDVGIVMDFEPVRPNDVVVSSAGTIHSHKLAAKAISRLQSLPGGDIGLLCDTVVEEVRELTGYDRVMAYKFHEDEHGEVLAEIRRSDLEPYLGLHYPATDIPQASRFLFMKNRFRMIVDCYAPPVKVIQDKDLRQPLTLAGSTLRAPHGCHAQYMGNMGSIASVTMAVVVNDQEDDGGSQKARRLWGLVVCHHTSARMISFPLRSACEFLMQVFGLQLNMEVELAAQLREKHILRTQTLLCDMLLRDAPIGIVSQSPNIMDLVKCDGAALYYGNRFWLLGITPSEQQIKEIADWLLECHKDSTGLSTDSLADAGYPGANLLGDAVCGMAAARITPKDFLFWFRSHTAKEIKWGGAKHDADDRDDGRKMTPRSSFNAFLEVVKRRSVPWEDIEMDAIHSLQLILRGSFQDIDDSDTKTMI

SEQ ID NO: 18 - protein, 593 aa, Artificial Sequence
  Synthetic Peptide
MSSTKLSYSSGTSVKSKHSVRVQQTTADAKLQAVYEESNDSGDSFDYSKSVGQATKSTVQQVPAQAVTAYLQRMQRGGLTQNFGCMLAVEESTFRVIAYSENAPEMLDLMPQAVPSVGLKEVLGIGTDARLLFTPSSASTLERAAATSDLTMVNPISVHSRNSGKPFYAIVHRIDVGIVIDFEPVRSNDVVISTAGALHSHKLAAKAVSRLQSLPGGDIGLLCDAVVEEVRELTGYDRVMAYKFHDDEHGEVLAEIRRSDLESYLGLHYPSTDIPQASRFLFMKNRVRMIADCCAPPVKVIQDKDLRQPITLAGSTLRAPHGCHAQYMGNMGSVASITLAVIVHDQEEDFGVQQKGRRLWGLVVCHHTSPRTISFPLRSACEFLMQVFGLQLNMEVELQAQMKEKHILRTQTLLCDMLLRDAPVGIVSQSPNIMDLVKCDGAALYYENQFWLLGTTPSEEQIEEIAAWLLEHHKDSTGLSTDSLADAGYPGANLLGDAVCGMAAARISSKDFLLWFRSHSAKEIKWGGAKHDAEDRDDSRKMTPRSSFNAFLEVVKRRSVPWEDIEMDAIHSLQLILRGSFQDVDGSGGKTMI

SEQ ID NO: 19 - protein, 593 aa, Artificial Sequence
  Synthetic Peptide
MTSTKLSYSLGTTVKSKHSVRVQQTTADAKLQAVYEESNDSGDAFNYSKSVGQAAKATVQQVPAQAVSSYLQRMQRGGLTQTFGCMLAVEESTFRVIAYSENAPEMLDLVPHAVPSVGQQDVLGIGADARSLFTPSSASALERAASTSDLSMVNPISVHSRSSGKPFYAIVHRIDVGVVIDFEPVRPNDVIISTAGALHSHKLAAKAVARLQSLPGGDIGLLCDAVVEEVRELTGYDRVMAYKFHDDEHGEVLAEIRRSDLESYLGLHYPSTDIPQASRFLFMKNRVRMIADCCAPPVKVIQDKDLRQPITLAGSTLRAPHGCHAQYMGNMGSVASITLAVIVNDQEEDFGVQQKGRRLWGLVVCHHTSARTISFPLRSACEFLMQVFGLQLNMEVELQAQVKEKHILRTQTLLCDMLLRDAPIGIVSQSPNIMDLVKCDGAALYYGKRFWLLGTTPTEQQITEIADWLLEYHKDSTGLSTDSLADAGYPGANLLGDAVCGMAAARITPKDFLFWFRSHTAKEIKWGGAKHDADEKDDGRKMAPRSSFNAFLEVVKRRSVPWEDIEMDAIHSLQLILRGSFQDIDDSDGKTMI

SEQ ID NO: 20 - protein, 639 aa, Artificial Sequence
  Synthetic Peptide
MASGSRATPTRSPSSARPAAPRHQHHHSQSSGGSTSRAGGGGGGGGGGGGGAAAAESVSKAVAQYTLDARLHAVFEQSGASGRSFDYTQSLRASPTPSSEQQIAAYLSRIQRGGHIQPFGCTLAVADDSSFRLLAYSENTADLLDLSPHHSVPSLDSSAVPPPVSLGADARLLFAPSSAVLLERAFAAREISLLNPLWIHSRVSSKPFYAILHRIDVGVVIDLEPARTEDPALSIAGAVQSQKLAVRAISRLQALPGGDVKLLCDTVVEYVRELTGYDRVMVYRFHEDEHGEVVAESRRNNLEPYIGLHYPATDIPQASRFLFRQNRVRMIADCHAAPVRVIQDPALTQPLCLVGSTLRSPHGCHAQYMANMGSIASLVMAVIISSGGDDDHNISRGSIPSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTEVQIKDIIEWLTMCHGDSTGLSTDSLADAGYPGAAALGDAVSGMAVAYITPSDYLFWFRSHTAKEIKWGGAKHHPEDKDDGQRMHPRSSFKAFLEVVKSRSLPWENAEMDAIHSLQLILRDSFRDSAEGTSNSKA

SEQ ID NO: 21 - protein, 629 aa, Artificial Sequence
  Synthetic Peptide
MASGSRATPTRSPSSARPEAPRHAHHHHHSQSSGGSTSRAGGGAAATESVSKAVAQYTLDARLHAVFEQSGASGRSFDYSQSLRAPPTPSSEQQIAAYLSRIQRGGHIQPFGCTLAVADDSSFRLLAFSENSPDLLDLSPHHSVPSLDSSAPPHVSLGADARLLFSPSSAVLLERAFAAREISLLNPIWIHSRVSSKPFYAILHRIDVGVVIDLEPARTEDPALSIAGAVQSQKLAVRAISRLQALPGGDVKLLCDTVVEHVRELTGYDRVMVYRFHEDEHGEVVAESRRDNLEPYLGLHYPATDIPQASRFLFRQNRVRMIADCHATPVRVIQDPGLSQPLCLVGSTLRAPHGCHAQYMANMGSIASLVMAVIISSGGDDEQTGRGGISSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTESQIKDIIEWLTVFHGDSTGLSTDSLADAGYLGAAALGEAVCGMAVAYITPSDYLFWFRSHTAKEIKWGGAKHHPEDKDDGQRMHPRSSFKAFLEVVKSRSLPWENAEMDAIHSLQLILRDSFRDAAEGTNNSKA

SEQ ID NO: 22 - protein, 631 aa, Artificial Sequence
  Synthetic Peptide
MASDSRPPKRSPSARRVAPRHAHHHHSQSSGGSTSRAGAGGGGGGAAATESVSKAVAQYNLDARLHAVFEQSGASGRSFDYSQSLRAPPTPSSEQQIAAYLSRIQRGGHIQPLGCTLAVADDSSFRLLAFSENAADLLDLSPHHSVPSLDSVALPPVSLGADARLYFSPSSAVLLERAFAAREISLLNPLWIHSRASSKPFYAILHRIDVGVVIDLEPARTEDPALSIAGAVQSQKLAVRAISRLQALPGGDVKLLCDTVVEHVRELTGYDRVMVYKFHEDEHGEVVAESRRDNLEPYLGLHYPATDIPQASRFLFQQNRVRMIADCHAIPVRVIQDPGLSQQLCLVGSTLRAPHGCHAQYMANMGSIASLVMAVIISSGGDDERTGRGAISSSMKLWGLVVCHHTSPRCIPFPLRYACEFLMQAFGLQLNMELQLAHQLSEKHILRTQTLLCDMLLRDSPAGIITQSPSVMDLVKCDGAALYYRGKYYPLGVTPTESQIKDIIEWLTVCHGDSTGLSTDSLADAGYLGAVALGDAVCGMAVAYITPSDYLFWFRSHTAKEIKWGGAKHHPEDKDDGQRMHPRSSFKAFLEVVKSRSLSWENAEMDAIHSLQLILRDSFRDAAEGTSNSKA

SEQ ID NO: 23 - protein, 605 aa, Artificial Sequence
  Synthetic Peptide
MASGSRTKHSHQSGQGQVQAQSSGTSNVNYKDSISKAIAQYTADARLHAVFEQSGESGKSFDYSQSIKTTTQSVVPEQQITAYLTKIQRGGHIQPFGCMIAVDEASFRVIAYSENACEMLSLTPQSVPSLERPEILTVGTDVRTLFTPSSSVLLERAFGAREITLLNPIWIHSKNSGKPFYAILHRVDVGIVIDLEPARTEDPALSIAGAVQSQKLAVRAISHLQSLPGGDVKLLCDTVVESVRELTGYDRVMVYKFHEDEHGEVVAESKIPDLEPYIGLHYPATDIPQASRFLFKQNRVRMIVDCHATPVRVVQDESLMQPLCLVGSTLRAPHGCHAQYMANMGSIASLTLAVIINGNDEEAVGGRSSMRLWGLVVGHHTSARCIPFPLRYACEFLMQAFGLQLNMELQLASQLSEKHVLRTQTLLCDMLLRDSPTGIVIQSPSIMDLVKCDGAALYCQGKYYPLGVTPTEAQIKDIVEWLLTYHGDSTGLSTDSLADAGYPGAALLGDAVCGMAVAYITSKDFLFWFRSHTAKEIKWGGAKHHPEDKDDGQRMHPRSSFKAFLEVVKSRSLPWENAEMDAIHSLLILRDSFKDAEASNSKAVV

SEQ ID NO: 24 - protein, 608 aa, Artificial Sequence
  Synthetic Peptide
MASQSQRQSNQRQHQNQAAQSSGTSNMRQHHHATESVSKAIAQYTVDAQLHAVFEQSGGSGKSFDYSQSVRTTSQSVPEEQITAYLSKIQRGGHIQPFGCMIAVDEGSFRVIAYSENAKEMLGLTPQSVPSLDKQEILSDGTDVRTLFRPSSSAMLEKAFGAREIILLNPIWIHSKNSGKPFYAILHRIDVGIVIDLEPARTEDPALSIAGAVQSQKLAVRSISQLQSLPGGDIKLLCDTVVESVRELTGYDRVMVYKFHEDEHGEVVAENKRADLEPYIGLHYPSTDIPQASRFLFKQNRVRMIVDCHATPVRVIQDEALMQPLCLVGSTLRAPHGCHAQYMANMGSIASLAMAVIINGNEEEAIGGRNSTRLWGLVVCHHTSARCIPFPLRYACEFLMQAFGLQLNMELQLASQLSEKHVLRTQTLLCDMLLRDSPTGIVTQSPSIMDLVKCDGAALYYQGQYYPLGVTPTEAQIKDIVEWLLALHGDSTGLSTDSLADAGYPGAASLGNAVCGMAVAYITKRDFLFWFRSHTAKEIKWGGAKHHPEDKDDGQRMHPRSSFKAFLEVVKSRSLLWENAEMDAIHSLQLILRDSFRDVEATNSKAVV

SEQ ID NO: 25 - protein, 606 aa, Artificial Sequence
  Synthetic Peptide
MASQSQRQSNQPVHNQAQSSGTSNMRQHHHATESVSKAIAQYTVDAQLHAVFEQSGGTGRSFDYSKSVRTTNQSVPEQQITAYLSKIQRGGHIQPFGCMIAADEQSFRVIAYSENAKDMLGLTPQSVPSLEKQEILFVGADVRILFRPSSAVLLEKAFGAREITLLNPIWIHSKNSGKPFYAILHRIDVGIVIDLEPARTEDPALSIAGAVQSQKLAVRAISQLQSLPGGDIKLLCDTVVDSVRELTGYDRVMVYKFHEDEHGEVVAENKRVDLEPYIGLHYPSTDIPQASRFLFKQNRVRMIVDCHAIPVRVIQDEALMQPLCLVGSTLRAPHGCHAQYMENMGSIASLAMAVIIYGNDEEAIGGRNSMRLWGLVVCHHTSARCIPFPLRYACEFLMQAFGLQLNMELQLASQLLEKHVLRTQTLLCDMLLRDSPTGIVTQSPSIMDLVKCDGAALYYQGQYYPLGVTPTETQIKDIVEWLLTLHGDPTGLSTDSLADAGYPGAAFLGDAVCGMAVAYIAERDFLFWFRSHTAKEVKWGGAKHHPEDKDDGQRMHPRSSFKAFLEVVKSRSLPWENAEMDAIHSLQLILRDSFRDAEATNSKAVV

SEQ ID NO: 26 - protein, 634 aa, Artificial Sequence
  Synthetic Peptide
MVSGGGSKTSGGEAASSGHRRSRHTSAAEQAQSSANKALRSQNQQPQNHGGGTESTNKAIQQYTVDARLHAVFEQSGESGKSFDYSQSLKTAPYDSSVPEQQITAYLSRIQRGGYTQPFGCLIAVEESTFTIIGYSENAREMLGLMSQSVPSIEDKSEVLTIGTDLRSLFKSSSYLLLERAFVAREITLLNPIWIHSNNTGKPFYAILHRVDVGILIDLEPARTEDPALSIAGAVQSQKLAVRAISHLQSLPSGDIKLLCDTVVESVRDLTGYDRVMVYKFHEDEHGEVVAESKRNDLEPYIGLHYPATDIPQASRFLFKQNRVRMIVDCYASPVRVVQDDRLTQFICLVGSTLRAPHGCHAQYMTNMGSIASLAMAVIINGNEEDGNGVNTGGRNSMRLWGLVVCHHTSARCIPFPLRYACEFLMQAFGLQLNMELQLALQVSEKRVLRMQTLLCDMLLRDSPAGIVTQRPSIMDLVKCNGAAFLYQGKYYPLGVTPTDSQINDIVEWLVANHSDSTGLSTDSLGDAGYPRAAALGDAVCGMAVACITKRDFLFWFRSHTEKEIKWGGAKHHPEDKDDGQRMNPRSSFQTFLEVVKSRCQPWETAEMDAIHSLQLILRDSFKESEAMDSKAAA

SEQ ID NO: 27 - protein, 20 aa, Artificial Sequence
  Synthetic Peptide
MGSSHHHHHHSSENLYFQGH

SEQ ID NO: 28 - protein, 8 aa, Artificial Sequence
  Synthetic Peptide
SLHHHHHH

SEQ ID NO: 29 - protein, 8 aa, Artificial Sequence
  Synthetic Peptide
KLHHHHHH

SEQ ID NO: 30 - protein, 11 aa, Artificial Sequence
  Synthetic Peptide
GGGDYKDDDDK

SEQ ID NO: 31 - protein, 10 aa, Artificial Sequence
  Synthetic Peptide
GGDYKDDDDK

SEQ ID NO: 32 - DNA, 3996 bp, Artificial Sequence
  Synthetic Oligonucleotide
cttcaatttattttattggtttctccacttatctccgatctcaattctccccattttcttcttcctcaagttcaaaattcttgagaatttagctctaccagaattcgtctccgataactagtggatgatgattcaccctaaatccttccttgtctcaaggtaattctgagaaatttctcaaattcaaaatcaaacggcatggtttccggagtcgggggtagtggcggtggccgtggcggtggccgtggcggagaagaagaaccgtcgtcaagtcacactcctaataaccgaagaggaggagaacaagctcaatcgtcgggaacgaaatctctcagaccaagaagcaacactgaatcaatgagcaaagcaattcaacagtacaccgtcgacgcaagactccacgccgttttcgaacaatccggcgaatcagggaaatcattcgactactcacaatcactcaaaacgacgacgtacggttcctctgtacctgagcaacagatcacagcttatctctctcgaatccagcgaggtggttacattcagcctttcggatgtatgatcgccgtcgatgaatccagtttccggatcatcggttacagtgaaaacgccagagaaatgttagggattatgcctcaatctgttcctactcttgagaaacctgagattctagctatgggaactgatgtgagatctttgttcacttcttcgagctcgattctactcgagcgtgctttcgttgctcgagagattaccttgttaaatccggtttggatccattccaagaatactggtaaaccgttttacgccattcttcataggattgatgttggtgttgttattgatttagagccagctagaactgaagatcctgcgctttctattgctggtgctgttcaatcgcagaaactcgcggttcgtgcgatttctcagttacaggctcttcctggtggagatattaagcttttgtgtgacactgtcgtggaaagtgtgagggacttgactggttatgatcgtgttatggtttataagtttcatgaagatgagcatggagaagttgtagctgagagtaaacgagatgatttagagccttatattggactgcattatcctgctactgatattcctcaagcgtcaaggttcttgtttaagcagaaccgtgtccgaatgatagtagattgcaatgccacacctgttcttgtggtccaggacgataggctaactcagtctatgtgcttggttggttctactcttagggctcctcatggttgtcactctcagtatatggctaacatgggatctattgcgtctttagcaatggcggttataatcaatggaaatgaagatgatgggagcaatgtagctagtggaagaagctcgatgaggctttggggtttggttgtttgccatcacacttcttctcgctgcataccgtttccgctaaggtatgcttgtgagtttttgatgcaggctttcggtttacagttaaacatggaattgcagttagctttgcaaatgtcagagaaacgcgttttgagaacgcagacactgttatgtgatatgcttctgcgtgactcgcctgctggaattgttacacagagtcccagtatcatggacttagtgaaatgtgacggtgcagcatttctttaccacgggaagtattacccgttgggtgttgctcctagtgaagttcagataaaagatgttgtggagtggttgcttgcgaatcatgcggattcaaccggattaagcactgatagtttaggcgatgcggggtatcccggtgcagctgcgttaggggatgctgtgtgcggtatggcagttgcatatatcacaaaaagagactttcttttttggtttcgatctcacactgcgaaagaaatcaaatggggaggcgctaagcatcatccggaggataaagatgatgggcaacgaatgcatcctcgttcgtcctttcaggcttttcttgaagttgttaagagccggagtcagccatgggaaactgcggaaatggatgcgattcactcgctccagcttattctgagagactcttttaaagaatctgaggcggctatgaactctaaagttgtggatggtgtggttcagccatgtagggatatggcgggggaacaggggattgatgagttaggtgcagttgcaagagagatggttaggctcattgagactgcaactgttcctatattcgctgtggatgccggaggctgcatcaatggatggaacgctaagattgcagagttgacaggtctctcagttgaagaagctatggggaagtctctggtttctgatttaatatacaaagagaatgaagcaactgtcaataagcttctttctcgtgctttgagaggggacgaggaaaagaatgtggaggttaagctgaaaactttcagccccgaactacaagggaaagcagtttttgtggttgtgaatgcttgttccagcaaggactacttgaacaacattgtcggcgtttgttttgttggacaagacgttactagtcagaaaatcgtaatggataagttcatcaacatacaaggagattacaaggctattgtacatagcccaaaccctctaatcccgccaatttttgctgctgacgagaacacgtgctgcctggaatggaacatggcgatggaaaagcttacgggttggtctcgcagtgaagtgattgggaaaatgattgtcggggaagtgtttgggagctgttgcatgctaaagggtcctgatgctttaaccaagttcatgattgtattgcataatgcgattggtggccaagatacggataagttccctttcccattctttgaccgcaatgggaagtttgttcaggctctattgactgcaaacaagcgggttagcctcgagggaaaggttattggggctttctgtttcttgcaaatcccgagccctgagctgcagcaagctttagcagtccaacggaggcaggacacagagtgtttcacgaaggcaaaagagttggcttatatttgtcaggtgataaagaatcctttgagcggtatgcgtttcgcaaactcattgttggaggccacagacttgaacgaggaccagaagcagttacttgaaacaagtgtttcttgcgagaaacagatctcaaggatcgtcggggacatggatcttgaaagcattgaagacggttcatttgtgctaaagagggaagagtttttccttggaagtgtcataaacgcgattgtaagtcaagcgatgttcttattaagggacagaggtcttcagctgatccgtgacattcccgaagagatcaaatcaatagaggtttttggagaccagataaggattcaacagctcctggctgagtttctgctgagtataatccggtatgcaccatctcaagagtgggtggagatccatttaagccaactttcaaagcaaatggctgatggattcgccgccatccgcacagaattcagaatggcgtgtccaggtgaaggtctgcctccagagctagtccgagacatgttccatagcagcaggtggacaagccctgaaggtttaggtctaagcgtatgtcgaaagattttaaagctaatgaacggtgaggttcaatacatccgagaatcagaacggtcctatttcctcatcattctggaactccctgtacctcgaaagcgaccattgtcaactgctagtggaagtggtgacatgatgctgatgatgccatattagtcacacttcagttggtatgagagtttgtatcattgtatgagtgtttgtgtgtctaacgacgtcggaggaggatagaaagttttttttttgtttccggtgagattagtagagaagagggagattatttgcgttcagctcagctcgccggaaaaaaaacgtaacagtagttgtagagaatttcaagacttttgtttgtgctgtgtaaattgacaactccgagagaaacaaaacaatgagataagaagagagcatattaatcgatgaccaatccttttaatt

SEQ ID NO: 33 - DNA, 1179 bp, Artificial Sequence
  Synthetic Oligonucleotide
gagccggctagtacggaggaccccgctctctccatcgccggtgcagtccagtcccagaaactggcggtccgcgccatctcccgcctccaggcgctacccggcggggacgtcaagcttctctgcgacacagtcgtggagcatgttcgcgagctcacgggttatgaccgtgtcatggtgtacaggttccatgaagacgagcacggggaagttgtcgccgagagccggcgcgacaaccttgagccttacctcggattgcattatcccgccacagatatcccccaggcgtcgcgcttcctgttccggcagaaccgcgtgcgaatgattgccgattgccatgccaccccggtgagagttattcaagatcctgggctgtcgcagcctctgtgtttggtaggctccacgctacgcgctccacacgggtgtcatgcacagtacatggcgaacatggggtcaattgcgtcgcttgttatggcagtcatcattagcagtggcggtgacgatgagcaaacaggtcggggtggcatctcgtcggcaatgaagttgtgggggttagtggtgtgccaccatacatcaccacggtgtatcccttttccattgaggtatgcttgcgagtttctcatgcaggcatttgggttgcagctcaacatggagttgcancttgcgcaccagctgtcagagaagcacattctgcgaactcagacgctattgtgtgacatgctactacgagattcaccaactggcatcgtcacgcagagccccagcatcatggaccttgtgaagtgcgacggggctgcactgtattatcatgggaaatactatccattgggtgtcactcccactgagtctcagattaaggatatcatcgagtggttgacggtgtttcatggggactcaacaggcctcagcacagatagcctggctgatgcaggctaccttggtgctgctgcactaggggaggctgtgtgtggaatggcggtggcttatattacaccgagtgattacttgttttggtttcggtcacacacagctaaagagatcaaatggggtggcgcaaagcatcaccctgaggataaggatgatggtcagaggatgcacccacggtcgtcattcaaggcatttcttgaagtggttaaaagcagaagcctgccatgggagaatgccgaaatggacgccatccat

SEQ ID NO: 34 - DNA, 3513 bp, Artificial Sequence
  Synthetic Oligonucleotide
atgggctcgggtagccgcgccacgcccacgcgctccccctcctccgcgcggcccgcggcgccgcggcaccagcaccaccactcgcagtcctcgggcgggagcacgtcccgcgcgggagggggtggcgggggcgggggagggggagggggcggcgcggccgccgcggagtcggtgtccaaggccgtggcgcagtacaccctggacgcgcgcctccacgccgtgttcgagcagtcgggcgcgtcgggccgcagcttcgactacacgcagtcgctgcgtgcgtcccccaccccgtcctccgagcagcagatcgccgcctacctctcccgcatccagcgcggcgggcacatacagcccttcggctgcacgctcgccgtcgccgacgactcctccttccgcctcctcgcctactccgagaacaccgccgacctgctcgacctgtcgccccaccactccgtcccctcgctcgactcctccgcggtgcctccccccgtctcgctcggcgcagacgcgcgcctccttttcgctccctcgtccgccgtcctcctcgagcgcgccttcgccgcgcgcgagatctcgctgctcaacccgctctggatccactccagggtctcctctaaacccttctacgccatcctccaccgcatcgatgtcggcgtcgtcatcgacctcgagcccgcccgcaccgaggatcctgcactctccatcgctggcgcagtccagtctcagaagctcgcggtccgtgccatctcccgcctccaggcgcttcccggcggtgacgtcaagctcctttgcgacaccgttgttgagcatgttagagagctcacaggttatgaccgcgttatggtgtacaggttccatgaggatgagcatggagaagtcgttgccgagagccggcgcagtaaccttgagccctacatcgggttgcattatcctgctacagatatcccacaggcatcacgcttcctgttccggcagaaccgtgtgcggatgattgctgattgccatgctgcgccggtgagggtcatccaggatcctgcactaacacagccgctgtgcttggttgggtccacgctgcgttcgccgcatggttgccatgcgcagtatatggcgaacatgggttccattgcatctcttgttatggcagtgatcattagtagtggtggggatgatgatcataacattgcacggggcagcatcccgtcggcgatgaagttgtgggggttggtagtatgccaccacacatctccacggtgcatccctttcccactacggtatgcatgcgagttcctcatgcaagcctttgggttgcagctcaacatggagttgcagcttgcacaccaactgtcagagaaacacattctgcggacgcagacactgctgtgtgatatgctactccgggattcaccaactggcattgtcacacaaagccccagcatcatggaccttgtgaagtgtgatggtgctgctctgtattaccatgggaagtactaccctcttggtgtcactcccacagaagttcagattaaggacatcatcgagtggttgactatgtgccatggagactccacagggctcagcacagatagccttgctgatgcaggctactctggtgctgctgcactaggagatgcagtgagcggaatggcggtagcatatatcacgccaagtgattatttgttttggttccggtcacacacagctaaggagataaagtggggtggtgcaaagcatcatccagaggataaggatgatggacaacgaatgcatccacgatcatcgttcaaggcatttcttgaagttgtgaagagtaggagcttaccatgggagaatgcagagatggatgcaatacattccttgcagctcatattgcgggactctttcagagattctgcagagggcacaagtaactcaaaagccatagtgaatggccaggttcagcttggggagctagaattacggggaatagatgagcttagctcggtagcgagggagatggttcggttgatcgagacagcaacagtacccatctttgcagtagatactgatggatgtataaatggttggaatgcaaaggttgctgagctgacaggcctctctgttgaggaagcaatgggcaaatcattggtaaatgatctcatcttcaaggaatctgaggaaacagtaaacaagctactctcacgagctttaagaggtgatgaagacaaaaatgtagagataaagttgaagacattcgggccagaacaatctaaaggaccaatattcgttattgtgaatgcttgttctagcagggattacactaaaaatattgttggtgtttgttttgttggccaagatgtcacaggacaaaaggtggtcatggataaatttatcaacatacaaggggattacaaggctatcgtacacaaccctaatcctctcatacccccaatatttgcttcagatgagaatacttgttgtttggagtggaacacagcaatggaaaaactcacaggatggtcaagaggggaagttgttggtaagcttctggtcggtgaggtctttggtaattgttgtcgactcaagggcccagatgcattaacgaaattcatgattgtcctacacaacgctataggaggacaggattgtgaaaagttccccttttcattttttgacaagaatgggaaatacgtgcaggccttattgactgcaaacacgaggagcagaatggatggtgaggccataggagccttctgtttcttgcagattgcaagtcctgaattacagcaagcctttgagattcagagacaccatgaaaagaagtgttatgcaaggatgaaggaattggcttacatttaccaggaaataaagaatcctctcaacggtatccgatttacaaactcgttattggagatgactgatctaaaggatgaccagaggcagtttcttgaaaccagcactgcttgtgagaaacagatgtccaaaattgttaaggatgctagcctccaaagtattgaggatggctctttggtgcttgagaaaggtgaattttcactaggtagtgttatgaatgctgttgtcagccaagtgatgatacagttgagagaaagagatttacaacttattcgagatatccctgatgaaattaaagaagcctcagcatatggtgaccaatatagaattcaacaagttttatgtgactttttgctaagcatggtgaggtttgctccagctgaaaatggctgggtggagatacaggtcagaccaaatataaaacaaaattctgatggaacagacacaatgcttttcctcttcaggtttgcctgtcctggcgaaggccttcccccagagattgttcaagacatgtttagtaactcccgctggacaacccaagagggtattggcctaagcatatgcaggaagatcctaaaattgatgggtggcgaggtccaatatataagggagtcggagcggagtttcttccatatcgtacttgagctgccccagcctcagcaagcagcaagtagggggacaagc

SEQ ID NO: 35 - DNA, 7285 bp, Artificial Sequence
  Synthetic Oligonucleotide
atggcgtcgggcagccgcgccacgcccacgcgctccccctcctccgcgcgacccgaggcgccgcgtcacgcgcaccaccaccaccaccaccactcgcagtcgtcgggcgggagcacgtcccgcgcgggcgggggaggtggaggaggaggaggtggcgggggcaccgcggccacggctacggccacggccacggagtcggtctccaaggccgtggcgcagtacaccctagacgcgcggctccacgcggtgttcgagcaatcgggcgcgtcgggccgcagcttcgactactcccagtcgctgcgcgcgccgcccacgccgtcctccgagcagcagatcgccgcctacctctcccgcatccagcgcggcggccacatccagcccttcggctgcacgctcgccgtcgccgacgactcctccttccgcctcctcgccttctccgagaacgccgccgacctgctcgacctgtcgccgcaccactccgttccctcgctcgattccgcggcgccgccccccgtttccctgggtgccgacgcgcgcctcctcttctccccctcgtccgcggtcctcctggagcgcgccttcgccgcgcgcgagatctcgctgctcaacccgctatggatccactccagggtctcttccaagccgttctacgccatcctccaccgcatcgacgtcggcgtcgtcatcgacctcgagcccgcccgcaccgaggaccccgctctctccatcgccggcgcagtccagtcccagaaactcgcggtccgtgccatctcccgcctccaggcgctacctggcggggacatcaagctcctctgcgacacagtcgtggagcatgttcgcgagctcacgggttacgaccgtgtcatggtgtacaggttccatgaagacgagcatggggaagttgtcgccgagagccggcgcgataaccttgagccttacctcggattgcattatcccgccacagatatcccccaggcatcgcgcttcctgttccggcagaaccgcgtgcggatgattgctgattgccatgccaccccggtgagagtcatacaagatcctgggatgtcgcagccactgtgtttggtaggctccacgcttcgtgctccacacgggtgccatgcgcagtacatggcgaacatggggtcaattgcatcacttgttatggcagtcatcattagcagtggtggtgatgacgagcaaacaggtcggggaggcatctcctcggcaatgaagttgtgggggttagtggtgtgtcaccatacgtcaccacggtgtatcccttttccattgaggtatgcttgcgagtttctcatgcaggcatttgggctgcagctcaacatggaattgcagcttgcgcatcagctgtcagagaagcacattttgcgaactcagacgctattgtgtgacatgctattgcgagattcaccaactggcatcgtcacgcagagccccagcatcatggaccttgtgaagtgtgatggggctgcactgtattatcatgggaagtactatccattgggtgtcactcccactgagtctcagattaaggatatcattgagtggttgacggtgtgtcatggggactcaacagggctcagcacagacagccttgctgatgcaggctaccttggtgctgctgcattaggggatgctgtgtgtggaatggcggtggcttatattacaccgagtgattacttgttttggtttcggtcacacacagctaaagagatcaaatggggtggcgcaaagcatcaccctgaggataaggatgatggtcagaggatgcacccacggtcatcattcaaggcatttcttgaagtggttaaaagcagaagcctaccatgggagaatgcagaaatggacgcgatacattccttgcaactcatattgcgtgactccttcagagatgctgcagagggcactagcaactcaaaagccattgtcaatggacaagctcagcttggggagctagaattgcgggggataaatgagcttagctctgtaccaagagagatggttcggttgatagagacagcaacagtacccatatttgcagtagatactgatggatgcataaatggttggaatgcgaaaattgctgagttgacaggcctttcagttgaggaggcaatgggcaaatctctggtaaacgatcttatcttcaaggaatctgaggagatagtcgaaaagctactctcacgagctttaagaggtattccatttttgtttcttacatggatgctattactttttatattatgttatatgagaattgcggagtgctgttgaggaaaagattatctgttcttggttgtatctcttaaggtatatataaaagaaaggtatatgccataacagaagtgtatggacatgatgtatacatgtttattagaccaaatattgaaacgtgtggtatatatactcttggaaaatggatatttgaaggagctaagcctagctcaattggtcgatggtgtggatgtacgcccaaacccagtcccccggttcaagtttgttgaggcgaatttttgcatgtttcttcttaattaaaaagctcccctatgttggtcaaggttttttaatagatatactcagtttcttagatgcacaaatatatagaaaatggggaccaactagagactaccatcaaatttactcaaccatttccacagtatttttagaatccggtgcttataactagcagataggaatgctcatatctgaaaagtaagcatatgaaacttataccaaaggttgtgttagctagtcacaggagtacattgtttctcgcccaggctgtttgaactattttaatctacttcccaatgcatgtagctaagctatgttagctttgatggatgtgcgattggagagtgctaatttgtgaagttgcttcccatacaggtgaggaagacaaaaatgtggagataaagttgaagacatttgggtcagagcaatctaacggagcaatatttgttattgtcaatgcttgttccagtagagattacacacaaaatattgttggtgtctgttttgttggacaagatgtcacaggacaaaaggtggtcatggataaatttatcaacatacaaggggactataaagctattgtacacaatcctaatcctctgatacccccaatttttgcatcagatgagaacacttcttgttcagaatggaacacagccatggaaaaacttacaggatggtcgagaggtgaagttgttggtaaatttcttattggagaggtgtttggaagtttttgtcgactcaagggcccagatgcattgacaaagttcatggttgtcattcacaatgctataggagggcaggattatgagaagttccctttttcatttttcgacaagaatggaaagtatgtgcaggccttattgaccgccaacacaaggagcaaaatggatggtaaatccattggcgccttttgttttttgcagattgcaagcgctgaaatacagcaagcctttgagattcagagacaacaagaaaagaagtgttatgcaaggatgaaagaattggcctatatttgccaggagataaagaatcctcttagtggcatccgatttaccaactctctgttgcaaatgactgatttaaatgatgatcagaggcagttccttgaaacttgctctgcttgtgagaaacagatgtccaagattgttaaggacgccactctccaaagtattgaggacgggtcagtactaagtttaccccttctgctcacccattctctaatatttgcattgaatatttacatttgatcattcatattcgtttaacttgagcctggagtagccttgatctaaattttaattggcgctaatcttgactggtagaggtacaaaatcggagtaggaattagagaagttcatcagtcattagactatcacagtttgttctactgtctttgcagttctttaatattttcccttgcacctaagttattgccaacattagacgtagatccttgttttttgtaccaccctttaagacacctttgttgaagggcgggcctggtgcaggcggtagagtactaccgactgtgaccagatcaccagaaggtcctgggttcgagccgcagcctccttgcatttgcacattgcaagggtaaggcttgccgctgatacccttccccagaccccgcacagtgcgggatgctctcagcactgggtacgcccttcctttaagacacctttgttattatcccttatggcacttctgtggactgcttctctctcagccaatatctgaaactgtgtaagatcagcaacatttgccaacatatattgttgcaaatgtgaaggcagtccagattgctgtacttcaaaagatgcattggtgttttagaagcttcagcatatcctactgtagtactgtgcttaaaaatactcctatgcgtttggaacctctttttatttgaaacaaagtaccataccatcatactgtacagtaactggcatgcgtctggctgattatgtacattgttatggttgtaattatctaatggttgacgatatggctactaaaccagaaccaagccagatttgatggttttcacagtttccagtggaacttggtgaatctggtgtctttttctccatttttttcaaggatagcatgaatactacacaagtacatacaataactactgtatatactgaatatgtcaaattatgttaaaaaagtcaacttctgtaaaaacaaaaaaagtgcatagtgggggttttctgcttaccaatgttagcatacttattactaccaggattgtgaaactttgtgcttcaaatgctatatcatggatttttgttagaattaaaggaatagccttgtgaactgataaccttaccacattttagaggggtgtcaccaaagttcaagcagctttgtctaacatgcctctgcctgtagttttgtttgaggagcttcataaattatttggagatgctgtatttcatttgcttgcacggaaactcaaatcccttctggttgatttttatacacttggtcaggtccatctgtcttgctattggtaatgaattcccagaaagtgacttaaggcatgttcaagatattagttgcttctcatttggcaaagtctaaacacaaacttaccaagatatcacttggaatagctacttacacaatcctatattattactgtcacattatttgcttgacctcattatctaaagaaggtatttgcttgacctaattttcatttaggttcattggtccagtgtttagactgtagacagtagtgttttctcacttctttgtaacctaagacataagtttgaatgctgctgttatttgccttatttccacgattgattaactgaaaatttgttgaattgtttctctttatgtaacagctctttggtacttgagaaaagtgagttttcttttggagacgttatgaatgctgttgtcagccaagcaatgttattgttgagggagagggatttacaacttattcgggatatccctgatgaaatcaaggatgcatcagcatatggtgatcaatttagaattcaacaagttttggctgacttcttgctaagcatggtgcgatctgctccgtccgagaatggctgggtagaaatacaagtcagaccaaatgtaaaacagaattctgacggaacagatacagagcttttcatcttcaggttagctgcttgctctttgattataattcgtcacaaaatgtttttttttgtttcttcaaggaatatttgttgatgaattttggttgaagacaagtaacatgaattacttcaacctttggctggttggccgcatatgtggtgtgatacctttcaaatatacaacaacaaagccttttagtcccaagcaagttggggtaggctaatacctttcaaatatacatccgtgagaatttcgagagaattatcctgtttgcgtcatgctatatttttcgttttcttctgtcgacttaatttctttaacttaggccttgtttagttacacccaaaaaccaaaaacttttcaagattccccatcacatcgaatcttgtggcacatgcatggagcattaaacatagataaaaataataactaattacataatttgcttgtaatttgcgagacaaatcttttgagcctagttaatccataattggacattaattaccaaatacaaacaaaatgctacagtaccccaaaaatttcaccccaacaactaaacaaggccttagtgtactgtttggctgtgggatttgatggtaacataaacagtatcagaaggcaacagtaatggaaaatagagaaacgaatactgacattatttggttgagctcacacattggatttatactgcagcttatacaaagtcgttacagctcctgattgaactgggttcaagtgtgtaacctacctccgtcccactaatatatattttaaattgctctcatatgatgtaccaactaatagatatcaccttttttttttgttacagaacgatatttagattagcataggcaaatacaaatctggatctatttgattagtactttaggatgtctgttacataccaaaactgctgcctgccatagagcattggtgagagaaactgagaattgacgaccttacctattgaagttcaaaactgtcctgtaatggtgccatggactgtaaatctgtaatcatctggatcttttagatgaattttactattttagtgtgctggcctaacttgagcaaaagtgattttcttttctgcaactcatattatttcaacaacctacattttttttaaaaaaaagagagaggaaaagacctctgcttttaagaaagcaacatcattcggtacaatactgggaacaaccagtttaacaagaaaagttcaaaagtcgacgttaaagcctattacagacaacaacctacatttgttgcattgtgcattaggtgattctggagaatcagcttccaaatgctaacaacggaaaactcaatgcctctattgggacttgagagtaccaataaatggaactaagtgatttccttttgatttttgtatgtccgtaggtttgcctgccctggtgagggccttcccgctgacattgtccaggatatgttcagcaattcccagtggtcaacccaagaaggcgtaggactaagcacatgcaggaagatcctcaaattgatgggcggtgaggtccaatacatcagggagtcagagcggagtttcttcctcatcgtcctcgagctgccccagcctcgtccagcagctgatagagaaatcagttga

SEQ ID NO: 36 - DNA, 3725 bp, Artificial Sequence
  Synthetic Oligonucleotide
atggcttcagcaagcggagcggcgaattcctccgttccgccgccgcaaatccacacctcacgaacaaagctgagccaccacagcagcaacaacaacaacaacatcgactccatgagcaaggccatcgcgcagtacacggaggacgcgcggctccacgccgtcttcgagcagtccggcgagtccgggaggtccttcaactactccgaatcaatccgcatcgcatcggaatccgtccccgagcagcagataacggcttaccttgtcaaaatccagcgcggcggcttcatccagcccttcggctccatgatcgccgtcgacgagccctccttccgcatcctcggttactccgacaacgcccgcgacatgctcggcattactccgcagtccgtcccttcgctcgacgacaagaacgacgccgccttcgctctcggcaccgatgtccgagccctcttcactcactccagcgccttactcctcgaaaaggccttctccgcacgcgaaattagcctcatgaaccctatctggatccactccagaacctccgggaagcctttctatggaatcctccaccgaattgacgtcggaattgtcatcgatttggagcctgcgcgtacggaggatcctgccctctctatcgctggagctgtccagtcgcagaagctcgcggttcgcgcgatttcgcagcttcaatctctccccggcggtgatgttaagcttctctgtgacactgttgtggaaagtgttagggaattgacgggttatgatagggttatggtttataagtttcatgaggatgagcatggagaggttgtttctgagagtaagaggcctgatttggagccttacattggattgcattatcctgctactgatattcctcaggcttctaggtttttgtttaagcaaaatagagttaggatgattgtggattgtcatgcttctgctgtgagggtggtgcaggatgaggctcttgtgcagcctttgtgtttggttgggtccacccttagggcacctcacggttgtcatgctcagtatatggctaacatgggctcgattgcgtctttggtgatggcagttattatcaatgggaatgacgaggaaggcgttggtggtcgcagttcgatgaggctgtgggggcttgttgtctgccaccatacctctgccaggtgtattccttttcccttgaggtatgcttgtgagtttctgatgcaggcgtttgggctgcagttgaacatggagcttcagttggccgcgcagtcgttggagaagcgggttttgaggacacagactctgttgtgtgatatgcttcttagggactcgcctactggcattgttactcagagtcctagtataatggacttggtgaagtgtgatggggctgccctttatttccaagggaactattatccgttgggtgtgactccaactgaagctcagattagggatattattgagtggttgttggccttccatggagattcgaccggtttgagtactgatagtctgggtgatgctggatatcccggggctgcctcgcttggggatgcagtttgtgggatggcggttgcttatattacagagaaggattttcttttctggttcaggtcgcacacggccaaagagatcaaatggggtggtgcaaagcatcatcctgaggacaaggatgatgggcagagaatgcatccccgttcttccttcaaggcgtttttagaagtggtgaaaagccgtagcttgccgtgggagaatgcggaaatggatgcaattcactctttgcagcttattctgcgtgactcgtttaaagatgctgagcatagaaattctaaggctgttgtggatccccatgtgtcagaacaagagttgcaaggggtggatgaactaagttctgtggccagagagatggttagattgatagaaacagccactgctccaatatttgctgttgatgtcgatggccacgtaaatgggtggaatgcaaaggtttcagaattaacaggactcccagttgaggaggctatggggaagtccttggttcacgatcttgtgtttaaggagtctgaagaaactatgaacaagcttctttctcgtgctttaaaaggtgaagaagataagaatgttgagataaaaatgaggacgtttggcccagaacatcaaaataaggcagtgtttttagtggtgaatgcttgctccagcaaggattttacaaataatgtagttggagtgtgctttgttggtcaggatgttactggtcaaaaaattgtaatggacaaattcatcaacatacaaggtgactacaaggctattgtacatagcccaaatcctttgatccctcccatttttgcatcggacgataacacatgttgcttagagtggaacactgcaatggaaaagcttactggttggggccgtgtggatgtcattggaaaaatgttggtgggagaggtttttggtagttgctgtcagttgaagggttcagattcaataacaaagttcatgattgtcttacacaatgcacttggtggacaagatacagataaattccctttctcatttcttgatcggcacggaaagtatgtacaaactttcctgactgcaaataagagggttaacatggagggtcagatcataggagctttttgctttttgcaaatcatgagtccggaacttcagcaggctcttaaggcacagagacaacaagaaaagaattcctttggtaggatgaaagagttagcttatatttgtcaaggagttaagaatcctttgagtggcatacgctttacaaactctcttttggaggctacaagcttgaccaatgagcaaaagcagtttcttgagactagtgttgcttgtgagaagcaaatgttaaagataatacgcgacgttgatcttgaaagcatcgaggatgggtccctggagcttgaaaagggggaattcttgcttggaaatgtcataaatgcagttgttagccaagtaatgttactgttaagagaaagaaatttacagttgattcgtgatattcctgaagaaatcaagacattggcagtttatggtgatcaattgaggattcaacaagtgttgtctgatttcttgttgaatatagtgcgctatgcaccatctccagatggctgggtagagattcatgtacgtccaagaataaaacaaatctcagatgggctcactcttctccatgctgaatttagaatggtatgtcctggtgaaggtcttcctcctgaattgattcaagacatgttcaataacagtcggtgggggactcaagaaggtttagggctgagcatgagcaggaagattctaaagctaatgaacggcgaagtgcagtatatcagggaggccgaacggtgctacttctatgttcttcttgaactacctgtgacacggagaagctctaaaaagtgttaaatatgtattctgtagctgtaagattaagtttaaagccctggcattgcccctcatgctgaccgtagatagtttgttgttatagattatgctctcacctaacaaggcaacttaaggggcaagcatttgaacaagctgatttggctagctatctcagtcacatgtaattctaccaaattggactgttgttttgcaccctgacaaaacttgttcggtacagcaagagtggctagttaatatgtggctaaatgtaaccttaaatcttactgataaagatatggcagagtagatgaaatgaagtttcgccaatatgc

SEQ ID NO: 37 - DNA, 3857 bp, Artificial Sequence
  Synthetic Oligonucleotide
atggcttcagcaagcggagcggagaattcctccgtcccgccgtcgccgttgccgcctccgccgccgccgcaaatccacacctcgcggacgaagctgagccaccaccaccacaacaacaacaacaacaacaacaacaacatcgactccacgagcaaggccatcgcgcagtacacggaggacgcgcggctccacgccgtctttgagcagtccggcgagtccgggaggtcctttgactactcccaatcaatccgcgtcacatcggaatccgtcccggagcagcagataacggcttaccttctcaaaattcagcgcggcggcttcatccagcccttcggctccatgatcgccgtcgacgagccctccttccgcatccttgcctactccgacaacgcccgtgacatgctcggcattactccacagtccgtcccttcgctcgacgacaagaacgacgccgccttcgcgctcggaaccgatatccgaaccctcttcactcactccagcgccgttctcctcgaaaaggccttctccgcgcgcgaaattagcctcatgaaccctatctggattcactccagaacctccgggaagcctttctatggaatcctccaccgaattgacgtcggaattgtcatcgatttggagcctgcgcggacggaggatcctgccctctccatcgccggagctgtccagtcgcagaagctcgcggttcgcgcgatttcgcagcttcaatctctccccggtggcgatgttaagcttctttgtgatactgttgttgagagtgtcagggaattgacagggtatgatagggttatggtttataggtttcatgaggatgagcatggggaggttgttgctgagactaagaggcctgatttggagccttacattggattgcattatcccgctactgatattcctcaggcttctaggtttttgtttaagcagaatagggttaggatgattgtggattgtcatgcttctgctgtgagggtggtgcaggatgaggctcttgtgcagcctctgtgtttggttgggtccacgctcagggcgcctcacggttgccatgctcagtatatggctaacatgggctcgactgcgtcgttggtgatggctgttattatcaatgggaatgatgaggaaggtgttggtggccgcacttcgatgaggttgtgggggcttgttatttgccaccatacctctgctaggtgtattccttttcccttgaggtatgcttgtgagtttctgatgcaggcgtttgggctgcagttgaacatggagcttcagttggccgcacagtcgttggagaagcgggttttgaggacacagactctgttgtgtgatatgcttctcagggactctcctactggcattgtaactcagagtcctagtattatggacttggtgaagtgtgacggagctgctctttattaccaagggaactattatccgttgggtgtgactccaactgaggctcagataagggatattattgagtggttgttggcctttcatagagattcgactggtttgagtactgatagtctggctgatgctggctatcctggggctgcctcgcttggggatgcagtttgtgggatggcggttgcttatattacagagaaggattttcttttctggttcaggtcgcacacggcgaaagagatcaaatggggtggtgcaaagcatcatcctgaggacaaggatgatgggcagagaatgcatccccgttcttccttcaaggcatttttagaagtggtgaaaagccgtagcttgccgtgggagaatgcggaaatggatgcaattcactctttgcagcttattctgcgtgactcgtttaaagatgctgagcatagcaattctaaggctgttttggatccccgtatgtcggaactagagttgcaaggggtcgatgaactaagttctgtagccagagagatggttagattgatcgaaacagccactgctccaatatttgctgttgatgttgatggccgcataaatgggtggaatgcaaaggtttcagaattgacaggactcccagttgaggaggctatggggaagtccttggttcgcgatcttgtgtttaaggagtctgaagaaactgtggacaagcttctttctcgtgctttaaaaggtgaagaagataagaatgttgagataaaaatgaggacgtttggcccagaacatcaaaataaggcagtttttgtagtggtgaatgcttgctccagcaaggattatacaaataatgtagttggagtgtgctttgttggtcaggatgttactggtcaaaaaattgtgatggacaaattcatcaacatacaaggcgactacaaggctattgtacataatccaaatcctttgatccctcccatttttgcatcggatgataacacgtgttgcttagagtggaacactgcaatggaaaagcttactggttggagccgcgcggatgtcattggaaaaatgttggtgggagaggttttcggcagttgctgtcagttgaagggttcagattcaataacaaagttcatgattgtcttacacaatgcgcttggtggacatgatacagatagattccctttttcatttcttgatcggtatggcaagcatgtgcaagctttcctgactgcaaataagagggttaacatggatggtcagatcattggggcattttgctttttgcaaattgtgagtccggaacttcaacaggctctgaaggcacagagacaacaagagaagaattcatttgctaggatgaaagagttagcttatatttgtcaaggagttaagaatcctttgagtggcatacgctttacaaactctcttttggaggctacatgcttgtccaatgagcaaaaacagtttcttgagactagtgctgcttgtgagaagcaaatgttaaagataatacacgatgttgatattgaaagcattgaggatgggtccctggagcttgaaaagggggaattcttgcttggaaatgtcataaatgcagttgttagccaagtaatgctactgttaagagaaagaaatttacagttgattcgtgatattcctgaagaaatcaagacattggctgtttatggtgatcaattgaggattcaacaagtgttgtctgatttcttattgaatatagtgcgctatgcaccatctccagatggctgggtagagattcatgtacatccaagaataaaacaaatctcagatgggctcactcttctccatgctgaatttagaatggtatgtcctggtgaaggtcttcctcctgaattgattcaaaacatgttcaataacagtgggtgggggactcaagaaggtttagggctgagcatgagcaggaagattctaaagctaatgaacggcgaagtgcagtatatcagggaggcccaacggtgctacttctatgttcttcttgaactacctgtgacacggagaagctctaaaaagtgttaaatatgtattctgtagctgtaagattaagtttaaaggtgctacttctatgttcttcttgaactacctgtgacacggagaagctctaaaaagtgttaaatatgtattctgtagctgtaagattaagtttaaagccctggcattgcccctcatgctgaccgtagatagtttgttgttatagattatgctctcacctaacaaggcaacttaaggggcaagcatttgaacaagctgatttggctagctatctcagtcacatgtaattctaccaaattggactgttgttttgcaccctgacaaaacttgttcggtacagcaagagtggctagttaatatgtggctaaatgtaaccttaaatcttactgataaagatatggcagagtagatgaaatgaagtttcgccaatatgc

SEQ ID NO: 38 - DNA, 3722 bp, Artificial Sequence
  Synthetic Oligonucleotide
atgcttcagcaagcggagcggcgaattcctccgttccgccgccgcaaatccacacctcacgaacaaagctgagccaccacagcagcaacaacaacaacaacatcgactccatgagcaaggccatcgcgcagtacacggaggacgcgcggctccacgccgtcttcgagcagtccggcgagtccgggaggtccttcaactactccgaatcaatccgcatcgcatcggaatccgtccccgagcagcagataacggcttaccttgtcaaaatccagcgcggcggcttcatccagcccttcggctccatgatcgccgtcgacgagccctccttccgcatcctcggttactccgacaacgcccgcgacatgctcggcattactccgcagtccgtcccttcgctcgacgacaagaacgacgccgccttcgctctcggcaccgatgtccgagccctcttcactcactccagcgccttactcctcgaaaaggccttctccgcacgcgaaattagcctcatgaaccctatctggatccactccagaacctccgggaagcctttctatggaatcctccaccgaattgacgtcggaattgtcatcgatttggagcctgcgcgtacggaggatcctgccctctctatcgctggagctgtccagtcgcagaagctcgcggttcgcgcgatttcgcagcttcaatctctccccggcggtgatgttaagcttctctgtgacactgttgtggaaagtgttagggaattgacgggttatgatagggttatggtttataagtttcatgaggatgagcatggagaggttgtttctgagagtaagaggcctgatttggagccttacattggattgcattatcctgctactgatattcctcaggcttctaggtttttgtttaagcaaaatagagttaggatgattgtggattgtcatgcttctgctgtgagggtggtgcaggatgaggctcttgtgcagcctttgtgtttggttgggtccacccttagggcacctcacggttgtcatgctcagtatatggctaacatgggctcgattgcgtctttggtgatggcagttattatcaatgggaatgacgaggaaggcgttggtggtcgcagttcgatgaggctgtgggggcttgttgtctgccaccatacctctgccaggtgtattccttttcccttgaggtatgcttgtgagtttctgatgcaggcgtttgggctgcagttgaacatggagcttcagttggccgcgcagtcgttggagaagcgggttttgaggacacagactctgttgtgtgatatgcttcttagggactcgcctactggcattgttactcagagtcctagtataatggacttggtgaagtgtgatggggctgccctttatttccaagggaactattatccgttgggtgtgactccaactgaagctcagattagggatattattgagtggttgttggccttccatggagattcgaccggtttgagtactgatagtctgggtgatgctggatatcccggggctgcctcgcttggggatgcagtttgtgggatggcggttgcttatattacagagaaggattttcttttctggttcaggtcgcacacggccaaagagatcaaatggggtggtgcaaagcatcatcctgaggacaaggatgatgggcagagaatgcatccccgttcttccttcaaggcgtttttagaagtggtgaaaagccgtagcttgccgtgggagaatgcggaaatggatgcaattcactctttgcagcttattctgcgtgactcgtttaaagatgctgagcatagaaattctaaggctgtcgcggatccccgtgtgtcagaacaagagttgcaaggggtggatgaactaagttctgtggccagagagatggttagattgatagaaacagccactgctccaatatttgctgttgatgtcgatggccacgtaaatgggtggaatgcaaaggtttcagaattaacaggactcccagttgaggaggctatggggaagtccttggttcacgatcttgtgtttaaggagtctgaagaaactatgaacaagcttctttctcgtgctttaaaaggtgaagaagataagaatgttgagataaaaatgaggacgtttggcccagaacgtcaaaataaggcagtgtttttagtggtgaatgcttgctccagcaaggattttacaaataatgtagttggagtgtgctttgttggtcaggatgttactggtcaaaaaattgtaatggacaaattcatcaacatacaaggtgactacaaggctattgtacatagcccaaatcctttgatccctcccatttttgcatcggacgataacacatgttgcttagagtggaacactgcaatggaaaagcttactggttggggccgtgtggatgtcattggaaaaatgttggtgggagaggtttttggtagttgctgtcagttgaagggttcagattcaataacaaagttcatgattgtcttacacaatgcacttggtggacaagatacagataaattccctttctcatttcttgatcggcacggaaagtatgtacaaactttcctgactgcaaataagagggttaacatggagggtcagatcataggagctttttgctttttgcaaatcatgagtccggaacttcagcaggctcttaaggcacagagacaacaagaaaagaattcctttggtaggatgaaagagttagcttatatttgtcaaggagttaagaatcctttgagtggcatacgctttacaaactctcttttggaggctacaagcttgaccaatgagcaaaagcagtttcttgagactagtgttgcttgtgagaagcaaatgttaaagataatacgcgacgttgatcttgaaagcatcgaggatgggtccctggagcttgaaaagggggaattcttgcttggaaatgtcataaatgcagttgttagccaagtaatgttactgttaagagaaagaaatttacagttgattcgtgatattcctgaagaaatcaagacattggcagtttatggtgatcaattgaggattcaacaagtgttgtctgatttcttgttgaatatagtgcgctatgcaccatctccagatggctgggtagagattcatgtacgtccaagaataaaacaaatctcagatgggctcactcttctccatgctgaatttagaatggtatgtcctggtgaaggtcttcctcctgaattgattcaagacatgttcaataacagtcggtgggggactcaagaaggtttagggctgagcatgagcaggaagattctaaagctaatgaacggcgaagtgcagtatatcagggaggccgaacggtgctacttctatgttcttcttgaactacctgtgacacggagaagctctaaaaagtgttaaatatgtattctgtagctgtaagattaagtttaaagccctggcattgcccctcatgctgaccgtagatagtttgttgttatagattatgctctcacctaacaaggcaacttaaggggcaagcatttgaacaagctgatttggctagctatctcagtcacatgtaattctaccaaattggactgttgttttgcaccctgacaaaacttgttcggtacagcaagagtggctagttaatatgtggctaaatgtaaccttaaatcttactgataaagatatggcagagtagatgaaatgaagtttcgccaatat

SEQ ID NO: 39 - DNA, 3951 bp, Artificial Sequence
  Synthetic Oligonucleotide
atgcttcagcaagcggagcggagaattcctccgtcccgccgtcgccgttgccgcctccgccgccgccgcaaatccacacctcgcggacgaagctgagccaccaccaccacaacaacaacaacaacaacaacaacaacatcgactccacgagcaaggccatcgcgcagtacacggaggacgcgcggctccacgccgtctttgagcagtccggcgagtccgggaggtcctttgactacccccaatcaatccgcgtcacatcggaatccgtcccggagcagcagataacggcttaccttctcaaaattcagcgcggcggcttcatccagcccttcggctccatgatcgccgtcgacgagccctccttccgcatccttgcctactccgacaacgcccgtgacatgctcggcattactccacagtccgtcccttcgctcgacgacaagaacgacgccgccttcgcgctcggaaccgatatccgaaccctcttcactcactccagcgccgttctcctcgaaaaggccttctccgcgcgcgaaattagcctcatgaaccctatctggattcactccagaacctccgggaagcctttctatggaatcctccaccgaattgacgtcggaattgtcatcgatttggagcctgcgcggacggaggatcctgccctctccatcgccggagctgtccagtcgcagaagctcgcggttcgcgcgatttcgcagcttcaatctctccccggtggcgatgttaagcttctttgtgatactgttgttgagagtgtcagggaattgacagggtatgatagggttatggtttataggtttcatgaggatgagcatggggaggttgttgctgagactaagaggcctgatttggagccttacattggattgcattatcccgctactgatattcctcaggcttctaggtttttgtttaagcagaatagggttaggatgattgtggattgtcatgcttctgctgtgagggtggtgcaggatgaggctcttgtgcagcctctgtgtttggttgggtccacgctcagggcgcctcacggttgccatgctcagtatatggctaacatgggctcgactgcgtcgttggtgatggctgttattatcaatgggaatgatgaggaaggtgttggtggccgcacttcgatgaggttgtgggggcttgttatttgccaccatacctctgctaggtgtattccttttcccttgaggtatgcttgtgagtttctgatgcaggcgtttgggctgcagttgaacatggagcttcagttggccgcacagtcgttggagaagcgggttttgaggacacagactctgttgtgtgatatgcttctcagggactctcctactggcattgtaactcagagtcctagtattatggacttggtgaagtgtgacggagctgctctttattaccaagggaactattatccgttgggtgtgactccaactgaggctcagataagggatattattgagtggttgttggcctttcatagagattcgactggtttgagtactgatagtctggctgatgctggctatcctggggctgcctcgcttggggatgcagtttgtgggatggcggttgcttatattacagagaaggattttcttttctggttcaggtcgcacacggcgaaagagatcaaatggggtggtgcaaagcatcatcctgaggacaaggatgatgggcagagaatgcatccccgttcttccttcaaggcatttttagaagtggtgaaaagccgtagcttgccgtgggagagtgcggaaatggatgcaattcactctttgcagcttattctgcgtgactcgtttaaagatgctgagcatagcaattctaaggctgttttggatccccgtatgtcggaactagagttgcaaggggtcgatgaactaagttctgtagccagagagatggttagattgatcgaaacagccactgctccaatatttgctgttgatgttgatggccgcataaatgggtggaatgcaaaggtttcagaattgacaggactcccagttgaggaggctatggggaagtccttggttcgcgatcttgtgtttaaggagtctgaagaaactgtggacaagcttctttctcgtgctttaaaaggtgaagaagataagaatgttgagataaaaatgaggacgtttggcccagaacatcaaaataaggcagtttttgtagtggtgaatgcttgctccagcaaggattatacaaataatgtagttggagtgtgctttgttggtcaggatgttactggtcaaaaaattgtgatggacaaattcatcaacatacaaggcgactacaaggctattgtacataatccaaatcctttgatccctcccatttttgcatcggatgataacacgtgttgcttagagtggaacactgcaatggaaaagcttactggttggagccgcgcggatgtcattggaaaaatgttggtgggagaggttttcggcagttgctgtcagttgaagggttcagattcaataacaaagttcatgattgtcttacacaatgcgcttggtggacatgatacagatagattccctttttcatttcttgatcggtatggcaagcatgtgcaagctttcctgactgcaaataagagggttaacatggatggtcagatcattggggcattttgctttttgcaaattgtgagtccggaacttcaacaggctctgaaggcacagagacaacaagagaagaattcatttgctaggatgaaagagttagcttatatttgtcaaggagttaagaatcctttgagtggcatacgctttacaaactctcttttggaggctacatgcttgtccaatgagcaaaaacagtttcttgagactagtgctgcttgtgagaagcaaatgttaaagataatacacgatgttgatattgaaagcattgaggatggataagtcataagatagtcctgaatcctgatactgttagataccgttgactggttgatgctaaagaatgagaacatgagacttacttaatatctggtccctggagcttgaaaagggggaattcttgcttggaaatgtcataaatgcagttgttagccaagtaatgctactgttaagagaaagaaatttacagttgattcgtgatattcctgaagaaatcaagacattggctgtttatggtgatcaattgaggattcaacaagtgttgtctgatttcttattgaatatagtgcgctatgcaccatctccagatggctgggtagagattcatgtacatccaagaataaaacaaatctcagatgggctcactcttctccatgctgaatttagaatggtatgtcctggtgaaggtcttcctcctgaattgattcaaaacatgttcaataacagtgggtgggggactcaagaaggtttagggctgggcatgagcaggaagattctaaagctaatgaacggcgaagtgcagtatatcagggaggcccaacggtgctacttctatgttcttcttgaactacctgtgacacggagaagctctaaaaagtgttaaatatgtattctgtagctgtaagattaagtttaaaggtgctacttctatgttcttcttgaactacctgtgacacggagaagctctaaaaagtgttaaatatgtattctgtagctgtaagattaagtttaaagccctggcactgcccctcatgctgaccattagatagtttgttgtcatagattatgctctcacctaacaaggcaacttaagggacaagtgtttgaacaagctgatttggcaagctatcccagtcacatgtaattctaccaaattggactgttgttttgcaccttgacaaaacttgttcggtaaagcaagagtggccatttaatatgtagctaaatgtaaccttaaatcttactggtaaagttatggctgagtagatgaaatgaagtttcgccaatatgc

SEQ ID NO: 40 - DNA, 3393 bp, Artificial Sequence
  Synthetic Oligonucleotide
atggcttctggaagtagaacaaagcattcccatcataattcatctcaagctcaatcttcaggtacaagtaatgtaaattacaaagattcaataagcaaagctatagcacagtacacagctgatgctaggcttcatgctgtgtttgaacaatctggtgagtctggaaagttttttgattattcagagtctgttaaaactactacacaatctgtgcctgaaaggcaaatcactgcttatttgactaaaattcaaagaggaggtcatattcagccttttggttgtatgatagctgtagatgaggctagttttcgtgtaatagcttatagtgaaaatgcctttgaaatgcttagtttaactccacaatctgttccaagccttgagaagtgtgagatcctcactattggaactgatgttaggaccctttttaccccttctagctctgttttgctagaaagagcatttggggcacgtgagatcactttactcaacccaatttggattcattccaagaattctggaaagcccttttatgcaattttgcacagggttgatgttggtattgccattgatttggagcctgctagaactgaggaccctgctttatctattgctggagcagtgcagtcacagaaacttgcagtgagggctatttctcatttgcaatcacttcctggtggggacattaagcttttgtgtgatactgttgttgagagtgtcagggagttaaccgggtatgaccgggttatggtatataaatttcatgaggatgagcatggagaggtagtggctgagagtaaaagatcagatttagagccctatatcggtttgcattatcctgctactgatattcctcaagcttcacggtttttgtttaagcagaacagggtgagaatgattgtggactgtcatgctacccctgtgcgggttactcaggatgaatcactgatgcagcctttatgtctagttggttccacacttagagcacctcatggttgccacgcacagtacatggcaaatatggggtctattgcctcattaacactggcagttattatcaacggaaatgatgaggaagctgtgggtggcggtcgaaattcaatgaggctatggggcttggttgttggacaccacacttctgttcggtccattcctttccctcttaggtatgcatgtgaattccttatgcaggcctttggactccaattgaacatggagttgcaattggcgtcacagttgtctgagaaacatgttttaaggacacaaacactgttatgtgacatgctccttcgagactctccaccggggattgttacccaaagccccagtattatggaccttgtgaagtgcgatggtgctgctctatactaccaggggaagtactatccattaggtgtcacaccaactgaagctcagataaaggacattgtggagtggttattggcttaccatggagactcaacaggtttaagtactgacagtttggctgatgctgggtatcctggagcagcttcacttggtgatgcagtttgtggtatggctgtcgcttatatatcttctaaagatttcttgttttggtttcgctcccacacagcgaaagaaataaagtggggtggtgcaaagcatcatcctgaagacaaggatgatggactgagaatgcatccacgttcttccttcaaggcatttctggaagttgttaaaagtcggagctcaccatgggaaaatgccgaaatggatgcaatccactctttgcagctaattctgcgagattcatttaaggatgctgaggcaagtaattctaaggctattgtgcatgctcatcttggggaaatggagttgcaagggatagatgaactgagttctgttgccagagaaatggttagattgatcgaaactgcaacagctcccatatttgctgttgatgtcgaaggtcgcataaatgggtggaatgcaaaggtcgctgaattgacaggtttatcagttgaagaagcaatggggaagtccttggttcatgagcttgtgtacaaagaatcacaggagactgctgagaagcttctgtataatgctctaagaggcgaggaagataaaaatgtagaaataaagttgaggacatttggagctgaacaactggagaaagctgtttttgtggtggttaatgcttgcgctagcaaagattacacaaacaacattgttggtgtttgctttgttgggcaggatgttactggggaaaaagttgttatggacaagtttattaacatccaaggtgattacaaggccattgtgcacagccccaatcctctgatccctccaatatttgcatcagatgagaacacttgttgctccgagtggaacactgccatggaaaaactcactggttggtctagaggggagattgttggaaaaatgttagttggtgagatttttggaagttgttgtcggctcaagggcccagatgccatgacaaagttcatgatcgtgttgcataatgcaattggaggacaggatacagacaagtttccattttccttttttgaccgaaatgggaaatatgtgcaagctcttttgactgctaacaagagagtcaatatggagggcaatactattggggctttctgtttcatacagatagccagtcccgaactgcagcaagctctaagagttcaaaggcaacaggaaaagaagtgttattctcagatgaaagagctggcatacatttgtcaggaaataaaaagtcctcttaatggtatacgctttacaaattcattgttggaggccacaaatttgacagaaaatcagaagcagtatctagagacaagtgctgcttgtgagaggcagatgtctaagatcattagggatgttgatctggaaaacattgaggacggttcactgacccttgagaaagaagatttttttcttgggagtgtaatagatgctgttgttagccaagtgatgttattgctgagggaaaaaggcgtgcagttaatccgtgatataccagaggaaattaagacattaacagtacatggtgatcaagtgagaattcaacaggtcttggcagatttcttgttgaacatggtacggtatgcaccatcacctgatgggtgggtagaaatccaacttcgaccaagtatgatgccaatatctgatggagtaactggtgtgcatattgaactcaggattatatgccctggcgaagggcttcctcctgaattggttcaagatatgttccacagcagtcggtgggtaactcaggaaggcctaggactgagcacgtgcagaaaaatgttaaagcttatgaatggagaaatccagtatatcagagaatcagaaagatgctatttcctgattgtccttgacctgccaatgacccgcaaaggtccaaagagtgttggctag

SEQ ID NO: 41 - DNA, 3648 bp, Artificial Sequence
  Synthetic Oligonucleotide
agcaacaacaataacaacagaaatattaaaagagaatcgttatcaatgagaaaagccatagctcagtacacagaagacgcaagnctccatgctgtttttgaaaaatccggtgactctttcgattatgcccaatccattcgcgtcacggcggctactgaatcagttcctgaacagcaaatcactgcttacttagccaaaatccaacgcggtggtttcattcaacctttcggttcaatgatcgccgtcgacgaaacttcttttcgcgttcttgcttactctgaaaacgcacgtgacatgcttggtatcgcgcctcaatcggttccttctatggaagatgattcttcttcttcttcgtttttctctttaggcgttgatgttcgttctctttttagtgcttccagttctgtacttcttgagaaagctttttcagctcgggagattagtttaatgaatcctatttggatccactctcgttctactggtaagcctttttatggaattcttcaccgaattgatattggtgttgttattgatttggagcctgcgagatctgaggatccagcgctttcgattgccggtgctgttcagtctcagaagcttgcggttcgtgcgatttcgcagctccaggcgcttcctggtggtgatgtcaagcttctttgtgatgctgttgttgagagtgttagggaattgactggttatgatagggttatggtttataagtttcatgaggatgagcatggtgaggttgttgctgagagtaagagggttgatttagagccttatattggtttgcattatcctgctactgatattcctcaggcttctaggtttttgtttaagcagaatagggttaggatgattgtggattgtaatgcttctcctgttagggtttttcaggatgaggcgcttgttcagcctgtttgtttggttgggagtactcttcgggctcctcatggttgtcatgctcagtacatggcaaatatgggttccattgcttctttggctatggctgttattattaatgggaatgatgaagacggtggtgggattggtggtgctgcacgtggctcgatgaggctttggggtcttgttgtttgtcatcatacttctgctaggtgtattcctttccctcttaggtatgcttgtgagtttctaatgcaggcttttgggcttcagttgaatatggagcttcagttagccgtgcagtcgttggagaaaagggttttgaagacacagactctgttgtgtgatatgttacttagggattctcatacagggattgttactcagagtcctagtattatggatttggttaagtgtgatggggctgctttgtattatcaaggaaactaccaccctttgggtgttactccgaccgagtctcagataagggatatcatagattggttgttggcctttcatagtgattcgacgggtttgagtactgatagtttggctgatgctggttatcctggggctgcttctcttggggatgcagtttgtggaatggctgttgcgtatattactgaaaaagactttcttttctggttcagatctcatacggctaaagaaattaaatggggtggtgcaaagcatcacccggaggataaggatgacgggcagaaaatgcatcctcgttcttctttcaaggcctttttagaagtggtgaagatccgtagtatgcagtgggataatgcagaaatggatgcaattcactccttgcagcttatcctgcgagactcgtttaaggaagctgagaataacgattcaaaggctgtcgtgcatacccatatggcagaactagagttgcaaggggtggatgaactgagttctgtggctagagaaatggttaggttgatagaaacagccactgctcccatatttgctgttgatgtcgatggtcgcatcaatgggtggaatgcaaaggtttctgaattgacaggacttctggtagaggaggctatgggcaagtctttggttcatgatctcgtgtataaggagtctcgagaaactgtggacaagcttctttctcatgctttaaaaggtgaagaagataaaaatgttgagataaaaatgaagacttttggcccggggaatcaaaataaggcagtttttatagtggtgaatgcttgctccagcaaggattatacaaataatatagttggagtgtgctttgttggccaggatattactggtcaaaaagttgtaatggacaaattcattaacatacaaggtgactacaaggctattgtacatagtccaaatccattgatccctcccatttttgcatcggatgacaacacatgttgcttagagtggaacaatgctatggaaaagctcagcggctggagccgtgcagatgtcattggcaaattgttagtgggagaggtttttggtagtttctgtcagttgaagggttcggatgctatgacaaaattcatgattgttttgcacaatgcacttggtggacacgacacagacaaattcccattgtcatttcttgacagacatggaaagtatgtgcatactttcttgaccgcaaataagagggttaacatggatggtcagatcattggcgcattttgctttttacaaattgtgaaccctgaacttcaacaggctttgacagtccagagacaacaggatagtagttccttagctagaatgaaggagttagcttatatttgtcaagaagtaaagaatcccttgagtggcatacgctttacaaactctcttttggagtctacatgcctgactgatgagcaaaagcagcttcttgagactagtgttgcttgtgagaagcaaatgctgaagatagtacgggacattgctctagaaagcatcgaggatgggtccctggagcttgaaaagcaggaattcttgctcgagaatgtcataaatgcagttgttagccaagtaatgctattgctaagagatagaaagttacagttaattcgtgatattcctgaagaaatcaaggcattggctgtttatggtgatcagttgaggattcaacaagtcttggctgatttcttaatgaatgtggtgcgctatgcaccatctccagatggttgggtagagattcatgtatttccaagaataaaacaaatttcagaggggctcactcttctgcatgctgaatttaggatggtgtgtcctggtgaaggtcttccacctgaattgattcaagacatgttccataacagtcggtgggtgactcaagaaggcttagggctgagcatgagcaggaagattataaagttaatgaacggcgaagtccagtatgtaagggaggcagaacggtgctacttcttagttcttcttgaactacccgtgacacggagaagctctaaagctattaattaggcatttcgcaactgtaaaattcaagtccaagctctatcactcatgttgaccattagatagtttgttgtcatatactcagtttgtgcttagcttaactaaccggacaacttagggtacaagcatttgaacaagctgattcggccacttatccatcctagttacatgaaattgtactgtcaaattagattgttgttttgctccctaaccaaacttgttcagttgtacaacacgatgagctattaatttgtagctaaatgtaacctaaaaccttgctgataatgt

SEQ ID NO: 42 - DNA, 8517 bp, Artificial Sequence
  Synthetic Oligonucleotide
agcaagaaaatggtcacgtccatggcccaaaaggagcccatttgctacaaagatcatcggaccagttgcgttctcttcacacacgcagagagcttttttacaactgctgaaagggtatcggaacccaaacaagagcagtggcaccaccacagctgctactccgtcaatcatcgtgtccgcctcctccttgatctgttctgagacttacccatttatcagaattgctgaatcttcatacccatttctctttttgatctataattttggaaagctgagaaaggtgtggagagtctgcgaaatgagttcaggaaacagaggaacgcagtcgcaccaccaagctcagtcgtcggggacaagcaatttgagagtttaccacactgattcaatgagcaaagccattgcgcaatatacaatggatgctcgcctccacgccgtatacgaacagtccggcgagtccggtaagtcattcgactactcgcagtcggttagaaccacaacgcaatcggtccctgagcaacaaatcactgcgtatttatcgaaaattcaacggggtggccatatacagccctttgggtgtatgcttgcggtcgatgaggccacttttcgggtcattgctttcagcgaaaatgcccgagaaatgctcggtctcactccgcaatcggttccgagccttgagaagcccgagatcctcctagtaggtactgatgttcgcacgcttttcactccctcgagcgcagttctcctcgaaaaggcgtttcgggctcgggaaattacgttgttaaatcccgtgtggattcattccaagaattctggaaaacccttttacgcaattttgcatagaattgatgtgggaattgtaattgatttggagcctgcaaggactgaggaccctgctctgtccattgctggggcggtgcagtcgcagaagttggccgttcgagcaatttcccatcttcaatctcttcccggtggtgatattaaccttttgtgtgaaactgtggttgagaatgtgagggagcttactgggtatgatcgggtcatggtttacaaatttcacgaggatgaacatggtgaggtcgtggctgagagcaagaggtctgatttggagccttatattgggttacactatcctgccacggacattccacaggcttcaaggtttttgtttaggcagaatcgggttaggatgatcgttgattgccatgccacgcctgttctggtgattcaagatgaagggcttatgcagcctctatgcttagttggttcaacccttcgggctcctcatggctgccatgcacagtatatggccaacatgggttcaactgcctcattagcgatggctgtcatcatcaatggaagtgatgaggaagctattggtgggcgaaacttgatgaggctatggggcctggttgtttgtcatcacacatctgctaggtgcattccatttcctcttcgatatgcctgtgagttcctaatgcaggcatttggactccaattgaacatggaactgcagttagcatcgcaattgtctgagaaacatgttttaaggacacagactctcttgtgtgacatgctccttcgtgattcccctactggaattgttacccaaagtcctagtattatggatcttgtgaagtgtgatggagcagcactttattaccaggggaagtattatccaactggggtgaccccgactgaagcccagataaaggatattgcagagtggttgttggcaaaccatgcggattcaacaggtttaagcactgacagtttggctgatgctggctaccctggggcagcctcacttggtgatgcagtttgtggaatggctgttgcttatatcacttcaagagattttctattctggtttcggtcccacacagcaaaagagatcaaatggggtggtgcaaagcatcatccagaggacaaggacgatgggcagaggatgcatcctcgttcttcattcaaggcatttttagaagtggtcaagagtcggagtttgccatgggagaatgcggaaatggatgcaattcattctctgcagcttattctgcgtgactcttttaaggatgctactgatggaagcaattctaaggctgtaatgcatgctcagctcggggagctagagttgcaagggatggatgagttgagctctgttgcaagagaaatggttaggttgattgaaactgcaacagctcccatatttgcggtcgatgttgatggctgcataaatggttggaatgcaaaggttgcggagttgacggggctttctgttgaggaagctatggggaagtccttggttcatgatcttgtttacaaggaatctgaagaaactgttgacaagcttcttcatcatgctctacgaggtagtcctctatctcctaaaatcttttgttcctgcaaaacccacaagttcattaatgggggccttttatttcctttggaaataaagtagctatattttgttttccatttcatttgaaggcaaaaatccatcataaataattaaactttttggaaagacaaatgatttggtaggcaaaagtaggaatcagaaaagtaaaaataaaaaaaatctaaagaaaatttttgagatcctgaaaattaaattatgcatgatattgaatgcagtcattcaaatactatgaatacccaaaataactaatttgatatgctaataatgtacatgggaatgatttcattgtactggctctgaatggcttctaagtcaagagcattaattaaatgaaataaacctatgaaaaaaaaaaattcaaaagatgtagattgttaatgaagttctctgaattttataagggaaagccaagtaattgtggtttttttaatctaccaaaaaaataaaaaaagaacctgttacatgttaccttgatgtatgtgcatctaatttatgtgagagaaggatattttgatacatcactgtatgcctaccccaaaatttgtttcgactaaaagttggataaatttgcactgtactctccaaaacaaacttactagatgcttttttatgtggttgcttatgtcaactttgttatatcatgttttttccagtgcatctaggacactgatacatttccactgtccaacacaggtatgaggttgccaagggccaatcttaaaactaaatgaataacgggagtgttagtgttgaaaatgatgttttacatgggaaaaaaacaactataataagaaatccttgttccatagttgaaggagtgatagtttaattcctggtattagtcagctatgaaattgatatgtgttgaaaaataataactcagcgctggattttgaaagttaattggttttacttaattttaattgatgcaggtgaagaagataagaatgtagagataaaattgaggacatttgactcacaacagcataagaaggctgtttttgtggtcgttaatgcttgctccagtagggattacacaaataatatagttggagtttgctttgttggtcaggatgttactggtcagaaagtggtaatggacaaatttatccatatacaaggtgattacaaagctattgtacatagtcccaaccctttgattcctcctatatttgcttcagatgagaacacagtttgctctgagtggaacactgccatggaaaagctcactgggtggagcaggggggacatcattgggaagatcttggttggggagatttttggcagtagctgtcggctgaagggtccggatgctctgacaaaattcatgattgtgttgcacaatgcaattggagggcaagacacagacaagtttccattttccttctttgaccagaatggaaaatatgtgcaagctcttttgacagcaaataagagagttaatattgagggccagattattggtgccttctgctttttgcagattgcaagtcctgaattgcagcaagctctcaaagtccaaaggcaacaggagaaaaaatgttttgcaaggatgaaagagttggcttacatttgtcaggaaataaagaaccctttaagtggcatacgtttcactaactctcttttggaggccactgacttaactgaagatcaaaagcagtttcttgagactagtgctgcttgtgagaagcagatgtcaaagatcataagggatgttgatctggacagcattgaggatgggtgagtttgtttttatttatgggtttatatttaaatggacgaaattcaagaagcaattttttctgctccttttgggggtgctaaattctgagacatgtctgtctgtctagaaggtatagctttacctatttttatttctctattgaatcccaaggggatgtagatctatgcaagtctgtttgctttttctcatctgtaacaccaacaaggaaaaagaaaatgaaacaagctagaagaagagtttgttggcttggttgattaatggaatagtgcaaaagacaatagaagaaatgccctcaagtctggtctgtaacttccagatcagtatcagatagtatgatgttgaagtaattatgtttttaaaataggttggagagattcatgtgataaatgaacagttactggaagggctgggtgaaccctgtaccattgtgtcaaccatcatttgactcaaacagttacatgattgcctcaactcaacaaaatcttatctaaactattttgggttgcctgtctgaatcatgttttggtattccatgtcatctaatgtcattgttaagataaatgtttgttgttacatataaaaattgtttagctttgatatttcaagctaggatttgttaacagctggcagccactaaatgaagacgttgaagctataaacaaacagcagatgctttgttgtggatcttttgtttcatgtttttagttgcatagaacttttgatttaggatattgttatcctccctctctctccctctctctcatttgctcaaacatttctaccatattcaagtgcttaattgtaatgctttcattcaaacctcttgcctagtttttctaattttagttgtcttctggtattagttcactggagcttgagagggctgaatttttacttggaagtgtcataaatgctgttgttagccaagtaatgatattgttgagggaaagagatttacaattgatccgggacattcctgaggaagtcaaaacactggctgtttatggcgatcaagtaagaattcaacaggttttggctgatttcttactgaatatggtgcgttatgcaccatccccagacggttggatagagattcaagtttgtccaagattgaagcaaatttctgaagaagtaaaacttatgcatattgaattcaggtatgctaatctcatatcctgttttctgtgtgtttttcttgtaagcattttcaccacaggagagaatcttttagttacgggtgctatttctgtataaatattgaaacatgctggtggcaactataggtcatagtttgcatgtgaaaggtgcattggagcagttatagtcaagcggcagctaaaagttgtggttaatggtttcaatgagaatggcatataaaattatagcaatacttgatctataccacatatgatataggatataattgccttttttttcctcgttgcacttctatggaaatggcattagaaaagttaaatggaaaaaaaatgatagtgagaattattaggaaactgtcatcttatcatgacttttttgtatcgattttctagtttctgtctaaagttgcttgcacttaagttagaccaatttcaaatagtatttggtttcccagtgatgtttgacctttctctcaagtgatagcaagacaagaactttaccacctagatttgatggtctccataaccaaccaagatataggttttacttagatcatctggggaaaatggagttggagaacagggaaatcttattcaattgttaattttgtaatgttgaatgactactgtttcatgatgttcacttaaattagtttttgaaaaaaccatctaattattcaaatctttgttgcagagtatataaattatacgccctttggttgaatcataatgacttgcaatttttacaccatcttgggactatgcagagtaattatgaactttattggcttggagggcattggtaagtttaagagaactgtcgggctcacaaagttactagttgggcatcggggaatgcaacagtagatcatgctggacagtattatgtgctattccagtaagttcaaggaacgggtctgggtctcttgacaaattaggaatatgattgtatagatttaagggtgggttgaatgaactgtgggggggaggaggttgtcaccatagatatctactaagggtggcacagatataaggacagccgagcaggattggtgatctttgaaaatgatttttgatctttaagtatagcacatcttggttagagaacaacatttcaggttcttctcataagttatgcagtgctagttatttcagtttcttctcataagttatgcaggtgcaagttatttaagagtaatgcagttgtcaagatgaattagaatctaattctttgattggagtcatggattaatgcattgagagagaaattctttgatgggatcaacacttcatagatctagttaagcctatctgtgatatctaattggttgttcaggtgctagcctggttcagcccaaggttctttgatttcccctactggaaaaatacaaatgatcttttattctcatgacatcctatttttagcctgacttttggagaaagttttctaaaaccagttaaccaacaatttttttaagcatttacccttttctagcatgtagagcagaaaatagttctgagttaatggaaccaaacatgctcatggattcatggttagttcaagatctttttttttgagttcgagtttaattgatcaataaatggaggtatgctgcgtgtggagaccgctttgaatagcaagagattttgtaaaattttggaaaaatatgaagaacttagataagcctgtaaatgataaaatcaatatattcagacccacccatatgtaaagacaaccatgtgagccaacagtatgctccaagacaggtggaaactctaatgaagtctaattgttatcctaataaaaactgtataaatgttaaaacacttgaatagtggtacaaaatgttgaccaaaaagttatggatgtagatcctttttttaataggtgaattagagatactttagataggcaccaatcaaaaagggaggtgcaacccatgttcttgtaattgatttatcattctttctttctcgtgaagagagtaataaattaaagcaaaatttgcttgatttagtacaagaattttaatttattattgttggtagtattgatctatggtatgttacctcttacttaaagaagaaagtcaagggctaagcgtattgtccaaaaaaatggtctcaggccaaactattaacatatacagaaagaagcatgtgatagtcacttgtaaattgcaatgagcagaaactgttgcttatattgtttaccttcatgtgataatatgctggaaatttgagttgctaatattgtgttttttatgaaggatggtatgccctggtgaaggtcttcctcctaatctgattcaagacatgttccatagcagtcgttggatgactcaggaaggtctagggctgagcatgtgcaggaagatcttaaagctcattaatggcgaagtccaatatatcagagaatcagaaagatgttattttctaatcagcatagaacttcctatacctcacagaggctcaaagagcgttgactaggttattggcaccaatgactctccaaacttcaggcccttctcgcttcaacttcagatgcaacttctggcatacacaacccacttaaaagcttcaggctcagtacaagagtcacccagatgggctaatgggtattcaataaaactacttcaacatccatttcggagtgtttcgtccctgctgtgttccaacacactctaatataatatacaccctggttgatttcacatctcagtgtagtcctaaagtgttctcaggaaccgggttatgccagatcctactgcaagggtgtgcctcaagtgatccatcaacagagtcggggtcaggggtcaattgcagattctttgataaccgggtttgcacttgcaggtaaatctgagtagggtttctgccttaagcatgttatactagatagaatatttgtattatgtcctctcaagttctccaagagtggctgtacaatatctacttcttcaggtctaagctattctttttctttccttttttggtgtgataaaacaaattctcttactctatgcttcaagattatgtaacatgagtactaataatctaatctaaatatactctaatttcccattctatgtttattttcgattcatcagttatttttatccatcttctattgtagaatagcttgggatgcctcacaggttatctttaagagttttatatttgttatgttatgctacatg

SEQ ID NO: 43 - DNA, 8510 bp, Artificial Sequence
  Synthetic Oligonucleotide
atcatccacactcatatttactctatatctcaactctataccaattatcatatataatattatttctaccacatattatattatttctctctcctgagcagtcttcctactgctcgcctccggtccgtctcgggacccacctttggcctgccagtagcacggaagggagaccctagaaatagcgttcgctcgctcgctctctacggtagagtagaggatacagcgcctcgctgcagtgatttacctcgctgcggtaatttcttactgcatatgtaactctatatgcatagcgttcgtcgtgcagtcgccgagtgcggctagcctaatacgctatgcacaagagctgactctctctagcttaacagcatggacgagtcatcaatcacatccatcctggtcttacggccgtacgtgccatactggttttcgttactactaactattaatagtactcctacctaataccagcaacggcacaagttgtttacgtgctgcttagattatccaaatcggtttaagtacactttacgttgcccatgtcggggtgtggaggacctcacatcgcatgtacagaggcaccacaagcagcagcacagcgcagcgcaacgcaggtgagcttgttgggcgacgagcctctagtgggcgcagcaccagtctactactaggcgtctagctagcccattgccgcattgtgtatttgcgttactactaaacatggtaaacgaagtggcgcctcgaatcagcgctcgacacctcccgctgcgcccccatccgcgccgggcaggcatgggcaccgccgggtcggcgcgcgcccccacctagccgtcgactcggcgctgctcactcgcgctccacgcccacgcccacgcccaccgccccccgctatttatgcgtacttgcttgccgggagagtcgccggagagctgggcgtcctcctcccgttccagagcctcactgcttcgctccacccacccgtaggtctcctcctcctccttcctccccatgttgctcagcttgctttacccgtcacctgttcaccaccatgttcttcttcttggacacggactgtggcgagggtcttggatcgatcactggatgcctgcctggatcctccgttgcgtctctcctgcccccatctgggggcctggcagtggcaggctcctcgatcgggggcagctagagcgcgataggtttttttttttgtttgtttgtttctggaccaatcagcaggagacatgattgctccttctgtccttcaacaccacaagcactgatagttactagctcgttggattagggatgaacagaagctgcaaaagctggcgtttttttttcactacttgttcttctgcgtttctggttagcttcgacaccatacgcatactcgatggctgcggttggcaaacttagcatatgctatagttttagaaaatgggaagatgaacaccccgagaaccttttttcctgcgatttgctcatacacaaaatatagatacaaaaaaaaaaagcatcccctttctggtttctggggctgctgatgacgagcgactggataacgtctgttggctgtcgctattcagctgatctggtaccgtcagagggaggagaaacgggttctgcaagtccagtcccgtagcttttgttttcggttgcctgtggttctttcttgcatacttgcattatcggcatgctccatgtcggcaggggcggagctacacattcagtggtgcgggtgcagcacccacgtaaaattttaaatacagtaggagtaggggacaaaaaatcaattgcactcgcaagtcacaaggcaacatgcaccctcctccctcgtatttgctctagctccgccccctgcatgtcggagagaccaaatttcgacgcgaaagattgggaacgaaacggatgtggaatagtagtgcgctgctataggccctgtttggcacaactattgcttgttaaaaaagcagcttatctaaaaagccggtgaaaagcagtttctacttgttggctgcttttttatgtattctgagaagcagctgaactgataagttgctgcagaggctgtctgtttggcagaacttctgcttaaatttgtgaataagctgaaaataagttgtgccaaacagaaccataatctctagatcatggggagtaggaaatggggatccgttgatctgcatttccttggctgttagtattcatgtctttgtaaagtagcatgatgttttctgttgtttgttagcatcggattgtcccggtaagttctccatgtaatctacacaggcttgtaatctgatcttttttctatctatcaatgaaacgacacacggctcttctgggagttcgggaaaagaaaataagctctctatgttaggatctagatgttctcctgaaatgttagtgaacatttagatccgattagaatatgtcccgttatagaacagcagaggcttttctcgtatcacagaactgaaaactcgtaatccccatctattggagaagaatgtgaaatgaaatcttgctccataaacccataagactgaaaattatcaaatgtgttattcatcattagtcttaggttgtttctttcatattttgattagtttggaggcaaagccatcagttgctttttaatctttctgagctattgcctgaaaatagtacatgtatactacaacagtttcatttttgttgagagaaaaaaaaggaattcatctaagaaccacatgttgtagcactggttctgttaataggcgtgggaagtgcaatagcccatggggcaaaatccatgatgcacagtgcgattagaattttaccattttttatcaaaactataacatggatcagtttagaaattttttcctaactaaacgtttttttgtggggtaaataaaaatttggcatactgctttggaaagattgaatataataactgaaacatagctttttgtgattcatttttaggcagtctatcaaaaattaaattgctattgtctatatttcctcaaggtatggaaagacgaaaaggaaaacaaatcatttcacggtgctaggatttggtattttctatatttcctcaaggtctgcaatcatattttggcaagtggctacaagaacttgtcttatgtgttgagtcattgtaaatattgcagtatattgactgaatattctgtttttgcatcatactggggtgttattactttatatattactgatttatgctttccactgcccttttaccagttaagcaagagtggtatctggtggttttgtttttcaaaagaagacagaaatgtcttccttgaggcctgcccagtcttctagttcatccagcaggactcggcagagctcccaggcacggatactagcacaaacaacccttgatgctgaactcaatgcagagtatgaagaatctggtgattcctttgattactccaagttggttgaagcacaacggagcactccacctgagcagcaagggcgatcgggaaaggtcatagcctacttgcagcatattcaaagaggaaagctaatccaaccattcggttgcttgttggcccttgacgagaagagcttcagggtcattgcattcagtgagaatgcacctgaaatgcttacaacggtcagccatgctgtgccgaacgttgatgatcccccaaagctaggaattggtaccaatgtgcgctcccttttcactgaccctggtgctacagcactgcagaaggcactaggatttgctgatgtttctttgctgaatcctatcctagttcaatgcaagacctcaggcaagccattctatgccattgttcatagggcaactggttgtctggtggtagattttgagcctgtgaagcctacagaatttcctgccactgctgctggggctttgcagtcttacaagcttgctgccaaggcaatctctaagattcaatcgctaccaggtggaagcatgcaggccttatgcaataccgtggttaaggaagtcttcgaccttacaggttatgacagggttatggcttacaagttccatgaagatgagcatggggaggtctttgctgagatcaccaaacctggtattgagccctatctaggcctgcactatccggccactgatatccctcaagctgccaggtttctcttcatgaagaacaaagtcaggatgatctgtgattgccgtgcaagatcggtgaagattattgaagatgaggcgctctccattgatattagcttgtgtggttcaactcttagagcaccacatagctgtcaccttcagtatatggaaaacatgaactcgatcgcatcccttgtcatggctgttgtggttaatgaaaatgaagacgatgacgaacccgagtctgaacaaccaccacaacagcagaagaggaagaaactgtggggtctcattgtttgccaccacgagagccccagatatgtgccgtttccactgcggtatgcctgtgaattcttggcccaggtgtttgctgtccatgtaaataaggagtttgaattggagaagcagatacgagagaaaagcattctgcgaatgcagacaatgctctctgacatgctattcaaggaatcatctcccttgagtatcgtatccgggagtccaaatatcatggacctcgttaagtgtgatggtgctgctcttttgtatggggacaaagtatggcggcttcaaacggctccaactgagtctcagatacgtgatattgccttctggctttcagaagttcatggggattccactggcttgagcactgatagcctccaggatgctggatatccaggagccgcttcccttggtgacatgatctgtggaatggcagtggctaagatcacgtccaaggacattcttttctggttcaggtcacatacagctgctgaaatcaagtggggaggtgcaaagcatgatccatctgatgaggatgacagcagaaggatgcaccctaggctgtcctttaaggctttcctcgaggttgtcaagatgaagagtttgccatggagtgactacgagatggatgctattcactcgttgcaacttattcttagaggtacactgaacgatgccttgaagccggcccagtcatctggtttagataaccagattggtgatctcaaacttgatgggctcgccgaactgcaagcggtgacaagtgaaatggttcgcctgatggaaacggcaactgttccgatcttggcggtagatggcaacggattggtcaacggatggaaccaaaaggtggcggacttgtcggggttgcgagttgatgaagctataggaagacacatacttacacttgtggaggattcttctgtaccaattgttcagaggatgctatacttagctctgcagggtgagtgatctgcaacatttgattgccgttcttgctagtaatagtcaaccttcagaaaaaagaaacgaacagcttattgtatttttttgaaagggtttattgtattgtgtattgatttctaactatttcatcgattgagcaggcagagaagagaaggaggttcgatttgagttgaaaacccatggctccaagagggacgatggccctgttatcttggttgtaaatgcttgtgccagccgtgacatgcatgaccatgttgttggggtgtgctttgtagcccaggatatgactgttcataagttggtcatggacaaatttacccgggttgagggggactacagggccatcattcacaacccgaacccgctcattcctccgatatttggcgccgaccagttcggatggtgctctgagtggaacgcagccatgaccaagcttactgggtggcacagagatgaggtgatcgacaggatgctccttggcgaggttttcgacagcagcaatgcttcctgccttctgaagagcaaagacgctttcgtacgtctttgcattatcatcaacagcgcattagctggtgaagaggcagagaaggctccaatcggtttctttgaccgcgatggcaaatatattgagtgccttctgtcagtgaacagaaaagtgaatgcagatggcgtcgtcaccggagtgttctgtttcattcatgttcctagtgatgacctccagcatgcgctacatgtgcagcaagcctctgagcagacagcactgagaaggctgaaggctttctcgtacatgcgacatgccatcgacaaacctctctcaggtatgctctattctagggaaacactcaagggcacagacctggatgaagagcagatgaggcaggttcgtgtcgcggataattgccatcgccagctaaacaagatactcgccgacttagatcaagataacattactgacaagtaaccttcttgccaagtctttttaaactgttctattaggttcttgtattcaataactactgaagcttatagccatattcgttactaccaccttaatatttctgtatcattatgtttcacaggtcgagttgcttggatttggacatggctgagtttgtgctgcaagacgtggtggtgtctgctgtaagtcaagtactgataggttgccagggtaaaggcatcagagttgcttgcaacctgccggagagatccatgaagcaaaaggtttacggggatggtatacggctccagcagatcctctccgacttcttattcgtttcggtgaaattctctcctgctggtggctctgttgatatctcttccaaactgaccaagaacagcattggcgaaaaccttcatctcatagacttcgaacttaggtacaccgtgcatgctaatttgcacgcatgcactcatttctggataggagattacaaggtatgaaggcttaaattactcctgtgttgatggctttatggtctttcaggatcaagcaccaaggagcaggagtcccagcagaaatactgtcacagatgtatggggaggacaatagagaacagtcggaggagggcttgagcctccttgtttctagaaaccttctgaggctcatgaatggcgacattcgtcacctcagggaagctggcatgtcaaccttcatcctcactgctgaactcgctgctgctccttcagcagctggacattgaagccgtcactgcaaacaagtgccaaatggtatgttcgctagtttgtgttaaggcagtctgcaatttcccaaatttccttagcttagaataaaataactctgcaatttcccaaatttcctactatggatatatgatgaataaataaggtaccatgttctttccatacgaggggtaatgatacatagatctcctgcgtatcctcggaaaacaatacagaacagaggataacaattatgcctaattccgattaattggtactgtgatgtatttttgtttcatgtgtacgtcaagtaacacagtcctcaagagtattatttctgcccttggtgccagctgcccagctccctcagagttcattcggaaagcaacggtggttcgcggagaatgggaaatgctgcagcctgtaggatcaccgaatgcataagaaataagattgacttgtatggttgtttgttaggcggggaagttgtgcaggcatagtaagactccatacttctgctcttttctgcctgtaaccaactgttttctatcagttattaggctatctcatcagtttaattatacttatccctcatatttaaatttcactttgcaaacaatacaaaacagtgttttgatgaccatatggatgaactgcgaagacaatttttttaggctgtctctaatatttcatccatatggtcacccaaaatactattttgtacggtagactacactgttagtagtgtggagtttaaatatggggatgaataacctgttggagataaccttacctatcttcagcaaatcttctatcatatctcttattttaaacttcactctacaaacaatatcatatacaattttgtaatgtcatatacagttccgtgtcccatgttttaccatatctattgatgatcatgattattaagacgtcgctaaagcgacgtttaaacggtaaagcgtatctagacttgtaaggcgtcctgtcgccttgcccgagagtaaggcgaggcactacggacgcacgcggcagcgacgcacagggagaagtgtggcggcgacgcgtaggatgtcagacctcgccgaagactcacctcctgttgccggcgagctccccctgcagcctttcacaaacacacagaactcacacgaatagaattggatacactctgatttttggtgctgcgtaaaactgcgcaacttttctgactctgtttactgaataaaaaggattcaacagaacgtggccaacgaccacccgagaagcacggcacgtccctccactttgtgtgccatcccacacataggtatgccggccacgactacacgtacagccaatagatgactatacgcctaacctgggaataaaaaaacaacagccnttaagtaaactaactaagacccatgcacaaggattatttcaacaattctcctcctaatacttgtgcatatactactgtgctgatcga

SEQ ID NO: 44 - DNA, 3900 bp, Artificial Sequence
  Synthetic Oligonucleotide
gctatttatttattcagctcgccactgccacccgagtgcggctgccttggctttgtctcgtctgcctcctcctcttcttcccgcacagtccacagctccgccactactggagcagagctgagctgagccgagcaggagtggtatacagcagataagactcttgagtgattcaggtaatcaaggcagaagatgtcttcttcaagacctactcaatgttccagttcatccagcaggacccgccaaagctcccgggcaaggatattagcacaaacaactcttgatgctgaactcaatgctgaatatgaagaatatggcgactcctttgattactccaaattggttgaagcacagagaactactggacctgagcagcaagctcgttctgagaaggtcatagcttacttgcatcacattcagagagcaaagctaatccaaccatttggttgcttgttggcccttgatgagaagaccttcaatgttatagcgctcagcgagaatgcaccagagatgcttacaactgtcagccatgcagtgccaagtgttgatgatcccccaaagctacgcattggcaccaatgtatggtctcttttcactgacccaggtgccacagcactgcagaaggcactgggatttgctgatgtttccttgctgaaccctatcctagttcaatgcaagacctcaggcaagcctttctatgccattgttcatcgggcaactggttgtttggtggtagactttgagcctgtgaaacctacagaatttccagcaactgccgctggggctttgcaatcttacaaacttgctgccaaggcaatctctaagatccagtcactgccaggtggaagcatggaggtgctatgcaatacggtggtcaaggaactctttgacctcacaggatatgatagagttatggcttataagttccatgaagatgaccatggtgaagtctttgctgagatcacaaagcctggtcttgaaccttatcttggcctgcattatccagctactgatatccctcaggcagccaggtttcttttcatgaagaacaaagtccggatgatttgtgattgccgtgcaagatctatcaagattatcgaagatgagtcgctccacttggatattagcttatgtggttcaacactgagggcaccacacagttgtcatcttcagtatatggagaacatgaactcgattgcatcccttgtcatggctgttgtggttaatgagaatgaggatgatgatgaagttggggctgatcaacctgcacaacagcagaagaggaagaaactatggggactccttgtttgccaccatgagagccccagatatgttcctttcccattgcggtatgcctgtgagttcttagcacaagtgtttgctgtccatgttaacaaggagtttgaattagagaggcaagtacgcgagaaaagcatattgaggatgcaaacaatgctctctgacatgcttctcagggaatcctctcctctgagtatagtatcagggactcccaacatcatggaccttgtgaaatgtgatggtgctgctcttttgtatgggggaaaagtgtggcggctacagaatgctccaactgagtctcagatacgtgatattgccttctggctgtcagatgtccacagggattccactggcctgagtactgatagcctacatgatgctggatatccaggagctgctgctcttggtgatatgatttgtggaatggcagtagctaaaataaattccaaggatatcctgttctggttcaggtcacatacagctgctgaaatcagatggggaggtgcaaaacatgatccatcagacaaggatgacagcagaagaatgcaccctaggctgtccttcaaggcattccttgaggttgtcaagatgaagagcttgccttggaatgactatgagatggatgctattcactcattacaacttatacttagagggacactgaatgatgacatcaagccaacaagggccgctagtttagataatcaggttggtgatctcaagcttgatgggcttgctgaattgcaggcagttacaagtgaaatggttcgtctcatggaaacagcaactgtcccaatcttggctgtagatagcaatggattggtcaatggatggaatcagaaggttgctgagttgacagggttgagagtagatgaggctattggaagacacatacttaccgttgtagaggaatcttctgtaccagttgtccagaggatgctgtatttagctttgcaaggcaaagaagagaaggaagtgaaatttgaggtgaaaactcatggctccaagagagatgatggccctgttatcttggttgtgaatgcctgtgccagccgggaccttcacgaccatgttgttggtgtgtgctttgttgcacaagatatgactgttcataagttggtcatggacaaatttactcgggttgagggagactacaaagcaattattcacaatccaagcccgcttattcctcccatatttggtgctgacgaatttggatggtgctctgagtggaatgctgccatgacgaaattgaccgggtggcatagagatgaggtgatcaataagatgcttcttggtgaggtgtttgatagcaccaacgcctcctgtcttgtgaagaataaagatgcatttgtaagtctctgcattcttatcaacagtgcattagctggtgatgaaacagaaaaggctccattcagcttcttcgaccggaacgggaagtatatcgagtgccttctttctgttaacagaaaagtaaatgcagatggtgtcatcactggagtattttgtttcattcaagttcctagtcatgagctgcaacatgcactacatgtgcagcaagcctcacagcagaatgcactaacaaagttgaaagcttactcctacatgagacatgcaatcaacaaccctctctcaggtatgctttactctaggaaagcactgaagaacacaggtctgaatgaagagcagatgaaggaggtcaatgttgcagatagttgtcaccgccagctgaataaaatactttctgacttggatcaagatagcgtcatgaacaagtctagttgcttggatttggagatggttgagtttgtattgcaagatgtgtttgtggctgctgtaagtcaagtactcataacttgccagggaaaagggattagagtctcttgcaacctaccggagagatatatgaagcaaacagtctacggggatggtgttcgactacagcagattctctctgacttcctattcgtctcagtgaagttctctcctgttgggggttctgttgagatctcttgtagcctgaccaagaacagcattggggaaaaccttcatctcatagacctagaacttaggatcaagcaccagggcaaaggagtcccagcagatctgctgtcacaaatgtacgaggatgacaataaggagcagtcggatgaaggcatgagtcttgcggtttctagaaacctgctgaggctcatgaatggcgatgtccgacatatgagggaagctggcatgtcaaccttcatcctcagcgtcgaacttgcttctgctccagcaaaataatcatgaaatgttcgttcgtatagccacgacggtggcttgtgcttaggattggacgtacttgcagactgaagattcactggctgcagataaatggttgactttacagataaatggtcgtatggcacatttggttatgtactgtcgtcttcttcaccatgctaatgtcagtatgtgtaatcttctaagttgcaaatgcgtgctgttgctcgcctgaactcattaagattggaagtgaaaacaccaatctgtaatatgctgtatcttgatgctgagagcattcaagttactactacatgaataaaatatggtttgactttactcact

SEQ ID NO: 45 - DNA, 3705 bp, Artificial Sequence
  Synthetic Oligonucleotide
caggagcgacacacggcacatacgactgtcgagtggttcaattacttgaggcaggcgatgtcttcctcaaggcctgcttccagttcttccagcaggaaccgccagagctcccgggcaagggtgttagcacaaacaacccttgatgccgaactcaatgctgaatatgaagaatctggtgactcctttgactactccaagctggttgaagcccagagggatggtccacctgtgcagcaagggcggtcagagaaggtcatagcctacttacagcacattcagaaaggaaagctaatccaaacatttggctgcatgttggcccttgatgagaagagcttcaatgttatcgcgttcagtgagaacgcgccagaaatgcttacaacggtcagccatgcggtgcccagtgttgatgatcccccaaggctggggattggcaccaatgtacggtctcttttcagtgaccaaggtgccacagcactgcataaggcactaggatttgctgatgtgtctttgctgaatcctatccttgttcagtgcaagacatcaggcaagcctttctatgccattgttcatcgagcaactggttgtttggtggtagattttgagcctgtaaagcctacagaatttcctgccactgctgctggggctttgcagtcctacaagcttgctgccaaggcaatatccaagatccagtcattgccaggtggaagcatggaggtgctatgcaatactgtggttaaggaagtctttgaccttaccgggtatgatagggttatggcttacaagttccatgaagatgaccatggtgaggtcttcgccgaaatcacaaagcctggtcttgagccttatctaggcctgcactatccagccactgatatccctcaagcagccaggtttcttttcatgaagaacaaagtacggatgatttgtgattgccgtgcgagatccataaaggtcatcgaagctgaggcactcccctttgatattagcctgtgtggttcagcactaagggcaccacatagttgtcaccttcagtatatggagaacatgaactcgattgcatcccttgtcatggctgttgtggttaatgagaatgaagaggatgatgaagctgagtctgaacaaccagcacagcagcagcagaagaagaaactatggggcctccttgtttgccaccatgagagccccagatatgtcccttttccgctgcgttatgcttgtgagttcttagcacaggtgtttgctgtccatgtcaacagggagtttgaattagagaaacagttgcgtgagaagagtatactgaagatgcaaacaatgctctctgatatgttgttcagagaagcctctcctctgactattgtatcaggggcacccaatatcatggacctagtcaaatgtgatggtgctgctcttctgtatgggggcaaagtatggcgtctgcgtaatgctccaaccgagtctcagatacatgatatcgccttctggctatcagatgttcacagggattccactggcctgagtactgacagcctccatgatgctggctatccaggagcttctgctcttggtgatatgatttgtggaatggcagtggctaagatcaactccaaggatattattttttggttcaggtcacatacagctgctgaaatcagatggggaggtgcaaagcatgattcgtcggacatggatgacagcagaaggatgcaccctaggctgtctttcaaagctttccttgaagttgtcaagatgaagagcttgccttggactgactatgagatggatgctattcactcattgcaacttatactccgagggacactaaatgatgccagcaagccaaagcgggaagctagtttagataaccagattggtgatctaaaacttgatgggcttgctgaactgcaggcggtgaccagtgaaatggttcgtctaatggaaacagcaactgttccaatcttggcagtagatggcaatggactggtcaacgggtggaatcagaaagcagcggagttgactgggctaagagttgatgatgcaataggaagacacatacttactcttgtggaggagtcctctgtaccagttgtccagaggatgctatatctagctctgcagggtaaagaagagaaagaagttcgatttgaggtaaagactcatggcccgaggagggatgatggtccagttatcttggttgtgaatgcttgtgccagtcgggaccttcatgatcatgttgttggagtgtgctttgttgcccaagatatgactgtccataagttggtgatggacaagtttactcgggttgagggtgactacaaggcgataattcacaacccgaacccactcattcctcctatatttggtgctgacgaatttggatggtgttcggaatggaatgctgcaatgaccaagttgactgggtggaatagagatgaagtgctcgataagatgcttctcggtgaagtgtttgacagtagcaatgcttcctgccctttgaagaacaaaaatgcatttgtaagtctttgtgttcttatcaacagtgcattagctggggaagaaacagaaaaggctccatttggcttcttcgaccgaagtggaaagtacattgagtgtcttctatcagcaaacagaaaagaaaatgagggtggtctcatcactggagtattctgttttattcatgttgctagtcatgagctgcaacatgcactacaggtgcagcaagcctcggagcagacgtcgctaaaaaggctgaaggctttctcctacatgagacatgcgatcaacaaccccctctcaggaatgctctactctagaaaagcattgaagaacacagatttgaatgaagaacagatgaagcagatccatgtcggagataattgtcaccaccagataaacaagatacttgcggacttggatcaggatagcatcagcgaaaaatctagctgcttggatttggagatggctgaatttgtgtttcaagatgtggtggtggctgctgtaagtcaagtactgataacctgccagggaaaagggatcagaatctcttgcaacctgccagagagatttatgaagcagtcagtctatggagatggtgttcggctccagcagatcctctctgacttcctgtttatttcagtgaagttctctcctgttggaggttctgttgagatttcttccaagctgacgaagaacagcatcggagaaaaccttcatcttattgacctagaacttaggatcaagcaccagggattaggagtcccagcagaactcatggaacagatgtttgaggaggacaacaaggagcagtcagatgagggcttgggcctcctagtttctagaaagctgctgaggctcatgaatggggatgttcggcatctaagggaagctggcgtgtcaaccttcatcctcaccgctgaacttgcttcggctccaacagcaatcggacaatgatgaagccagtgcaacatatggtcatcagacggtttgtttcaatttcgaggatagccgtgagcaatggtgaagttggtgacattttccgaggaggatggaatgtgctgcaacctgttgcggcgtcagggtgaaaagttgtgcggcacagcttggttatgtacttgtgctgtgttacgtttctgcaatcctactgtgtaaggttcaactttaattttttacatgaataaagtgtgcagatgtacctgcaccgttggtctc

SEQ ID NO: 46 - DNA, 3759 bp, Artificial Sequence
  Synthetic Oligonucleotide
gcgtagcacaagagagaaggtggtgatcgaggccaagatttagaatttaactataacaaaaagcctctgacgagtgtgactagtcacaagatctgatcatggcttcttgaaacttcttcttcttctttcttctctttaaaggaaaaaaatgtcaggctctaggccgactcagtcctctgagggctcaaggcgatcaaggcacagcgctaggatcattgcgcagaccactgtagatgcgaaactccatgctgattttgaggagtcaggcagctcctttgattactcaacctcagtgcgtgtcactggcccggttgtggagaatcagccaccaaggtctgacaaagttaccacgacttatcttcatcatatacagaagggaaagctgattcagcccttcggttgtttacttgccttggatgagaagaccttcaaagttattgcatacagcgagaatgcatctgagctgttgacaatggccagtcatgcagttcctagtgttggcgaacaccctgttctaggcattgggacagatataaggagtcttttcactgctcctagtgcgtctgcattgcagaaagcccttggatttggagatgtctctcttttgaatcccattcttgtgcactgcaggacttctgcaaagcccttttatgcgattatccacagggttacagggagcatcatcatcgactttgaacccgtgaagccttatgaagtccccatgacagctgctggtgccttacaatcatacaagctcgctgccaaagcaatcactaggctgcaatctttacccagcgggagtatggaaaggctttgtgatacaatggttcaagaggtttttgaactcacggggtatgacagggtgatggcttataagtttcatgaagatgatcacggtgaggttgtctccgaggttacaaaacctgggctggagccttatcttgggctgcattatcctgccaccgacatccctcaagcagcccgttttctgtttatgaagaacaaggtccggatgatagttgattgcaatgcaaaacatgctagggtgcttcaagatgaaaagctttcctttgaccttaccttgtgtggctccacccttagagcaccgcacagctgccatttgcagtacatggccaacatggattcaattgcatctctggttatggcggttgtagttaacgaggaagatggagaaggggatgctcctgatgctactacacagcctcaaaagagaaagagactatggggtttagtggtttgtcacaatacgactccgaggtttgttccatttcctctcaggtatgcctgtgagtttctagctcaagtgtttgccatacacgtcaataaggaggtggaactcgataaccagatggtggagaagaacattttgcgcacgcagacactcttgtgcgatatgctgatgcgtgatgctccactgggtattgtgtcgcaaagccccaacataatggaccttgtgaaatgtgatggagcagctctcttgtataaagacaagatatggaaactgggaacaactccaagtgagttccacctgcaggagatagcttcatggttgtgtgaataccacatggattcaacgggtttgagcactgatagtttgcatgacgccgggtttcctagggctctatctctcggggattcggtatgtgggatggcagctgtgaggatatcatcgaaagacatgattttctggttccgttctcataccgctggtgaagtgagatggggaggtgcgaagcatgatccagatgatagggatgatgcaaggagaatgcacccaaggtcatcgttcaaggctttccttgaagtggtcaagacaaggagtttaccttggaaggactatgagatggatgccatacactccttgcaacttattttgaggaatgctttcaaggatagtgaaactactgatgtgaatacaaaggtcatttactcgaagctaaatgatctcaaaattgatggtatacaagaactagaagctgtgaccagtgagatggttcgtttaattgagactgctacggtgccaatattggcggttgattctgatggactggttaatggttggaacacgaaaattgctgagctgactggtctttcggttgatgaagcaatcgggaagcatttcctcacacttgttgaagattcttcagtggaaatcgttaaaaggatgctagagaacgcattagaaggaactgaggagcagaatgtccagtttgagatcaagacacatctgtccagggctgatgctgggccaataagtttagttgtaaatgcatgcgcaagtagagatctccatgaaaacgtggttggggtgtgttttgtagcccatgatcttactggccagaagactgtgatggacaagtttacgcggattgaaggtgattacaaggcaatcatccaaaatccaaacccgctgatcccgccaatatttggtaccgatgagtttggatggtgcacagagtggaatccagcaatgtcaaagttaaccggtttgaagcgagaggaagtgattgacaaaatgctcttaggagaagtatttgggacgcagaagtcatgttgtcgtctaaagaatcaagaagcctttgtaaaccttgggattgtgctgaacaatgctgtgaccagtcaagatccagagaaagtatcgtttgctttctttacaagaggtggcaagtatgtggagtgtctgttgtgtgtgagtaagaaactggacagggaaggtgtagtgacaggtgtcttctgtttcctgcaacttgccagccatgagctgcagcaagcgctccatgttcaacgtttagctgagcgaaccgcagtgaagagactaaaggctctagcatacataaaaagacagatcaggaatccgctatctgggatcatgtttacaaggaaaatgatagagggtactgaattaggaccagagcaaagacggattttgcaaactagcgcgttatgtcagaagcaactaagcaagatcctcgatgattcggatcttgaaagcatcattgaaggatgcttggatttggaaatgaaagaattcaccttaaatgaagtgttgactgcttccacaagtcaagtaatgatgaagagtaacggaaagagtgttcggataacaaatgagaccggagaagaagtaatgtctgacactttgtatggagacagtattaggcttcaacaagtcttggcagatttcatgctgatggctgtaaactttacaccatccggaggtcagctaactgtttcagcttccctgaggaaggatcagctcgggcgttctgtgcatcttgctaatctagagatcaggttaacgcataccggagctgggatacctgagtttttactaaaccaaatgtttgggactgaggaagatgtgtcagaagaaggattgagcttaatggttagccggaaactggtgaagctgatgaatggagatgttcagtacttgagacaagctgggaaatcaagtttcattatcactgcggaactcgctgcagcaaacaagtagtccccaaaagaaaaggggtctggcttgatataaaatagtcactggttgttctttgcttgtaactttccttatcgcttttgttttcgttttcaaatttcagtaacgatgaaatatccatccatttacatcttctgttgaactcttttctgaagctgtaaatatggatgcatatctaatctcctcctgagtgttttggtttcatgatgtatcattcaaaataagtaatataggagaaatgagct

SEQ ID NO: 47 - DNA, 6180 bp, Artificial Sequence
  Synthetic Oligonucleotide
agtactactatccactcacgaattatatattatcgtacttcgtggtcttaattgggggtattttttagatttcaactgattataattattgtagacctggaggacaaatcaagaataagggttatgctaattttggggtacatcaattgacaccatcaaactatgtttggaaagagaacaactgggcaactcactgcttctagtaaagtctctctcacttcctatttatagaaacgcgtatctactatggctatttgtgtagagttgtcttgcatgaactttcacaagtgtgtgttttaatgaatcttttaaagggttaagtacttctcttgagttgaagctgtgatcgataccttggctgttgagcttttgttggcctaaggaaggaccagttttgttatacatttctttgctttcagattcagacttctatatatcaagtattgatcagctgaaagctaagttgttcttgcctctgaaatcttgtaaaaggtgaggcctttctcttgttgtgtttatcttgaaagttttctctccgcaaataatgtgttggttgtaatagcttattggtttctgttaaagagaaaatgacttgcatctttatctaattttgctactactataggataataaagaactcgaattcccaataagctgtgcctacttttgtttatctagctgaaagagctgttagcttagtgtattatgcctatttctctgcatgttgtgacttctttattatattagtacatagttgaaatatctattttgcacatatgcaacctccttcctgctttttgttttgcagagtatgttgattgtttgggggttttggggggtatattcttaagtggatgtagattctatgctgcaaaatgggggtgtgatattcatattgataaaatgtggagactctagaatgaatccttgcctgctaagttgctgatacaaaaagttaacaactctgttctattttgacagtgcctgaccgtgacggttcctaacttcctattataattttgtttttgttgatcatcggtggtaattgcttttgatgttctatttcagatttgagtcgaaatgtcatcttcaagaccaagccaatcttccaccacttcagcaagatcaaagcatagtgctaggatcattgcacagaccactatagatgcaaagcttcatgcagatttcgaggagtcgggtgattcctttgactattcaagctcagtgagggttacaagtgtagctggggatgagcgaaagcccaagtcggacagagtaactaccgcttacctcaatcagatccagaagggtaagtttatccagccatttggctgtttgttagcccttgatgagaaaacatttaaggtcatagcattcagcgagaatgcccccgaaatgctgaccatggttagccatgctgttccaagtgtgggtgagcttccagctcttggcattgggactgatatcagaacgatcttcactggtcctagcgcagctgcattgcagaaggctttggggttcggagaggtttctctgttaaatccggtcctcgttcactgcaaaacttctgggaagccatattatgcaattgttcatagggttactggtagcttaatcattgatttcgagcctgtgaagccctatgaagtacccatgactgctgcaggggccctgcagtcgtataaacttgcagccaaagccattactcgcttgcaggccttgcccagcggcagtatggaaagactttgtgacactatggttcaggaggttttcgaactcacaggctatgacagggtgatgacgtataagtttcacgatgatgatcatggagaggtggtggccgagatcacgaagcctggccttgatccttaccttggtttacactatcctgctacggatatcccacaagctgcacgctttttgttcatgaagaataaggtccgaatgatttgtgattgccgagcaaaacatgtgaaggtagtccaagatgagaagcttccatttgatttaacattgtgcggctctactcttagggctcctcactactgccatctacagtatatggagaacatgagttcaattgcatcccttgtaatggcagttgtggtcaatgacggggacgaagagggagaaagctctgattcgacacaatctcaaaagagaaaaaggctttggggcctggtggtttgccacaacaccaccccgaggtttgttcccttccctctgaggtatgcatgtgaatttcttgcgcaagtctttgccatacacgtcaacaaggaactggaattggaaagtcagattcttgagaaaaatatcctgcgtactcagactctcttgtgtgatatgctgatgcgagtagctcccttaggtatagtgtcacagagtccaaatattatggatcttgtcaaatgtgatggtgctgctttgctctataagaataagatacatcgacttggaatgaccccaagcgactttcagctgcacgatattgtctcgtggctttctgagtatcatacagattccacaggtttgagtacagacagcttgtatgatgctggtttccctggggctcttgctcttggtgatgtagtgtgtggtatggcagctgttagaatatctgataagggctggctgttctggtataggtcacacactgctgctgaagttagatggggtggagcaaagcatgaacctggtgaaaaggatgatggcaggaaaatgcatcctaggtcatcattcaaagcattcctggaagttgtcaagactagaagtgtaccatggaaggactatgaaatggacgcaatccattctttgcagcttatactaagaaatgcatccaaggatgccgatgccatggattcaaataccaatattatccacacgaaacttaacgatcttaagattgatgggttgcaggagctagaagcagtgactgctgaaatggtccgcttgattgaaacagcttcagttcctatcttcgcagttgatgttgatgggcagcttaatggctggaacacaaaaattgctgaattaaccggtcttcctgttgatgaagcaattgggaatcatttgctcacactcgtggaggattcctcagttgataccgtgagtaaaatgttggaattagcattgcaaggtaagttgcttttgcgtacttccctttccgtgttattgttaatgttataactcttttcgtgaagataattttttagcctttgtacgccattttattatattatttttagtagtattggtttacataagttttgtctatccattcagttaaatgaattatgaaattcctgtatatttagccgaatatcctataccttttaccagggaaagaggaaagaaatgtagagtttgaaataaaaacacatgggccgtcaggagattctagtccaatcagcttaattgtgaatgcatgtgcaagcagggatgttggagatagtgttgtgggtgtatgttttattgctcaggatataacaggacaaaaaaatattatggacaagttcacccggattgaaggtgactatagagctattatccaaaatcctcacccattgatcccaccaatatttggcactgatcaattcggctggtgttctgagtggaactcggcaatgacaaagttaactggatggcggcgtgatgatgttattgataaaatgctattaggggaggtttttgggacacaggcagcttgttgccgtctcaagaatcaagaagcttttgtaaattttggagttgtactaaacaatgctatgactggtcaagagtgtgcgaagatatcttttggtttctttgcacgtaatgggaaatacgtagagtgcttactttgtgtgagcaaaaggttggatagagagggtgcagtcacgggactcttttgtttcctgcagcttgcaagccatgagctgcaacaagctcttcacattcaacgattatcagaacaaactgcattgaagaggttgaaagtattagcttacataaggaggcagattagaaaccctctttctggaattatattctctcggaaaatgctggaggggactaacttgggcgaagagcagaaaaatatactgcgtactagttcccagtgtcagcgtcagctcaacaaaattcttgatgatacagatcttgatagcatcattgatgggtatgttataatcattttactatactacatgtattttagtttttatgtttatggctttaaaggatttaggatttgctgcattgttatatcagtttatactatcaacaaatttgttcttttttattctcttaccagttatttggatctggagatgctcgagttcaagctgcacgaagtattagtggcatctattagtcaaatcatgatgaagagcaatggaaagaatataatgattgtgaatgacatggttgaagatcttctcaatgaaactttatacggagatagtccgaggcttcaacaggtcttagctaactttttgttagtatgcgtgaattctacaccaagtggtggtcagcttagtatttcaggcacattaacaaaagatcgcataggagaatctgttcagcttgctctcttggaagtcaggtaattaaattcatatgatagaacaatctaaactggctttttcaccttgaccagatttctactgtccaggttagcatctccagcagttatttttgttcgattgtgtgcctattctctccagccgagaggttgtgagttcgagtctccccaagagcaaggtgggaagttcttggagggaaggatgtcgggggtctatttggaaacagtctctctaccctagggtaggggtaaggtctgcatacacactaccctccccagaccccactaagtgggattatactgggttattgttgttgttgttgttgtgtgcctattctctaaaacaaaatcaggaccagaaattttagcttgaactcaatgtctgaatgtttgaagtcctatggttgcaatagcatttgatctttaacagtagaagctgctagtctgctactgctctctgtcttctctcctccattggttttgtaaatattaatttttcagccaagacgtgttcatgtggtcattatataaaagataaaatggattaatttaaaaatcactcatctgcttgcaggataagccacacagggggaggagtgccagaagaactgctaagccaaatgttcggtactgaggccgaagcatctgaagaagggatcagcttactcatcagcagaaagctggtgaagctgatgaatggggaagttcagtacctaagagaggcggggcgatcaactttcattatatccgttgaacttgcagtggctaccaagtcaagctgctgatgatttcaggaacctttcaaactagtagatctgtatagtgaaaaaggatttcaagttatctgtgtttggagatcttgctgtcttgtgatgttcctcctctttggcaccctcgggttctttttaataagtttcttgaactattgcatcttcaacatttcagcatttccagtttcattataattccatgtgcaatcagaaatttttatcacttttgggaggcagaatttcaagatttcctatttctaagataccatctttccttttcatcaagaaaagataaaggatgatcgaaatggtaaagaacaatcttacgaatgaagataatatatccataccagcattatctaggcaaaaaatttaccctaggtcattgctgttcttttacatcttactctcttgaggtgcggtacttcctcgtgtggcatgtttcgtgcattactgttctttttattgtcaaagtatttctttcaattcaaattgtattaatcatagcaatgaaagagagattggaaggtgagactatgagaaatgaaatagagatttaaaaggatacgattccttaacagggtttgaacgtgcaactatgagaacaactggggaattgtgaattcttgaagggagttgggagataagtgtaatgatcggatcaaaacctcatcttgggaggtgcttacgccttgaaataatgttctcattctatcatataattaatggttagacctagaaaaagaatcaaagattaagggtgtatttggtacgaaggaaaatatttttcaattttttcaatgtttggttaaatgttgtgaaaaacatttttctcatgaactcaattga

SEQ ID NO: 48 - DNA, 3673 bp, Artificial Sequence
  Synthetic Oligonucleotide
tgaaggaagcaaaacaaaacatgaaaagaagaatcttgaggtaaaaacgaatcccaaatatcaaaaaggaaactttttttacttctttctttctcttgctgcaaatgatacagaatcagcttcttcttcttcttcttctggtcgaaactcactccacactttcccctaagctatgggattcgagagttcaagctcagctgcaagcaacatgaaacctcaacctcaaaaatccaacactgctcaatactctgttgatgctgctctctttgctgatttcgctcaatccatttacaccggcaagtcttttaactactccaaatctgttatttcacctcccaatcacgttcctgatgaacacatcacagcttacttgtctaacatccaaagaggcggtctagttcagccctttggttgtttgattgctgtcgaagaacctagttttaggatacttggtcttagtgacaactcttctgactttcttggtttgttgtctcttccttccacctcccattctggtgagtttgataaagtcaagggtttgattggaatcgatgcaaggacgctttttactccttcctctggagcttctttgtctaaagctgcttcctttactgagatttccttgttgaatcctgttttggtccattctaggacgacccagaagcctttttatgctattcttcacaggattgatgcagggattgtcatggatttggagcctgctaaatcaggtgatccggctttgacccttgcaggcgcagttcagtctcagaagctagccgttagggccatttctaggctgcagtcacttcccggaggagatattggtgccttgtgtgatactgttgtggaagatgttcagagacttaccggttatgaccgtgttatggtctatcagtttcatgaagatgatcatggtgaagttgtttctgagattagaaggtctgatttggagccttatttgggtttacattatcctgcaacagatattcctcaggctgctcggttcttgttcaaacagaaccgtgtccgaatgatttgtgactgcaatgcaactccggttaaggttgttcagagtgaggaactcaagagaccactttgtttagttaattctactctaagagctcctcatggctgccatacgcagtatatggcgaatatgggctctgtagcttctcttgcactcgcaattgtagtaaaaggcaaagattcgagcaagctttggggattagttgttggtcatcattgttctcctagatacgttccattcccgttgcggtatgcttgtgagtttctgatgcaagcatttgggcttcagcttcaaatggaacttcagttagcatcacagttagccgagaagaaggctatgcggacgcagaccttgttgtgcgatatgcttctccgtgatactgtttccgctattgttacacaatctccgggtattatggaccttgtgaaatgtgatggagctgcgttatattacaaggggaaatgttggttggttggtgttactcctaatgagtcacaagttaaagacttggtgaattggctggtggagaatcacggtgatgattcgacgggtttaaccactgatagtttggtggatgcgggataccctggtgccatctcacttggagatgccgtttgtggtgtggctgccgcagaattttcttcaaaagattacttactttggttcaggtccaatactgcaagtgcaatcaaatggggaggagctaaacatcatccaaaggataaagatgatgccggaagaatgcatccgaggtcatcgtttacagcctttcttgaagttgcgaagagcaggagcttgccgtgggaaatctcagaaattgatgctatccattccctgagacttataatgagagagtcatttaccagctctaggccagttttgtctggtaatggcgtagcaagagatgctaatgagcttacttcttttgtgtgtgaaatggttagggtgattgaaaccgcaactgcacctatttttggggttgattcatccggatgtattaatggttggaacaagaaaaccgctgaaatgacgggattactagctagtgaagcaatggggaagtcacttgccgatgagattgttcaagaggaatcacgcgcagctcttgaaagtctcttgtgcaaagccctacaaggtgaagaggagaaaagtgtaatgctgaaactgagaaagtttggtcaaaacaatcatccggattattcttctgatgtgtgtgttctcgttaactcctgcacgagtcgggattatactgaaaatatcatcggtgtctgcttcgttggtcaagacatcactagtgagaaagcaataacagatagattcatcagattgcaaggagattacaagactattgttcaaagcttaaatcctttgattccaccgatatttgcttcagatgaaaatgcttgttgttctgagtggaacgcagcaatggaaaagcttacgggatggtcaaaacacgaggtgattgggaaaatgctacccggtgaagtctttggagtcttttgtaaagtgaaatgccaagattcgctcacaaagttcttgatctctctgtaccaaggaattgctggtgataatgttcccgagagttcactggttgagttctttaataaggaagggaagtacatagaagcatccttaaccgcgaacaagagtacaaacatcgaaggaaaagttataagatgtttcttcttcttgcagattatcaataaagaatcggggttgagctgcccagaactgaaagagagcgctcaaagcctcaacgaattaacttacgtaagacaagaaatcaagaatcctctcaacggtatccgatttgcacataagcttcttgaatcctcagagatttcagctagccaaaggcagtttctggagactagtgatgcttgtgagaagcaaatcacgacaataatcgaaagcacggacttgaaaagcattgaggaaggcaagttgcaattggaaacagaagagtttcgacttgaaaacatcttggacacaatcattagccaagtgatgattatattgagagagaggaactcacaactaagggttgaagtcgccgaggagatcaaaactctgcctctcaatggtgacagagtcaagctccagcttattcttgctgatcttctacgcaacattgtgaatcatgcgccgtttccaaatagttgggtaggtatcagtatctcaccagggcaggagctttcacgtgacaatggtcgctatatccatctacagttcaggatgatacatccggggaaaggacttccttcagagatgctaagtgatatgtttgagactcgagatggatgggtcacccctgatggtttagggcttaagctttcgcggaaactattggagcagatgaatggccgtgtgagttatgtccgagaagacgaacggtgtttctttcaggtggatcttcaagtgaagacaatgttaggtgttgagtcaagaggaacagagggtagttcaagcataaagtagaaattttgagttacaaggcttataaaatttaatgaaatttggtctcttaccaaatgtattaattagaacaaaggtacattatttattaccaccaataagattgtatcttaactatcctatgtatacaatttggagagatcatttggccaaccagacaaaagt

SEQ ID NO: 49 - DNA, 3704 bp, Artificial Sequence
  Synthetic Oligonucleotide
atcttcttcttcccccttcctctctcagcttctctcccaccactaaggaggaacgttccaaagatccctttctcagagaattcccagaaaaatcttcaacaattgaaaccctaatggagaatcattcggatccttgaatcctttggtttgtctctcacctccatttctgaaatttcattgctttgtgattcttctgcagattcgttttgaagagaagaaagaaatgtcatcgaacacttcacgaagctgttctactagatctagacaaaactctcgagtttcttcacaagttctcgtcgacgcaaagctacacggaaacttcgaagaatctgagcgtttatttgactattcagcttcaataaacttgaacatgccaagttcttcctgtgagattccttcttcagctgtctcaacgtacttacagaagattcagagagggatgttgattcaaccctttggttgtttaatcgttgttgatgagaaaaaccttaaagtcattgcctttagtgaaaacactcaagagatgttgggtttgattccacatacagtaccaagtatggagcagcgtgaagctttgactataggaactgatgtgaaatcattgtttctgtctccaggttgttctgctttggagaaagctgttgactttggtgagattagtattttgaatcctatcacgcttcattgtaggtcttcaagtaagcctttttatgcgattctgcatcggattgaggaaggtcttgttatagatttggagcctgtgagtcctgatgaggtgcctgtgactgctgccggggctttaagatcgtataagcttgcggcgaaatcgatttcgaggttgcaggcattgcctagtgggaatatgttgttgttgtgtgatgctttggttaaggaagttagtgaattaactggttatgatagggtgatggtgtataagttccatgaggatgggcatggggaagtgattgctgaatgctgccgggaagatatggaaccttatcttgggttgcattactccgctactgatataccgcaagcttcgagatttctgtttatgagaaacaaggttaggatgatttgtgattgttcagcggttccggttaaagtcgttcaagataagagtctctcacagccaataagtctttctggatctactttgagagctcctcatggttgtcacgcacagtatatgagtaatatgggatcagtggcgtctcttgtcatgtctgtaactatcaatggtagtgatagtgatgagatgaacagagatttacagactggcagacacttatggggcttggtggtttgtcatcacgcaagtcctagatttgttccgtttccattacgatatgcttgtgaattcttgactcaagtatttggcgtgcagatcaacaaagaagcggaatcagctgttctgttgaaagagaagcgtattttgcaaactcagagtgtgctatgtgacatgcttttccgcaatgcaccaataggtatagtcactcaatcaccaaatataatggatcttgttaaatgtgatggagcagcattatattacagagacaacctctggtctctaggagttactcccacagagacacaaattagagatctaattgactgggttctcaaaagtcatggaggaaacactggctttaccactgaaagtctaatggagtctggctatccggatgcttctgttcttggggagtcaatatgtggaatggctgccgtatatatttccgaaaaagatttccttttctggttccggtctagcactgcaaaacagatcaagtggggtggtgcaagacacgatcctaatgacagagatggtaagagaatgcatcctagatcctcattcaaggcttttatggaaatagtcaggtggaaaagtgtgccctgggatgacatggaaatggatgcaattaattctctgcagctaataataaaaggctcattgcaagaggagcattcaaagactgttgtggatgtcccacttgtggataatagggttcagaaggtagatgaattgtgtgttatcgtgaatgaaatggtgcggttgattgatacagcagctgttcccatctttgcggttgatgcctctggtgttataaatggttggaattctaaagcggctgaggtaacaggattggcagttgaacaagcaataggcaaacctgtatcagatctcgttgaggacgattctgtagaaaccgtgaagaacatgttagccttggctctcgaaggtagtgaagaacgtggtgctgagatcaggatcagagcatttggtcctaaaaggaaaagcagtccggttgagttagttgtcaacacttgttgtagcagagatatgacgaataatgttcttggtgtatgcttcattggacaagatgttacaggccagaaaacgcttactgaaaactatagccgcgtgaaaggagattatgcccgaatcatgtggagcccttccacactcattccaccaatttttataaccaatgaaaatggggtatgctcagagtggaacaacgcaatgcagaagctctctgggataaagagagaagaagttgtcaataaaattcttctcggggaggtttttaccacagatgattatggttgctgccttaaagaccatgacactttaacgaagctgagaataggtttcaatgctgtgatttctggccaaaagaacatagagaagcttttatttggcttttaccatcgtgatggtagcttcatcgaggcattgctttctgcaaacaaaaggactgatattgagggaaaggttaccggggttttatgctttttgcaagtacctagtccagaactccaatatgctctacaggttcagcaaatatcagagcatgcaattgcctgtgccctcaacaaattggcatatctccgccatgaagtgaaggaccccgaaaaggcaatatccttccttcaagatttgctccattcatctggattaagtgaagaccaaaagcggctcctgaggacaagcgttttatgcagggagcagttagccaaagtcataagcgactcagacatagagggaatcgaagaaggctatgtggaactggattgcagcgaattcggcctgcaggaatccctggaagcagttgtaaaacaagtgatggagctgagcatagaacgtaaagtacaaatcagctgcgattatcctcaagaagtttcatcaatgagattgtatggagacaacttaaggcttcagcaaatcctttcagagacactattaagcagcatacgcttcacgcctgcattgagaggattgtgtgtctcattcaaggtaattgcacggatagaagctataggaaaaagaatgaaaagagtcgaacttgagttcaggataatacacccggcaccaggactgcctgaggatctggtaagagagatgtttcagcctttgagaaagggaacatcaagggaaggtttgggattacacattacccagaagctggtgaaactcatggagagaggaacattgagatacctcagagagtctgaaatgtcagcctttgtgatcctcacagaatttcccttgatttgaagctgaagacctgtctacaagatttacattttattgaataagtgtggtgtttttgaaagtcttcatttttttttctcacatatataaatgtatgtttagttaccgtaacttaattacatatatatatataggaaagttaaatttg

SEQ ID NO: 50 - DNA, 3980 bp, Artificial Sequence
  Synthetic Oligonucleotide
ggagatattatcttgggctgccttgccgtgcagtgtggctcgctgtcccactcaggtgcggcgctattccaatccgtgcttatccccggcagaatccaatccccctcgcggcttttctccaaaagcgctccgccttctcgcctatttcgctcgctgccgcggccatgattccccgctgatacctccgcgcgcccatttggttcctcctcgccgaatcccccttcgcagccggtggttctagttgaggaggaggaggaggggatttggttgggggcgagggggaggagggggttgtggagatgtcgtcgtcgcggtcgaacaaccgggcgacgtgctcgcggagcagctcggcgcggtccaagcacagcgcgcgggtggtggcgcagacgccgatggacgcgcagctgcacgcggagttcgaggggtcgcagcgccacttcgactactcgtcgtcggtgggcgcggccaatcggtcgggcgccaccaccagcaacgtctccgcctacctccagaacatgcagcgcggccgcttcgtccagcccttcgggtgcctgctcgccgtccacccggagacgttcgcgctgctcgcctacagcgagaacgccgccgagatgctcgacctcacgccgcacgccgtgcccaccattgaccagcgcgaggcgctcgccgtcggcaccgacgtgcgcacgctcttccgctcgcacagcttcgtcgcgctgcagaaggccgccaccttcggggacgtcaacctgctcaaccccatcctcgtccacgccaggacctccgggaagcccttctacgccatcatgcaccgcatcgacgtcggcctcgtcatcgacctcgagccggtcaaccccgtcgacctgcccgtcaccgccacaggcgcgatcaagtcgtacaagctcgctgccagggccatcgccaggctgcagtccctgcccagtgggaacctctccctgctgtgcgacgtgctggtccgcgaggtgagcgagctcactggctatgacagggtgatggcgtacaagttccatgaggatgagcatggtgaggtgattgctgagtgcaagagatctgatttggagccgtatcttggcctgcactacccagccactgacattcctcaggcatccaggtttttgttcatgaagaacaaagtgcggatgatatgcgattgctccgcaacgcctgtgaagatcatccaagatgacagcctaacacaacctataagcatatgcggatctactctcagggcaccccatggttgccatgcacagtacatggcaagcatgggctccgttgcatcacttgtgatgtcggtcactataaatgaggatgaggatgatgatggagacactgggagtgaccagcagccgaaagggaggaagctgtgggggttgatggtctgccatcacacaagcccgaggtttgtccctttcccgcttaggtatgcttgcgagtttctcttgcaagtatttgggatacagatcaacaaggaggtggaactggctgctcaggcaaaggagaggcacatcctccgcacgcagactcttctctgtgatatgctccttcgagatgctcctgttgggatatttacccaatcacctaacgtaatggatctagtgaaatgtgatggtgcagcattgtattaccaaaaccagctttgggtgctaggatcaacgccctctgaagcagagataaaaaacattgttgcttggttgcaggagtaccatgacggttctactggattgagtaccgacagcttagttgaagcaggttatcctggcgctgctgcacttggtgatgttgtgtgtggcatggcagctataaagatctcttcaaaagatttcatcttctggttccgatcccacacggcaaaggagattaaatggggaggagctaagcatgaaccaattgatgcagatgacaatggtaggaagatgcatccacgatcttcattcaaagccttcttggaggtagttaaatggaggagtgttccttgggaggatgttgaaatggatgctatccattctctgcagctaatattacgtggctccttgcaagatgaagatgccaacaagaacaacaatgcaaagtccattgttacagctccatctgatgatatgaagaagattcaggggctccttgaactgagaaccgttacaaacgagatggtgcgcctaattgagacagcaactgcgcctatcttggctgttgacatcactgggagcataaacggatggaataataaggctgcagaactcactggattacctgtcatggaagccatagggaagcctctggtcgatctcgtcatcgatgattctgttgaagtggttaagcaaattttaaattcagctttacaaggaatagaagagcaaaatctgcaaattaagcttaaaacatttaatcaccaggaaaataatggacctgtaattttgatggttaacgcctgctgcagtcgtgacctttcagagaaagttgtgggggtttgctttgtagcacaagatatgacagggcagaacattatcatggataagtacactcggatacaaggggactatgttgctatagtaaagaacccttcggagctcatcccccctatatttatgatcaatgatcttggttcctgcttagagtggaatgaagctatgcaaaaaattactggtataaagagggaagatgcagtagataaattgctaatcggggaagttttcacccaccatgagtatggctgtagggtgaaagaccatggtactctgaccaaacttagcatattaatgaacacagtgatatctggtcaagatcctgagaagcttctttttggtttcttcaacaccgatggcaagtacatagagtcactgatgacagcaaccaagaggacagatgctgagggtaagatcactggcgccctttgctttcttcatgtggccagcccagagcttcaacatgctctgcaggtgcagaagatgtctgaacaagctgctatgaacagctttaaggaactgacatacatacgtcaagaattaaggaacccactcaatggcatgcaatttactcgaaatttgttggaaccttctgacttgactgaggagcagaggaaacttctagcatcaaatgtcctctgtcaagaacagctgaaaaagattttacatgacactgatctagaaagcattgaacagtgctacacggagatgagcaccgtagatttcaacctggaggaagccctgaatacagtcctaatgcaagccatgccccagagcaaggagaaacaaatttccattgaccgtgattggcctgcagaagtatcatgtatgcacctctgcggggacaatttaaggcttcaacaagtcctagcagacttcctggcatgcatgcttcaatttacacagccagctgaagggcctattgtgctccaagtcatccccaggatggaaaatattggatctggaatgcagattgctcatctagagttcaggcttgtccatccagctccgggcgttccagaggctctgatacaagagatgttccgccacagcccaggtgcatctcgagagggccttggcctttacataagccagaagctcgtgaagacgatgagcggcacggttcagtacctccgggaatcagagagctcgtcgttcatcgtcctggtagagttcccggtcgcccagctcagcaccaagaggtgcaaggcttccacgagtaaattctgaacttagggctatggccaaaattcttgtgggtgctgtattgttgttgttgctgctgctgctattagcagatcaggcagtctgaggagtacgttagctgaagatgtatcagatgtgtgtagcttgtgaaagtgaaggtagtggttggatagatgtatttgtcacgttgatgtctggagagctagtgagcgagctactaggtgttagtgtaatttaaccggcaaatgtatgaacgaattatgaataaaatcttcacttgtgttgtcatga

SEQ ID NO: 51 - DNA, 6176 bp, Artificial Sequence
  Synthetic Oligonucleotide
aagagaaaaaaggttgaaagagacccccggttctttgtgacccaccttcaaacggggagtagggttttgcgaggaaccggaacctccagtaaaaacaaatcccacgtgtctcactcccttattcgcttgcaaatttgttttgcaccctctctcgcggactcgattatatttctaacgctaacccggcttgtagttgtgattatttttgagaatttcagtttcgccctattcaccccctctaggcgactttcagagagacgtgtctgacttagcctcgccgaagctgatgaagaccaagtaggagacgaagcgactgtaccaggcccgtgacgcctgcttgaggccgtatagggagcggttcagccggcagaccagattcgggcgagcagcgtcgacgaagccggtgggctggtagtatacagtctctgaaagagaaataggcttaaaacattttctataatttgtttgacgtccatcacaaatcactataagtgtaagacatatctagatttgtccttaaataaatggggtggcaagattttcatttgggaaatgctccagttatgcatccacccactgtgcagtttctggcgattgctgccgaaccctactcggaggggttcgcgatacaggtgttcgtgaagccgtgacgaggggcttcaccttcacaggacacatgctaacaagtaggagtaatctaatcatagggcaaccactgtaataatcgagtacgtacgatataactaatctagcataatccatcgttaccagagatggaaatggcggcgacttctgcggagacaacaacccagatgacgctagactactttccactctcggatccctgactaccactcgtcagtcgtccccaactgcagtgcgggtctaatctagccccgcgaattccccaattttcgccgctccaattcgctcatccgtccgcgcgcccctcgccgaccggccggcgcggcgccggccgacgtcctctctccggggtagtcagtcgcgcactgcgcatcgctgttgttccttccggttcctttcgcgtccccccttcggagcagggcaacgtccaagagatattatcgctgccgctgccccgcgcgggccgctcgctgtcccactcgtgcgccacgcctcggactcggaggtgtattccctcttctccccccgtgcttatccgcgacggaatccaatcccttcgctcaagtccttctcctaagcaaagccccctcgcttttggcgtatgcccccgctggcgcgcccatgattcccgccgattcctccgcccgttcagtccagcaacccttgctgctgcagtgctgcggctagttcggtggcggggaaggaggaggaggcgagatcttggagtggggcgaagcggagatgtcgttgccgtcgaacaaccggaggacgtgctcccggagcagctctgcgcggtccaagcacagcgcgcgggtggtggcacagacgcccgtggacgcgcagctgcacgccgagttcgagggctcccagcgccacttcgactactcctcgtcggtgggcgccgccaaccgcccgtcggcaagcaccagcaccgtctccacctacctccagaacatgcagcggggccgctacatccagcccttcggctgcctgctcgccgtccacccggacaccttcgcgctgctcgcctacagcgagaacgcgcccgagatgctcgacctcacgccacacgccgtccccaccatcgaccagcgggatgcgctcggcatcggcgtcgatgtgcgcacgctcttccgctcgcagagctccgtcgcgcttcacaaggccgccgccttcggggaggtcaacctactcaaccccatcctcgtgcacgccaggacgtcggggaagcccttctacgccatattgcaccggatcgacgtcggccttgtcatcgaccttgagccggtcaatccagccgacgtgccagtcaccgccgcgggcgcgctcaagtcgtacaagctcgctgccaaggccatctccaggctgcagtcgctgcccagcgggaacctgtcgttgctgtgcgatgtgcttgtccgtgaggtgagcgaactcacgggctatgaccgggtcatggcctacaagttctatgaggatgagcatggcgaggtcatttccgaatgcaggaggtccgatctggagccgtatcttggactgcactacccagccaccgacatcccgcaggcgtccaggttcctgtttatgaagaacaaagtgcggatgatatgtgattgctgtgccactccagtgaaggtcattcaggatgatagcctagcacaacctctcagcctctgtggttccacactcagggcttcccatggttgccatgcacagtacatggcaaacatgggttctgttgcatcacttgcgatgtcagtcactataaacgaggatgaggaggaagatggggataccgggagtgaccaacaaccgaaaggcaggaagctgtgggggcttgtcgtctgccatcatacaagcccgaggttcgtccctttcccactaaggtatgcttgtgagtttctcttgcaagtatttggcatacagctcaacaaggaggtggaattggctgctcaggcaaaggagaggcacatcctcagaacgcaaacccttctttgtgatatgctcctgcgggatgctcctgttgggatatttactcggtcacctaatgtgatggatctagtaaagtgcgatggagctgcattgtattaccagaaccagcttttggtgcttggatcaacaccctctgagtcagagataaagagcattgcaacatggctgcaggataatcatgatggttcaactgggctgagtactgacagcttagtggaagcgggttatcctggtgctgttgcacttcgtgaagttgtgtgtggcatggcggccataaagatctcttccaaagattttatcttctggttccgatcgcacacaacaaaggagatcaagtggggtggggctaagcatgaaccggttgatgcagatgacgatggcaggaggatgcatccacgatcttcattcaaggccttcttggaggtggttaaatggagaagtgttccctgggaagatgttgaaatggatgctatccattctttgcagttaatattacgtggctccctgccagatgaagatgccaacagaaacaatgtaaggtccattgtaaaagctccatctgatgatatgaagaagatacaggggctacttgaactgagaacagttacaaatgagatggtccgcttaattgagacagcaactgcccctgtcttggctgtcgacattgccggtaacataaatggatggaacaataaagctgcagaactaacaggtttacctgtaatggaagccatagggaggcccctgatagatcttgttgttactgattctatagaagtggttaagcagattttggactcagctttacaaggtttgtcactttgctttcagtcgatttttttgtggtccccagcttagtctgtgaatgtctcctttttttctatgacaacctagtgcctccttgttttcaggaattgaagagcaaaatatggaaatcaagcttaaaacattccatgaacatgaatgcaatggtccagtaatcttgaaggttaactcctgctgtagtcgggacctttcagagaaagtcattggagtttgctttgtagcacaagatttgaccaggcagaagatgattatggataagtatactaggatacaaggagactatgttgccatagtaaagaaccccactgagctcatccctcccatatttatgatcaatgatcttggttcctgcttagagtggaataaagctatgcagaagattaccggtataaagagggaagatgcgataaacaaattgttaattggggaggtcttcacgcttcatgattatggctgtagggtgaaagatcatgcaactctaacgaaacttagcatactgatgaatgcagtgatttctggtcaggatcctgagaagctcttttttggtttcttcgacacagatgggaagtatattgaatccttgctgacagtgaacaagaggacagatgctgagggtaagatcactggtgctctttgctttctgcatgtggccagtccagagcttcagcatgctctccaggtgcagaaaatgtcggaacaagctgcgacaaacagctttaaggaattaacttacattcgtcaagaattaaggaacccactcaatggtatgcaatttacttgtaacttattgaagccttctgaattgacagaggaacagcggcaacttctttcatctaatgttctctgtcaggaccagctgaaaaagattttacatgacactgatcttgaaagcattgaacagtggtaagttctatattgtctgaatcatctgtattaatgttgatttaattatttgtgttgtttgtgtggtaattctagaagggatctatgaaacaccatacactatacataaagatatatttgctttcatattcacatgatggttagaattgctcagacatgaattttttgtccattgtgctaatgctttgtagtgctaaagatgcatgcatccgacaaggctgaccagtgaccagttcagttgtacatgcatgagaatttttttgactattgataaacaaaattcttgctgttacagctatatggagatgaacacagtagagttcaaccttgagcaagctctgaatacggttctgatgcaaggcattcctttgggcaaggaaaaacagatttcaattgaacgtaattggcctgtggaagtatcatgcatgtacctttatggggacaatttaaggcttcagcagatcctagcagactatctagcatgcgcccttcaattcacacaaactgctgaaggacctatcgtgctccaggtcatgtctaagaaggaaaacattggatctggcatgcagattgctcatttggagttcaggtaatacaccaatctcatcttggtctttcgatagccgttgttgaactgaaaggatgatttcacaaataagtgaaccactaaattgtacatctagtttctatgaacccagcacgactccaacagtatgtttctaataactctggcactgctcctaatcgtgttagttgtaatccacgaaaaggactgtatgttgctgccaaggaacagataagaccttatgctagcaaattcacttctgaacatcttaactcctggtagttttgtatcctgtcactgcaccagccaagatgcagtgacacatgtcacttcaggcatatagttacactttgcagtttgaaactgtggaaatgatccatgtgtaaagtccatgctgtccctgccattctatttacactgcagtgtggtgaatgtaagtaatggtggtaatgggagccgtgtaactgattgactctgtgcaggattgtccatccagctccaggcgttccagaggccctgatacaggagatgttccagcacaacccaggggtgtccagggagggcctcggcctgtacataagccagaagctggtgaaaacgatgagcggcacggtacagtaccttcgagaagccgacacctcgtcgttcatcatcctgatggagttcccggtcgcccagctcagcagcaagcggtccaagccttcgacgagtaaattctgaccctcatgctgctggaactgcagcgagtgttctgccgtgtcgaagtctcggttgtagctagccaagtggctgctactgaaacatcggttggtcggttctggtatctagcacggcctgtgtagtgcggatagatttggttgtttggctttggagagataagctagctggctaggtgtaacgtaacgtcgaccgataactgtatgtgaatgatgatgagctgtgaggacgaataaaggcttcacttgtgttctctattgctcttcttagagtatgatggccaacagctgtacaggaaagcatccacctaaatctgggctcgctgaatgacggctaaaagttgtttggtggctttaaactcttttatgctttgtaatcccttacagtcggatgttggtctttaaactgtcagttaaatgctggtacggcggtaggtctgttttcttgatctctctgttgagcaaggccccccttttgttcggttagattacatgtaaccgttccgtttcttctgtttcttccgccctatatc

SEQ ID NO: 52 - DNA, 3408 bp, Artificial Sequence
  Synthetic Oligonucleotide
atgtcgtcgccgtcgaacaaccgtgggacgtgctcccggagcagctctgcgcggtccaagcacagcgcgcgggtggtggcgcagacgcccgtggacgcgcagctgcacgccgatttcgagggctcccagcgccacttcgactactcatcctcggtgggcgccgccaaccgcccgtcggccagcacgagcaccgtctccacctacctccagaacatgcagcggggccgctacatccagcccttcggctgcctgctcgccgtccacccggacaccttcgcgctgctcgcctacagcgagaacgcgccggagatgctcgacctcacgccacacgcggtcccaaccatcgaccagcgggacgcgctcaccatcggcgccgacgtgcgcacgctcttccgctcgcagagctccgtcgcgcttcacaaggccgccaccttcggggaggtcaacctgctcaaccccatcctcgtgcacgccaggacgtcggggaagcccttctacgccatattgcaccggatcgacgtcggccttgtcatcgaccttgagccgttcaacccagcagacgtgccagtcacggccgcgggcgcgcttaagtcgtacaagctcgccgccaaggccatctccaggctgcagtcgctgcccagcgggaacctgtcgttgctgtgcgatgtgcttgtccgtgaggtgagcgagctcacgggctatgaccgggtcatggcgtacaagttccatgaggatgagcacggtgaggtcatttctgagtgcaggaggtccgatctggagccgtatcttggcctgcactacccagccaccgacatcccgcaggcgtccaggtttctgtttatgaagaacaaaatgcggatgatatgtgatttctctgccactccagtgctgatcattcaggatggcagccttgcacagcccgtcagcctctgtggttctaccctcagggcttcccatggttgccatgcacagtacatggcaaacatgggttctgttgcatcgcttgtgatgtcagtcactataaacgacgatgaggaggaagatggggataccgacagtgaccaacaaccgaaaggcaggaagctgtgggggctggtcgtctgccatcatacaagcccgaggtttgtccctttcccgctaaggtacgcttgcgagtttctcttgcaagtatttggcatacagctcagcaaggaggtggaactggctgctcaggcaaaggagaggcacatcctcagaacgcaaacccttctttgtgatatgctcctgcgggatgctcttgttgggatatttacccagtcacctaatgtgatggatctagtaaagtgcgatggagctgcattgtattatcagaaccaggttttggtgctcggatcaacaccgtccgagtcagagattaagagcattgccacatggctgcaggagaaccatgatggttcaactgggctgagtactgacagcttagtggaagcgggttatcctggtgctgctgcactccgtgaagtcgtgtgtggcatggtggccattaagatctcttccaaaaattttatcttctggttccgatcacacacaacaaaggagatcaagtggagtggggctaagcatgaaccgtttgacgcagatgacaatggcaggaagatgcatccacgatcttcattcaaggccttcttggaggtggttaaatggagaagtgttccctgggaggatgttgaaatggatgctatccattctttgcagttaatattacgtgactccctgcaaggtgaagatgccaacagaaacaacatcaggtccattgtaaaagctccatctgatgatatgaagaagttacaggggctacttgaactaagaacagttacaaacgagatggtccgcttaattgagacagcaactgcccctgtcttggctgttgacattgccggtaacataaatggatggaacaaaaaagctgcagaactaacagggttacctgtaatggaagccatagggaggcctctgatagatcttgttgttgctgattctgttgaagtggttaagcagattttggactcagctttacaaggaattgaagagcaaaatctggaaatcaagcttaaaacattccatgaacaggagtgttgtggtccagttatcttgatgattaactcctgctgtagtcgggatctttcagagaaagtcattggagtttgctttgtagcacaagatttgaccaggcagaagatgattatggataagtatactaggatacaaggagactatgttgccataataaagaaccccagtgagctcatccctcccatatttatgatcaatgatcttggttcctgcttagagtggaataaagctatgcagaagattactggtatgaagagggaagacgcgataaataagttgttaattggggaggtcttcacgctccatgattatggctgtagggtgaaagatcatgctactctaacgaaacttagcatactgatgaatgcagtgatttctggtcaggatccagagaagctcctgtttggtttcttcggcacaggtgggaagtatattgaatccttgctgacagtgaacaagagaacaaatgctgagggtaaaatcactggcgctctttgctttctgcatgtggctagcccagagcttcagcatgctcttgaggtccagaaaatgtctgaacaagctgctacaaacagctttaaagaattaacttacattcgtcaagaattaaggaacccactcaatggcatgcaatttacttataacttattgaagccttccgaattgacagaggatcagcggcaacttgtttcatctaatgttctgtgtcaggaccagctgaaaaagattttacatgacactgatcttgaaagcattgaacagtgctatatggagacgaacacagtagagttcaaccttgaggaagctctgaatacggtcctgatgcaaggcattcctttgggcaaggaaaaacgtatttctattgaacgtgattggcctgtggaagtgtcacacatgtacatttacggggacaatataaggcttcagcaggtcctagcagattatctggcttgcgcccttcaattcacacaaccagctgaaggacatatcgtgctccaggtcattcccaagaaggaaaacattgggtctggcatgcagattgctcatttggaattcaggattgtccatccagctccaggcgttccagaggccctgatacaggagatgttccagcacaacccaggggtgtccagggagggtctcggcctgtacataagccagaagctggtgaaaacgatgagcggcacgttgcagtacctacgagaagccgacacctcttcgttcatcatcctgatagagttcccggtcgcccagctcagcagcaagcggtccaagccttcgccaagtaaattctga

SEQ ID NO: 53 - DNA, 4366 bp, Artificial Sequence
  Synthetic Oligonucleotide
gaatggaattggtgaggtggagtggtgtgtgcaggctggtgctgatccgaggtgcttgtggtagttcaagactcagtagattgccaagttcgggtcctcgtactgtgtgagtgcaagagagggaggaggtagggaggtagggaaagagagatagagagaacagtggtgttgtttccgtggtgtgattggagtgttgctgttgcgaatgtgggttctgtttgatgatttctatctaattagtgaggagaaggaaacgtgctctagatatgggggagatacggggaccctgccgtcagcggaagcatatgttaggtatttgtgcggcaggttgaaaactatgtgtggtcaagacgttgctgcgaaggcgttctctggtatgtagtggagcacgccgtgagtcaattttggccgtaatggtgtgttttggctttacaggtggagtcctggagtttcgagattctgtgaaaattttcttttcctaacgtcgggcaattgaagcttttcggctacctttctgctaccggatctcggagttggttcgtgcacttgagccttggatgcaggaggggctgcacgacagctatgatgctatcgcggatccaaaagaagattcttcaaacctaaattttttgagtaatttagcaagatgtcgactcccaagaagacatactcctcaacaagctcagcaaagtccaaggcgcatagtgtgagggtcgcccaaacaaccgcagatgcagcacttcaagctgtgttcgaaaagtccggagattctggagattcattcgattattcaaaatctgtcagcaaatcaacagctgagtcgcttccttcaggggcagtgacggcctaccttcagcgtatgcagaggggaggtctcactcaaagtttcgggtgtatgatagcagttgaaggaacaggatttcgtgtgatagcgtacagtgaaaatgcacccgagattctagacctggtgccacaggccgttccaagcgtgggggagatggacacgctacgaatcgggacggatgtgagaacgctgttcacagcttcaagtgtcgcctctcttgagaaggcagctgaagcacaagaaatgagcctactcaatccaatcaccgttaactgcagaagatctgggaagcaattatatgccattgctcatcgcattgacataggtatagtgatcgactttgaggcagtaaaaacagacgatcatttagtttcagctgctggtgcactgcaatctcataagcttgcagccaaggcaatcacacggcttcaagcattacctggaggtgacataggattgctctgtgacactgttgtcgaagaggttcgggagctcactggttatgacagggtaatggcttatagatttcatgaggatgagcatggagaggttgtggctgagatacgtcgcgcggatcttgagccttacttaggtcttcattatccgggcactgatattccccaggcgtcccggtttctgtttatgaagaacaaggtgcggataattgctgattgttccgcacctccagtgaaagttatacaagatccaaccttgaggcagccggtcagcttggcaggttcgactttacgttctcctcacggatgccatgcccagtacatgggcaatatggggtccattgcgtccctagtcatggctgtgatcatcaacgataacgaggaagattcgcatggctcagtccaaagaggtagaaagctatggggacttgtagtgtgtcatcatacatctccacgaactgtaccgtttccccttcggtccgcgtgtgggtttttgatgcaggtgttcggattacagcttaacatggaggtcgagttagccgctcagctaagggaaaaacatattctcagaactcaaactctactttgcgacatgcttcttcgagatgctcctattggtattgtttctcagattccaaatattatggaccttgtgaaatgcgatggagctgctctttattatgggaagcgattctggcttctgggtacgactcctactgagagtcagattaaagatattgcagaatggttgctagaataccacaaggactcaacaggtctgagtactgatagtttagcggatgccaattatccagccgcacacctgctcggggatgctgtgtgtggtatggcagctgcgaagatcactgcgaaggattttcttttttggttcaggtctcatactgctaaagagattaagtggggtggtgctaaacacgatccaggcgaaaaggacgatggtcgaaaaatgcaccctagaagctcttttaaagcctttttggaggtcgtgaagcgacgaagtctaccctgggaagatgtagaaatggatgctatacattctcttcagctcattctgcgtggatcctttcaagatattgatgacagcgatacgaagactatgattcatgcacgcctgaatgacttgaagcttcatgacatggatgagctcagcgtagtggcgaatgaaatggtacgcttgatagaaactgcaactgctccgatactggcagttgattcaaacgggatgatcaatggctggaatgcaaaaatagcccaggtaacaggacttcccgttagtgaggccatgggtagatccttggtgaaagatctcgttacggatgagtcagtggcggttgtcgagagattactttatctggcactgcgaggtgaggaagaacagaatgtggaaatcaagttgaagacctttggcactcagactgaaaaaggagtggttattctgattgttaacgcctgctcgagtagagacgtttcggagaatgttgttggtgtttgctttgtgggtcaggatgtgactgggcaaaaaatgttcatggacaagtttactcgtatacagggtgattacaagactatagtccagaatcctcaccccctcattcctcctatttttggtgctgacgaatttgggtactgtttcgagtggaatccagccatggagggcttgactggttggaaaaaagacgaggtggtcggaaaactccttgtgggagaaatcttcggcatgcaaatgatgtgttgtcggatgaagagccaagatgccatgacgaaattcatgatagcattgaacacagctatggacggccaaagtacagataaatttactttctcctttttcgatcgagaagggaaatacgttgacgttctactctccaccaataaacgtaccaatgctgatggagtcataactggcgtgttttgcttcttacaaattgcaagttcggagctacaacaggctctcaaagtacaacgggctacggagaaggtcgcagtagctaagctgaaggaacttgcatatattcgtcaggagattaaaaatccactatgtggcattacattcacacgacaattgttagaagatactgatctatcagacgatcagcagcagtttctagacacgagtgccgtatgtgagcagcagttacaaaaagttctgaatgacatggatttggagagtatcgaagacgggtacttggagttggatactgccgagttcgagatgggcacagtgatgaacgccgtcatcagtcaaggtatgacaacatcccgtgagaaggggcttcagatttttcgagagacacctcgagaaataaatacaatgcgtctgcttggagatcagattcggctccagcaagttctttctgattttctattaaacacagtgcgatttacgccttcacctgaaggttgggtcaaaatcaaggtggtccccactaggaaacgtcttggcgggagtgtgcatgtcgtgcatttagaattcagggtaagtcatcctggagctgggcttcctgaagagcttgtgttggagatgtatgatagaggcaaaggcatgactcaagaaggactaggtttgaacatgtgtcggaagctcgttaggttgatgaatggggatgttcattacgtacgggaagctatgcagtgttattttgttgtaaatgtggagctcccaatggctcagcgggatgatgcttcaagtcaaatgtaggagtttatactccctatttgcttgcttagatactcttataggtggttctttgggacctctctctagtaacgagagagtttaagggtgaaagtagagaagtttgactttacttttttagatgtccagttcaactttacttaggtgtctccaaatgagacaatcttgcctctaagcccggcaaatcaaagtgcctacgttgatacacaagctatccttttgagggaattagctatgtcgtctacgatgtacagcaatttccttttcttgcaaattcagttcaactccttttcacggtttgcattgaagcgcttcaatttttggaaacctggagttgacacttgttttcca

SEQ ID NO: 54 - DNA, 3369 bp, Artificial Sequence
  Synthetic Oligonucleotide
atgtcggctccgaagaagacttactcctccacaagttcggcgaagtcgaaggcccacagcgtgagggtcgctcaaactacggcagatgcagcccttcatgctgtgtttgaaaagtccggagtttctggagataattttgattactcaaaatctgtcagcaaatcgacagcagggtctcttcatactggggcagtgactgcctacctccaacgtatgcagaggggaggactcactcaatcctttgggtgcatggttgcagttgaggagacagggtttcgtgtcatagcgtacagtgaaaatgcacccgagttcctagatctgatgccgcaagctgtaccaaacattggggaaataaacacgctaggaatcgggacggatgtgagaacgctgttcaccccttcaagtgctgcctctctcgagaaggcggccgaaacacaagaaataagcttactcaatccgatcaccgtctattgcagatctaaaaaacctttgtatgccattgcacatcgcatagacataggcatagtcatcgattttgaggcggtgaatatgaacgatgttacaatatcagctgatggtgcattgcaatctcataaacttgcggcgaaggcaattacacggctccaagcactacctgggggtgacataggactgctctgtgacactgttgttgaggaggttcgagagctcactgggtatgacagggtaatggcctacaaatttcatgaggatgagcatggtgaggtggtcgcagaaattcgtcgcacggatcttgagccctacttgggtcttcattacccggccactgacattccccaggcgtctcggtttctatttatgaagaacagggtccggatgattggtgattgttctgctcctccagtgaaaattgtacaagatccaaacttaaggcagccagtcagcttggcaggttcgactttacgttcccctcacggatgccatgcccaatacatgggcaacatggggtccatttcatcgatagtcatggccgtgatcatcaatgataacgaggacgattctcgtggctcagttcaaaggggtagaaagctatgggggctagtagtgtgccatcacacatctccacgtactgtgccatttccacttcggtctgcgtgtgagtttttaatgcaggtgtttggtttacagcttaacatggaggtcgagttagccgctcagcttagggaaaaacatattctcagaactcaaactcttttatgcgacatgcttcttcgagatgcccccattggtattgtgtctcaggttccaaacattatggatcttgtgaagtgtgatggagctgctctttattatgggaaacgattctggcttttgggcacgaccccgaccgaaagtcaaattaaagatattgcagaatggttgctagagtaccacaaggactcaacgggactcagtactgatagcttagcggatgccaattacccagccgcacacctccttggggatgctgtgtgtggtatggcagctgcaaagatcacttccaaagattttctgttttggttcagatctcacactgcgaaagagataaagtggggtggtgccaaacacgacccaggcgaaaaagatgataatcgaaaaatgcaccccagaagctctttcaaagcctttttggaggtcgtgaaacgacgaagtctaccctgggaggatgtagaaatggacgctatacattctcttcagcttattctacgtggatcctttcaggacatcgatgacagcgacacaaagactatgattcatgctcgtctgaatgatttgaagcttcatgatatggatgagcttagcattgtagcgaatgaaatggtacggttgatagaaactgcgaccgcccctattctggcagttgattcaaatggaatgataaatggctggaatgccaaaatagcccaagaaacagggcttccggttgctgaggctatgggcagatctttggtgaaagacctcgtcatggatgagtcattggaggttgttgaaagactactttatctggctttgcgaggtgaagaagagcagggggtggaaatcaaattgaagacctttggcgctcaaactgtgaaaggagcggttattctgattgttaatgcctgcgctagtagagacgtgtcggagaatgttgttggcgtttgttttgttggtcaggatgtaactggacaaaaaatgtttatggacaagttcactcgtattcagggtgattacaaaaccatagtgcagaatcctcaccctctcatccctcctatttttggtgcggacgaatttggatactgtttcgaatggaatccggccatggagggtttgactggttggaagcgagatgaagtgatcggaaaacttcttgtaggagagatttttggcatgcaaaagatgtgttgccagatgaagagtcaagacgccatgaccaagttcatgatatcattgaactcggccatggatgggcaaaacacagataaattttccctctccttttttgatcgagaagggaggtatgttgatgctctgctctcaaccaacaaacgcactaatgctgatggagctataactggcgtcatttgcttcttacaaattgcgagttcggagctacaacaggcccttagagtacagcaagctacagagaaggtcgcaatagcgaagctgaaggagcttgcatacattcgtcaggagataaagaatccactatgcgggatcacattcactcggcaactgctagaagacactgatctctcaaatgatcagaagcagtttctagacacaagtgctgtgtgcgagcagcagttgcaaaaagtattgaatgacttggacttggagagtatcgaagacgggtacttggagttggatactgctgaatttgagatgcgtacagtgatggacgccgttatcagtcaaggtatgacgatatcccgtgagaaggggcttcagatcattcgagagactcctcgggaaataatcactatgcgtttgtttggagaccaggttcggctccagcaagttctctctgatttcttattaaacgcagtgcgttttacgccttcctctgaaggctgggtcaaaatcaaggtggtccctacaaggaaacggcttggtgggaatgagcatgtcatgcacttggaattcagagtaagccatcctggagctgggcttcctgaagagcttgtgctggagatgtttgataaaggcaaaggcatgacccaagaaggactaggtttgaacatatgtcggaagctggttaggttgatgaatggggacgttcagtacgtgcgtgaagctatgcagtgttactttgttctatatgtggagcttcctttggctcagcaggatgatgctgcaagtcaaatgtag

SEQ ID NO: 55 - DNA, 4191 bp, Artificial Sequence
  Synthetic Oligonucleotide
ccgcagctgctgtgcctccttgcttggcgcgctcgctcttcgctctcttgaggctggggcgagatttagatttgggttgaagcccacccacaattggattggtactgtttaggatagcacagccacatagatagcctctaggccgttcctcctctgagcagagcgacgacgagagcatcctgcgtgagtgcgtgcgcgtgatgcacatcttaggtaagtaagcaagggggagagcaaaaatgtctactaccaagctgacgtactcgtcgggcagctccgccaagtccaagcacagcgtccgagttgcccagacgactgccgatgccaagctccacgccgtgtacgaggagtctggcgagtccggggactccttcgactacagcaagtccatcaatgccaccaagtcgacgggcgagacgatcccagcgcaggccgtgacggcgtacctgcagcggatgcagcggggtggtttggtgcagccgtttggctgcatgctggcggtggaggagggcagcttccgtgtgattgccttcagtgacaatgcgggggagatgctggacctcatgcctcagtcggttccaagcctgggcagcgggcagcaggacgtgctgaccatcgggaccgacgcccgcaccctcttcacggcagcagccagcgcgctggagaaggcggcaggagccgtcgacttgagcatgctcaacccgatatgggtacagtccaagacgtcggccaagcccttctacgccatcgtgcaccgcatcgacgtgggcctggtcatggacctggagcccgtcaaggccagcgacacgagggtgggatccgctgccggggctctgcagtcccacaagctggcggccaaggccatatcgcgcctccagtcgctgccgggcggcgacatcggcctgttgtgcgacacggtggtggaggaggtgcgggacgtgaccggttacgacctggtcatggcatacaagtttcacgaagacgagcatggcgaggtggtggcggagattcggcgctccgacctggaaccgtacctcgggctgcactacccagccacggacataccccaggcttctcgattcctcttcatgaagaaccgggtacgcatgatctgtgactgctccgccccgcccgtgaagatcacccaggacaaggagctgaggcaaccgatcagcctggctggctccacgctgcgagcaccgcacggatgccatgcgcagtacatgggcaacatgggctctgttgcgtccctggtcatggccatgatcatcaacgacaacgatgaaccgtctggcggcggcggcggcggcgggcagcacaagggaagaaggctgtgggggctggtggtgtgtcaccacacgtccccgcgatcggtgcccttcctgcgctcggcgtgcgagttcctcatgcaggtgtttggcctgcagctcaacatggaggcggcagtggcggcgcacgtgcgggagaagcacatactgcgcacccagacgctcttgtgcgacatgctgctgcgcgacgctcccatcggcattgtttctcagtcgccaaacatcatggacctggtcaagtgcgacggtgccgcactctactacggcaaacgcttctggttactgggcatcacgcccagcgaggcacagatcaaagacatcgccgagtggctgttggagcatcacaaagactcaaccggcctgagcaccgacagcctggctgacgcaggctacccgggtgctgcgtcgctaggggacgaggtgtgcggcatggctgccgccaagatcaccgcgaaggatttcctgttttggttccggtcgcacactgccaaggaggtgaagtggggtggtgccaagcacgacccggacgacaaagatgatgggcgcaagatgcatccccgctcctccttcaaggcctttctggaggtggtgaagcgccgaagcctgccgtgggaggacgtggagatggatgccatacactcgctgcagctcatcctccgtggctccttccaggacattgacgacagcgacaccaagaccatgatccatgcgcggctcaacgacctcaagctccagggcatggacgagctcagcaccgtggccaacgagatggtgcgcctcattgagaccgccacggcacccatccttgccgtagactccagtggcttcataaacggctggaacgcaaaggtggcggacgtcaccggcctgcccgtgaccgaggccatgggacggtccctggccaaggaactggtgctccacgagtccgccgacatggtggagaggctgctgtacctggcgcttcaaggtctataacagacagtatcgtatatgtcaactaacgttcgtgccttgcatgcccaggtgacgaggagcagaacgttgagctcaagctcaagacttttggtggccaaaaggacaaggaggcggtcattctggttgtcaacgcgtgcgccagcagggacgtgtcggacaacgtcgttggcgtgtgcttcgttgggcaggacgtgactgggcagaaggtggtcatggacaagttcacgcgcatccagggcgattacaaggctattgtgcagaatccgaatccgctcataccgccgatcttcggggcggacgagtttggatactgctctgagtggaatccggccatggagaagctgtctgggtggagacgtgaggaagtactaggtaagatgctggtgggagaaatatttggcatccagatgatgtactgccgccttaagggccaggatgccgtcaccaagttcatgatcgtcctcaacagtgcagcagatggccaggacactgaaaagttcccgtttgcgttcttcgatcgacagggaaagtatgttgaggcgttactcactgccaccaagcgggcagatgccgagggctccatcactggagtgttttgcttcctacacattgccagtgcggagttgcaacaggccttgacagtccagagagccactgagaaggttgcattatccaagctcaaggagctggcttacatccgccaggagataaagaatccgctctatggtattatgttcacccggactctcatggaaactaccgacctgtccgaggaccagaagcagtatgttgagactggagccgtgtgcgagaagcaaatccgcaagatcctggacgacatggatctggagagcatcgaagacgggtaagtgacaattgtgtgtgcattggtcttctcattcatgataatgtcaggtacctggagctggatacaaccgagttcatgatgggaacggttatggacgctgtgataagtcagggcatgatcacgtccaaagagaagaacttgcagctcatccgggaaactccaaaggagatcaaggcaatgttcctttatggagatcaggtgcgcctccagcaggttctggcagattttctgctcaatgcaatccgcttcacaccgtcgtcagagaactgggtggggataaaggtggccacatctagaaagcgcttgggtggtgtggtgcacgtgatgcacctcgaattcaggtgagagtccaagggtttctgtttccgtttgtttgtccttcccgtttctcactcttgctatgcattgcaggataactcatccgggggttggccttccggaagagcttgtgcaagagatgtttgacaggggccgaggcatgacacaggagggcctggggcttagtatgtgccgaaaactcgtcaagctcatgaacggagaggttgaatatatcagagaagctggcaaaaactacttcttagtcagcctggagctccctcttgcacaacgggatgatgctggcagtgtcaagtttcaggcttccagctagctaattctctatggcgtgtaccacaggcatcgtcgtagtgatgtagagacttggaatacctgccgtggatgttgggccaccagacagatccctgcacatgccatggcaaagcgactgcgaccttaacaatggtctttctcttgtatctcaggcgagtgaactgcagaaggtacgacctctgtggaggaggtggagatgcatctggcacaacctggaataaatgcaagttagcaaatgacgctgtagaagtacacgcaccttagaaaagcattcattctgaaaaagtagtagagcattgatgatgtctgccctctgtgcattactgctagttggagagcgttctggagatcaacccgatgtgcttgccctgc

SEQ ID NO: 56 - DNA, 3850 bp, Artificial Sequence
  Synthetic Oligonucleotide
atgtcgacccccaagttggcttactcgtcagggagttctgtgaagtcaaagcatagtgtccgggtggcgcaaactacggcagatgccaaactgcaggctgtgtacgaagaatctggagattcaggagattcatttgattactctaaatcagttcatgcgtctaaatccacgggtgagaatgtttctgctcaagcagttaccgcataccttcagcgtatgcaaagagggggtctgatgcagactttcgggtgtatgttatgcgtggaagagagtaattttagggttattgccttcagtgagaatgccccagagatgcttgatttgatgccacaagctgtgccaagtgtgggccaacaggaagttctgggcattggaacggatgccagaactttgttcaccccttccagcgccgctgccttggagaagtgcgcaggagctgtggatgtgactatgctgaaccctatctcagtgcactgtcgaagctcggggaagccgttctatgcaattctgcaccgtatcgatgtgggactggtgatcgattttgagccagtgcggtcgaacgatgccattgtgtcctccgctggggtgttacagtcgcataagttggctgcaaaggcgatttctcggctgcaggcacttcctggcggagatattggtctgctttgtgatattgttgttcaggaagtgcgcgagctttcaggctacgatcgtgttatggcttacaaattccacgaagacgaacacggtgaggtcttagcagaaattcggagatcagatttagaaccttacttgggcttacattacccagcaacagatatcccacaggcatcaagattcctgtttatgaaaaataaagttcgaatgattggtgattgctttgcttctcctgtgaaagtgattcaggacaaggatttgaggcagcctatcagtcttgcgggatctacattacgcgctcctcacggttgccatgcgcagtatatgggtaatatgaactctattgcttccttagtgatggctgtgattgtgaatgaccctgacgaggatcctaatgcacgcgggggtcagcaaagagggcgcaagctctggggattggtagtttgtcaccacacgtctccgaggaccgtctcttttcccttgagatcggcttgtgagttcttaatgcaggtatttggtttgcagctgaatatggaggttgagcttgcagctcagctaagagagaagcatattttgagaactcaaaccctcctctgtgatatgttgttgcgggatgcacctataggaatcgtatcccaatcacccaatatcatggatttggtcaaatgcgacggtgcagcactgtactatgggaaacggttttggctcttgggaattacacctaacgatgctcagattaaggaaattgctgattggctcttggaacatcatcaggattcgaccggtttgagtacagacagtctggccgatgcaggttaccctggcgctgcgcagcttggtgatgctgtgtgtggaatggcagcagcgaagattacctctaaagactttcttttctggttcaggtcgcacaccgccaaggagatcaaatggggaggtgccaagcacgatcctgatgagaaggatgacggccgaaaaatgcatccccggtcttcgtttaaggcctttctagtagttgtgaagagaagaagtttaccatgggaagacatagaaatggacgcaattcactctcttcaactcattttgagaggttcatttcaggatattgacgatagtgacaccaaaactatgatacatgcccgactgaatgacttgaagctccagggcatggatgagctcagcacagtcgctaatgaaatggttagattgatagagacagcgaccgcacctattctcgctgtggactcaggtggattcattaatggttggaacgccaaagtagctgaactaacagggcttccagtcgaagaagccatgggtcggtcgctggtaaaggatttaattttaaatgaatcaatcgatgtcgtgcagcggcttctccacctcgcactacaaggtgacgaagaacaaaacattgagattcaattgaagaccttcggtccacaaaaagagaaaggcgcggtgattttgatagtgaatgcttgctctagtagagatgttcaagacaatgtggtcggagtatgttttgttggacaggatgtgacaggtcaaaaacaggttttggataaattcactcggatacaaggggactataaggctatagttcagaatccgaaccctctgatccctcccatattcggtactgatgagtatggatactgttctgagtggaatccttcaatggaaaagcttactggatggaagagagaagaggtgattggaaaactgctagttggcgaaatttttggtattcaacttatgtgttgccgactcaagagccaagatgcaatgacgaaattcatgattgtactaaatggtgccatggacggtcaagacacagatcgatttcccttctccttttttgatagacaagggaagtacgtggatcccttactcacagtgaacaagagaactgatgcagaaggaagcatcactggcgtattttgctttttgcacacaaccagtgtggaattgttacaagcattgactgttcaacgttcgactgagaaagttgctttcgcaaaactcaaggaattggcttacattcgacaggagatcaagaatcctttgtatggtatagtattcacacgtaatttgatggaagacacagatttgtctgtagaccaaaggcaattcgtggagactagcgcggtctgtgaacgtcaactgcgcaaaatattggatgatttggatcttgagagtatcgaggacggttacttagagctggatacaactgagtttgagatgggaacagtgatggacgctgttgtcagccaaggaatgatcacatcaagagaaaagggtcttcaattaatccgggagactcccagtgagattaagaacatgtgtttatatggggaccaagtccggttacaacaagtgcttgcagactttttgttaaatgctgtgcgattcaccccatcctctgaaggctgggtcgggatcaaggtggtcccaacaaagaagcgcctgggtcgaggcgttcatgtcatgcacctagaattcagagttacacatcctggactgggtcttcctgaagagctggttcatgaaatgtttggtcgaggacgaggcatgactcaagaaggcctgggtctgagtatgtgtcgtaaacttgtaaagctaatgaatggtactgttcaatatatcagagaaacaggtaagagctgctttttagttgaagtagagcttcccttggcacagagagatgatgcaggtagtgtgagatctactgttgtctagtttattagagcaacaatctatgagtactttttgtcatatatcacttttcacattttggtatgtgctaggggtcaatacacaggcttgtcaaattaagtgtttagtttggtgaccggtcctatcattctcttctattgatggtttcaagtataatcttgcgatttgtgacgtgttcttccgactttaggttatacttgatgaagctttcaacaagatattagactctgatcttttccttgtagtgcgaagtgtaagtatattgacgaagacgatctatatgaccatggtcgtcttggtagatcgtgaagatatttgttctgtgtagaagttaaataacaactttgccagtacaattgatgacatgcttctcgggacagcatgcagtgcttatattttttgcgtagaagtttgtatttctcaagttttttccaattacatgtacttgcccttgcttctc

SEQ ID NO: 57 - DNA, 3778 bp, Artificial Sequence
  Synthetic Oligonucleotide
atgtcgaccaccaagttggcatactcgtcagggagttctgtgaagtcgaagcatagtgtacgggttgcgcaaactacagcagatgccaagctgcaggcagtgtatgaagaatccggagattcgggagactcatttgattattccaaatcagttcatgcgtccaaatccactggcgagaatgtccctgccctagcagtcaccgcgtaccttcagcgcatgcaaagagggggtttggtgcagacattcgggtgtatgctatgtgtggacgagagtagttttagggttattgcttacagcgagaatgcgccagagatgcttgacctgatgccacaagcggtgcccagtgtgggtcagcaggaagttctgggcattggaaccgatgcaagaactttgttcaccccttccagtgcggccgccctggagaaatgtgccggagcggtggatgtgaccatgctgaaccctatctcagtgcactgtcgaagctcggggaagccgttctatgcgattctgcaccgtattgacgtaggactggtaattgattttgaaccagtgcgcccgaatgatgccgttgtgtcctccgcaggggctttgcagtcgcataagttggctgcaaaagcgatatctcggctgcaggcacttcctggcggagacattggcctcctttgtgatacagttgtagaagaggtacgccagctttccggttacgatcgtgtcatggcatacaaattccacgaagacgaacatggtgaggtcttggcggagattcgaagatcagatttagaaccttacttaggtctacattacccagcaacagatatcccacaggcgtcaagattcctgtttatgaagaacagggtgcgaatgataggtgattgctatgctcctcctgttaaagtggttcaggacaaggatttgaggcagcctattagtctggccgggtctactttacgggcccctcatggctgccatgcgcaatacatgggcaacatgaactccatcgcttcgttggtgatggcagttatcgtgaatgatccggatgaggatcctaattcacgtgggggccagcagagagggcgcaagctctggggattggtggtttgtcaccacacgtctccgaggaccgtctcctttcctttgagatcggcctgtgagttcctgatgcaggtgttcggtctgcagctgaacatggaagttgaacttgcagctcagctaagagaaaaacatattttgagaacacaaacccttctctgtgacatgttattgcgggatgcacccatcggaattgtttctcagtcacccaatatcatggacttggttaaatgcgacggtgcagcactttactatgggaaaaggttttggcttttgggaatcacacctaacgaggtccagattaaggaaatcgcggactggctcctagaacatcatcaagattcgaccggtttgagtacagacagcctggccgatgctggttaccctggcgcagcgcaacttggtgatgctgtgtgtggaatggctgcggcaaagatcactccaagggactttcttttctggttcaggtcacacaccgccaaggagattaagtggggtggtgccaagcacgatcctgatgaaaaggatgatgggaggaaaatgcatccccgctcctcgttcaaggcctttctagaagttgtgaagagaagaagtttgccatgggaggacatagaaatggacgcaattcattctcttcaactcattttgagaggttcattccaggatatagacgacagtgataccaaaactatgatacacgcccgactcaacgacttaaagctccagggcatggatgaactcagtacagttgctaatgaaatggttagattgatagagacagcgactgcaccaattctcgctgtcgattcgagtggattcatcaatggttggaatgccaaagtagctgaactgactggacttccagttggagaagccatgggtcggtcgctggttaaagatttaattttagaggaatcaattgatgttgttcagcgacttctctaccttgcattacaaggtgaagaagaacaaaatattgagattcagttgaagacctttgggccacaaaaagagaagggcgcagtgattttgatagtgaatgcgtgctccagcagggatgtacaagacaatgtcgttggagtttgttttgtggggcaagatgtgacaggtcaaaaacaagttttggataaatttactcggatacaaggcgactacaaagcaatagttcagaacccaaaccctttgattcctcctatatttggcactgatgaatatggatattgctctgagtggaatccttcaatggagaagctgactggatggaagagagaagaggtgcttgggaaactgctagtgggtgaaatttttggaatgcaacttatgtgttgccgactgaaaggtcaagatgcaatgacgaaatttatgattgcattaaatagtgcaatggatggtcaagatacagatcggtttcccttttccttctttgatcgacaagggaaatacgtagatgccctactcacagtcaataaaagaacggatgcagagggaagcatcactggcgtattctgctttttgcacacaaccagtgtggaattattgcaagcattgactgtccaacgtgcgacggagaaagtagccttcgcaaaactcaaggagttggcttacattcgacaggagatcaagaatcctttgtatgggattatgtttacacgtaatttaatggaagatacggacttatctgaggaccaaaggcagtttgtggagactagtgctgtgtgtgaacgtcaattgcgcaaagtattggatgatatggatcttgagagtattgaggacgggtacttagagctggatacaaatgaatttgtgatgggaacagtgatggatgccgttgtaagtcaaggaatgatcacatcaagagaaaaaggtctgcaattgatacgggagactcccagggagataaagaacatgtgtttatttggggaccaagtccgtttacaacaagtgctcgcggactttttgttgaatgcagtgagattcaccccatcctctgaaggctgggtcgggatcaaggtggtcccaacgaagaagcgcctgggtggaggcattcatgtcatgcatctggaattcagggttacacattcaggaatgggacttcctgaggagcttgtgcatgagatgtttgatcgaggacgaggcatgactcaagaaggcctggggctgagtatgtgtcgtaaacttgtaaagctaatgaatggtaatgttcaatatattagagaaacaggtaagagctactttttagttgaagtggagcttcctttggcacaacgagatgatgcaggcagtgtgagatagagctttatgatagtgggcaatgagcccctttgccaatgaaatgcgtctcttgcaagattttatcctgtccacaccagatttctgagctgcagtacagagagctatgctagtaacccttgtttgtaggagcaggaagagaatgaactgctgtcttggttcatgagagcaacaaactataagtattttcttattttcgaataccactttccacattttggtatgctttaggcgtcaacatacagacttgataacatgagagtcttagtttggtgactaattgctccattactttcttctattgctggtttaaagttcaatcttgcgacttgtgacttgttacttgcgacttcaggatttactgaatgaaaccttcgggcaggatattagattctgatct

SEQ ID NO: 58 - DNA, 3977 bp, Artificial Sequence
  Synthetic Oligonucleotide
atggcgtccgccaagttggcctactcgacgggaagtggcatcaagtcgaagcacagcgtacgcgtgcagcaaaccacagcagatgcgaagcttcaagccgcctacgaggaatcaaacgactctggggattcatttgattactcgaaatcagtcggccaggccgccaaatccactgtgcaacaggttcctgctcaagcagtgacagcctacctgcagcggatgcagcgagggggtcttacacagacttttgggtgtatgctggccgtggaggagaacacatttagggtcattgcttacagcgagaatgctctggatatgcttgacttaatgccacaagctgtaccgagtgtggggcaacaggatgttctcggaattggaaccgactcgagatcattgttcactccttcgagtgcggctgcgttagaacgagctacacaacaatcagatttgagcatggtgaaccctgtctctgtccactctagaagctcagggaagccattttacgccattttacatcgcattgacgtagggatagtgatggactttgagcctgttcgaccaaatgatgtggtagtgtcctcggctggaaccattcactctcacaagcttgcagcaaaagccatttctcgccttcagtcgctgcctgggggcgacattggtcttttgtgcgataccgtagtcgaagaggttcgtgagctcacgggttatgaccgggtaatggcgtacaaatttcatgaagacgagcatggggaagttctggctgagattcgaagatcagacttagaaccttacttagggctacattatcctgcaacagatattccgcaagcttctaggttcctcttcatgaagaataggtttcggatgatcgttgattgctatgcgccccctgtgaaggttattcaagacaaggacttacgacagccgctcactcttgcaggatccactctgcgagctcctcatggttgccatgcgcagtacatggggaacatgggatctattgcgtctgttacaatggctgtcgttgtgaatgatcaggaagacgacggtggatcgcagaaagcgcgtagattgtggggattggtggtgtgtcaccatacatctgccaggatgatctccttccctctgcgttcggcgtgcgagtttctgatgcaggtgttcggcttgcaactaaacatggaagtggagctcgcagctcagttgagagagaaacatatcttaaggacacagacgttgctttgtgacatgctcttgcgggatgcacccataggtattgtgtcacagtctcccaacattatggatttggttaagtgtgacggagcggccctttattatgggaatcggttctggcttctaggaattacaccatccgagcagcagattaaggagatagcggactggctgttagagtgtcataaggactcaactgggttgagcacggatagcctagcagatgctggctatccaggtgcgaacctgctaggtgatgctgtatgtgggatggcagctgcaaggatcactcccaaagatttcctgttttggttcaggtctcacactgccaaggaaattaagtggggaggcgcaaagcatgacgcggatgatagggacgacggaaggaagatgactcctcggtcctcttttaatgcctttctagaagtggttaagaggcgaagtgtcccttgggaggacattgaaatggatgcaattcactctctgcagctgatcttgagggggtcttttcaagacattgatgacagcgatactaaaacaatgatacatgcacggctgaacgatttgaagttgcagggtatggatgagcttagcacagttgccaatgaaatggttcggctgattgaaacagcgacaacacccattcttgcggttgactctagtgggtttattaatgggtggaacgcgaaagtagcagaattgactggactttctgttggtgacgctatgggccgatccttggttaaggatctgattttggaggagtcagtggaggatgtgcagaggctgctgtatcttgccttacagggagaagaagagcaaaacgttgagatccgattgagaacatttgggccgcagaaggcgaaaggtgcagtgattttgatagtaaatgcctgttcaagcagggatgtacaggagaacgtggtcggagtgtgtttcgtagggcaggacgtgacagggcaaaagatgttacatgacaaattcacacgtattcatggtgactacaaaagtatcgtccagaatccaaatccattgattccacctatttttggaagtgatgacttaggttattgtaccgagtggagtccttcaatggagaagctaacaggatggaagagagatgaggtacttggaaaaatgctggttggggaagtatttggtatgcatctaatgtgttgccgccttaagggccaggacgcagtgactaaattcatgatagttcttaataacgctatggatggtcaagatacagacaaatatccattctcctttttcgatcgtcaggggaagtttgtggaagctctgcttacggctaacaagagaacggatgcagatggatacatcacaggtgttttttgctttctgcacattgcaagcccagaattgttacaggcactaactgttcaacgagctacagagaaggtggcttttgccaaactcaaggaacttgcttatatccggcaagaaatcaagaatcctctgtatggaatcatgttcactcgaaatttgatggaggatacagatttgtcggatgaccaaaagcaatttatggacactagtgcagtatgtgagcgacaattgcgtaaaatattagatgacatggatctcgagagcattgaagatgggtatttggagttggaaacagcagaatttgagatggggtccatgatggacgctgttgtgagtcagggaatggttacgtcaagagagaaaggccttcagttaattcgtgaaacgcctcgggaaatcaaggatatgtgcttattcggagatcaagttcgattacaacaagttcttgcagactttttgctaaatgcagtgcgttttactccctcttccgagggttgggtcggcatcaaggtggtcgcaacgaaaaggcgccttggtggtggctttcacgtcgtgcacttagaattcagagtaacacatcctggttcagggcttccagaggagctcatccatgagatgtttgaccggggtcgtggtatgactcaagaagggcttgggctgaatatgtgccgcaaacttgtgaaactgatgaatggtaatgtacaatacaagagagagacaggaaaaagttactttttggtcacactggagcttccgttggctgtacgagatgataccggtaccatgcggtaaatggcctctagaatgccaatgaaccttgtttgtatacaggttgataacttacttctccccacaccggttcatgtatatctggaagacaaaagtctcggatcagcagagcagctttattcttctcttgtggaaattgaggtcgtgaaactgctcactttgtattgatctcgagcaacaccacaagtctcttctcttcattttttgagatggtttacagagaaggagactaatcactggaaagatagtgatgaatggcggaggcttatactcgcggactaagccgggttcatactcctttggaaccttgctccggaccacctgccacatggttgtttttctttttttttttctctcattttctcacgggtgacgaacagtatttgggcaaaacatgtccaaagcagcgtgttaattttcttgatgactaatgcaattttgatttttagcctagaaacagaactgtcttgtgtatgtttcatataagctgtagatgaacagctttgtatgcgtcatgaaggccctatgcattcgtatatatctctgacgagtggtactcgattggcgattcatgaatgctctctgttctacgaccaatttagatccag

SEQ ID NO: 59 - DNA, 3884 bp, Artificial Sequence
  Synthetic Oligonucleotide
atgtcgtccactaagttgtcgtactcgtcgggaacctctgtcaagtcgaagcacagcgtgcgcgtgcagcaaaccaccgcagatgccaagcttcaagccgtctacgaagagtcaaatgactccggagactcgtttgattactcgaaatcagttggtcaggccaccaaatccactgtgcaacaggtccccgcgcaagctgtgaccgcttatctccagcgcatgcagagagggggtctcactcagaatttcgggtgcatgctcgccgtcgaagagagtaccttcagggtcattgcatacagtgagaatgcgcccgaaatgcttgatttaatgccacaagctgtgccaagtgtcggtctaaaggaagtcctcggaatcggaaccgatgcgaggttgttgttcactccctccagcgcgtccacgttagaaagagctgcggcaacgtccgacttgaccatggtcaaccccatctctgttcactctcgcaattcagggaagccattttacgcaattgtgcaccgcattgatgttggaatagtgatagatttcgagcctgttcgatcgaacgatgtcgtcatctccacggctggggctctccattctcataaattggcagcgaaagctgtttcgcggttgcaatcccttcccggaggcgacatcggcctcttgtgcgacgcagttgtggaggaagtacgtgagctcacgggctacgaccgagtgatggcgtacaaattccacgatgacgagcacggggaggtcttagccgagattcgaagatcagatttagaatcctacttagggttgcattacccttcaactgacataccgcaagcatcgaggttcctcttcatgaagaatagggtgcggatgattgcggattgctgcgcgcctcccgtgaaagtgatacaggacaaggacctacggcagccaattacgcttgcgggatccactctgcgagcgcctcacggctgccatgctcagtacatgggcaacatggggtcagtcgcgtcaattactttggccgtcattgtgcatgatcaggaggaggatttcggagtgcaacagaaaggccgtcggctttgggggctggtggtttgtcaccacacgtcccctcggacgatctccttccctctccgatcggcttgcgaattcctgatgcaggtgtttggattgcaactcaacatggaagtggagcttcaggctcaaatgaaagagaagcacatcctgagaacgcagacactgctttgtgacatgctcttgcgggatgctcctgtaggtattgtgtcgcagtctcccaatattatggatctggtgaagtgcgacggtgcagctctctattacgagaatcagttctggctcttggggaccacaccgagtgaggagcaaatcgaggagattgcagcttggctattagagcatcataaggattcaacaggattaagcacagatagtctggcagacgcgggctacccaggcgcgaatctgctcggtgacgctgtttgtggcatggcagctgcaagaatcagctccaaagattttctgttatggttcaggtctcacagtgctaaagagatcaagtggggtggtgccaagcacgatgccgaggacagggacgacagtcggaagatgactcctcgttcttcattcaatgctttcctagaagttgtcaagagacgcagtgttccttgggaagacatcgaaatggacgcaatacactctctgcagcttatcttgaggggctcctttcaagatgttgacggtagtggtgggaagacgatgatccattcgcggctccacgatttgaaactgcagggcatggacgagcttagcacggttgcgaatgaaatggtgaggttgatagaaacagcgactgcgcccatccttgcggtggactcaaatgggttcattaatggatggaatgcgaaagtggccgaactgacgggacttccggttggagaggccatgggtcgatccttagttaaagatttgattttggaagagtatgtggaggttgtcgagcggctattgtatcttgcacttcaaggagaagaagagcaaaacatcgaggttagtttgaagacgtttggagcgcaaaaggcgaaaggtgcagtgatactagtagtgaatgcctgttcgagtcgggatgtacaagagaatgtggttggtgtgtgcttcgtcggccaggacgtgacaggacagaagatggttcatgataagtttacacgcattcacggtgactacaaaagcatcgtgcagagcccaaatcctttgattccacccatctttggaagcgatgaactcggatactgcatcgagtggagtccttcaatggagaagttaacaggatggaagagggaagaggttctcggaaaaatgcttgttggagaaatttttggtgtgcatcaaatgtgctgtcgtctgaagggtcaagatgcagtaactaaattcatgatagtcctcaataacgctatggatggtcaagacaccgacaagtatccgttttccttccatgaccgccaagggaagttagtggaagctctcctgacggccaacaagaggacggatgcagatgggtacatcacaggagttttctgcttcctgcatatcgcaagcccagaactgttacaggcactaactgttcaacgagcaacggagaaagtggcttttgccaaactcaaggagcttgcctacattcgacaagagataaagaatcccctatacggtatcatgtttactcgaaatctgatggaggatacagatttgtcggatgaacaaaagcagtacatggacactagtgcagtttgtgagcggcagttgcggaaaatattggatgatatggaccttgagagtattgaggacgggtacttggagttggagacgatggagttcgagatgggatctatgatggacgctgtagtgagtcaaggcatggtcacgtcaagagagaaggggcttcagctgattcgtgagactcctcgggagattcagggcatgtgcttgttcggagatcaagttcgtttacaacaggtcctcgcagatttcttattgaacgcagtgcgtttcaccccttcttcggagggctgggtcggcatcaaggtggtcccaaccaggaaacgccttggtggtggttttcacgttgtgcacttagagttcagagtaacacatccgggttcagggcttccagaggagctcgtccatgagatgttcgaccggggccgcggcatgacccaagaaggacttgggctgagcatgtgtcgcaaacttgtaaaactaatgaacggtgaagtgaaatacataagggacacaggcaaaagctgctttctagtcagcctggagctgccattgactgttagcgatgatgctggcagcgtgaggcagtcgggccctttgaattgctgatgatcctgtttttgaagactgatggacagctcgcttgttcctgtaccgctacaaacgtatttggcagaggcttgttggagcagtgtgatgaagttgaatggagatgagatggaaattcgaatcgtttttttccacctctgcattgctgctacgggagaggttgttgaagtgaagcgtctcccccacccttgacaacaaaggagaaggaacaaccatcgggttgcaggtgatcaataccctgagctgaagcttacaaaccctttatttcaactatccgactgggcgtttcgctctgagtctgatcgctgggcgcatggatccaatcatacgggtgagaagtaggatctggatcctgtacaatgtaatttactgctgtgtgcctgttggtacatgcagttttgaaacttgagtgtagacacacaattgatattactattttttaatgtatgaaccataaaactgctagatgagcaggtttcttcgtgttc

SEQ ID NO: 60 - DNA, 3375 bp, Artificial Sequence
  Synthetic Oligonucleotide
atgacgtccactaagttatcctactcgttgggaaccaccgtgaagtcgaagcacagcgtgcgagtgcaacaaaccaccgcagatgctaagcttcaagctgtgtacgaggagtcgaatgactccggagacgcgtttaattactcgaaatcagtcggtcaagctgccaaagcaaccgtgcagcaggttcccgcacaagctgtgtcatcatatcttcagcgcatgcagagagggggtctcacccaaaccttcggatgcatgctcgccgtcgaggagagtaccttcagggttattgcatacagtgagaatgctcccgaaatgctcgatttagtgccacacgctgtaccgagcgttggacaacaggatgtactcggaattggagctgatgcaagatcgttgttcactccctcgagcgcatccgcgttagaaagagccgcatcaacatccgatttgagcatggtaaaccccatctctgttcactctcgaagctcagggaagccattttacgctattgtgcaccgcattgatgtgggggtagtaatagactttgagcctgttcgaccgaacgatgtcattatctccacagctggggctctccattcccacaaattggcagctaaagcagttgcgcggctacaatcccttcccggaggcgacattgggctactgtgtgatgcagttgtggaagaggtccgtgagctcacaggctacgaccgagtgatggcgtacaaatttcacgatgacgagcatggggaggtcctcgccgagattcggagatcagatttggaatcctacctaggcttgcattatccctccacagatataccgcaagcatcgaggtttctgttcatgaaaaacagggtgcggatgattgcggattgctgcgcgcctcctgtgaaggtaattcaagacaaggacttgcgtcagccaatcactcttgcgggatccactttgcgagcgcctcacggctgccatgctcagtacatgggcaatatgggttcagtcgcttcaattactctggctgtcatcgtaaatgaccaagaagaggacttcggagtgcagcagaaaggccgtcgactgtgggggctggttgtctgtcatcacacgtccgctcggacaatctccttccctctccgatcggcgtgtgagttcttgatgcaggtgttcggattgcaacttaatatggaagtagagcttcaagctcaggtgaaagagaagcacatattacgaacgcagacattgctatgtgacatgctcttgcgtgatgctcctatcggcattgtgtcgcaatctcccaacattatggatctagtcaagtgcgacggagcagctctctattacgggaaacgtttctggctcctggggaccacaccaactgagcagcaaatcaccgagattgcagactggctgttggagtatcacaaggattctacaggattgagtacagatagcctggcagatgcaggctaccctggcgcaaacctgcttggtgacgccgtatgtggtatggctgctgcaagaatcactcccaaggattttctgttttggttcagatctcatactgctaaagagattaagtggggtggcgcaaagcacgacgcggatgagaaggacgatggtcggaagatggctccccgatcttcgttcaacgctttcttagaagtcgtaaagaggcgtagtgttccttgggaagacattgaaatggatgcgatccactctctgcagctcatattgagggggtctttccaagacattgacgatagcgatgggaaaacgatgattcatgcgcggcttcacgatttgaagctccagggtatggacgagcttagcaccgttgctaatgaaatggtcaggttgatagagactgcaactgtgcccattcttgcggtggactcaagtgggttcatcaatggatggaatgcgaaagtagcagaactgacaggactcccagttggagatgccatgggccgctccttggtaaaggatttgatcttggaagagtccgtggaggctgtcgagcggctgctgtacctagcgcttcaaggtgaagaagagcaaaatgttgaggttcgtttgaagacatttgggcagcaaaagtccaaaggcgtattgatattaatagtcaatgcctgttcaagtcgggatgtgcaagagaatgtggttggcgtatgtttcgtgggccaggacgtgacagggcaaaagatggtgcatgataagtttacacgcattcatggtgactacaaaagcattgtgcagaacccaaaccctttgatcccacccatctttggaggcgatgaactaggatactgcaccgaatggagcccttcaatggagaagttgacagggtggaagagggaggaggttctcgggaaaatgctagttggagaagtctttggtgtacaactcatgtgttgccgtctgaagggccaagatgcagtaacgaaattcatgatcgtcctcaacaatgccatggatggtcaggacacagacaagtttccgttttcgttttttgaccgtcaggggaagtttgtggaagctcttctgacagccaacaagaggacggatgcagatgattacatcacaggagtgttctgttttctacacactgcaagcccagagttattgcaggcattaactgttcaacgagctactgagaaagtagcgtttgccaaactcaaggagcttgcgtacattcgacaggagattaagaatcccttgtatggaatcatgtttactcgaaatttgatggaggacacagatttgtcagaagaacaaaaacagtttatggacactagcgcagtctgtgagcggcagttgcggaaaattttggatgatatggaccttgaaagtattgaagacgggtatttggagttggagacagccgagtttgacatgggatctatgatggacgctgtggtgagtcaaggaatgataacatcgagggagaaaggccttcagttaattcgtgaaacccctcgggagatcaaaggcatgtgcttatttggggaccaagttcgattacaacaagtcctcgcagactacttgttgaatgcagtgcgtttcaccccttcttctgagggctgggttggcatcaaagtggtctcaacgaagaaacgcctagttggtggcttccacgttgtgcatttagaattcagagtcacacatccgggatcaggacttccagaggagcttgtctgtgagatgttcgaccggggccgtggcatgactcaagaaggactcgggctgagtatgtgtcgcaagctcgtaaaactaatgaacggtgaagtgaaatacatcagagacgccggcaaaagctatttcttagtcaacctggagcttccattggccggaagagacgattctggcaacgcgaggtag

SEQ ID NO: 61 - DNA, 3571 bp, Artificial Sequence
  Synthetic Oligonucleotide
catcgtcatcgccttgcgcgcaatatccttggctccacgccccagtaagcgaccagtgacagtgagagcggtgaagcaaaccaaagcaaagctatcggaagaacctcagtcgagggggcgagcgagccgacgagacgactgacgagcgcacaagaccggacggggccgcctaatccagagagagccccaggtctcccccaaccccgccatggcgtcgggcagccgcgccacgcccacgcgctccccctcctccgcgcggcccgaggcgccgcgtcacgcgcaccaccaccaccactcccagtcgtcgggcgggagcacgtcccgcgcgggcgggggagccgcggccacggagtcggtctccaaggccgtcgcccagtacaccctagacgcgcgcctacacgcggtgttcgagcaatcgggcgcgtcgggccgcagcttcgactactcccaatcgctgcgcgcgccgcccacgccgtcctccgagcagcagatcgccgcctacctctcccgcatccagcgcggcggccacatccagcccttcggctgcacgctcgctgttgccgacgactcctccttccgcctcctcgccttctccgagaactcccccgacctgctcgacctgtcgcctcaccactccgttccctcgctggactcctctgcgccgccccacgtttccctgggtgccgacgcgcgcctcctcttctccccctcgtccgcggtcctcctagagcgcgccttcgccgcgcgcgagatctcgctgctcaacccgatatggatccactccagggtctcctccaagccgttctacgccatcctccaccgcatcgacgtcggcgtcgtcatcgacctcgagcccgcccgcaccgaggaccccgctctctccatcgccggtgcagtccagtcccagaaactggcggtccgcgccatctcccgcctccaggcgctacccggcggggacgtcaagcttctctgcgacacagtcgtggagcatgttcgcgagctcacgggttatgaccgtgtcatggtgtacaggttccatgaagacgagcacggggaagttgtcgccgagagccggcgcgacaaccttgagccttacctcggattgcattatcccgccacagatatcccccaggcgtcgcgcttcctgttccggcagaaccgcgtgcgaatgattgccgattgccatgccaccccggtgagagttattcaagatcctgggctgtcgcagcctctgtgtttggtaggctccacgctacgcgctccacacgggtgtcatgcacagtacatggcgaacatggggtcaattgcgtcgcttgttatggcagtcatcattagcagtggcggtgacgatgagcaaacaggtcggggtggcatctcctcggcaatgaagttgtgggggttagtggtgtgccaccatacatcaccacggtgtatcccttttccattgaggtatgcttgcgagtttctcatgcaggcatttgggttgcagctcaacatggagttgcagcttgcgcaccagctgtcagagaagcacattctgcgaactcagacgctattgtgtgacatgctactacgagattcaccaactggcatcgtcacgcagagccccagcatcatggaccttgtgaagtgcgacggggctgcactgtattatcatgggaaatactatccattgggtgtcactcccactgagtctcagattaaggatatcatcgagtggttgacggtgtttcatggggactcaacagggctcagcacagatagcctggctgatgcaggctaccttggtgctgctgcactaggggaggctgtgtgtggaatggcggtggcttatattacaccgagtgattacttgttttggtttcggtcacacacagctaaagagatcaaatggggtggcgcaaagcatcaccctgaggataaggatgatggtcagaggatgcacccacggtcgtcattcaaggcatttcttgaagtggttaaaagcagaagcctaccatgggagaatgcagaaatggacgcaatacattccttgcagctcatattgcgtgactccttcagggatgctgcagagggcaccaacaactcaaaagccattgtcaatggacaagttcagcttcgggagctagaattgcgggggataaatgagcttagttccgtagcaagagagatggttcggttgatagagacagcaacagtacccatatttgcagtagatactgatgggtgtataaatggttggaatgcaaagattgctgagttgacagggctttcagttgaggaggcaatgggcaaatctctggtaaatgatcttatcttcaaggaatctgaggcgacagttgaaaaactactctcacgagctttaagaggtatttccatttctgtttcttatatggatgctattgcctttatactacgtttcatgaaaactgcggagtgctgttgaggaaaatattatgttcttggttgtatctcttaaggtatatggcataacagaagcttatggacatgatgtacacatgtttattagaccaaatatggaaatgggtggtaatatactcctggaaaatggatatttgaaggagctgagcctatctcagtttgttgacagtgtggatgtatgcctagacccagacccccaagttcaagtccttgtcgaggcgatttttttgcatatttcttctaattaaaaagcccttctatgttggtcaaggttttttaatagatatactcagtttcttaggcggacttctttttgtgcacaaatatatagaaaatgggggatcaactagagactaccaacaaattcattcaaccatttctagagtatttttagaacctggtgcttataactagcagacatgaatactcattagtcatatgtgaaaagtaagcatatgaaacttataaaaaaggtcatgttagttgttgaagtgtatgagtggattaactatcttttcccatcagcttaagcttttgggttcaactggttagcgcgtccactccaacatggtaccaaagacaaaggtctcgaattcgaattctggcaaaggctttatttatgcctccacccatttatttccacgtttgcgcccctctctctctctctctggctgcatttgcgcctttctctctggctacacgtaagtgggggtgttgaagtgtataagtggattgactaccttctcccatcagccttaagcttttgggttgaactgtttagtgcgttcactctaacattagctactcacaggagtacaatgtttctccccaggctgtttgaactattttaatgtacttcccaatgcatgtagctaagctatgttagctttaatggatgtgagattggagagtgctaatttgtgaagttgcttcccatacaggtgaggaagacaaaaatgtggagataaagctgaagacatttgggtcagagcaatctaagggaccaatatttgttgttgtcaatgcttgttctagtagagattacacacaaaatattgttggtgtctgttttgttggacaagatgtcacaggacaaaaggtggtcatggataaatttgttaacatacaa

SEQ ID NO: 62 - DNA, 3501 bp, Artificial Sequence
  Synthetic Oligonucleotide
atggcgtcggacagtcgcccccccaagcgctccccctccgcgcgacgcgtggcgccgcgtcacgcgcaccaccaccactcgcagtcgtcgggcgggagcacgtcccgcgcaggcgcgggaggaggtggcgggggcgctgcggccacggagtcggtctccaaggccgtcgctcagtacaacctagacgcgcggctccacgcggtgttcgagcagtcgggcgcgtcgggccgcagcttcgactactcccagtcgctgcgcgcgccgcccacgccgtcctccgagcagcagatcgccgcctacctctcacgcatccagcgcggcggccacatccagcccttgggctgcacgctcgccgtcgccgacgactcctccttccgcctcctcgccttctctgagaacgccgccgacctgctcgacctgtcgccgcaccactccgttccctcgctcgactccgtggcgctgccccctgtttcccttggtgccgacgcacgcctctacttctccccctcgtccgcggtcctgctggagcgcgccttcgccgcgcgcgagatatcgctgctcaacccgctatggatccactccagggcctcctccaagccgttctacgccatcctccaccgcatcgacgtcggcgtcgtcatcgacctcgagcccgcacgcaccgaggaccccgctctctccatcgccggcgcagtccagtcccagaaactcgcggtccgcgccatctcccgcctccaggcgctacccggcggggacgtcaagctcctatgcgacacagtcgtggagcatgttcgcgagctcactggttacgaccgtgtcatggtgtacaagttccatgaagacgagcacggggaagttgtcgcagagagccggcgcgataaccttgagccttacctcggattgcattatcccgccacagatatcccccaggcgtcgcggttcctgttccagcagaaccgcgtgcgaatgatcgcagactgccatgccatcccggtgagagtcatacaagatcctgggctgtcgcagcagctgtgtttggtaggctccacgctacgcgctccgcacgggtgccatgcacagtacatggcgaacatggggtcaattgcgtcgcttgttatggcagtcatcattagcagtggtggtgacgacgagcgaacaggtcggggtgccatctcctcatcaatgaagttgtgggggttagtggtgtgccaccatacatcaccacggtgtatcccttttccattgaggtatgcttgcgagtttctcatgcaggcatttgggctgcagctcaacatggagttgcagcttgcgcaccagctgtcagagaagcacattttgcgaactcagacgctattgtgtgacatgctactgcgagattcaccagctggcatcatcacgcagagccccagcgtcatggaccttgtgaagtgcgatggggctgcactgtattatcgtgggaagtactacccattgggtgtcactcccaccgagtctcagattaaggatattatcgagtggttgacggtgtgtcatggggactcaacagggctcagcacagatagccttgctgatgcaggctaccttggtgctgttgcattaggggatgctgtgtgtggaatggcggtggcttatataacaccaagtgattacttgttttggtttaggtcacacacagctaaagagatcaaatggggtggcgcaaaacatcaccctgaggataaggatgatggtcagaggatgcacccacggtcatcattcaaggcatttcttgaagtggttaaaagcagaagcctgtcctgggagaatgcagaaatggacgcaatacattccttgcagctcatattgcgtgactccttcagagatgctgcagagggcactagcaactcaaaagccattgtcaatggacaacgtcaacttggggagctagaattgcgggggataaatgagcttagctctgtagcaagagagatggttcgattgatagagacagcaacagtacccatatttgcagtagatactgatggatgcataaatgggtggaatgcaaagattgccgagttgacaggcctttcagttgaggaggcaatgggcaaatctctggtaaatgatcttatcttcaaggaatgtgatgatatagtcgaaaagctactctcgcgagctttaagaggtgaggaagacaaaaatgtggagataaagctgaagacatttgggtcagagcaatctaagggtgcaatatttgttattgtcaatgcttgttccagcagagattacacacaaaatattgttggtgtctgttttgttggacaagatgttacaggacaaaaggtggtcatggataaatttatcaacatacaaggggactacaaagctattgtacacaatcctaatcctctgctacccccaattttcgcatcagatgagaacacttcttgttcagaatggaacacagccatggaaaaacttacaggatggtctagagaggaagttgtcggtaagtttcttattggagaagtgtttggaaattgttgccgactcaagggcccagatgcattgacaaagttcatggttgtcattcacaatgctatagaaggacatgattctgagaagttccctttttcatttttcgacaagaatggaaagtatgtgcaggccttattgacggccaacacaaggagcaaaatggatggtaaatctattggagccttttgtttcttgcagattgcaagcgctgaaatacagcaggcatttgagattcagagacaacaagaaaagaagtgttatgcaaggatgaaagaattggcctatatttgccaggagataaagaatcctcttagtggcatccggtttaccaactctctattgcagatgactgatttaaatgatgaccagaggcagttccttgaaactagctctgcttgtgagaaacagatgtccaagattgttaaggatgccagtctcaaaagtattgaggatggctctttggtgcttgagaaaagtgagttttctcttggagacgttatgaatgctgttgtcagccaaacaatgtcattgttgagggagagggatttacaacttattcgagatatccctgatgaaatcaaggatgcatcagcatatggtgatcaatttagaatccaacaagttttggctgacttcttgctaagcatggcacagtctgctccatccgagaatggctgggtagaaatacaagtcagaccaaatgtaaaacagaattatgacggaacagatacagagcttttcatcttcaggtttgcctgccctggtgagggcctccccgctgacattgtccaggatatgttcagcaattcccaatggtcaacccaagaaggcgtaggactaagcacatgcaggaaaatcctcaaattgatgggcggcgaggtccaatacatcagggagtcagagcggagtttcttcctcatcgtccttgagctgccccaacctcgtctagcagctggtagagaaaatcagctgatatgttaa

SEQ ID NO: 63 - DNA, 4293 bp, Artificial Sequence
  Synthetic Oligonucleotide
aatattaatctaataaatacccaaatattattaaaaaattcactgcagaataagaaattttattttgcccaatcaaagcaatgggttgggtcaaaccaacatagaccttttttttttatcaaaccaaaccagatagtagttcttttttgctagcatattccattttcacgaaaatttataatccaccatattactcacaactagtactagtataaaattgtcacaaccccatttctaaattcaacccaaagttcaatctcttttcagcttactactctggtagaacttcccaaaggtcaaatctcttttagggttatcaatccatttctttttccatgttaaaccaagaaaagttgtgagaaaaaatggcttctggaagtagaacaaagcattctcatcagtcaggtcaaggtcaagttcaagctcaatcttcaggcacaagtaatgttaattacaaagattcaataagcaaagccatagcacagtacacggctgatgctaggcttcatgctgtgtttgaacaatctggtgagtctggcaagtcttttgattattcacagtctattaaaactactacacaatctgttgttcctgaacagcaaattactgcttatttgactaaaatccaaagagggggtcatattcagccttttggttgtatgatagctgtagatgaggctagttttcgtgttattgcttacagcgaaaatgcgtgcgaaatgcttagtctaactccacaatcagttccaagccttgagcggcctgagatcctcactgttggaactgatgttaggaccctttttactccttctagctctgttttgctagaaagagcatttggggcgcgcgagatcactttgctgaatcctatttggattcattccaagaattctggcaagccattttacgcaattttgcatagggttgatgtcgggattgtaattgatttggagcctgctagaacagaggaccctgctttatccattgctggcgcagtgcagtcacaaaaacttgctgtgagggctatttctcatttgcaatcacttcctggtggggatgttaagcttttgtgtgatactgtggttgagagtgtgagggagttaactgggtatgatcgggttatggtatataaatttcatgaggatgagcacggggaggtagtggctgagagcaaaataccagatttagagccctatattggtttgcattatcctgctaccgacattcctcaagcttcgcggtttttgtttaagcagaacagggtaagaatgattgtggactgccatgccacccctgtgcgggttgttcaggatgaatcactgatgcagcctttatgtttagttggttccacacttagagcccctcatggttgccacgcgcagtacatggcaaatatggggtctattgcgtcattaacactagcagttattatcaatggaaacgatgaggaagctgttgggggccgaagttcaatgaggctgtggggcttggttgttggacaccatacttctgctaggtgcattccattccctcttcggtatgcctgtgaattccttatgcaggcctttggactccaattgaacatggagttgcaactggcatcacagttgtctgagaaacatgttttgaggacacaaacactgttatgtgacatgctccttcgagactcacctacggggattgttatccagagccccagtattatggaccttgtgaagtgcgatggcgctgctctgtactgccaggggaagtactatccattaggcgttacaccaactgaagctcagataaaggacattgtggagtggttattgacttaccatggggactcaacaggtttaagtactgacagtttggctgatgcaggatatcctggggcagctttgcttggtgatgcagtttgtggtatggctgttgcttatataacttctaaagatttcttattttggtttcgctcccatacagcgaaagagataaagtggggtggtgcaaagcatcatcctgaagacaaggatgacgggcagagaatgcatccacgttcttccttcaaggcatttctggaagttgttaaaagccggagcttaccatgggaaaatgcagaaatggatgcaattcactctctgcttattctgcgagattcatttaaggatgccgaggcaagtaattctaaggctgttgtgcatgctcagcttggggaaatggagttgcaagggatagatgaactgagttctgttgccagagaaatggttagattgatagagactgcaactgctcccatatttgctgttgatgtcgaaggtcgcattaatgggtggaatgcaaaggtcgctgaattgacagatttatctgttgaagaagcaatggggaagtccttggttcatgatcttgtgcataaagagtcacaggagactgctgagaagcttctcttcaatgctctgagaggcgaagaagataaaaatgtagaaataaagttaaggacatttggacccgagcaactgaagaaggctgtttttgtggtggttaatgcttgctctagcaaagattacacaaacaacattgttggtgtttgttttgttggccaggatgttactgggcaaaaagttgtaatggacaagtttattcacatccaaggtgattacaaggccattgtgcacagccccaatcctctgatcccacccatatttgcgtcagatgagaacacttgttgctctgagtggaacactgccatggaaaagctcactggttggtccagaggggagatcattggaaaaatgttagttggtgagatttttggaagttgctgtcggctcaagggtccagacgccatgacaaagttcatgatcgtgttgcataatgcgattggagtccaggatacggacaagtttccattttccttttttgaccgaaatgggaaatatgtgcaagctcttttgactgcgaacaagagagtcaatatggagggccagattatcggggctttctgtttcatacagatagccagtcctgaattgcagcaagctctaagagttcaaaggcaacaggaaaagaagtgttattctcagatgaaagagttggcatacctttgtcaggaaataaagagtcctttgaatggtatacgctttacaaattcattgttggaagcgacagatttgacagaaaaccagaagcagtatctggagacaagtgctgcttgtgagaggcagatgtctaagatcataagggatgttgacctggaaaacattgaggatggctcactgacccttgagaaagaagaatttttccttgggagtgtaatagatgctgttgttagccaagtgatgttattgctgagggaaagaagtgtgcaattaatcagggatattccagaggaaattaagaccttaacagtacatggtgatcaagtgagaattcaacaggtcttggcagatttcttgcttaacatggtacggtatgcaccatcacctgatggttgggtagagatccaacttcagccaaatatgaagcaaatatctgatgaagtaactgttgtgcatattgaattcaggattgtatgccctggtgaagggcttcctcctgaattggttcaagacatgttccacagcagtcggtgggtaaccaaggaaggcctaggactgagcatgtgcagaaaaatcttaaagcttatgaatggagatatccagtatatcagagaatcagaaagatgttatttcctgatcatccttgacctaccaatgacccgcagaggttcaaagagtcttggctaggcttacaggagtaagaaatgaacctgtaatatctcgtaaaattggaataagggcaagctgttgaaattctggtaatgcttctgatcctttaatggaacttgacacagggttacacaagagagcaagtatggggaactaatcgccatcatgctaatagttttgaacgagctgtttcatacattttgtgacttctgtttgctggcgtcttttacgactcctacctagtattcacataaagatggtggtttgaagcttaagcaagggagtaaggtgcacgagagctgttgcaccttagcgcatgcttttttgccaagattaattcttgactagagtaaatggctaattgccaaataattagaatatagttagcattattaaagaagtttccatgattaattgatcccaaggctgttctgggtaacatctattctgaagaattgtgacaagtatattgctgtataatatgtattacaaggttcctgcgaaataaatatcatcgtttccagttgtcaggcaaaaaaaaaaaaaa

SEQ ID NO: 64 - DNA, 7285 bp, Artificial Sequence
  Synthetic Oligonucleotide
atggcatcacaatcacaaagacaaagcaaccagcggcagcaccagaatcaagcggcgcagtcttctggtaccagtaacatgaggcaacatcatcacgccacagaatcagtaagcaaagcaattgcacaatacactgtcgatgcacaactccatgcagtttttgaacaatctggcgggtctggtaagtcgtttgattattcacagtcagttagaactactagtcagtcagtacccgaggaacaaatcactgcttatttatcaaaaatccaaaggggtggtcatatccagccatttgggtgcatgattgctgtagatgaagggtcgtttagggtcattgcatatagcgaaaatgccaaagaaatgcttggtttaactccacagtcagtcccttctctagataaacaggaaattctctctgatgggactgatgtaaggactctttttaggccttcgagctccgctatgcttgaaaaagcatttggtgcaagggaaataattttgttgaatccaatttggatccattccaagaattccgggaagcctttttacgcaattttgcataggattgatgttggtattgttattgatttagagcctgctaggactgaagaccctgctttatccatagcaggggctgtacagtctcagaaattagcagttcgttcgatttcacaattacaatcacttcctggtggggatattaagttattgtgtgatactgtagtggagagtgtcagagagcttaccgggtatgatagagttatggtttataagtttcatgaagatgagcatggtgaggttgtggctgagaataaaagggctgatttggaaccttatattggtttgcattatccttctacggatataccacaagcttcaaggtttttgttcaagcagaatagggttaggatgattgtggattgccatgctacacctgtccgtgttatccaggatgaggcgcttatgcagcctttatgcttggttggttcaactcttcgagctcctcatggttgtcatgctcagtatatggcgaatatggggtcaattgcttcattggccatggctgttattattaatggaaatgaggaagaagctattggtgggaggaattcaacgaggctttggggtttggttgtttgccatcacacttctgctaggtgtattccatttccgcttcgttatgcatgtgagtttttaatgcaggcttttggacttcaattgaacatggaattgcagttagcgtcacagttgtcagagaaacatgtcttgaggactcagactctcttgtgtgatatgcttctccgtgactctcctactggcattgtcactcaaagtcccagtatcatggatcttgtgaagtgtgacggggcagctctttattaccaaggacagtattatccattgggcgtgaccccaaccgaagcccaaataaaagatattgtggagtggttgttggcccttcacggagactcaactggtttaagcacagacagtttagctgatgctgggtatcctggggcagcctcacttggcaatgcagtttgtggaatggctgttgcttatattactaagagagattttcttttctggtttcggtctcacactgcaaaggagatcaaatggggtggtgcgaagcatcacccagaggacaaggatgatgggcagaggatgcatccacgttcttcattcaaggcatttttggaggtggtgaagagccggagtttactgtgggagaatgcagaaatggatgccattcattctttgcagcttattttgcgagactcatttagggacgttgaagcaaccaattctaaggcagttgtacatgcccagctcgaggatacagaattgcaagggatggatgagctcagttcggtcgcaagagaaatggtgagactaatagagactgcaactgctccgatatttgctgtagatgttgatggctgcataaatgggtggaatgcaaaggtcgctgagttgactgggctctcagttgacaaggccatggggaagtctttggttcacgatcttgtttataaggaatatgaagaaacagttgacaaactccttcatcgtgctttaagaggtacctgctttatatctcacatatttaagttgtttatcaagtaagattgtcatctcatggcaatgaactttttttctaaagttgcatctgagcatgcagtcccctttaatagaaataaatcttggaaaacgtatatgggttttgcaaaaaactggaattaaaaaagttgataaatggaaatgtaaaagaaggtcaaactcaaatcctggaaataattctaatagaaactttcatcaaactaagaatacaattggctcactcataagttttacatgaagtacattgtaatagtttcatctgcatgggatacctgactagtcttctgatcaaatgacttgaatgaagtaacgtgtataattgcttctcttgaatgctctctgttgacaaagacaaaatgaagtgctgatgcatcaaaggctgtggcaatgcacaatttattcaaaattagcaataaataaaaaggaaattaagggttttgggcttttaagaggaatcagctagtcaggaatttgaatactgcaatggagaagaatatattctacagacatcattcactaagatcttgggtgcctcatcttgggcaaaatccagtagacattcgccattaagaagagcctagaggctgaagcatttctcatcgaactttctttgaaagtggattactgcagtcttgtgtatgacttgaccattgagatgagtccattttataactagcagcttagaagcaaagccctagcttccctgttattgtaagactcttaaggactgagaggctgctgacgatatagctctgtgttcataagatccagccttcaagtaaatctctaggtccctgaatgttgaggtagctccaaggactgagtctctacagccctaaagaaactgctgatccagttactataatttccagggaacaaacttgcactcaacacctctcttgtaagaaaatagttgttcacacatgctttaaactctaaaagttagtgaagagatcagttccctcttccaagtactttgtaactgttgggtggatccaaacagaacatgttcgaaacgactcctttaatcatgtttcaagcatgtgatgggcctccataatatgtgcttattgcttacattacctttatctatgcatttttttaggtgtcaaagagagtataacctagaacattgtatgctatatcgctacaataacacagttttgattacaaaaaaaaagaacagaaaagataatgctcacgcaatggtttgaaatagaaatttgatagatgcctcatcctacagtgctaatatggacagtgtatgtgatcattcttagcagcactttaacataacagtaagacaagctttgatgtagttttgaggaactgaaacacagcaacataaaacaaggccacttattgtggtttgatattctattaattttttataatctattgcttcaattatgtttaactgatgcaggtgaggaggataagaacgtggagataaaattgaggacatttggctctgaacaccaaaaaaaggccctttttgtggtggtaaatgcttgttctagcaaggattacatgaataatatagttggagtctgctttgtcggtcaggatgttacaggtcaaaaggtggtaatggacaaatatgtccatatacaaggtgattataaggctattgtacacagccccaatcctttgatccctcctatatttgcttcagatgaaaacacatgttgcttggagtggaacactgccatggaaaaattcacggggtggtcccggggggaagttattgggaagatgttggttggggaggtttttggcagttgctgtcagctcaagggttcagatgcactgacaaaattcatgattgctctgcacaatgcaattggagggcaagatacagacaagctaccattttcattttttgaccggaatggaaagtatgtgcaagctctcttgacagcgaacaagagggtaaatatggagggagagattgttggagccttctgcttcttgcagattgcaagtaatgagttgcagcaagctttgaaagtccagagacagcaggaaaagaagtgctctgcaaggatgaaagagttggcttacatctgccaggaaataaggaatcctttaagcggtctacgctttaccaactcgcttttggagaacacggacttgactgaagatcaaaagcagtttcttgagactagtgctgcttgtgaaaagcagatattgaagatcactcgagatgttgatcttgagagcatcgaaaatgggttagttttcctctaacaaattatcaaaacttaatattgatggtcaggtgattatttatgctttggtactattttttgagaaatgtctgttagttgagaaatactactttaaaggtatggacataatcattttggcactaggtgaatgtctttctcatgcagtttcattcccttgagttcatttggagattttttatgtctcttctacacatttcctgattatgaaccactaagactttaaattcttgactcggatgttactatactggatctgcttttaagtaatcgacactattgacttgaccacctgtggatgccaaggcggtaaacaatatgagtttcttttctgtaattccattttaagtctttttagtaatcccaggtgatagctttgcttttgacaacatctataaggaacaagatgtagggacacaaatcatttcgtaaatccattgctcacaaatgctgaattacattttgatccataactttttatgactgtgtgattaggtccaaaaacaactaaaaaataaaactctccttttataacccttttgcccctgaagtcttgatgcatcaacttaattttacaacatagataagcaatgcataatactgcagttcactttttgatagaccttagaaaaaaaattaattaatctgtaaagaaaatagatacaaccaatcaataaaactataagaattagatcatccaatccaaatatcacgaattagaaagtaaaaggcggagaaaactcaagatctacaattagagaaactaaaaaatcttttaatggtttcattagagaggagatgtcaaaaaattggatcctcttgctattgatagttgaatgtatatgaaaagattaagttaccaacaaaattacaaggaaaactcatatttttaattaaataatttggatggattcatgtgattataatttcatatttgaaattgaatttctcaagtaagatgaaatcggtcatgttactcattgataaattggaaaaaagcatagcagacaaggtaaggtgcgctgatattatttcaagtatataagttgactcgatattttaaagaacactgtttaggtattgttgttactgtgaaagaagagtgcaaatgttagaaagattatcaatataaagaagccaagaattttatcaaatgatagaaaaactgggatgaaactaaagcgataaagaattattactaggataaataacactatcatgtaaaattcatgaaattcaattagaaacaaaagatattatctttgacaacattgaaaaatgtaatagacttgagaaacaatttcaatctatgataattaatcatatcatgtgagtttgattattagttaaaaaattggttatttttgcttatattgataaattgagaaaatttaaggatcaatatggattctagcatcaaatccaaggacaaatgaaattactagaagaagacaaaaataacaatctaatggttgcaattcaatttattgtaaaagtgtgttatacacgtgtgacatggtatcatggtcgtacatatgattttcttttaaattaattattgtgcattcaagtgcattttattttatttctcatctcaacctctagcagacatatcctaacgagagggtgtctccatttgcttttgaataagtttactggagcttgagaaggctgaattcttattcgggagtgtcataaatgctgttgttagccaagcaatgctattgctgagggaaagaaatctgcaattgcttcgtgatattccagaagaaataaaaacgctggtggtttatggtgatcaggcaagaattcaacaagtactggctgatttcttgttgaatatggtgcgttatgctccatcttcagcaggttgggtagagattcatgtttgtccaacactgaagcaaatctctgatggacacactctcgtgcatatggagttcaagtatgctctcctcaactcttttgcatgttgttgataacattatctatattgataaaaggaaaatccaaaaaattgatgatgatagttacctgtgttcctttttccttctctattttcttttaaaccccttttttgtggtattctaatagctgcagtaatgttatcctggagtttcattataaatgccatctccttattctattattttcttctgtgaaactgtgatggtttctacaattttttctttagctcagtctttttgtatcttgtttgttcttccccactcctcatgattggttgcacatcctaataatctggtctccaagttgtatagaattcaatttttagatttgatctgccataaccatgcttctcaccatgcacagagctatagttgtagaattttgacatctaaactggttgcttcctcgaatttgtagaatattgggttgtatgaacaaggatggttgatgatgctgtaatagagagttatctgtgctaactgtttgttgtttaaatgataatgtggtttatatgaagtccaattcatgcttcttcattttatgcttcttctgttcatcgtatcatatagaccaaggaaatatagatgatagttgttaatcacattgcaatttgctcctccccctcttcaatgcgctttggtactaaattaactaatattatttattatcatttttcttgaaggattgtttgccctggtgaaggtcttcctcctgaattagttcaagacatgttccatagtagtagatgggttactcaagagggcctagggctcagcatgtgcaggaagattttaaagctaatgaatggtgaggtccaatatattagagagtcagaaagatgctatttcttagttatcctcgaagtacccatgcctaataagtgtgagaggtataactgtaaaaagtgctgcagactaggatgcttggtctgtaacgtctattaa

SEQ ID NO: 65 - DNA, 6600 bp, Artificial Sequence
  Synthetic Oligonucleotide
atggcatcacaatcacaaaggcaaagcaatcagccggtacataatcaagctcagtcttcgggtaccagtaacatgaggcaacaccatcacgccacagaatcagtaagcaaagcaattgcacaatacacagtcgatgcacaactccatgcagtttttgaacaatctggtggcactggtaggtcatttgattactcaaagtctgttagaactactaaccagtcagtacctgaacaacaaatcacagcgtatttgtcaaaaattcaaaggggtggtcatattcagccatttgggtgcatgattgctgcagatgaacagtcttttagggtcattgcgtatagcgaaaacgccaaagatatgcttggtttaactccacaatcagtcccttctttagagaaacaagaaattctctttgttggggctgatgtaaggattctttttaggccttccagcgctgttttgcttgaaaaagcatttggtgcaagggaaataacattgttgaatccaatttggattcattccaagaattccgggaagcccttttacgcaattttgcataggattgatgttggtattgttattgatttagagcctgctaggactgaagaccctgctttatctatagcaggggctgttcagtctcagaagttagcagttcgtgcgatttcacaattacaatcacttcctggtggggatattaagttattatgtgatactgttgtggacagtgtgagagagcttaccgggtatgatagagttatggtttataagtttcatgaggacgagcatggtgaggttgtcgctgagaataaaagggttgatttggaaccttatatcggtttgcattatccttccacggatataccacaagcttcgaggtttttgttcaagcagaatagggttaggatgattgtggattgtcatgctataccggtccgtgttattcaggatgaggcgcttatgcagcctttatgcttggttggttcgactcttcgtgctcctcacggttgtcatgctcagtatatggagaatatggggtcgattgcttcattggccatggctgttattatttatggaaatgatgaagaagctatcggtgggaggaactcgatgaggctttggggtttggttgtttgccatcacacttctgctaggtgtattccatttccgcttcgttatgcgtgtgagttcttaatgcaggctttcgggcttcaattgaacatggaattgcagttggcatcgcagttgttggagaaacatgtcttgaggactcagactctcttatgtgatatgcttctccgcgactctcctactggcattgttactcaaagtccaagtatcatggaccttgtgaagtgtgatggggcagctctttattaccaggggcagtattaccctttaggcgtgaccccaactgaaacccaaataaaagatattgtggagtggttgttgacccttcatggagacccaactggattaagcactgacagtttggccgatgctgggtatcctggggcagcctttctcggcgatgcagtttgtgggatggctgttgcttatattgctgagagagattttcttttttggtttcggtctcacactgcaaaggaggtcaaatggggtggtgcgaagcatcacccagaggacaaggatgatgggcagaggatgcatccgcgttcatcattcaaggcattcttagaggtggtgaagagccggagtttaccgtgggagaatgcagaaatggatgccattcattctttgcagcttattttgcgagactcatttagggatgctgaagcaaccaattctaaggctgttgtacatacccagctcaaggatatggaattgcaaggtatggatgagctcagttcggttgcaagagaaatggttagactaatagagactgcaactgctccgatatttgctgtagacgttgatggccgcataaacgggtggaatgcaaaggttgctgagttgactggactctcagttgaggaagccatggggaagtctttggttcacgatcttgtttataaggaatatgaagaaattgttgacaagcttattcatcgtgcagtaaaaggtacccactttatatctcacatatctcaatgaatctttttaagttgcatccgagcttacaggtttgctcctttactagaaataaaccttggaaaacatacatggttttgcaaaaagctggaattataaaagccatgaattgaaatgtaaaagaatttcaaactttcaaatcctggaattaattctatgcaataaaaggtttcatcaaattaagaaaacagctggctcactcagaagtgtcgcgtacagaaccttgtaatagtttcatttgcacagcatgcctgaatagtctcctgattaaatgatttcaatgaagtgatgtgtataattacttcttcttgaatgcactccattgataaggcaagatgaaacgttgatgcaaaagcactggtggtaacgcacaatttcttcaaaatcagagataaataaacaaggaaattaagggtcgtgggcttttcttaaggaaccagctagttgggaattgcaatactacaaatggagaacatttttcgtagatatcattcactaggatcttgggcaccacatctaggtcaaaatggctcaggcattcaccactcaatagccttgcggctgaagtattctttgtcaaactatctcttaaagtggataactggatagctgcagtcttgtattaagttatccagtgagcttagttcattgtatagctagcagcttgggagcaaatacctagcttccttattactgtagaattcataaggactgagatgcattgctttgcgttcatgagatctagctttcaagtaaatccctagggtcctgaatgttgaggcactcccaagaactgagtttccatgtctaaagaagctgctggttgaattactctaatttccagggaacttgcacattacatctcatctgtatgaaaatagtggttcatgcatttttctaaatctaaaaagttagtgaagagatcacttcaaaatggctccttcaattatgcttcgagtgtatgctctccgtaaaagaatgtgtttgttgcttatattacctttatctatgcacatttttaggtgtaccagagagtatacccgacgcaggacaatggtaggaattgaagaaagagataggaaaagcggtagaaatcctagaaatagggttcttaagatccacaacaacaagcaattatgtcaaatagcaattctcaaattcattccacgttctcccaaacaccctaaaggctctaattaaaataggaaaggcctaaaactcctaatagtactaattatatctaatgttactttaccattaacttccaagataataataaagtctgaactaataaaaaaatttaaaaaagaaagatagtagcaccaaaatgcttgttcctttggctgcatcaataacctagaatattatgtgttattactacaataactcagttgtgattaaaatttaaacaatgaaaatgcacatgctatggttttaaatagaaatttgatagatgcctcaccacacggtgctaatttggacagtgccttgatcattctaatcagcagtttaaccataacagtaggactagctttaatataggtttgaggaactgaaaacacataaaacatgaaaatttagtttgttttcgaaggttttttggatttttgatatcctattataccacttatgtttaactaatgcaggcgaagaagataagaacgtggagataaaattgaggaccttttgctctgaacaccaaaagaaggctgtttttgtggtggtaaatgcttgttcgagcaaggattacatggataatatagttggggtttgctttgttggtcaggatattacaggtcaaaaagtggtaatggacaaatacgtccttatacaaggtgattataaggctattgtacacagtcccaatccttcgatccctccgatttttgcttcagatgagaacacatgttgcttggagtggaacactgccatggaaaaactcacaggatggtccaggggcgaagttgttgggaagatgttggttggggaggtttttggcagttgctgtaggctcaagggtccagatgcactgacaaaattcatgattgccctgcacaatgcaattggagggatagatacagacaagttacccttttcattctttgaccggaatgaaaaaaatgtgcaaactctcttgacagctaacaagagggttaatatggagggagatattattggagccttctgcttcttgcagattgcaagtcctgagttgcagcaaactttgaaagttcagaaacagcaggaaaaaaaaagctttgcaaggatgaaagagttggcttacatttgccaggaaataaaaaatcctttaagtggtatacactttaccaactcgcttttggagaacacagacttgactgaagatcaacagcagtttctcgagactagtgctgcatgtgaaaaacagatattgaagatcatacgagacattgatcttgagagcattgaaaatgggtgagctttcttaaagaaattatcataacttaatattgacagtgaggtgagtatttctgctttggtaccattcttttggaaatatatatatgacatgaaatccaatagatgatgattatccttgaatgttcaaacactatactaaagttatcgacatatttgttttggcactaggtgatgtcagtttgatgcaggttccattcccttgagtccattcccttaacaaaatagccttcaaatttgactgcggtgctctgtttttagtcgtctaatactactgaatttatcatccatggatgccgagattgtaaacaatattgatttcttttctgtaattttattttgggtctttttagtaattccaggtgataacttcgcttttgacaatctgtataatcttttttaagttaatcatgtgcattcaagtgcattttcattaatttttcatctcaatctccagcatacgtacattcctaattagagagagtctccatttgcttttgaattagttcactggagcttgagaaggctgaatttttacttgggagtgtcataaatgctgttgttagccaagcaatgctattgctgagggaaagaaatctacaattgcttcgtgatattccagaagaaataaaaacgctggcagtttatggtgatcaggcaagaattcaacaagtattggctgatttcttgttgaatatggtgcgttatgctccatcttcagcaggttgggtggagattcatgtttgtccaacactgaagcaaatctctgatggacacactctcgtgcatacagagttcaagtatgttctcctcaactctcttgcaagttaatgataacaatatttatactgatagaggaaaagccaaaaatttacgatggctgttactttttttctttattttcatgtaaaccccccttttgatattctaatagctacataatgttttccttgagtttaattataaatgccacttccttattcaagtatttttcttttgtgagactctgatgatttttacagtatcttctttagttaagtacactcgaatcttgtttattcttccccactcctcatgaccagccatgcatcctaatctgacttccaagctgtaaatctcaaccagaaggtaggtttgagtcagtcaacaggtatagaattagacttgatctgccattaatcatgacaatcaccattccacagaacaataattttacaatggtgagatccaaactggctgcttccttgaatctgtcgactattggttgtatgaacaaggaaggttgatgatgctgtagtgaagagctatctgtgatttctgtttgttgtcgacatgataaagtggtttatatgaagtccaatttatggtgcttcagtttgtacgcctctgttggaaaactgtatctcctcctttgctgtccatcatgttatatacaccaagaaaatttagatgatggttgaaaactacattgtgctacttgttcctttctttctcctcctttgttagagaacggaagtctcgttagtgcacttcattactaaattgactaatattatttattattattttcttttgaaggattgtttgccctggtgaaggtcttcctcctgaattaagttcaagacatgttccatagtagtagatgggttactcaagagggcctagggctcagcatgtgcaggaagatcttaaagctaatgaatggtgaagtccaatatattagagagtcagaaagatgctatttcttagttgtcctcgaagtacccatgcctcaaaaagttggaaagggtgctgcagactaggatgcttggcctgcaacatccactaaggagctttctcaatttcaaactgacactcgttatgctgcaccgtgaggggatggttccaaaaatccagtttcaaggtttaagcgtgtgccagggccgcctgatttggttaagttatcctgaaaatacaactgttactctctatccacttacaatatggtttttgcttctttatcggcgccagtccagatcaataactcacacatga

SEQ ID NO: 66 - DNA, 3495 bp, Artificial Sequence
  Synthetic Oligonucleotide
atggtctccggaggtggtagcaaaaccagcggtggagaggcagcttcctcaggccatcgccgaagtcgtcacaccagcgctgcagaacaagctcagtcgtcagcaaacaaagccctaaggtcacagaatcagcagccacaaaaccacggtggcggaacagagtccacaaacaaagctattcaacagtacactgtcgacgcgagactccacgccgtcttcgaacaatccggagagtcaggtaagtcgtttgattactcacagtctcttaaaacggcgccgtacgattcctccgtaccagagcagcagatcacagcttatctctcccggatccaacgcggtggctatacccagccttttggctgcttgatcgccgtcgaagaatccactttcacaatcatcggttacagtgaaaatgcgcgggaaatgctagggctcatgtctcaatctgtaccaagcatcgaggacaaatcagaggttttaacgattggtacggatttgcgatctctcttcaagtcatcgagctaccttctcctcgagcgcgcgttcgtggctcgagagatcacgcttctgaatcctatttggattcactctaacaacactggtaaacctttctacgcgattctccacagggttgatgttggaattttgatcgatttagagccggctcgaaccgaagatccggcactttcaatcgccggagcagtccaatcgcagaaacttgcggtacgtgcgatttctcatttacaatcgttgcctagcggcgacattaagcttctatgtgacactgttgtggaaagcgttagagatcttactggctacgaccgcgttatggtgtacaagtttcatgaagatgaacatggtgaagtcgtagccgagagtaaacggaacgatttagagccttacattggtctgcattatcccgctactgatattcctcaggcatctcggttcttgttcaagcaaaaccgtgttaggatgatagtagattgctatgcgtcaccggttcgtgtggttcaagacgataggctcacgcagtttatatgcttggtgggttcgactttgcgagctcctcatggctgtcatgctcaatacatgactaacatgggctctattgcgtcgttagctatggcagttataataaatggaaacgaagaagatggtaatggggttaatactggaggaagaaactcgatgaggctttggggtttagttgtttgccatcacacatcagctcgttgcataccttttcctttgaggtacgcttgtgagtttcttatgcaggcctttggcttacagctaaacatggagttgcagttagccttgcaggtgtctgaaaaacgcgttctgagaatgcagacactattatgtgatatgcttctacgtgactcaccagcggggattgtcacgcagaggcctagtatcatggatttagtaaaatgtaatggtgcggcatttctttaccaagggaagtattatccgttgggtgtgactccaactgattctcagattaatgacattgtggagtggttggttgctaaccattctgattctaccgggttaagcacagatagtttaggcgatgcgggttatcctcgggcagctgctttgggagatgctgtgtgcggtatggcagtcgcgtgtatcacaaaaagggacttccttttctggtttcggtctcatactgagaaagaaatcaaatggggaggggctaagcaccatcctgaggacaaagatgatggtcagcggatgaatccgcgttcttcgttccagacttttctcgaagttgttaagagccgatgtcagccatgggaaactgctgaaatggacgccattcactcgctccagcttattctaagagactctttcaaagagtctgaagcgatggactctaaagctgctgcagctggggcggttcagccacatggagatgatatggtacagcaagggatgcaggagataggtgcagttgcaagagagatggttaggctcattgagactgcgacggttcctatatttgctgtggacatagacggttgcatcaatgggtggaacgccaagatcgcagagctgaccggtctttctgttgaagacgctatgggaaagtcgctggttcgcgaattgatatacaaagagtacaaagaaacagttgataggcttctttcttgtgctctcaaaggggatgaaggcaagaatgtggaggtcaagctgaaaacttttggttccgagctacaaggaaaagcaatgtttgtggttgtcaacgcatgttcaagcaaggactacttaaacaacatcgttggagtctgctttgttggacaagatgtaactggtcataaaattgttatggacaagttcatcaacatacaaggtgattacaaggccatcatccatagcccgaaccctctgatccctccaatctttgcagcggatgagaatacgtgctgccttgagtggaacactgcaatggaaaagctcacaggctggcctcgcagcgaagtgattggaaaattacttgttagggaagtatttgggagctattgcagactaaagggtcctgatgcgttaactaagttcatgatcgtcttgcataacgcgatcggtggccaagatactgataaattcccattcccgttctttgatcgcaaaggggaattcattcaggctctcctgactttgaacaaacgggtcagcatcgatggcaaaatcattggggctttctgttttttgcagataccgagtcccgagctgcagcaagctctagaagttcagaggaggcaggagagtgaatatttctcaaggaggaaagagttggcttacattttccaagttataaagaatccattgagtggattgcgtttcacaaattcattgctggaagacatggatttaaacgaggatcagaagcagcttcttgaaacgagtgtttcatgtgagaagcagatctcaaagattgtaggagacatggacgtcaaaagcatagatgacggttcatttctgctagagagaacagagttcttcattggcaatgtcacaaatgcagtggtaagccaagtcatgttggtggtgagagagagaaatctccagctgatccgtaacattcccacggaggtcaaatccatggctgtctacggtgaccagataaggctccaacaggttctcgcagaatttctgctaagtattgtccgttatgcacccatggaaggctcggtagagctccatctatgcccgactctgaaccaaatggctgacggattctccgctgtacgtttggagttcagaatggcgtgtgcaggggaaggtgtgccgccagagaaagtgcaagacatgttccatagtagccgatggacaagtccagaaggattaggactaagcgtttgcagaaagattttgaagctgatgaacggaggggttcagtacataagagaattcgaacgctcttatttcctaatcgttatcgaactcccggttcctctaatgatgatgatgccctcttcatga

SEQ ID NO: 67 - protein, 1172 aa, Artificial Sequence
  Synthetic Peptide
MVSGVGGSGGGRGGGRGGEEEPSSSHTPNNRRGGEQAQSSGTKSLRPRSNTESMSKAIQQYTVDARLHAVFEQSGESGKSFDYSQSLKTTTYGSSVPEQQITAYLSRIQRGGYIQPFGCMIAVDESSFRIIGYSENAREMLGIMPQSVPTLEKPEILAMGTDVRSLFTSSSSILLERAFVAREITLLNPVWIHSKNTGKPFYAILHRIDVGVVIDLEPARTEDPALSIAGAVQSQKLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEKRVLRTQTLLCDMLLRDSPAGIVTQSPSIMDLVKCDGAAFLYHGKYYPLGVAPSEVQIKDVVEWLLANHADSTGLSTDSLGDAGYPGAAALGDAVCGMAVAYITKRDFLFWFRSHTAKEIKWGGAKHHPEDKDDGQRMHPRSSFQAFLEVVKSRSQPWETAEMDAIHSLQLILRDSFKESEAAMNSKVVDGVVQPCRDMAGEQGIDELGAVAREMVRLIETATVPIFAVDAGGCINGWNAKIAELTGLSVEEAMGKSLVSDLIYKENEATVNKLLSRALRGDEEKNVEVKLKTFSPELQGKAVFVVVNACSSKDYLNNIVGVCFVGQDVTSQKIVMDKFINIQGDYKAIVHSPNPLIPPIFAADENTCCLEWNMAMEKLTGWSRSEVIGKMIVGEVFGSCCMLKGPDALTKFMIVLHNAIGGQDTDKFPFPFFDRNGKFVQALLTANKRVSLEGKVIGAFCFLQIPSPELQQALAVQRRQDTECFTKAKELAYICQVIKNPLSGMRFANSLLEATDLNEDQKQLLETSVSCEKQISRIVGDMDLESIEDGSFVLKREEFFLGSVINAIVSQAMFLLRDRGLQLIRDIPEEIKSIEVFGDQIRIQQLLAEFLLSIIRYAPSQEWVEIHLSQLSKQMADGFAAIRTEFRMACPGEGLPPELVRDMFHSSRWTSPEGLGLSVCRKILKLMNGEVQYIRESERSYFLIILELPVPRKRPLSTASGSGDMMLMMPY

SEQ ID NO: 68 - protein, 1131 aa, Artificial Sequence
  Synthetic Peptide
MSSLRPAQSSSSSSRTRQSSQARILAQTTLDAELNAEYEESGDSFDYSKLVEAQRSTPPEQQGRSGKVIAYLQHIQRGKLIQPFGCLLALDEKSFRVIAFSENAPEMLTTVSHAVPNVDDPPKLGIGTNVRSLFTDPGATALQKALGFADVSLLNPILVQCKTSGKPFYAIVHRATGCLVVDFEPVKPTEFPATAAGALQSYKLAAKAISKIQSLPGGSMQALCNTVVKEVFDLTGYDRVMAYKFHEDEHGEVFAEITKPGIEPYLGLHYPATDIPQAARFLFMKNKVRMICDCRARSVKIIEDEALSIDISLCGSTLRAPHSCHLQYMENMNSIASLVMAVVVNENEDDDEPESEQPPQQQKRKKLWGLIVCHHESPRYVPFPLRYACEFLAQVFAVHVNKEFELEKQIREKSILRMQTMLSDMLFKESSPLSIVSGSPNIMDLVKCDGAALLYGDKVWRLQTAPTESQIRDIAFWLSEVHGDSTGLSTDSLQDAGYPGAASLGDMICGMAVAKITSKDILFWFRSHTAAEIKWGGAKHDPSDEDDSRRMHPRLSFKAFLEVVKMKSLPWSDYEMDAIHSLQLILRGTLNDALKPAQSSGLDNQIGDLKLDGLAELQAVTSEMVRLMETATVPILAVDGNGLVNGWNQKVADLSGLRVDEAIGRHILTLVEDSSVPIVQRMLYLALQGREEKEVRFELKTHGSKRDDGPVILVVNACASRDMHDHVVGVCFVAQDMTVHKLVMDKFTRVEGDYRAIIHNPNPLIPPIFGADQFGWCSEWNAAMTKLTGWHRDEVIDRMLLGEVFDSSNASCLLKSKDAFVRLCIIINSALAGEEAEKAPIGFFDRDGKYIECLLSVNRKVNADGVVTGVFCFIHVPSDDLQHALHVQQASEQTALRRLKAFSYMRHAIDKPLSGMLYSRETLKGTDLDEEQMRQVRVADNCHRQLNKILADLDQDNITDKSSCLDLDMAEFVLQDVVVSAVSQVLIGCQGKGIRVACNLPERSMKQKVYGDGIRLQQILSDFLFVSVKFSPAGGSVDISSKLTKNSIGENLHLIDFELRIKHQGAGVPAEILSQMYGEDNREQSEEGLSLLVSRNLLRLMNGDIRHLREAGMSTFILTAELAAAPSAAGH

SEQ ID NO: 69 - protein, 1128 aa, Artificial Sequence
  Synthetic Peptide
MSSSRPTQCSSSSSRTRQSSRARILAQTTLDAELNAEYEEYGDSFDYSKLVEAQRTTGPEQQARSEKVIAYLHHIQRAKLIQPFGCLLALDEKTFNVIALSENAPEMLTTVSHAVPSVDDPPKLRIGTNVWSLFTDPGATALQKALGFADVSLLNPILVQCKTSGKPFYAIVHRATGCLVVDFEPVKPTEFPATAAGALQSYKLAAKAISKIQSLPGGSMEVLCNTVVKELFDLTGYDRVMAYKFHEDDHGEVFAEITKPGLEPYLGLHYPATDIPQAARFLFMKNKVRMICDCRARSIKIIEDESLHLDISLCGSTLRAPHSCHLQYMENMNSIASLVMAVVVNENEDDDEVGADQPAQQQKRKKLWGLLVCHHESPRYVPFPLRYACEFLAQVFAVHVNKEFELERQVREKSILRMQTMLSDMLLRESSPLSIVSGTPNIMDLVKCDGAALLYGGKVWRLQNAPTESQIRDIAFWLSDVHRDSTGLSTDSLHDAGYPGAAALGDMICGMAVAKINSKDILFWFRSHTAAEIRWGGAKHDPSDKDDSRRMHPRLSFKAFLEVVKMKSLPWNDYEMDAIHSLQLILRGTLNDDIKPTRAASLDNQVGDLKLDGLAELQAVTSEMVRLMETATVPILAVDSNGLVNGWNQKVAELTGLRVDEAIGRHILTVVEESSVPVVQRMLYLALQGKEEKEVKFEVKTHGSKRDDGPVILVVNACASRDLHDHVVGVCFVAQDMTVHKLVMDKFTRVEGDYKAIIHNPSPLIPPIFGADEFGWCSEWNAAMTKLTGWHRDEVINKMLLGEVFDSTNASCLVKNKDAFVSLCILINSALAGDETEKAPFSFFDRNGKYIECLLSVNRKVNADGVITGVFCFIQVPSHELQHALHVQQASQQNALTKLKAYSYMRHAINNPLSGMLYSRKALKNTGLNEEQMKEVNVADSCHRQLNKILSDLDQDSVMNKSSCLDLEMVEFVLQDVFVAAVSQVLITCQGKGIRVSCNLPERYMKQTVYGDGVRLQQILSDFLFVSVKFSPVGGSVEISCSLTKNSIGENLHLIDLELRIKHQGKGVPADLLSQMYEDDNKEQSDEGMSLAVSRNLLRLMNGDVRHMREAGMSTFILSVELASAPAK

SEQ ID NO: 70 - protein, 1129 aa, Artificial Sequence
  Synthetic Peptide
MSSSRPASSSSSRNRQSSRARVLAQTTLDAELNAEYEESGDSFDYSKLVEAQRDGPPVQQGRSEKVIAYLQHIQKGKLIQTFGCMLALDEKSFNVIAFSENAPEMLTTVSHAVPSVDDPPRLGIGTNVRSLFSDQGATALHKALGFADVSLLNPILVQCKTSGKPFYAIVHRATGCLVVDFEPVKPTEFPATAAGALQSYKLAAKAISKIQSLPGGSMEVLCNTVVKEVFDLTGYDRVMAYKFHEDDHGEVFAEITKPGLEPYLGLHYPATDIPQAARFLFMKNKVRMICDCRARSIKVIEAEALPFDISLCGSALRAPHSCHLQYMENMNSIASLVMAVVVNENEEDDEAESEQPAQQQQKKKLWGLLVCHHESPRYVPFPLRYACEFLAQVFAVHVNREFELEKQLREKSILKMQTMLSDMLFREASPLTIVSGAPNIMDLVKCDGAALLYGGKVWRLRNAPTESQIHDIAFWLSDVHRDSTGLSTDSLHDAGYPGASALGDMICGMAVAKINSKDIIFWFRSHTAAEIRWGGAKHDSSDMDDSRRMHPRLSFKAFLEVVKMKSLPWTDYEMDAIHSLQLILRGTLNDASKPKREASLDNQIGDLKLDGLAELQAVTSEMVRLMETATVPILAVDGNGLVNGWNQKAAELTGLRVDDAIGRHILTLVEESSVPVVQRMLYLALQGKEEKEVRFEVKTHGPRRDDGPVILVVNACASRDLHDHVVGVCFVAQDMTVHKLVMDKFTRVEGDYKAIIHNPNPLIPPIFGADEFGWCSEWNAAMTKLTGWNRDEVLDKMLLGEVFDSSNASCPLKNKNAFVSLCVLINSALAGEETEKAPFGFFDRSGKYIECLLSANRKENEGGLITGVFCFIHVASHELQHALQVQQASEQTSLKRLKAFSYMRHAINNPLSGMLYSRKALKNTDLNEEQMKQIHVGDNCHHQINKILADLDQDSISEKSSCLDLEMAEFVFQDVVVAAVSQVLITCQGKGIRISCNLPERFMKQSVYGDGVRLQQILSDFLFISVKFSPVGGSVEISSKLTKNSIGENLHLIDLELRIKHQGLGVPAELMEQMFEEDNKEQSDEGLGLLVSRKLLRLMNGDVRHLREAGVSTFILTAELASAPTAIGQ

SEQ ID NO: 71 - protein, 1122 aa, Artificial Sequence
  Synthetic Peptide
MSGSRPTQSSEGSRRSRHSARIIAQTTVDAKLHADFEESGSSFDYSTSVRVTGPVVENQPPRSDKVTTTYLHHIQKGKLIQPFGCLLALDEKTFKVIAYSENASELLTMASHAVPSVGEHPVLGIGTDIRSLFTAPSASALQKALGFGDVSLLNPILVHCRTSAKPFYAIIHRVTGSIIIDFEPVKPYEVPMTAAGALQSYKLAAKAITRLQSLPSGSMERLCDTMVQEVFELTGYDRVMAYKFHEDDHGEVVSEVTKPGLEPYLGLHYPATDIPQAARFLFMKNKVRMIVDCNAKHARVLQDEKLSFDLTLCGSTLRAPHSCHLQYMANMDSIASLVMAVVVNEEDGEGDAPDATTQPQKRKRLWGLVVCHNTTPRFVPFPLRYACEFLAQVFAIHVNKEVELDNQMVEKNILRTQTLLCDMLMRDAPLGIVSQSPNIMDLVKCDGAALLYKDKIWKLGTTPSEFHLQEIASWLCEYHMDSTGLSTDSLHDAGFPRALSLGDSVCGMAAVRISSKDMIFWFRSHTAGEVRWGGAKHDPDDRDDARRMHPRSSFKAFLEVVKTRSLPWKDYEMDAIHSLQLILRNAFKDSETTDVNTKVIYSKLNDLKIDGIQELEAVTSEMVRLIETATVPILAVDSDGLVNGWNTKIAELTGLSVDEAIGKHFLTLVEDSSVEIVKRMLENALEGTEEQNVQFEIKTHLSRADAGPISLVVNACASRDLHENVVGVCFVAHDLTGQKTVMDKFTRIEGDYKAIIQNPNPLIPPIFGTDEFGWCTEWNPAMSKLTGLKREEVIDKMLLGEVFGTQKSCCRLKNQEAFVNLGIVLNNAVTSQDPEKVSFAFFTRGGKYVECLLCVSKKLDREGVVTGVFCFLQLASHELQQALHVQRLAERTAVKRLKALAYIKRQIRNPLSGIMFTRKMIEGTELGPEQRRILQTSALCQKQLSKILDDSDLESIIEGCLDLEMKEFTLNEVLTASTSQVMMKSNGKSVRITNETGEEVMSDTLYGDSIRLQQVLADFMLMAVNFTPSGGQLTVSASLRKDQLGRSVHLANLEIRLTHTGAGIPEFLLNQMFGTEEDVSEEGLSLMVSRKLVKLMNGDVQYLRQAGKSSFIITAELAAANK

SEQ ID NO: 72 - protein, 1124 aa, Artificial Sequence
  Synthetic Peptide
MSSSRPSQSSTTSARSKHSARIIAQTTIDAKLHADFEESGDSFDYSSSVRVTSVAGDERKPKSDRVTTAYLNQIQKGKFIQPFGCLLALDEKTFKVIAFSENAPEMLTMVSHAVPSVGELPALGIGTDIRTIFTGPSAAALQKALGFGEVSLLNPVLVHCKTSGKPYYAIVHRVTGSLIIDFEPVKPYEVPMTAAGALQSYKLAAKAITRLQALPSGSMERLCDTMVQEVFELTGYDRVMTYKFHDDDHGEVVAEITKPGLDPYLGLHYPATDIPQAARFLFMKNKVRMICDCRAKHVKVVQDEKLPFDLTLCGSTLRAPHYCHLQYMENMSSIASLVMAVVVNDGDEEGESSDSTQSQKRKRLWGLVVCHNTTPRFVPFPLRYACEFLAQVFAIHVNKELELESQILEKNILRTQTLLCDMLMRVAPLGIVSQSPNIMDLVKCDGAALLYKNKIHRLGMTPSDFQLHDIVSWLSEYHTDSTGLSTDSLYDAGFPGALALGDVVCGMAAVRISDKGWLFWYRSHTAAEVRWGGAKHEPGEKDDGRKMHPRSSFKAFLEVVKTRSVPWKDYEMDAIHSLQLILRNASKDADAMDSNTNIIHTKLNDLKIDGLQELEAVTAEMVRLIETASVPIFAVDVDGQLNGWNTKIAELTGLPVDEAIGNHLLTLVEDSSVDTVSKMLELALQGKEERNVEFEIKTHGPSGDSSPISLIVNACASRDVGDSVVGVCFIAQDITGQKNIMDKFTRIEGDYRAIIQNPHPLIPPIFGTDQFGWCSEWNSAMTKLTGWRRDDVIDKMLLGEVFGTQAACCRLKNQEAFVNFGVVLNNAMTGQECAKISFGFFARNGKYVECLLCVSKRLDREGAVTGLFCFLQLASHELQQALHIQRLSEQTALKRLKVLAYIRRQIRNPLSGIIFSRKMLEGTNLGEEQKNILRTSSQCQRQLNKILDDTDLDSIIDGYLDLEMLEFKLHEVLVASISQIMMKSNGKNIMIVNDMVEDLLNETLYGDSPRLQQVLANFLLVCVNSTPSGGQLSISGTLTKDRIGESVQLALLEVRISHTGGGVPEELLSQMFGTEAEASEEGISLLISRKLVKLMNGEVQYLREAGRSTFIISVELAVATKSSC

SEQ ID NO: 73 - protein, 1112 aa, Artificial Sequence
  Synthetic Peptide
MGFESSSSAASNMKPQPQKSNTAQYSVDAALFADFAQSIYTGKSFNYSKSVISPPNHVPDEHITAYLSNIQRGGLVQPFGCLIAVEEPSFRILGLSDNSSDFLGLLSLPSTSHSGEFDKVKGLIGIDARTLFTPSSGASLSKAASFTEISLLNPVLVHSRTTQKPFYAILHRIDAGIVMDLEPAKSGDPALTLAGAVQSQKLAVRAISRLQSLPGGDIGALCDTVVEDVQRLTGYDRVMVYQFHEDDHGEVVSEIRRSDLEPYLGLHYPATDIPQAARFLFKQNRVRMICDCNATPVKVVQSEELKRPLCLVNSTLRAPHGCHTQYMANMGSVASLALAIVVKGKDSSKLWGLVVGHHCSPRYVPFPLRYACEFLMQAFGLQLQMELQLASQLAEKKAMRTQTLLCDMLLRDTVSAIVTQSPGIMDLVKCDGAALYYKGKCWLVGVTPNESQVKDLVNWLVENHGDDSTGLTTDSLVDAGYPGAISLGDAVCGVAAAEFSSKDYLLWFRSNTASAIKWGGAKHHPKDKDDAGRMHPRSSFTAFLEVAKSRSLPWEISEIDAIHSLRLIMRESFTSSRPVLSGNGVARDANELTSFVCEMVRVIETATAPIFGVDSSGCINGWNKKTAEMTGLLASEAMGKSLADEIVQEESRAALESLLCKALQGEEEKSVMLKLRKFGQNNHPDYSSDVCVLVNSCTSRDYTENIIGVCFVGQDITSEKAITDRFIRLQGDYKTIVQSLNPLIPPIFASDENACCSEWNAAMEKLTGWSKHEVIGKMLPGEVFGVFCKVKCQDSLTKFLISLYQGIAGDNVPESSLVEFFNKEGKYIEASLTANKSTNIEGKVIRCFFFLQIINKESGLSCPELKESAQSLNELTYVRQEIKNPLNGIRFAHKLLESSEISASQRQFLETSDACEKQITTIIESTDLKSIEEGKLQLETEEFRLENILDTIISQVMIILRERNSQLRVEVAEEIKTLPLNGDRVKLQLILADLLRNIVNHAPFPNSWVGISISPGQELSRDNGRYIHLQFRMIHPGKGLPSEMLSDMFETRDGWVTPDGLGLKLSRKLLEQMNGRVSYVREDERCFFQVDLQVKTMLGVESRGTEGSSSIK

SEQ ID NO: 74 - protein, 1111 aa, Artificial Sequence
  Synthetic Peptide
MSSNTSRSCSTRSRQNSRVSSQVLVDAKLHGNFEESERLFDYSASINLNMPSSSCEIPSSAVSTYLQKIQRGMLIQPFGCLIVVDEKNLKVIAFSENTQEMLGLIPHTVPSMEQREALTIGTDVKSLFLSPGCSALEKAVDFGEISILNPITLHCRSSSKPFYAILHRIEEGLVIDLEPVSPDEVPVTAAGALRSYKLAAKSISRLQALPSGNMLLLCDALVKEVSELTGYDRVMVYKFHEDGHGEVIAECCREDMEPYLGLHYSATDIPQASRFLFMRNKVRMICDCSAVPVKVVQDKSLSQPISLSGSTLRAPHGCHAQYMSNMGSVASLVMSVTINGSDSDEMNRDLQTGRHLWGLVVCHHASPRFVPFPLRYACEFLTQVFGVQINKEAESAVLLKEKRILQTQSVLCDMLFRNAPIGIVTQSPNIMDLVKCDGAALYYRDNLWSLGVTPTETQIRDLIDWVLKSHGGNTGFTTESLMESGYPDASVLGESICGMAAVYISEKDFLFWFRSSTAKQIKWGGARHDPNDRDGKRMHPRSSFKAFMEIVRWKSVPWDDMEMDAINSLQLIIKGSLQEEHSKTVVDVPLVDNRVQKVDELCVIVNEMVRLIDTAAVPIFAVDASGVINGWNSKAAEVTGLAVEQAIGKPVSDLVEDDSVETVKNMLALALEGSEERGAEIRIRAFGPKRKSSPVELVVNTCCSRDMTNNVLGVCFIGQDVTGQKTLTENYSRVKGDYARIMWSPSTLIPPIFITNENGVCSEWNNAMQKLSGIKREEVVNKILLGEVFTTDDYGCCLKDHDTLTKLRIGFNAVISGQKNIEKLLFGFYHRDGSFIEALLSANKRTDIEGKVTGVLCFLQVPSPELQYALQVQQISEHAIACALNKLAYLRHEVKDPEKAISFLQDLLHSSGLSEDQKRLLRTSVLCREQLAKVISDSDIEGIEEGYVELDCSEFGLQESLEAVVKQVMELSIERKVQISCDYPQEVSSMRLYGDNLRLQQILSETLLSSIRFTPALRGLCVSFKVIARIEAIGKRMKRVELEFRIIHPAPGLPEDLVREMFQPLRKGTSREGLGLHITQKLVKLMERGTLRYLRESEMSAFVILTEFPLI

SEQ ID NO: 75 - protein, 1137 aa, Artificial Sequence
  Synthetic Peptide
MSSSRSNNRATCSRSSSARSKHSARVVAQTPMDAQLHAEFEGSQRHFDYSSSVGAANRSGATTSNVSAYLQNMQRGRFVQPFGCLLAVHPETFALLAYSENAAEMLDLTPHAVPTIDQREALAVGTDVRTLFRSHSFVALQKAATFGDVNLLNPILVHARTSGKPFYAIMHRIDVGLVIDLEPVNPVDLPVTATGAIKSYKLAARAIARLQSLPSGNLSLLCDVLVREVSELTGYDRVMAYKFHEDEHGEVIAECKRSDLEPYLGLHYPATDIPQASRFLFMKNKVRMICDCSATPVKIIQDDSLTQPISICGSTLRAPHGCHAQYMASMGSVASLVMSVTINEDEDDDGDTGSDQQPKGRKLWGLMVCHHTSPRFVPFPLRYACEFLLQVFGIQINKEVELAAQAKERHILRTQTLLCDMLLRDAPVGIFTQSPNVMDLVKCDGAALYYQNQLWVLGSTPSEAEIKNIVAWLQEYHDGSTGLSTDSLVEAGYPGAAALGDVVCGMAAIKISSKDFIFWFRSHTAKEIKWGGAKHEPIDADDNGRKMHPRSSFKAFLEVVKWRSVPWEDVEMDAIHSLQLILRGSLQDEDANKNNNAKSIVTAPSDDMKKIQGLLELRTVTNEMVRLIETATAPILAVDITGSINGWNNKAAELTGLPVMEAIGKPLVDLVIDDSVEVVKQILNSALQGIEEQNLQIKLKTFNHQENNGPVILMVNACCSRDLSEKVVGVCFVAQDMTGQNIIMDKYTRIQGDYVAIVKNPSELIPPIFMINDLGSCLEWNEAMQKITGIKREDAVDKLLIGEVFTHHEYGCRVKDHGTLTKLSILMNTVISGQDPEKLLFGFFNTDGKYIESLMTATKRTDAEGKITGALCFLHVASPELQHALQVQKMSEQAAMNSFKELTYIRQELRNPLNGMQFTRNLLEPSDLTEEQRKLLASNVLCQEQLKKILHDTDLESIEQCYTEMSTVDFNLEEALNTVLMQAMPQSKEKQISIDRDWPAEVSCMHLCGDNLRLQQVLADFLACMLQFTQPAEGPIVLQVIPRMENIGSGMQIAHLEFRLVHPAPGVPEALIQEMFRHSPGASREGLGLYISQKLVKTMSGTVQYLRESESSSFIVLVEFPVAQLSTKRCKASTSKF

SEQ ID NO: 76 - protein, 1135 aa, Artificial Sequence
  Synthetic Peptide
MSLPSNNRRTCSRSSSARSKHSARVVAQTPVDAQLHAEFEGSQRHFDYSSSVGAANRPSASTSTVSTYLQNMQRGRYIQPFGCLLAVHPDTFALLAYSENAPEMLDLTPHAVPTIDQRDALGIGVDVRTLFRSQSSVALHKAAAFGEVNLLNPILVHARTSGKPFYAILHRIDVGLVIDLEPVNPADVPVTAAGALKSYKLAAKAISRLQSLPSGNLSLLCDVLVREVSELTGYDRVMAYKFYEDEHGEVISECRRSDLEPYLGLHYPATDIPQASRFLFMKNKVRMICDCCATPVKVIQDDSLAQPLSLCGSTLRASHGCHAQYMANMGSVASLAMSVTINEDEEEDGDTGSDQQPKGRKLWGLVVCHHTSPRFVPFPLRYACEFLLQVFGIQLNKEVELAAQAKERHILRTQTLLCDMLLRDAPVGIFTRSPNVMDLVKCDGAALYYQNQLLVLGSTPSESEIKSIATWLQDNHDGSTGLSTDSLVEAGYPGAVALREVVCGMAAIKISSKDFIFWFRSHTTKEIKWGGAKHEPVDADDDGRRMHPRSSFKAFLEVVKWRSVPWEDVEMDAIHSLQLILRGSLPDEDANRNNVRSIVKAPSDDMKKIQGLLELRTVTNEMVRLIETATAPVLAVDIAGNINGWNNKAAELTGLPVMEAIGRPLIDLVVTDSIEVVKQILDSALQGIEEQNMEIKLKTFHEHECNGPVILKVNSCCSRDLSEKVIGVCFVAQDLTRQKMIMDKYTRIQGDYVAIVKNPTELIPPIFMINDLGSCLEWNKAMQKITGIKREDAINKLLIGEVFTLHDYGCRVKDHATLTKLSILMNAVISGQDPEKLFFGFFDTDGKYIESLLTVNKRTDAEGKITGALCFLHVASPELQHALQVQKMSEQAATNSFKELTYIRQELRNPLNGMQFTCNLLKPSELTEEQRQLLSSNVLCQDQLKKILHDTDLESIEQCYMEMNTVEFNLEQALNTVLMQGIPLGKEKQISIERNWPVEVSCMYLYGDNLRLQQILADYLACALQFTQTAEGPIVLQVMSKKENIGSGMQIAHLEFRIVHPAPGVPEALIQEMFQHNPGVSREGLGLYISQKLVKTMSGTVQYLREADTSSFIILMEFPVAQLSSKRSKPSTSKF

SEQ ID NO: 77 - protein, 1135 aa, Artificial Sequence
  Synthetic Peptide
MSSPSNNRGTCSRSSSARSKHSARVVAQTPVDAQLHADFEGSQRHFDYSSSVGAANRPSASTSTVSTYLQNMQRGRYIQPFGCLLAVHPDTFALLAYSENAPEMLDLTPHAVPTIDQRDALTIGADVRTLFRSQSSVALHKAATFGEVNLLNPILVHARTSGKPFYAILHRIDVGLVIDLEPFNPADVPVTAAGALKSYKLAAKAISRLQSLPSGNLSLLCDVLVREVSELTGYDRVMAYKFHEDEHGEVISECRRSDLEPYLGLHYPATDIPQASRFLFMKNKMRMICDFSATPVLIIQDGSLAQPVSLCGSTLRASHGCHAQYMANMGSVASLVMSVTINDDEEEDGDTDSDQQPKGRKLWGLVVCHHTSPRFVPFPLRYACEFLLQVFGIQLSKEVELAAQAKERHILRTQTLLCDMLLRDALVGIFTQSPNVMDLVKCDGAALYYQNQVLVLGSTPSESEIKSIATWLQENHDGSTGLSTDSLVEAGYPGAAALREVVCGMVAIKISSKNFIFWFRSHTTKEIKWSGAKHEPFDADDNGRKMHPRSSFKAFLEVVKWRSVPWEDVEMDAIHSLQLILRDSLQGEDANRNNIRSIVKAPSDDMKKLQGLLELRTVTNEMVRLIETATAPVLAVDIAGNINGWNKKAAELTGLPVMEAIGRPLIDLVVADSVEVVKQILDSALQGIEEQNLEIKLKTFHEQECCGPVILMINSCCSRDLSEKVIGVCFVAQDLTRQKMIMDKYTRIQGDYVAIIKNPSELIPPIFMINDLGSCLEWNKAMQKITGMKREDAINKLLIGEVFTLHDYGCRVKDHATLTKLSILMNAVISGQDPEKLLFGFFGTGGKYIESLLTVNKRTNAEGKITGALCFLHVASPELQHALEVQKMSEQAATNSFKELTYIRQELRNPLNGMQFTYNLLKPSELTEDQRQLVSSNVLCQDQLKKILHDTDLESIEQCYMETNTVEFNLEEALNTVLMQGIPLGKEKRISIERDWPVEVSHMYIYGDNIRLQQVLADYLACALQFTQPAEGHIVLQVIPKKENIGSGMQIAHLEFRIVHPAPGVPEALIQEMFQHNPGVSREGLGLYISQKLVKTMSGTLQYLREADTSSFIILIEFPVAQLSSKRSKPSPSKF

SEQ ID NO: 78 - protein, 1123 aa, Artificial Sequence
  Synthetic Peptide
MSTPKKTYSSTSSAKSKAHSVRVAQTTADAALQAVFEKSGDSGDSFDYSKSVSKSTAESLPSGAVTAYLQRMQRGGLTQSFGCMIAVEGTGFRVIAYSENAPEILDLVPQAVPSVGEMDTLRIGTDVRTLFTASSVASLEKAAEAQEMSLLNPITVNCRRSGKQLYAIAHRIDIGIVIDFEAVKTDDHLVSAAGALQSHKLAAKAITRLQALPGGDIGLLCDTVVEEVRELTGYDRVMAYRFHEDEHGEVVAEIRRADLEPYLGLHYPGTDIPQASRFLFMKNKVRIIADCSAPPVKVIQDPTLRQPVSLAGSTLRSPHGCHAQYMGNMGSIASLVMAVIINDNEEDSHGSVQRGRKLWGLVVCHHTSPRTVPFPLRSACGFLMQVFGLQLNMEVELAAQLREKHILRTQTLLCDMLLRDAPIGIVSQIPNIMDLVKCDGAALYYGKRFWLLGTTPTESQIKDIAEWLLEYHKDSTGLSTDSLADANYPAAHLLGDAVCGMAAAKITAKDFLFWFRSHTAKEIKWGGAKHDPGEKDDGRKMHPRSSFKAFLEVVKRRSLPWEDVEMDAIHSLQLILRGSFQDIDDSDTKTMIHARLNDLKLHDMDELSVVANEMVRLIETATAPILAVDSNGMINGWNAKIAQVTGLPVSEAMGRSLVKDLVTDESVAVVERLLYLALRGEEEQNVEIKLKTFGTQTEKGVVILIVNACSSRDVSENVVGVCFVGQDVTGQKMFMDKFTRIQGDYKTIVQNPHPLIPPIFGADEFGYCFEWNPAMEGLTGWKKDEVVGKLLVGEIFGMQMMCCRMKSQDAMTKFMIALNTAMDGQSTDKFTFSFFDREGKYVDVLLSTNKRTNADGVITGVFCFLQIASSELQQALKVQRATEKVAVAKLKELAYIRQEIKNPLCGITFTRQLLEDTDLSDDQQQFLDTSAVCEQQLQKVLNDMDLESIEDGYLELDTAEFEMGTVMNAVISQGMTTSREKGLQIFRETPREINTMRLLGDQIRLQQVLSDFLLNTVRFTPSPEGWVKIKVVPTRKRLGGSVHVVHLEFRVSHPGAGLPEELVLEMYDRGKGMTQEGLGLNMCRKLVRLMNGDVHYVREAMQCYFVVNVELPMAQRDDASSQM

SEQ ID NO: 79 - protein, 1122 aa, Artificial Sequence
  Synthetic Peptide
MSAPKKTYSSTSSAKSKAHSVRVAQTTADAALHAVFEKSGVSGDNFDYSKSVSKSTAGSLHTGAVTAYLQRMQRGGLTQSFGCMVAVEETGFRVIAYSENAPEFLDLMPQAVPNIGEINTLGIGTDVRTLFTPSSAASLEKAAETQEISLLNPITVYCRSKKPLYAIAHRIDIGIVIDFEAVNMNDVTISADGALQSHKLAAKAITRLQALPGGDIGLLCDTVVEEVRELTGYDRVMAYKFHEDEHGEVVAEIRRTDLEPYLGLHYPATDIPQASRFLFMKNRVRMIGDCSAPPVKIVQDPNLRQPVSLAGSTLRSPHGCHAQYMGNMGSISSIVMAVIINDNEDDSRGSVQRGRKLWGLVVCHHTSPRTVPFPLRSACEFLMQVFGLQLNMEVELAAQLREKHILRTQTLLCDMLLRDAPIGIVSQVPNIMDLVKCDGAALYYGKRFWLLGTTPTESQIKDIAEWLLEYHKDSTGLSTDSLADANYPAAHLLGDAVCGMAAAKITSKDFLFWFRSHTAKEIKWGGAKHDPGEKDDNRKMHPRSSFKAFLEVVKRRSLPWEDVEMDAIHSLQLILRGSFQDIDDSDTKTMIHARLNDLKLHDMDELSIVANEMVRLIETATAPILAVDSNGMINGWNAKIAQETGLPVAEAMGRSLVKDLVMDESLEVVERLLYLALRGEEEQGVEIKLKTFGAQTVKGAVILIVNACASRDVSENVVGVCFVGQDVTGQKMFMDKFTRIQGDYKTIVQNPHPLIPPIFGADEFGYCFEWNPAMEGLTGWKRDEVIGKLLVGEIFGMQKMCCQMKSQDAMTKFMISLNSAMDGQNTDKFSLSFFDREGRYVDALLSTNKRTNADGAITGVICFLQIASSELQQALRVQQATEKVAIAKLKELAYIRQEIKNPLCGITFTRQLLEDTDLSNDQKQFLDTSAVCEQQLQKVLNDLDLESIEDGYLELDTAEFEMRTVMDAVISQGMTISREKGLQIIRETPREIITMRLFGDQVRLQQVLSDFLLNAVRFTPSSEGWVKIKVVPTRKRLGGNEHVMHLEFRVSHPGAGLPEELVLEMFDKGKGMTQEGLGLNICRKLVRLMNGDVQYVREAMQCYFVLYVELPLAQQDDAASQM

SEQ ID NO: 80 - protein, 1134 aa, Artificial Sequence
  Synthetic Peptide
MSTTKLTYSSGSSAKSKHSVRVAQTTADAKLHAVYEESGESGDSFDYSKSINATKSTGETIPAQAVTAYLQRMQRGGLVQPFGCMLAVEEGSFRVIAFSDNAGEMLDLMPQSVPSLGSGQQDVLTIGTDARTLFTAAASALEKAAGAVDLSMLNPIWVQSKTSAKPFYAIVHRIDVGLVMDLEPVKASDTRVGSAAGALQSHKLAAKAISRLQSLPGGDIGLLCDTVVEEVRDVTGYDLVMAYKFHEDEHGEVVAEIRRSDLEPYLGLHYPATDIPQASRFLFMKNRVRMICDCSAPPVKITQDKELRQPISLAGSTLRAPHGCHAQYMGNMGSVASLVMAMIINDNDEPSGGGGGGGQHKGRRLWGLVVCHHTSPRSVPFLRSACEFLMQVFGLQLNMEAAVAAHVREKHILRTQTLLCDMLLRDAPIGIVSQSPNIMDLVKCDGAALYYGKRFWLLGITPSEAQIKDIAEWLLEHHKDSTGLSTDSLADAGYPGAASLGDEVCGMAAAKITAKDFLFWFRSHTAKEVKWGGAKHDPDDKDDGRKMHPRSSFKAFLEVVKRRSLPWEDVEMDAIHSLQLILRGSFQDIDDSDTKTMIHARLNDLKLQGMDELSTVANEMVRLIETATAPILAVDSSGFINGWNAKVADVTGLPVTEAMGRSLAKELVLHESADMVERLLYLALQGDEEQNVELKLKTFGGQKDKEAVILVVNACASRDVSDNVVGVCFVGQDVTGQKVVMDKFTRIQGDYKAIVQNPNPLIPPIFGADEFGYCSEWNPAMEKLSGWRREEVLGKMLVGEIFGIQMMYCRLKGQDAVTKFMIVLNSAADGQDTEKFPFAFFDRQGKYVEALLTATKRADAEGSITGVFCFLHIASAELQQALTVQRATEKVALSKLKELAYIRQEIKNPLYGIMFTRTLMETTDLSEDQKQYVETGAVCEKQIRKILDDMDLESIEDGYLELDTTEFMMGTVMDAVISQGMITSKEKNLQLIRETPKEIKAMFLYGDQVRLQQVLADFLLNAIRFTPSSENWVGIKVATSRKRLGGVVHVMHLEFRITHPGVGLPEELVQEMFDRGRGMTQEGLGLSMCRKLVKLMNGEVEYIREAGKNYFLVSLELPLAQRDDAGSVKFQASS

SEQ ID NO: 81 - protein, 1130 aa, Artificial Sequence
  Synthetic Peptide
MSTPKLAYSSGSSVKSKHSVRVAQTTADAKLQAVYEESGDSGDSFDYSKSVHASKSTGENVSAQAVTAYLQRMQRGGLMQTFGCMLCVEESNFRVIAFSENAPEMLDLMPQAVPSVGQQEVLGIGTDARTLFTPSSAAALEKCAGAVDVTMLNPISVHCRSSGKPFYAILHRIDVGLVIDFEPVRSNDAIVSSAGVLQSHKLAAKAISRLQALPGGDIGLLCDIVVQEVRELSGYDRVMAYKFHEDEHGEVLAEIRRSDLEPYLGLHYPATDIPQASRFLFMKNKVRMIGDCFASPVKVIQDKDLRQPISLAGSTLRAPHGCHAQYMGNMNSIASLVMAVIVNDPDEDPNARGGQQRGRKLWGLVVCHHTSPRTVSFPLRSACEFLMQVFGLQLNMEVELAAQLREKHILRTQTLLCDMLLRDAPIGIVSQSPNIMDLVKCDGAALYYGKRFWLLGITPNDAQIKEIADWLLEHHQDSTGLSTDSLADAGYPGAAQLGDAVCGMAAAKITSKDFLFWFRSHTAKEIKWGGAKHDPDEKDDGRKMHPRSSFKAFLVVVKRRSLPWEDIEMDAIHSLQLILRGSFQDIDDSDTKTMIHARLNDLKLQGMDELSTVANEMVRLIETATAPILAVDSGGFINGWNAKVAELTGLPVEEAMGRSLVKDLILNESIDVVQRLLHLALQGDEEQNIEIQLKTFGPQKEKGAVILIVNACSSRDVQDNVVGVCFVGQDVTGQKQVLDKFTRIQGDYKAIVQNPNPLIPPIFGTDEYGYCSEWNPSMEKLTGWKREEVIGKLLVGEIFGIQLMCCRLKSQDAMTKFMIVLNGAMDGQDTDRFPFSFFDRQGKYVDPLLTVNKRTDAEGSITGVFCFLHTTSVELLQALTVQRSTEKVAFAKLKELAYIRQEIKNPLYGIVFTRNLMEDTDLSVDQRQFVETSAVCERQLRKILDDLDLESIEDGYLELDTTEFEMGTVMDAVVSQGMITSREKGLQLIRETPSEIKNMCLYGDQVRLQQVLADFLLNAVRFTPSSEGWVGIKVVPTKKRLGRGVHVMHLEFRVTHPGLGLPEELVHEMFGRGRGMTQEGLGLSMCRKLVKLMNGTVQYIRETGKSCFLVEVELPLAQRDDAGSVRSTVV

SEQ ID NO: 82 - protein, 1126 aa, Artificial Sequence
  Synthetic Peptide
MSTTKLAYSSGSSVKSKHSVRVAQTTADAKLQAVYEESGDSGDSFDYSKSVHASKSTGENVPALAVTAYLQRMQRGGLVQTFGCMLCVDESSFRVIAYSENAPEMLDLMPQAVPSVGQQEVLGIGTDARTLFTPSSAAALEKCAGAVDVTMLNPISVHCRSSGKPFYAILHRIDVGLVIDFEPVRPNDAVVSSAGALQSHKLAAKAISRLQALPGGDIGLLCDTVVEEVRQLSGYDRVMAYKFHEDEHGEVLAEIRRSDLEPYLGLHYPATDIPQASRFLFMKNRVRMIGDCYAPPVKVVQDKDLRQPISLAGSTLRAPHGCHAQYMGNMNSIASLVMAVIVNDPDEDPNSRGGQQRGRKLWGLVVCHHTSPRTVSFPLRSACEFLMQVFGLQLNMEVELAAQLREKHILRTQTLLCDMLLRDAPIGIVSQSPNIMDLVKCDGAALYYGKRFWLLGITPNEVQIKEIADWLLEHHQDSTGLSTDSLADAGYPGAAQLGDAVCGMAAAKITPRDFLFWFRSHTAKEIKWGGAKHDPDEKDDGRKMHPRSSFKAFLEVVKRRSLPWEDIEMDAIHSLQLILRGSFQDIDDSDTKTMIHARLNDLKLQGMDELSTVANEMVRLIETATAPILAVDSSGFINGWNAKVAELTGLPVGEAMGRSLVKDLILEESIDVVQRLLYLALQGEEEQNIEIQLKTFGPQKEKGAVILIVNACSSRDVQDNVVGVCFVGQDVTGQKQVLDKFTRIQGDYKAIVQNPNPLIPPIFGTDEYGYCSEWNPSMEKLTGWKREEVLGKLLVGEIFGMQLMCCRLKGQDAMTKFMIALNSAMDGQDTDRFPFSFFDRQGKYVDALLTVNKRTDAEGSITGVFCFLHTTSVELLQALTVQRATEKVAFAKLKELAYIRQEIKNPLYGIMFTRNLMEDTDLSEDQRQFVETSAVCERQLRKVLDDMDLESIEDGYLELDTNEFVMGTVMDAVVSQGMITSREKGLQLIRETPREIKNMCLFGDQVRLQQVLADFLLNAVRFTPSSEGWVGIKVVPTKKRLGGGIHVMHLEFRVTHSGMGLPEELVHEMFDRGRGMTQEGLGLSMCRKLVKLMNGNVQYIRETGKSYFLVEVELPLAQRDDAGSVR

SEQ ID NO: 83 - protein, 1123 aa, Artificial Sequence
  Synthetic Peptide
MASAKLAYSTGSGIKSKHSVRVQQTTADAKLQAAYEESNDSGDSFDYSKSVGQAAKSTVQQVPAQAVTAYLQRMQRGGLTQTFGCMLAVEENTFRVIAYSENALDMLDLMPQAVPSVGQQDVLGIGTDSRSLFTPSSAAALERATQQSDLSMVNPVSVHSRSSGKPFYAILHRIDVGIVMDFEPVRPNDVVVSSAGTIHSHKLAAKAISRLQSLPGGDIGLLCDTVVEEVRELTGYDRVMAYKFHEDEHGEVLAEIRRSDLEPYLGLHYPATDIPQASRFLFMKNRFRMIVDCYAPPVKVIQDKDLRQPLTLAGSTLRAPHGCHAQYMGNMGSIASVTMAVVVNDQEDDGGSQKARRLWGLVVCHHTSARMISFPLRSACEFLMQVFGLQLNMEVELAAQLREKHILRTQTLLCDMLLRDAPIGIVSQSPNIMDLVKCDGAALYYGNRFWLLGITPSEQQIKEIADWLLECHKDSTGLSTDSLADAGYPGANLLGDAVCGMAAARITPKDFLFWFRSHTAKEIKWGGAKHDADDRDDGRKMTPRSSFNAFLEVVKRRSVPWEDIEMDAIHSLQLILRGSFQDIDDSDTKTMIHARLNDLKLQGMDELSTVANEMVRLIETATTPILAVDSSGFINGWNAKVAELTGLSVGDAMGRSLVKDLILEESVEDVQRLLYLALQGEEEQNVEIRLRTFGPQKAKGAVILIVNACSSRDVQENVVGVCFVGQDVTGQKMLHDKFTRIHGDYKSIVQNPNPLIPPIFGSDDLGYCTEWSPSMEKLTGWKRDEVLGKMLVGEVFGMHLMCCRLKGQDAVTKFMIVLNNAMDGQDTDKYPFSFFDRQGKFVEALLTANKRTDADGYITGVFCFLHIASPELLQALTVQRATEKVAFAKLKELAYIRQEIKNPLYGIMFTRNLMEDTDLSDDQKQFMDTSAVCERQLRKILDDMDLESIEDGYLELETAEFEMGSMMDAVVSQGMVTSREKGLQLIRETPREIKDMCLFGDQVRLQQVLADFLLNAVRFTPSSEGWVGIKVVATKRRLGGGFHVVHLEFRVTHPGSGLPEELIHEMFDRGRGMTQEGLGLNMCRKLVKLMNGNVQYKRETGKSYFLVTLELPLAVRDDTGTMR

SEQ ID NO: 84 - protein, 1131 aa, Artificial Sequence
  Synthetic Peptide
MSSTKLSYSSGTSVKSKHSVRVQQTTADAKLQAVYEESNDSGDSFDYSKSVGQATKSTVQQVPAQAVTAYLQRMQRGGLTQNFGCMLAVEESTFRVIAYSENAPEMLDLMPQAVPSVGLKEVLGIGTDARLLFTPSSASTLERAAATSDLTMVNPISVHSRNSGKPFYAIVHRIDVGIVIDFEPVRSNDVVISTAGALHSHKLAAKAVSRLQSLPGGDIGLLCDAVVEEVRELTGYDRVMAYKFHDDEHGEVLAEIRRSDLESYLGLHYPSTDIPQASRFLFMKNRVRMIADCCAPPVKVIQDKDLRQPITLAGSTLRAPHGCHAQYMGNMGSVASITLAVIVHDQEEDFGVQQKGRRLWGLVVCHHTSPRTISFPLRSACEFLMQVFGLQLNMEVELQAQMKEKHILRTQTLLCDMLLRDAPVGIVSQSPNIMDLVKCDGAALYYENQFWLLGTTPSEEQIEEIAAWLLEHHKDSTGLSTDSLADAGYPGANLLGDAVCGMAAARISSKDFLLWFRSHSAKEIKWGGAKHDAEDRDDSRKMTPRSSFNAFLEVVKRRSVPWEDIEMDAIHSLQLILRGSFQDVDGSGGKTMIHSRLHDLKLQGMDELSTVANEMVRLIETATAPILAVDSNGFINGWNAKVAELTGLPVGEAMGRSLVKDLILEEYVEVVERLLYLALQGEEEQNIEVSLKTFGAQKAKGAVILVVNACSSRDVQENVVGVCFVGQDVTGQKMVHDKFTRIHGDYKSIVQSPNPLIPPIFGSDELGYCIEWSPSMEKLTGWKREEVLGKMLVGEIFGVHQMCCRLKGQDAVTKFMIVLNNAMDGQDTDKYPFSFHDRQGKLVEALLTANKRTDADGYITGVFCFLHIASPELLQALTVQRATEKVAFAKLKELAYIRQEIKNPLYGIMFTRNLMEDTDLSDEQKQYMDTSAVCERQLRKILDDMDLESIEDGYLELETMEFEMGSMMDAVVSQGMVTSREKGLQLIRETPREIQGMCLFGDQVRLQQVLADFLLNAVRFTPSSEGWVGIKVVPTRKRLGGGFHVVHLEFRVTHPGSGLPEELVHEMFDRGRGMTQEGLGLSMCRKLVKLMNGEVKYIRDTGKSCFLVSLELPLTVSDDAGSVRQSGPLNC

SEQ ID NO: 85 - protein, 1124 aa, Artificial Sequence
  Synthetic Peptide
MTSTKLSYSLGTTVKSKHSVRVQQTTADAKLQAVYEESNDSGDAFNYSKSVGQAAKATVQQVPAQAVSSYLQRMQRGGLTQTFGCMLAVEESTFRVIAYSENAPEMLDLVPHAVPSVGQQDVLGIGADARSLFTPSSASALERAASTSDLSMVNPISVHSRSSGKPFYAIVHRIDVGVVIDFEPVRPNDVIISTAGALHSHKLAAKAVARLQSLPGGDIGLLCDAVVEEVRELTGYDRVMAYKFHDDEHGEVLAEIRRSDLESYLGLHYPSTDIPQASRFLFMKNRVRMIADCCAPPVKVIQDKDLRQPITLAGSTLRAPHGCHAQYMGNMGSVASITLAVIVNDQEEDFGVQQKGRRLWGLVVCHHTSARTISFPLRSACEFLMQVFGLQLNMEVELQAQVKEKHILRTQTLLCDMLLRDAPIGIVSQSPNIMDLVKCDGAALYYGKRFWLLGTTPTEQQITEIADWLLEYHKDSTGLSTDSLADAGYPGANLLGDAVCGMAAARITPKDFLFWFRSHTAKEIKWGGAKHDADEKDDGRKMAPRSSFNAFLEVVKRRSVPWEDIEMDAIHSLQLILRGSFQDIDDSDGKTMIHARLHDLKLQGMDELSTVANEMVRLIETATVPILAVDSSGFINGWNAKVAELTGLPVGDAMGRSLVKDLILEESVEAVERLLYLALQGEEEQNVEVRLKTFGQQKSKGVLILIVNACSSRDVQENVVGVCFVGQDVTGQKMVHDKFTRIHGDYKSIVQNPNPLIPPIFGGDELGYCTEWSPSMEKLTGWKREEVLGKMLVGEVFGVQLMCCRLKGQDAVTKFMIVLNNAMDGQDTDKFPFSFFDRQGKFVEALLTANKRTDADDYITGVFCFLHTASPELLQALTVQRATEKVAFAKLKELAYIRQEIKNPLYGIMFTRNLMEDTDLSEEQKQFMDTSAVCERQLRKILDDMDLESIEDGYLELETAEFDMGSMMDAVVSQGMITSREKGLQLIRETPREIKGMCLFGDQVRLQQVLADYLLNAVRFTPSSEGWVGIKVVSTKKRLVGGFHVVHLEFRVTHPGSGLPEELVCEMFDRGRGMTQEGLGLSMCRKLVKLMNGEVKYIRDAGKSYFLVNLELPLAGRDDSGNAR

SEQ ID NO: 86 - protein, 1171 aa, Artificial Sequence
  Synthetic Peptide
MASGSRATPTRSPSSARPAAPRHQHHHSQSSGGSTSRAGGGGGGGGGGGGGAAAAESVSKAVAQYTLDARLHAVFEQSGASGRSFDYTQSLRASPTPSSEQQIAAYLSRIQRGGHIQPFGCTLAVADDSSFRLLAYSENTADLLDLSPHHSVPSLDSSAVPPPVSLGADARLLFAPSSAVLLERAFAAREISLLNPLWIHSRVSSKPFYAILHRIDVGVVIDLEPARTEDPALSIAGAVQSQKLAVRAISRLQALPGGDVKLLCDTVVEYVRELTGYDRVMVYRFHEDEHGEVVAESRRNNLEPYIGLHYPATDIPQASRFLFRQNRVRMIADCHAAPVRVIQDPALTQPLCLVGSTLRSPHGCHAQYMANMGSIASLVMAVIISSGGDDDHNISRGSIPSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTEVQIKDIIEWLTMCHGDSTGLSTDSLADAGYPGAAALGDAVSGMAVAYITPSDYLFWFRSHTAKEIKWGGAKHHPEDKDDGQRMHPRSSFKAFLEVVKSRSLPWENAEMDAIHSLQLILRDSFRDSAEGTSNSKAIVNGQVQLGELELRGIDELSSVAREMVRLIETATVPIFAVDTDGCINGWNAKVAELTGLSVEEAMGKSLVNDLIFKESEETVNKLLSRALRGDEDKNVEIKLKTFGPEQSKGPIFVIVNACSSRDYTKNIVGVCFVGQDVTGQKVVMDKFINIQGDYKAIVHNPNPLIPPIFASDENTCCSEWNTAMEKLTGWSRGEVVGKLLVGEVFGNCCRLKGPDALTKFMIVLHNAIGGQDCEKFPFSFFDKNGKYVQALLTANTRSRMDGEAIGAFCFLQIASPELQQAFEIQRHHEKKCYARMKELAYIYQEIKNPLNGIRFTNSLLEMTDLKDDQRQFLETSTACEKQMSKIVKDASLQSIEDGSLVLEKGEFSLGSVMNAVVSQVMIQLRERDLQLIRDIPDEIKEASAYGDQYRIQQVLCDFLLSMVRFAPAENGWVEIQVRPNIKQNSDGTDTMLFLFRFACPGEGLPPEIVQDMFSNSRWTTQEGIGLSICRKILKLMGGEVQYIRESERSFFHIVLELPQPQQAASRGTS

SEQ ID NO: 87 - protein, 783 aa, Artificial Sequence
  Synthetic Peptide
MASGSRATPTRSPSSARPEAPRHAHHHHHSQSSGGSTSRAGGGAAATESVSKAVAQYTLDARLHAVFEQSGASGRSFDYSQSLRAPPTPSSEQQIAAYLSRIQRGGHIQPFGCTLAVADDSSFRLLAFSENSPDLLDLSPHHSVPSLDSSAPPHVSLGADARLLFSPSSAVLLERAFAAREISLLNPIWIHSRVSSKPFYAILHRIDVGVVIDLEPARTEDPALSIAGAVQSQKLAVRAISRLQALPGGDVKLLCDTVVEHVRELTGYDRVMVYRFHEDEHGEVVAESRRDNLEPYLGLHYPATDIPQASRFLFRQNRVRMIADCHATPVRVIQDPGLSQPLCLVGSTLRAPHGCHAQYMANMGSIASLVMAVIISSGGDDEQTGRGGISSAMKLWGLVVCHHTSPRCIPFPLRYACEFLMQAFGLQLNMELQLAHQLSEKHILRTQTLLCDMLLRDSPTGIVTQSPSIMDLVKCDGAALYYHGKYYPLGVTPTESQIKDIIEWLTVFHGDSTGLSTDSLADAGYLGAAALGEAVCGMAVAYITPSDYLFWFRSHTAKEIKWGGAKHHPEDKDDGQRMHPRSSFKAFLEVVKSRSLPWENAEMDAIHSLQLILRDSFRDAAEGTNNSKAIVNGQVQLRELELRGINELSSVAREMVRLIETATVPIFAVDTDGCINGWNAKIAELTGLSVEEAMGKSLVNDLIFKESEATVEKLLSRALRGEEDKNVEIKLKTFGSEQSKGPIFVVVNACSSRDYTQNIVGVCFVGQDVTGQKVVMDKFVNIQ

SEQ ID NO: 88 - protein, 1166 aa, Artificial Sequence
  Synthetic Peptide
MASDSRPPKRSPSARRVAPRHAHHHHSQSSGGSTSRAGAGGGGGGAAATESVSKAVAQYNLDARLHAVFEQSGASGRSFDYSQSLRAPPTPSSEQQIAAYLSRIQRGGHIQPLGCTLAVADDSSFRLLAFSENAADLLDLSPHHSVPSLDSVALPPVSLGADARLYFSPSSAVLLERAFAAREISLLNPLWIHSRASSKPFYAILHRIDVGVVIDLEPARTEDPALSIAGAVQSQKLAVRAISRLQALPGGDVKLLCDTVVEHVRELTGYDRVMVYKFHEDEHGEVVAESRRDNLEPYLGLHYPATDIPQASRFLFQQNRVRMIADCHAIPVRVIQDPGLSQQLCLVGSTLRAPHGCHAQYMANMGSIASLVMAVIISSGGDDERTGRGAISSSMKLWGLVVCHHTSPRCIPFPLRYACEFLMQAFGLQLNMELQLAHQLSEKHILRTQTLLCDMLLRDSPAGIITQSPSVMDLVKCDGAALYYRGKYYPLGVTPTESQIKDIIEWLTVCHGDSTGLSTDSLADAGYLGAVALGDAVCGMAVAYITPSDYLFWFRSHTAKEIKWGGAKHHPEDKDDGQRMHPRSSFKAFLEVVKSRSLSWENAEMDAIHSLQLILRDSFRDAAEGTSNSKAIVNGQRQLGELELRGINELSSVAREMVRLIETATVPIFAVDTDGCINGWNAKIAELTGLSVEEAMGKSLVNDLIFKECDDIVEKLLSRALRGEEDKNVEIKLKTFGSEQSKGAIFVIVNACSSRDYTQNIVGVCFVGQDVTGQKVVMDKFINIQGDYKAIVHNPNPLLPPIFASDENTSCSEWNTAMEKLTGWSREEVVGKFLIGEVFGNCCRLKGPDALTKFMVVIHNAIEGHDSEKFPFSFFDKNGKYVQALLTANTRSKMDGKSIGAFCFLQIASAEIQQAFEIQRQQEKKCYARMKELAYICQEIKNPLSGIRFTNSLLQMTDLNDDQRQFLETSSACEKQMSKIVKDASLKSIEDGSLVLEKSEFSLGDVMNAVVSQTMSLLRERDLQLIRDIPDEIKDASAYGDQFRIQQVLADFLLSMAQSAPSENGWVEIQVRPNVKQNYDGTDTELFIFRFACPGEGLPADIVQDMFSNSQWSTQEGVGLSTCRKILKLMGGEVQYIRESERSFFLIVLELPQPRLAAGRENQLIC

SEQ ID NO: 89 - protein, 1132 aa, Artificial Sequence
  Synthetic Peptide
MASGSRTKHSHQSGQGQVQAQSSGTSNVNYKDSISKAIAQYTADARLHAVFEQSGESGKSFDYSQSIKTTTQSVVPEQQITAYLTKIQRGGHIQPFGCMIAVDEASFRVIAYSENACEMLSLTPQSVPSLERPEILTVGTDVRTLFTPSSSVLLERAFGAREITLLNPIWIHSKNSGKPFYAILHRVDVGIVIDLEPARTEDPALSIAGAVQSQKLAVRAISHLQSLPGGDVKLLCDTVVESVRELTGYDRVMVYKFHEDEHGEVVAESKIPDLEPYIGLHYPATDIPQASRFLFKQNRVRMIVDCHATPVRVVQDESLMQPLCLVGSTLRAPHGCHAQYMANMGSIASLTLAVIINGNDEEAVGGRSSMRLWGLVVGHHTSARCIPFPLRYACEFLMQAFGLQLNMELQLASQLSEKHVLRTQTLLCDMLLRDSPTGIVIQSPSIMDLVKCDGAALYCQGKYYPLGVTPTEAQIKDIVEWLLTYHGDSTGLSTDSLADAGYPGAALLGDAVCGMAVAYITSKDFLFWFRSHTAKEIKWGGAKHHPEDKDDGQRMHPRSSFKAFLEVVKSRSLPWENAEMDAIHSLLILRDSFKDAEASNSKAVVHAQLGEMELQGIDELSSVAREMVRLIETATAPIFAVDVEGRINGWNAKVAELTDLSVEEAMGKSLVHDLVHKESQETAEKLLFNALRGEEDKNVEIKLRTFGPEQLKKAVFVVVNACSSKDYTNNIVGVCFVGQDVTGQKVVMDKFIHIQGDYKAIVHSPNPLIPPIFASDENTCCSEWNTAMEKLTGWSRGEIIGKMLVGEIFGSCCRLKGPDAMTKFMIVLHNAIGVQDTDKFPFSFFDRNGKYVQALLTANKRVNMEGQIIGAFCFIQIASPELQQALRVQRQQEKKCYSQMKELAYLCQEIKSPLNGIRFTNSLLEATDLTENQKQYLETSAACERQMSKIIRDVDLENIEDGSLTLEKEEFFLGSVIDAVVSQVMLLLRERSVQLIRDIPEEIKTLTVHGDQVRIQQVLADFLLNMVRYAPSPDGWVEIQLQPNMKQISDEVTVVHIEFRIVCPGEGLPPELVQDMFHSSRWVTKEGLGLSMCRKILKLMNGDIQYIRESERCYFLIILDLPMTRRGSKSLG

SEQ ID NO: 90 - protein, 1151 aa, Artificial Sequence
  Synthetic Peptide
MASQSQRQSNQRQHQNQAAQSSGTSNMRQHHHATESVSKAIAQYTVDAQLHAVFEQSGGSGKSFDYSQSVRTTSQSVPEEQITAYLSKIQRGGHIQPFGCMIAVDEGSFRVIAYSENAKEMLGLTPQSVPSLDKQEILSDGTDVRTLFRPSSSAMLEKAFGAREIILLNPIWIHSKNSGKPFYAILHRIDVGIVIDLEPARTEDPALSIAGAVQSQKLAVRSISQLQSLPGGDIKLLCDTVVESVRELTGYDRVMVYKFHEDEHGEVVAENKRADLEPYIGLHYPSTDIPQASRFLFKQNRVRMIVDCHATPVRVIQDEALMQPLCLVGSTLRAPHGCHAQYMANMGSIASLAMAVIINGNEEEAIGGRNSTRLWGLVVCHHTSARCIPFPLRYACEFLMQAFGLQLNMELQLASQLSEKHVLRTQTLLCDMLLRDSPTGIVTQSPSIMDLVKCDGAALYYQGQYYPLGVTPTEAQIKDIVEWLLALHGDSTGLSTDSLADAGYPGAASLGNAVCGMAVAYITKRDFLFWFRSHTAKEIKWGGAKHHPEDKDDGQRMHPRSSFKAFLEVVKSRSLLWENAEMDAIHSLQLILRDSFRDVEATNSKAVVHAQLEDTELQGMDELSSVAREMVRLIETATAPIFAVDVDGCINGWNAKVAELTGLSVDKAMGKSLVHDLVYKEYEETVDKLLHRALRGEEDKNVEIKLRTFGSEHQKKALFVVVNACSSKDYMNNIVGVCFVGQDVTGQKVVMDKYVHIQGDYKAIVHSPNPLIPPIFASDENTCCLEWNTAMEKFTGWSRGEVIGKMLVGEVFGSCCQLKGSDALTKFMIALHNAIGGQDTDKLPFSFFDRNGKYVQALLTANKRVNMEGEIVGAFCFLQIASNELQQALKVQRQQEKKCSARMKELAYICQEIRNPLSGLRFTNSLLENTDLTEDQKQFLETSAACEKQILKITRDVDLESIENGLLELEKAEFLFGSVINAVVSQAMLLLRERNLQLLRDIPEEIKTLVVYGDQARIQQVLADFLLNMVRYAPSSAGWVEIHVCPTLKQISDGHTLVHMEFKYALLNSFACLPPELVQDMFHSSRWVTQEGLGLSMCRKILKLMNGEVQYIRESERCYFLVILEVPMPNKCERYNCKKCCRLGCLVCNVY

SEQ ID NO: 91 - protein, 1146 aa, Artificial Sequence
  Synthetic Peptide
MASQSQRQSNQPVHNQAQSSGTSNMRQHHHATESVSKAIAQYTVDAQLHAVFEQSGGTGRSFDYSKSVRTTNQSVPEQQITAYLSKIQRGGHIQPFGCMIAADEQSFRVIAYSENAKDMLGLTPQSVPSLEKQEILFVGADVRILFRPSSAVLLEKAFGAREITLLNPIWIHSKNSGKPFYAILHRIDVGIVIDLEPARTEDPALSIAGAVQSQKLAVRAISQLQSLPGGDIKLLCDTVVDSVRELTGYDRVMVYKFHEDEHGEVVAENKRVDLEPYIGLHYPSTDIPQASRFLFKQNRVRMIVDCHAIPVRVIQDEALMQPLCLVGSTLRAPHGCHAQYMENMGSIASLAMAVIIYGNDEEAIGGRNSMRLWGLVVCHHTSARCIPFPLRYACEFLMQAFGLQLNMELQLASQLLEKHVLRTQTLLCDMLLRDSPTGIVTQSPSIMDLVKCDGAALYYQGQYYPLGVTPTETQIKDIVEWLLTLHGDPTGLSTDSLADAGYPGAAFLGDAVCGMAVAYIAERDFLFWFRSHTAKEVKWGGAKHHPEDKDDGQRMHPRSSFKAFLEVVKSRSLPWENAEMDAIHSLQLILRDSFRDAEATNSKAVVHTQLKDMELQGMDELSSVAREMVRLIETATAPIFAVDVDGRINGWNAKVAELTGLSVEEAMGKSLVHDLVYKEYEEIVDKLIHRAVKGEEDKNVEIKLRTFCSEHQKKAVFVVVNACSSKDYMDNIVGVCFVGQDITGQKVVMDKYVLIQGDYKAIVHSPNPSIPPIFASDENTCCLEWNTAMEKLTGWSRGEVVGKMLVGEVFGSCCRLKGPDALTKFMIALHNAIGGIDTDKLPFSFFDRNEKNVQTLLTANKRVNMEGDIIGAFCFLQIASPELQQTLKVQKQQEKKSFARMKELAYICQEIKNPLSGIHFTNSLLENTDLTEDQQQFLETSAACEKQILKIIRDIDLESIENGSLELEKAEFLLGSVINAVVSQAMLLLRERNLQLLRDIPEEIKTLAVYGDQARIQQVLADFLLNMVRYAPSSAGWVEIHVCPTLKQISDGHTLVHTEFKYLERVLQTRMLGLQHPLRSFLNFKLTLVMLHREGMVPKIQFQGLSVCQGRLIWLSYPENTTVTLYPLTIWFLLLYRRQSRSITHT

SEQ ID NO: 92 - protein, 1164 aa, Artificial Sequence
  Synthetic Peptide
MVSGGGSKTSGGEAASSGHRRSRHTSAAEQAQSSANKALRSQNQQPQNHGGGTESTNKAIQQYTVDARLHAVFEQSGESGKSFDYSQSLKTAPYDSSVPEQQITAYLSRIQRGGYTQPFGCLIAVEESTFTIIGYSENAREMLGLMSQSVPSIEDKSEVLTIGTDLRSLFKSSSYLLLERAFVAREITLLNPIWIHSNNTGKPFYAILHRVDVGILIDLEPARTEDPALSIAGAVQSQKLAVRAISHLQSLPSGDIKLLCDTVVESVRDLTGYDRVMVYKFHEDEHGEVVAESKRNDLEPYIGLHYPATDIPQASRFLFKQNRVRMIVDCYASPVRVVQDDRLTQFICLVGSTLRAPHGCHAQYMTNMGSIASLAMAVIINGNEEDGNGVNTGGRNSMRLWGLVVCHHTSARCIPFPLRYACEFLMQAFGLQLNMELQLALQVSEKRVLRMQTLLCDMLLRDSPAGIVTQRPSIMDLVKCNGAAFLYQGKYYPLGVTPTDSQINDIVEWLVANHSDSTGLSTDSLGDAGYPRAAALGDAVCGMAVACITKRDFLFWFRSHTEKEIKWGGAKHHPEDKDDGQRMNPRSSFQTFLEVVKSRCQPWETAEMDAIHSLQLILRDSFKESEAMDSKAAAAGAVQPHGDDMVQQGMQEIGAVAREMVRLIETATVPIFAVDIDGCINGWNAKIAELTGLSVEDAMGKSLVRELIYKEYKETVDRLLSCALKGDEGKNVEVKLKTFGSELQGKAMFVVVNACSSKDYLNNIVGVCFVGQDVTGHKIVMDKFINIQGDYKAIIHSPNPLIPPIFAADENTCCLEWNTAMEKLTGWPRSEVIGKLLVREVFGSYCRLKGPDALTKFMIVLHNAIGGQDTDKFPFPFFDRKGEFIQALLTLNKRVSIDGKIIGAFCFLQIPSPELQQALEVQRRQESEYFSRRKELAYIFQVIKNPLSGLRFTNSLLEDMDLNEDQKQLLETSVSCEKQISKIVGDMDVKSIDDGSFLLERTEFFIGNVTNAVVSQVMLVVRERNLQLIRNIPTEVKSMAVYGDQIRLQQVLAEFLLSIVRYAPMEGSVELHLCPTLNQMADGFSAVRLEFRMACAGEGVPPEKVQDMFHSSRWTSPEGLGLSVCRKILKLMNGGVQYIREFERSYFLIVIELPVPLMMMMPSS

(19) United States
(12) Patent Application Publication
(10) Pub. No.: US 2015/0307565 A1
(43) Pub. Date: Oct. 29, 2015

(54) STRATEGY TO ENGINEER PHYTOCHROMES WITH IMPROVED ACTION IN CROP FIELDS

(71) Applicant: WISCONSIN ALUMNI RESEARCH FOUNDATION, MADISON, WI (US)

(72) Inventors: Ernest Sethe Burgie, Sun Prairie, WI (US); Adam Nicholas Bussell, Verona, WI (US); Richard David Vierstra, Madison, WI (US); Joseph Walker, Madison, WI (US)

(21) Appl. No.: 14/677,942

(22) Filed: Apr. 2, 2015

Related U.S. Application Data
(60) Provisional application No. 61/995,101, filed on Apr. 2, 2014.

(51) Int. Cl. C07K 14/415; C12N 15/82

(52) U.S. Cl. CPC C07K 14/415; C12N 15/8261

(57) ABSTRACT

Disclosed herein are to isolated polynucleotide sequences encoding modified phytochrome polypeptides wherein the modified phytochrome polypeptide has at least one of an altered thermal reversion rate, an altered photoconversion rate, an altered absorption spectrum, or an altered signal output compared to the unmodified phytochrome polypeptide. Also disclosed are transgenic plants comprising said modified phytochrome polypeptides and methods of producing said transgenic plants.

### CROSS REFERENCE TO RELATED APPLICATIONS

This application claims priority to U.S. Provisional Application No. 61/995,101, filed Apr. 2, 2014, which is incorporated herein by reference in its entirety.

### STATEMENT OF GOVERNMENT INTEREST

This invention was made with government support under 1329956 awarded by the National Science Foundation and 14-CRHF-0 awarded by the USDA/NIFA. The government has certain rights in the invention.

### TECHNICAL FIELD

The present invention relates to isolated polynucleotide sequences encoding modified phytochrome polypeptides. The modified phytochrome polypeptide has at least one of an altered thermal reversion rate, an altered photoconversion rate, an altered absorption spectrum, or an altered signal output compared to the unmodified phytochrome polypeptide. The present invention also relates to transgenic plants comprising said isolated polynucleotide sequences and/or modified phytochrome polypeptides and methods of producing said plants.

### BACKGROUND

Given the importance of sunlight to their survival and growth, plants have adopted a collection of photoreceptors and interconnected signaling cascades to optimize their photosynthetic potential and to synchronize their life cycles with circadian and seasonal rhythms. Chief among these are the phytochromes (Phys), a family of bilin (or open chain tetrapyrrole)-containing, red/far-red light-absorbing photoreceptors that provide spatial and time-dependent information by sensing the fluence rate, direction, duration, and color of a plant's light environment. This information is then used to regulate numerous morphogenic and growth processes, including seed germination, leaf development, pigmentation, shade avoidance, and the photoperiodic control of flowering time. Notably, seed plants typically express three Phy isoforms (PhyA, B, and C) that control distinct and overlapping photoresponses. PhyB has a dominant role in green tissues.

Both the continuing rise of the global population and the new demands for carbon-neutral biofuels have accelerated the need to continually improve agricultural productivity. One emerging strategy is to control plant reproduction and architecture to better fit specific environments and to dramatically increase crop densities, the latter of which will require the redesign of plants that perform well in more competitive environments. Because plant architecture, timing of reproduction, and the response to competition are often controlled by the phytochrome family of photoreceptors, their re-engineering for these new cropping strategies might provide novel solutions.

Although recent reports of atomic-level structures of bacterial phytochromes have provided useful models for understanding phytochrome signaling, given the low sequence identity of these relatives to plant phytochromes and notable divergences in domain structure, the lack of direct structural information about plant phytochromes has hindered progress in the field. It remains unclear how well these bacterial structures functionally mimic plant Phys given significant differences in sequence and domain architecture. Examples of structural differences include a long N-terminal extension (NTE) in plant Phys and extensive sequence variations within the Per/Arnt/Sim (PAS) domain, the cGMP phosphodiesterase/adenylyl cyclase/Fh1A (GAF) domain and the Phy-specific (PHY) domain domains, and the downstream output module (OPM). There is a need to generate phytochromes with altered characteristics to alter crop development, architecture, and reproduction.

### SUMMARY

The present invention is directed to an isolated polynucleotide encoding a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1. The unmodified phytochrome polypeptide has an amino acid sequence selected from SEQ ID NOs: 1-26 or 67-92. The modified phytochrome polypeptide may comprise an amino acid other than tyrosine at the residue corresponding to position 104 of SEQ ID NO: 1, an amino acid other than isoleucine or methionine at the residue corresponding to position 108 of SEQ ID NO: 1, an amino acid other than glycine at the residue corresponding to position 284 of SEQ ID NO: 1, an amino acid other than histidine at the residue corresponding to position 358 of SEQ ID NO: 1, an amino acid other than valine at the residue corresponding to position 401 of SEQ ID NO: 1, an amino acid other than histidine at the residue corresponding to position 403 of SEQ ID NO: 1, an amino acid other than tryptophan at the residue corresponding to position 563 of SEQ ID NO: 1, an amino acid other than glycine at the residue corresponding to position 565 of SEQ ID NO: 1, an amino acid other than serine at the residue corresponding to position 584 of SEQ ID NO: 1, or combinations thereof. The modified phytochrome polypeptide may comprise a substitution corresponding to at least one of Y104-A, I108-A, I108-Y, G284-V, H358-A, V401-S, H403-A, W563-S, G565-E, S584-A, S584-E, or a combination thereof, of SEQ ID NO:1. The modified phytochrome polypeptide may have at least one of an altered thermal reversion rate, an altered photoconversion rate, an altered absorption spectrum, an altered signal output compared to the unmodified phytochrome polypeptide, or combinations thereof. The modified phytochrome polypeptide may have an altered thermal reversion rate compared to the unmodified phytochrome polypeptide. The rate of thermal reversion of the modified phytochrome polypeptide may be decreased compared to the unmodified phytochrome polypeptide. The rate of thermal reversion of the modified phytochrome polypeptide may be decreased at least 0.5 fold compared to the unmodified phytochrome polypeptide. The rate of thermal reversion of the modified phytochrome polypeptide may be increased compared to the unmodified phytochrome polypeptide. The rate of thermal reversion of the modified phytochrome polypeptide may be increased at least 0.5 fold compared to the unmodified phytochrome polypeptide. The modified phytochrome polypeptide may have an altered photoconversion rate compared to the unmodified phytochrome polypeptide. The photoconversion rate from the Pfr form to the Pr form of the modified phytochrome polypeptide may be increased compared to the unmodified phytochrome polypeptide. The photoconversion rate from the Pfr form to the Pr form of the modified phytochrome polypeptide may be decreased compared to the unmodified phytochrome polypeptide. The photoconversion rate may be determined at a wavelength of about 720 nm. The photoconversion rate from the Pr form to the Pfr form of the modified phytochrome polypeptide may be increased compared to the unmodified phytochrome polypeptide. The photoconversion rate from the Pr form to the Pfr form of the modified phytochrome polypeptide may be decreased compared to the unmodified phytochrome polypeptide. The photoconversion rate may be determined at a wavelength of about 660 nm or about 720 nm. The photoconversion rate of the modified phytochrome polypeptide may be increased at least 0.5 fold compared to the unmodified phytochrome polypeptide. The photoconversion rate of the modified phytochrome polypeptide may be decreased at least 0.5 fold compared to the unmodified phytochrome polypeptide. The modified phytochrome polypeptide may have an altered absorption spectrum compared to the unmodified phytochrome polypeptide. The altered absorption spectrum may be a shift in an absorption peak wavelength. The modified phytochrome polypeptide may have a Pr absorption spectrum that is shifted to a longer wavelength compared to the unmodified phytochrome polypeptide. The modified phytochrome polypeptide may have a Pr absorption spectrum that is shifted to a shorter wavelength compared to the unmodified phytochrome polypeptide. The modified phytochrome polypeptide may have a Pfr absorption spectrum that is shifted to a longer wavelength compared to the unmodified phytochrome polypeptide. The modified phytochrome polypeptide may have a Pfr absorption spectrum that is shifted to a shorter wavelength compared to the unmodified phytochrome polypeptide. The modified phytochrome polypeptide may have an altered signal output compared to the unmodified phytochrome polypeptide. The modified phytochrome polypeptide may further comprise at least one amino acid substitution at a position corresponding to position 276, 307, 322, 352, 361, 564, 582, or a combination thereof, of SEQ ID NO:1. The modified phytochrome polypeptide may further comprise a substitution corresponding to at least one of Y276-H, D307-A, R322-A, R352-A, Y361-F, G564-E, R582-A, or a combination thereof, of SEQ ID NO:1.

The present invention is also directed to a vector comprising said isolated polynucleotide. The present invention is also directed to an isolated polynucleotide construct comprising a promoter not natively associated with said polynucleotide operably linked to said polynucleotide.

The present invention is also directed to a plant cell comprising said isolated polynucleotide operably linked to a promoter not natively associated with said polynucleotide.

The present invention is also directed to a plant comprising said plant cell. The plant may exhibit increased light sensitivity relative to a control plant lacking the polynucleotide. The plant may exhibit a decreased height, decreased diameter or a combination thereof, relative to a control plant lacking the polynucleotide. The plant may exhibit at least one characteristic selected from, increased hyponasty, decreased petiole length, decreased internode length, and decreased hypocotyl length under an R fluence rate of less than 1 μmole m⁻² sec⁻¹, relative to a control plant lacking the polynucleotide. The plant may exhibit enhanced germination relative to the control plant. The plant may be corn, soybean or rice. The plant may be an ornamental plant.

The present invention is also directed to a method of producing a transgenic plant. The method comprises (a) introducing into a plant cell an isolated polynucleotide encoding a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92; and (b) regenerating the transformed cell to produce a transgenic plant. The transgenic plant may exhibit increased light sensitivity relative to a control plant lacking the isolated polynucleotide. The transgenic plant may exhibit decreased height, decreased diameter, or a combination thereof, relative to a control plant lacking the polynucleotide. The transgenic plant may exhibit at least one characteristic selected from decreased petiole length, decreased internode number, increased hyponasty, and decreased hypocotyl length under an R fluence rate of less than 1 μmole m⁻² sec⁻¹, relative to a control plant lacking the polynucleotide. The transgenic plant may exhibit enhanced germination relative to the control plant. The transgenic plant may be a corn, soybean or rice plant. The transgenic plant may be an ornamental plant. The present invention is also directed to a transgenic plant produced by said method.

The present invention is also directed to an isolated polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show the crystallographic structure of the photosensory module (PSM) from Arabidopsis PhyB as Pr and its comparison with that from Synechocystis (Syn) Cph1. FIG. 1A: Ribbon diagram of the PSM dimer (resides 90-624) in front and side views. For subunit A, the PAS, GAF, and PHY domains are colored in blue, green, and orange, respectively. The knot lasso (yellow), hairpin, helical spine, and NTE are indicated. PΦB (cyan) with its linkage to Cys357 is shown in stick form with the oxygens colored in red. The proposed connectivities of polypeptide regions not resolved in the crystal structure are indicated by the dashed lines for subunit A. FIG. 1B: Superposition of PSMs from PhyB (blue) (PDB ID code 4OUR) and Syn-Cph1 (gray) (PDB ID code 2VEA). The extended α5/α1 helix shared by the PAS and GAF domains in PhyB is shown.

FIGS. 2A-2C show the conformation of PΦB and its surrounding amino acids within the bilin-binding pocket of Arabidopsis PhyB. FIG. 2A: Top view of PΦB superimposed on a fo-fc omit map of the chromophore region contoured at 3σ. Carbons, cyan; oxygens, red; nitrogens, blue; sulfurs, yellow. FIG. 2B: Top and side views of PΦB in a ZZZssa configuration and linked via a thioether bond between the C3¹ carbon and C357. Pyrrole rings A-D, the C3² methyl, and the C18² carbon of the D-ring vinyl, are labeled. FIG. 2C: Top and side views of the bilin-binding pocket of PhyB highlighting the positions of key amino acids. Residues from the PAS-knot, PAS and NTE, GAF, and hairpin regions are colored in yellow, blue, green, and orange, respectively. pw, pyrrole water. Dashed lines indicate hydrogen bonds.

FIGS. 3A-3D show the structural and mutational analysis of key amino acids surrounding the bilin and the PHY domain hairpin in Arabidopsis PhyB. FIGS. 3A, 3C: Close-up views of the GAF domain (FIG. 3A) and hairpin (FIG. 3C) in PhyB and the bathyphytochrome from P. aeruginosa (Pa-BphP) as Pfr (BV) (PDB ID code 3C2W). The bilin, NTE, GAF, knot lasso, and PHY domain features and associated residues are colored in cyan, blue, green, yellow, and orange, respectively. The comparable amino acids between At-PhyB and Pa-BphP are labeled. Nitrogens and oxygens are colored in blue and red, respectively. pw, pyrrole water. BV, biliverdin. Dashed lines indicate hydrogen bonds. Hairpin is indicated by bracket. The βₑₙₜ and βₑₓᵢₜ strands connecting the hairpin to the rest of the PHY domain are labeled. FIGS. 3B, 3D: UV-visible spectroscopy of selected PhyB(PSM) mutants within the GAF domain (FIG. 3B) and at the hairpin (FIG. 3D). Absorption and difference spectra were measured at 25° C. as Pr and following saturating red light irradiation (RL). Absorption maxima are indicated. Photoconversion and thermal reversion kinetics are shown in FIG. 10 and Table 4.

FIGS. 4A-4B show the half-lives (T_{1/2}) of thermal reversion for Arabidopsis PhyB PSM mutants and truncations. FIG. 4A shows the half-lives (T_{1/2}) of Pfr→Pr thermal reversion for Arabidopsis PhyB PSM mutants and truncations. Rates were measured at 25° C. using absorbance at 660 nm (See FIG. 10 and Table 4). With the exception of H385A, each bar represents the average of three separate measurements (±SD). Dashed line indicates the t_{1/2} of unmodified PhyB(PSM) (t_{1/2}=83 min). *The Y276-H mutant failed to photoconvert. FIG. 4B shows the half-lives (t/2) for the Pr→Pfr and Pfr→Pr photoconversion reactions, and Pfr→Pr thermal reversion for Arabidopsis PhyB PSM mutants and truncations. Rates were measured at 25° C. using the absorbance at 660 nm or 730 nm. Standardized red (660 nm) or far-red light (730 nm) fluence rates were used for photoconversion. With the exception of H385A, each square represents the average of three separate measurements. Dashed lines indicate the t/2 of unmodified PhyB(PSM). Mutants without data points represent those that failed to generate normal Pfr spectra upon photoconversion with red light. Grey boxes indicate the full-length PhyB PSM or truncations. Blue boxes indicate NTE and PAS mutants. Green boxes indicate GAF mutants. Orange boxes indicate PHY hairpin mutants. Y276H was omitted due to impaired photochemistry.

FIG. 5 shows the Toggle model for Phy photoconversion that translates light into a conformational signal.

FIGS. 6A-6G show the absorption and photochemical properties of Arabidopsis PhyB(90-624) assembled with PΦB. FIG. 6A: Domain architecture of PhyB in comparison to its bacterial relatives Cph1 from Synechocystis and BphP from D. radiodurans. The glycine/serine-rich N-terminal extension NTE, the invariant DIP (Asp-Ile-Pro) and PRXSF (Pro-Arg-X-Ser-Phe) motifs, and the H, N, D/F and G regions characteristic of two-component histidine kinases are shown. The cysteine that binds the chromophore are indicated by the arrowheads. FIG. 6B: Diagram of PΦB and its thioether linkage to C357 of Arabidopsis PhyB via the C3¹ carbon. The A-D pyrrole rings are labeled. FIG. 6C: Assembly of PhyB(90-624) with PΦB as compared to the entire PhyB PSM (residues 1-624). Purified recombinant proteins were subjected to SDS-PAGE and either stained for protein with Coomassie blue (Prot) or assayed for the bound bilin by Zn-induced fluorescence (Zn). FIG. 6D: UV-visible absorption spectroscopy of the PhyB(90-624) fragment. Absorption and difference spectra measured at 25° C. as Pr and following saturating red light (RL) irradiation (mostly Pfr). Absorption maxima are indicated. FIG. 6E: Time course for Pr→Pfr photoconversion by red light for the PhyB(90-624) truncation versus the entire PSM with best fits to simple two exponential functions. The reactions in the left and right panels were monitored at 660 and 730 nm, respectively. FIG. 6F: Thermal reversion at 25° C. of Pfr back to Pr for the PhyB(90-624) truncation versus the entire PSM. FIG. 6G: Normalized time course of Pfr→Pr photoconversion at 25° C. by far-red light for the PhyB(90-624) truncation versus the entire PSM. The reactions in the left and right panels were monitored at 730 and 660 nm, respectively. Data were fit to a single exponential. The inset shows the Pfr→Pr photoconversion rate of each corrected for the different rates of thermal reversion. The traces shown in FIGS. 6E-6G represent the average of three separate measurements

FIG. 7 shows the effects of acidic denaturation of the absorption spectra of PhyB. Purified PhyB(PSM) was either kept in the dark (Pr) or irradiated with saturating red light irradiation (RL) (mostly Pfr) were either kept in native buffer (left panel) or exchanged into an acidic denaturing buffer (right panel). UV-visible absorption spectra were then recorded at 25° C. Absorption maxima are indicated

FIGS. 8A-8B show the size determination of Arabidopsis PhyB PSM (residues 1-624). FIG. 8A: Apparent size of PhyB(PSM) was measured by size exclusion chromatography (SEC) as either Pr (red circle) or following saturating red light irradiation (mostly Pfr) (purple circle). The column was calibrated with Bio-Rad size-exclusion protein standards and blue dextran (open circles). The partition coefficient, Kₐᵥ, was calculated using the equation, Kₐᵥ=(Vₑ−V₀)/(Vₜ−V₀), where Vₑ, V₀, and Vₜ are the elution, void and total volumes, respectively. V₀ and Vₜ were measured using blue dextran and vitamin B12. FIG. 8B: Equilibrium sedimentation was used to measure the mass of PhyB(PSM) as Pr using two concentrations and three rotor speeds. Lines represent global fits of the data. The calculated ratio of the measured molecular weight to that calculated from the sequence is shown.

FIGS. 9A-9E show the secondary structure diagram of the Arabidopsis PhyB(PSM) and its superposition with the 3-D structures of bacterial relatives. FIG. 9A: A secondary structure representation of the PhyB structure is provided with α-helices as cylinders, and β-strands as arrows. The NTE, PAS, GAF, and PHY domains and the OPM are colored brown, blue, green, orange, and magenta, respectively. Exceptions to the color scheme are the NTE α-helix and knot region of the GAF domain, which are colored blue and yellow, respectively. Strands and helices are labeled as they are named in the text. Phytochromobilin (PΦB) is colored cyan and its linkage with Cys 357 is emphasized in red. Other motifs are highlighted with circles. The labeled “β-turn” motif is a special case where a β-turn-like structure connects a loop proceeding from strand β2 to strand β3. The conserved PHY domain residue Trp599 is shown to illustrate the position of the loop connecting strand βₑₓᵢₜ and helix α6 of the PHY domain. Due to diffuse density in the PHY domain loops helix α4 was not clearly visible, thus its position was left open. FIG. 9B: Superposition of Arabidopsis PhyB PAS (blue) and GAF (blue) domains with bacterial PAS an GAF domains of Synechocystis sp. PCC 6803 Cph1 (green, PDB: 2VEA), D. radiodurans BphP (yellow, PDB: 209C), and P. aeruginosa BphP (red, PDB: 3C2W, subunit A). Structures are shown in stereo view, with strands and helices labeled as they are named in panel A. Superposition statistics are shown in Table 3. FIGS. 9C-9E: Representative electron density is shown for the hairpin/GAF domain interface near the A pyrrole ring (FIG. 9C), the PΦB-binding pocket near the D pyrrole ring (FIG. 9D), and the PHY-domain near the conserved residue, Trp599 (FIG. 9E). Here 2Fo-Fc maps were contoured at 1σ. Blue, green, and orange represents NTE/PAS, GAF, and PHY/hairpin residues, respectively. Specific amino acids are labeled along with the A, C, and D pyrrole rings of the bilin.

FIGS. 10A-10C show the effects of various mutations on the photochemical properties of Arabidopsis PhyB. FIG. 10A: Kinetics of Pr→Pfr photoconversion at 25° C. by red light monitored at 660 and 730 nm (left and right panels, respectively. FIG. 10B: Kinetics of Pfr→Pr photoconversion at 25° C. by far-red light monitored at 730 and 660 nm (left and right panels, respectively. Each kinetic in panels A and B represents the average of three separate measurements. FIG. 10C: Rates of Pfr→Pr thermal reversion at 25° C. for PhyB(PSM) and representative mutants monitored at 660 nm. Each rate represents the average of three separate measurements.

FIGS. 11A-11B show the UV-visible absorption spectroscopy of selected Arabidopsis PhyB(PSM) mutants. FIG. 11A: SDS-PAGE analysis of all PhyB(PSM) mutants (see FIG. 11B and FIGS. 3B and 3D). Purified PhyB(PSM) biliproteins were subjected to SDS-PAGE and either stained for protein with Coomassie Blue (Prot) or assayed for the bound bilin by zinc-induced fluorescence (Zn). FIG. 11B: UV-visible absorption spectra. Absorption and difference spectra were measured at 25° C. as Pr and following saturating red light irradiation (mostly Pfr). Absorption maxima are indicated.

FIGS. 12A-12F show an alignment of N-terminal region of various plant phytochromes. Identical amino acids are shaded black and similar amino acids at 80% conservation among the sequences are shaded grey.

### DETAILED DESCRIPTION

The present invention provides modified phytochrome polypeptides with altered photochemistry and altered signaling properties, and polynucleotide sequences encoding said polypeptides. These modified phytochrome polypeptides were generated based on the atomic perspective of plant Phy signaling through a crystal structure of the photosensing module from Arabidopsis thaliana PhyB assembled with its native chromophore phytochromobilin (PΦB). The crystal structure of the photosensing module as Pr was determined. Although its overall architecture and chromophore/protein contacts are reminiscent of bacterial relatives, significant structural differences are seen within the prominent knot, hairpin, and helical spine features. Subsequent mutational analyses lend support to a ‘toggle’ model for how Phys reversibly switch between their Pr and Pfr endstates. The availability of this 3-D model along with the identified suite of photochemical variants allow the rational redesign of Phy signaling for improved crop performance, as it allows the ability to predict which amino acid changes might impact the photochemistry and stability of plant phytochromes, and their subsequent ability to alter plant photomorphogenesis. Ths 3-D structure also provides a rational redesign of Phy signaling for optogenetic application.

The present invention also provides methods of generating transgenic plants that express these modified phtyochrome polypeptides. As the set of mutations demonstrates their potential to dramatically alter stability of the photoactivated Pfr form, thus prolonging or diminishing signaling by phytochromes after light absorption, this collection of mutations greatly expands the toolbox of phytochrome variants with which to alter crop development, architecture, and reproduction. Consequently, this invention enhances the facile and general-use strategies available to plant breeders attempting to engineer crops with improved shade tolerance needed for increased planting density or with altered flowering times. These mutations allow the generation of smaller plants to allow increase in planting density, and also the generation of plants more tolerant to low light conditions that stimulate the shade avoidance syndrome.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Bathochromic shift” as used herein refers to a change of spectral band position in the absorption, reflectance, transmittance, or emission spectrum of a molecule to a longer wavelength (lower frequency). The bathochromic shift may be a shift in the absorption peak wavelength, i.e., the absorption maximum.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual plant or animal cell to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

As used herein, a “control plant” is a plant that is substantially equivalent to a test plant or modified plant in all parameters with the exception of the test parameters. For example, when referring to a plant into which a polynucleotide according to the present invention has been introduced, in certain embodiments, a control plant is an equivalent plant into which no such polynucleotide has been introduced. In certain embodiments, a control plant is an equivalent plant into which a control polynucleotide has been introduced. In such instances, the control polynucleotide is one that is expected to result in little or no phenotypic effect on the plant.

A “functional homolog,” “functional equivalent,” or “functional fragment” of a polypeptide of the present invention is a polypeptide that is homologous to the specified polypeptide but has one or more amino acid differences from the specified polypeptide. A functional fragment or equivalent of a polypeptide retains at least some, if not all, of the activity of the specified polypeptide.

A “fusion protein” as used herein refers to an artificially made or recombinant molecule that comprises two or more protein sequences that are not naturally found within the same protein. The fusion protein may include non-proteinaceous elements as well as proteinaceous elements. For example, a fusion protein may comprise a modified plant phytochrome, or fragment thereof, PIF3, or fragment thereof, and/or a chromophore.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Hypsochromic shift” as used herein refers to a change of spectral band position in the absorption, reflectance, transmittance, or emission spectrum of a molecule to a shorter wavelength (higher frequency). The hypsochromic shift may be a shift in the absorption peak wavelength, i.e., the absorption maxima.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

Optimal alignment of sequences for comparison may be conducted by methods commonly known in the art, for example by the search for similarity method described by Pearson and Lipman 1988, Proc. Natl. Acad. Sci. USA 85: 2444-2448, by computerized implementations of algorithms such as GAP, BESTFIT, BLAST, FASTA, and TFASTA in the Wisconsin Genetics Software Package, Genetics Computer Group (GCG), Madison, Wis., or by inspection. In a preferred embodiment, protein and nucleic acid sequence identities are evaluated using the Basic Local Alignment Search Tool (“BLAST”), which is well known in the art (Karlin and Altschul, Proc. Natl. Acad. Sci. USA 87: 2267-2268 (1990); Altschul et al., Nucl. Acids Res. 25: 3389-3402 (1997)), the disclosures of which are incorporated by reference in their entireties. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990). The BLAST programs can be used with the default parameters or with modified parameters provided by the user.

The terms “isolated,” “purified” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

The specificity of single-stranded DNA to hybridize complementary fragments is determined by the “stringency” of the reaction conditions (Sambrook et al., Molecular Cloning and Laboratory Manual, Second Ed., Cold Spring Harbor (1989)). Hybridization stringency increases as the propensity to form DNA duplexes decreases. In nucleic acid hybridization reactions, the stringency can be chosen to favor specific hybridizations (high stringency), which can be used to identify, for example, full-length clones from a library. Less-specific hybridizations (low stringency) can be used to identify related, but not exact (homologous, but not identical), DNA molecules or segments.

DNA duplexes are stabilized by: (1) the number of complementary base pairs; (2) the type of base pairs; (3) salt concentration (ionic strength) of the reaction mixture; (4) the temperature of the reaction; and (5) the presence of certain organic solvents, such as formamide, which decrease DNA duplex stability. In general, the longer the probe, the higher the temperature required for proper annealing. A common approach is to vary the temperature; higher relative temperatures result in more stringent reaction conditions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. Since the target sequences are generally present at excess, at Tm, 50% of the probes are occupied at equilibrium.

“Stringent hybridization conditions” are conditions that enable a probe, primer, or oligonucleotide to hybridize only to its target sequence (e.g., SEQ ID NO:1). Stringent conditions are sequence-dependent and will differ. Stringent conditions comprise: (1) low ionic strength and high temperature washes, for example 15 mM sodium chloride, 1.5 mM sodium citrate, 0.1% sodium dodecyl sulfate, at 50° C.; (2) a denaturing agent during hybridization, e.g. 50% (v/v) formamide, 0.1% bovine serum albumin, 0.1% Ficoll, 0.1% polyvinylpyrrolidone, 50 mM sodium phosphate buffer (750 mM sodium chloride, 75 mM sodium citrate; pH 6.5), at 42° C.; or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a wash at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

“Moderately stringent conditions” use washing solutions and hybridization conditions that are less stringent, such that a polynucleotide will hybridize to the entire, fragments, derivatives, or analogs of the target sequence (e.g., SEQ ID NO:1). One example comprises hybridization in 6×SSC, 5×Denhardt's solution, 0.5% SDS and 100 μg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions have been described (Ausubel et al., Current Protocols in Molecular Biology, Volumes 1-3, John Wiley & Sons, Inc., Hoboken, N.J. (1993); Kriegler, Gene Transfer and Expression: A Laboratory Manual, Stockton Press, New York, N.Y. (1990); Perbal, A Practical Guide to Molecular Cloning, 2nd edition, John Wiley & Sons, New York, N.Y. (1988)).

“Low stringent conditions” use washing solutions and hybridization conditions that are less stringent than those for moderate stringency, such that a polynucleotide will hybridize to the entire, fragments, derivatives, or analogs of the target sequence (e.g., SEQ ID NO:1). A nonlimiting example of low stringency hybridization conditions includes hybridization in 35% formamide, 5×SSC, 50 mM Tris HCl (pH 7.5), 5 mM EDTA, 0.02% PVP, 0.02% Ficoll, 0.2% BSA, 100 μg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency, such as those for cross-species hybridizations, are well-described (Ausubel et al., 1993; Kriegler, 1990).

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “optogenetic” as used herein refers to the combination of genetics and optics to control well-defined events within specific cells of living tissue.

The term “photoconversion” as used herein refers to the conversion of phytochrome from the Pr form to Pfr form and/or the Pfr form to Pr form upon absorption of light. The Pr form may be converted to the Pfr form after it absorbs red light (e.g., 660 nm). The Pfr form may be converted to the Pr form after it absorbs far-red light (e.g., 720 nm).

“Phytochrome”, “Phy” or “phy” as used interchangeably herein refers to a photoreceptor that is a pigment protein that absorbs red light and far red light and initiates physiological responses governing light sensitive processes. Phytochrome exists in two forms, the Pr and Pfr forms that are interconverted by light. Plant phytochromes include phyA, phyB, phyC, phyD, and phyE.

“Phytochrome response” or “photoresponse” as used interchangeably herein refers to a biological response in plants due to a phytochrome molecule absorbing light and transducing a signal. Phytochrome responses may include photoperiodic induction of flowering, chloroplast development (not including chlorophyll synthesis), leaf senescence and leaf abscission.

As used herein, the term “plant” includes reference to whole plants, plant organs (e.g., leaves, stems, roots, etc.), seeds, plant cells, and progeny of same. Parts of transgenic plants comprise, for example, plant cells, protoplasts, tissues, callus, embryos as well as flowers, ovules, stems, fruits, leaves, roots originating in transgenic plants or their progeny previously transformed with a DNA. As used herein, the term “plant cell” includes, without limitation, protoplasts and cells of seeds, suspension cultures, embryos, meristematic regions, callus tissue, leaves, roots, shoots, gametophytes, sporophytes, pollen, and microspores.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

The term “PIF3” as used herein refers to the transcription factor phytochrome-interacting factor 3 that interacts with photoreceptors phyA and phyB. PIF3 forms a ternary complex in vitro with G-box element of the promoters of LHY, CCA1. PIF3 acts as a negative regulator of phyB signaling and degrades rapidly after irradiation of dark grown seedlings in a process controlled by phytochromes. PIF3 binds to G- and E-boxes, but not to other ACGT elements (ACEs). PIF's function as a transcriptional activator can be functionally and mechanistically separated from its role in repression of PhyB mediated processes.

The term “phytochrome domain-interacting peptide” or “PIP” as used interchangeably herein refers to any protein sequence that can bind selectively to one conformeric state of a phytochrome, such as a modified plant phytochrome, but not the other. For example, the PIP may bind to the Pfr state but not to the Pr state of the phytochrome.

The term “substantial identity” of polynucleotide sequences means that a polynucleotide comprises a sequence that has at least 25% sequence identity compared to a reference sequence as determined using the programs described herein; preferably BLAST using standard parameters, as described. Alternatively, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein of the present invention include nucleic acid sequences that have substantial identity to the nucleic acid sequences that encode the polypeptides of the present invention. Polynucleotides encoding a polypeptide comprising an amino acid sequence that has at least about: 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

The term “substantial identity” of amino acid sequences (and of polypeptides having these amino acid sequences) normally means sequence identity of at least 40% compared to a reference sequence as determined using the programs described herein; preferably BLAST using standard parameters, as described. Preferred percent identity of amino acids can be any integer from 40% to 100%. More preferred embodiments include amino acid sequences that have at least about: 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine. Accordingly, polypeptides or proteins of the present invention include amino acid sequences that have substantial identity to the amino acid sequences of the polypeptides of the present invention, which are modified phytochromes that result in plants having altered sensitivity compared with plants.

The term “thermal reversion” as used herein refers to reversion of the far-red absorbing form of phytochrome (Pfr) to the red absorbing form (Pr) typically in the dark.

“Transgenic plant” as used herein refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode a modified phytochrome polypeptide, as disclosed herein. Alternatively, the vector may comprise a polynucleotide sequence encoding a modified phytochrome polypeptide, as disclosed herein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

### 2. MODIFIED PLANT PHYTOCHROMES

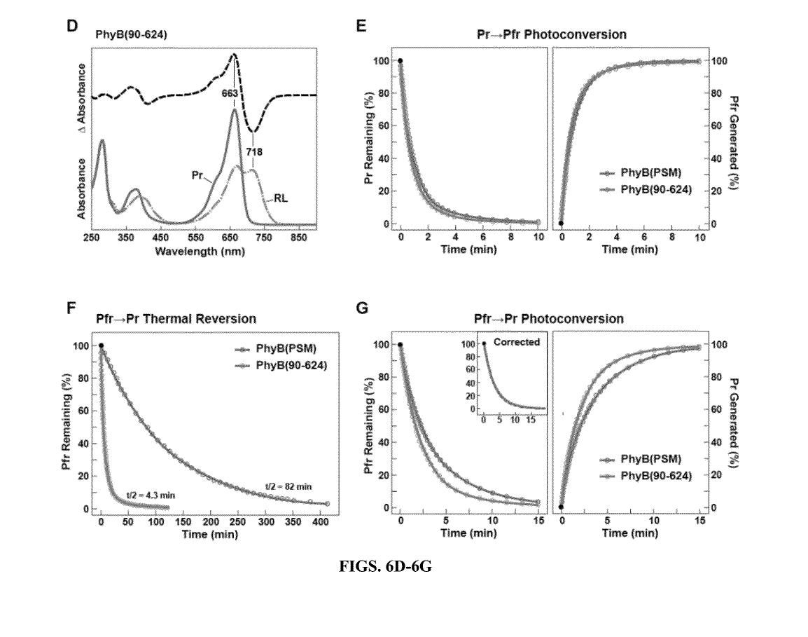

The present invention is directed to modified plant phytochrome polypeptides, functional fragments thereof, and polynucleotides encoding said polypeptides. Phytochromes are red/far-red light-absorbing photoreceptors that play key roles in the assessment of light photosynthetic potential and duration in plants, enabling the attunement of photomorphogenesis and reproduction with circadian and seasonal rhythms in their environment. Phytochromes encompass a diverse collection of biliproteins that enable cellular light perception by photoconverting between a red-light (R)-absorbing ground state, the Pr form, and a far-red light (FR)-absorbing active state, the Pfr form. In Arabidopsis thaliana there are five phytochromes, designated phytochrome A (phyA) to phytochrome E (phyE). Phytochrome B (phyB) is the predominant phytochrome regulating de-etiolation responses in red light and shade avoidance. Phytochromes are synthesized in the cytosol as an inactive Pr form. Light irradiation converts the phytochromes to the biologically active Pfr form, which then is translocated into the nucleus. Phytochromes play fundamental roles in photoperception by a plant and adaptation of its growth to the ambient light environment. Phys exist as homodimers with each sister polypeptide divided into an N-terminal PSM that embraces the bilin, followed by an OPM that promotes dimerization and presumably relays the light signals. The PSM sequentially contains a PAS domain of unknown function, a GAF domain that cradles the bilin, and a PHY domain that stabilizes the photoactivated state (FIG. 6). The OPM harbors consecutive PAS, PAS, and histidine kinase-related domains that may participate in signaling through interactions with downstream effectors and/or by a currently enigmatic kinase activity.

Plant Phys utilize phytochromobilin (PΦB) as the chromophore, which binds via a thioether linkage to a conserved GAF domain cysteine using an intrinsic lyase activity. They are synthesized in a biologically inactive, red light-absorbing Pr form, which converts upon photoexcitation to a far-red light-absorbing Pfr form that is biologically active. A proposed key step involves a red-light driven Z to E isomerization of the C15=C16 bond in PΦB that rotates the D pyrrole ring, which presumably initiates conformation changes within the bilin-binding pocket that reverberate to the OPM. Pfr can rapidly convert back to Pr with far-red light, or slowly by spontaneous thermal reversion, thus allowing Phys to act as both short- and long-lived photoswitches. Pr→Pfr photoconversion also triggers movement of Pfr from the cytosol to the nucleus where it extensively reprograms plant gene expression mainly by promoting turnover of a family of Phy-Interacting Factor transcriptional repressors.

The present disclosure describes the crystal structure of a Phy PSM from a seed plant in the Pr state, i.e., the PSM of the PhyB isoform from Arabidopsis thaliana, and extensively characterizes its solution and photochemical properties. Although the PhyB PSM behaves as monomer in solution, it crystallized as a head-to-head dimer via a helical interface involving sister GAF domains. PhyB retained many features common to its bacterial progenitors, including a ZZZssa configuration of the phytochromobilin chromophore buried within the GAF domain and a well-ordered hairpin protruding from the PHY domain toward the bilin pocket, thus implying a similar photochemistry. However, its PAS domain, knot region, and helical spine show distinct structural differences potentially important to signaling. Included is an elongated helical spine, an extended β sheet connecting the GAF domain and hairpin stem, and novel interactions between the region upstream of the PAS domain knot and the bilin A and B pyrrole rings. These differences include changes to the lasso motif and helical spine that are likely involved in signal transmission to the PhyB output module. The structure also reveals the positions of extensive, conserved loops in both the PAS and PHY domains, which are not found in bacterial phytochromes and may be important for binding of associated factors. Comparisons of this structure with those from bacterial Phys combined with mutagenic studies support a ‘toggle’ model for photoconversion that engages multiple features within the PSM to stabilize the Pr and Pfr end states after rotation of the D pyrrole ring. The analyses identified features of PhyB in photoconversion that are contributed by the GAF domain, hairpin and unique N-terminal extension. Current mechanistic models of phytochrome photoconversion are extended by identifying a conserved feature, heretofore unappreciated in phytochromes, the extension of the GAF domain β-sheet by the PHY domain hairpin in the dark-adapted form of canonical phytochromes. This Arabidopsis PhyB structure may facilitate mechanistic insights into plant Phy signaling and provide an essential scaffold to redesign their activities for agricultural benefit. The Arabidopsis PhyB structure may also enable molecular insights into plant Phy signaling and provide a scaffold to redesign of their activities for optogenetic reagents. The analysis provides a coherent view of photoconversion that allows manipulation of phytochrome and hence plant photomorphogenesis and growth for agricultural benefit.

When compared to the collection of 3-D models generated with bacterial relatives, a common architecture for Phy-type photoreceptors emerges along with the identification of plant Phy-specific features. Collectively, these comparisons combined with mutagenic studies extends the “tryptophan switch” model for photoconversion to include a dynamic interplay of additional features within the PSM in driving photoconversion and stabilizing the Pr and Pfr endstates. Presumably, the resulting ‘toggle’ between these endstates encourages differential signaling output that provides light-dependent information related to a plant's environment.

a. Modification of Phytochrome Domains

Phytochrome domains from a variety of organisms may be used as starting points (see e.g., Table 1) for modifications that will generate the modified phytochrome polypeptides of the present invention, and isolated polynucleotides encoding said modified polypeptides. Nucleotide sequences encoding various phytochromes from a variety of species are listed in Table 1.

TABLE 1
Phy from a variety of species

| | Accession | Nucleotide |
|---|---|---|
| Species | Number | SEQ ID NO: |
| Arabidopsis | NM_127435 | SEQ ID NO: 32 |
| Zea mays(maize) phyB | GRMZM2G124532 | SEQ ID NO: 33 |
| | (AF137332) | |
| Oryza sativa (rice) phyB | JN594210 | SEQ ID NO: 34 |
| Sorghum bicolor (sorghum) | AY466089 | SEQ ID NO: 35 |
| phyB | | |
| Glycine max (soybean) phyB1 | EU428749 | SEQ ID NO: 36 |
| G. max phyB2 | EU428750 | SEQ ID NO: 37 |
| G. max phyB3 | EU428751 | SEQ ID NO: 38 |
| G. max phyB4 | EU428752 | SEQ ID NO: 39 |
| Solanum tuberosum L. | DQ342235 | SEQ ID NO: 40 |
| (potato) phyB | | |
| Pisum sativum (pea) phyB | AF069305 | SEQ ID NO: 41 |
| Vitis vinifera (grape) phyB | EU436650 | SEQ ID NO: 42 |
| Z. mays phyA1 | AY234826.1 | SEQ ID NO: 43 |
| O. sativa phyA | NM_001057631.1 | SEQ ID NO: 44 |
| Avena sativa phyA4 | P06594.3 | SEQ ID NO: 45 |
| | (X03243) | |
| A. thaliana phyA | NM_100828.3 | SEQ ID NO: 46 |
| Nicotiana tabacum phyA1 | P33530.1 | SEQ ID NO: 47 |
| | (X66784) | |
| A. thaliana phyE | X76610.1 | SEQ ID NO: 48 |
| A. thaliana phyC | NM_122975.2 | SEQ ID NO: 49 |
| O. sativa phyC | NM_001057831.1 | SEQ ID NO: 50 |
| Z. mays phyC1 | AY234829.1 | SEQ ID NO: 51 |
| Z. mays phyC2 | NM_001138150.1 | SEQ ID NO: 52 |
| Physcomitrella patens phy1 | XM_001778103.1 | SEQ ID NO: 53 |
| P. patens phy3 | AB275306.1 | SEQ ID NO: 54 |
| Selaginella martensii phy1 | Q01549.1 | SEQ ID NO: 55 |
| | (X61458.1) | |
| P. patens phy2 | XM_001782287.1 | SEQ ID NO: 56 |
| P. patens phy4 | XM_001773498.1 | SEQ ID NO: 57 |
| P. patens phy5a | XM_001761093.1 | SEQ ID NO: 58 |
| P. patens phy5b3 | XM_001767172.1 | SEQ ID NO: 59 |
| P. patens phy5c | XM_001754314.1 | SEQ ID NO: 60 |
| Z. mays phyB1 | DQ307579.1 | SEQ ID NO: 61 |
| Z. mays phyB2 | NM_001174606.1 | SEQ ID NO: 62 |
| N. tabacum phyB | P29130.2 | SEQ ID NO: 63 |
| | (L10114.1) | |
| Populus balsamifera phyB1 | AF309806.1 | SEQ ID NO: 64 |
| P. balsamifera phyB2 | AF309807.1 | SEQ ID NO: 65 |
| A. thaliana phyD | NM_117721.1 | SEQ ID NO: 66 |

In certain embodiments, the modified phytochrome may include a modified plant phyB, a modified PSM, a modified chromophore binding domain (CBD) of phyB, a modified PAS domain, a modified GAF domain, and/or a modified PHY domain. Modification of phytochromes and/or phytochrome domains can be performed by methods known in the art, e.g., site-directed mutations, additions, deletions, and/or substitutions of one or more amino acid residues of existing phytochromes and/or phytochrome domains. Alternatively, modified phytochromes and/or phytochrome domains can be synthesized de novo, for example by synthesis of novel genes that would encode phytochrome domains with desired modifications

In certain embodiments, an isolated polynucleotide may encode a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92. The polynucleotide encoding a polypeptide comprising a sequence may have at least about 80%, at least about 85%, at least about 90%, at least about 91%, at least about 92%, at least about 93%, at least about 94%, at least about 95%, at least about 96%, at least about 97%, at least about 98%, or at least about 99% identity to at least one amino acid sequence of unmodified phytochrome polypeptide of any one of SEQ ID NO: 1-26 or 67-92.

The polypeptide may have an amino acid other than tyrosine at the residue corresponding to position 104 of SEQ ID NO: 1, an amino acid other than isoleucine or methionine at the residue corresponding to position 108 of SEQ ID NO: 1, an amino acid other than glycine at the residue corresponding to position 284 of SEQ ID NO: 1, an amino acid other than histidine at the residue corresponding to position 358 of SEQ ID NO: 1, an amino acid other than valine at the residue corresponding to position 401 of SEQ ID NO: 1, an amino acid other than histidine H at the residue corresponding to position 403 of SEQ ID NO: 1, an amino acid other than tryptophan at the residue corresponding to position 563 of SEQ ID NO: 1, an amino acid other than glycine at the residue corresponding to position 565 of SEQ ID NO: 1, an amino acid other than serine at the residue corresponding to position 584 of SEQ ID NO: 1, or a combination thereof. The modified phytochrome polypeptide may comprise a substitution corresponding to at least one of Y104-A, I108-A, I108-Y, G284-V, H358-A, V401-S, H403-A, W563-S, G565-E, S584-A, S584-E, or a combination thereof, of SEQ ID NO:1. The modified phytochrome polypeptide may further comprise at least one amino acid substitution at a position corresponding to position 276, 307, 322, 352, 361, 564, 582, or a combination thereof, of SEQ ID NO:1. The modified phytochrome polypeptide may further comprise a substitution corresponding to at least one of Y276-H, D307-A, R322-A, R352-A, Y361-F, G564-E, R582-A, or a combination thereof, of SEQ ID NO:1. The modified phytochrome polypeptide may have at least one, at least two, at least three, at least four, at least five, at least six, at least seven, at least eight, at least nine or at least ten amino acid substitutions. In particular, the modified phytochrome may have an amino acid substitution of W563-S, G564-E, and G565-E.

b. Altered Photoconversion Rate

The modified phytochrome polypeptide may have altered photoconversion rate as compared to an unmodified phytochrome polypeptide. The rate of photoconversion from the Pr form to the Pfr form of the modified phytochrome polypeptide may be increased compared to the unmodified phytochrome polypeptide. The rate of photoconversion from the Pr form to the Pfr form of the modified phytochrome polypeptide may be decreased compared to the unmodified phytochrome polypeptide. The rate of photoconversion from the Pfr form to the Pr form of the modified phytochrome polypeptide may be increased compared to the unmodified phytochrome polypeptide. The rate of photoconversion from the Pfr form to the Pr form of the modified phytochrome polypeptide may be decreased compared to the unmodified phytochrome polypeptide.

The modified phytochrome may have an increased Pfr to Pr photoconversion rate compared to an unmodified phytochrome when determined at a particular wavelength. For example, the wavelength may be about 710 nm to about 850 nm. The wavelength may be about 720 nm. The modified phytochrome may have an increased Pr to Pfr photoconversion rate compared to an unmodified phytochrome when determined at a particular wavelength. For example, the wavelength may be about 620 to about 740 nm. The wavelength may be about 660 nm or about 720 nm.

The modified phytochrome may have an increased photoconversion rate of at least about 0.1 fold to at least about 10 fold, at least about 0.2 fold to at least about 10 fold, at least about 0.3 fold to at least about 10 fold, at least about 0.4 fold to at least about 10 fold, at least about 0.5 fold to at least about 10 fold, at least about 0.6 fold to at least about 10 fold, at least about 0.7 fold to at least about 10 fold, at least about 0.8 fold to at least about 10 fold, at least about 0.9 fold to at least about 10 fold, at least about 1.1 fold to at least about 10 fold, at least about 1.2 fold to at least about 10 fold, at least about 1.3 fold to at least about 10 fold, at least about 1.4 fold to at least about 10 fold, at least about 1.5 fold to at least about 10 fold, at least about 1.6 fold to at least about 10 fold, at least about 1.7 fold to at least about 10 fold, at least about 1.8 fold to at least about 10 fold, at least about 1.9 fold to at least about 10 fold, at least about 2.0 fold to at least about 10 fold, at least about 3.0 fold to at least about 10 fold, at least about 4.0 fold to at least about 10 fold, at least about 5.0 fold to at least about 10 fold, at least about 6.0 fold to at least about 10 fold, at least about 7.0 fold to at least about 10 fold, at least about 8.0 fold to at least about 10 fold, or at least about 9.0 fold to at least about 10 fold compared to an unmodified phytochrome. The modified phytochrome may have an increased photoconversion rate of at least about 0.1 fold, at least about 0.2 fold, at least about 0.3 fold, at least about 0.4 fold, at least about 0.5 fold, at least about 0.6 fold, at least about 0.7 fold, at least about 0.8 fold, at least about 0.9 fold, at least about 1.0 fold, at least about 2.0 fold, at least about 3.0 fold, at least about 4.0 fold, at least about 5.0 fold, at least about 6.0 fold, at least about 7.0 fold, at least about 8.0 fold, at least about 9.0 fold, or at least about 10.0 fold compared to an unmodified phytochrome.

The modified phytochrome may have a decreased Pfr to Pr photoconversion rate compared to an unmodified phytochrome when determined at a particular wavelength. For example, the wavelength may be about 710 nm to about 850 nm. The wavelength may be about 720 nm. The modified phytochrome may have a decreased Pr to Pfr photoconversion rate compared to an unmodified phytochrome when determined at a particular wavelength. For example, the wavelength may be about 620 to about 740 nm. The wavelength may be about 660 nm or about 720 nm.

The modified phytochrome may have a decreased photoconversion rate of at least about 0.1 fold to at least about 10 fold, at least about 0.2 fold to at least about 10 fold, at least about 0.3 fold to at least about 10 fold, at least about 0.4 fold to at least about 10 fold, at least about 0.5 fold to at least about 10 fold, at least about 0.6 fold to at least about 10 fold, at least about 0.7 fold to at least about 10 fold, at least about 0.8 fold to at least about 10 fold, at least about 0.9 fold to at least about 10 fold, at least about 1.1 fold to at least about 10 fold, at least about 1.2 fold to at least about 10 fold, at least about 1.3 fold to at least about 10 fold, at least about 1.4 fold to at least about 10 fold, at least about 1.5 fold to at least about 10 fold, at least about 1.6 fold to at least about 10 fold, at least about 1.7 fold to at least about 10 fold, at least about 1.8 fold to at least about 10 fold, at least about 1.9 fold to at least about 10 fold, at least about 2.0 fold to at least about 10 fold, at least about 3.0 fold to at least about 10 fold, at least about 4.0 fold to at least about 10 fold, at least about 5.0 fold to at least about 10 fold, at least about 6.0 fold to at least about 10 fold, at least about 7.0 fold to at least about 10 fold, at least about 8.0 fold to at least about 10 fold, or at least about 9.0 fold to at least about 10 fold compared to an unmodified phytochrome. The modified phytochrome may have a decreased photoconversion rate of at least about 0.1 fold, at least about 0.2 fold, at least about 0.3 fold, at least about 0.4 fold, at least about 0.5 fold, at least about 0.6 fold, at least about 0.7 fold, at least about 0.8 fold, at least about 0.9 fold, at least about 1.0 fold, at least about 2.0 fold, at least about 3.0 fold, at least about 4.0 fold, at least about 5.0 fold, at least about 6.0 fold, at least about 7.0 fold, at least about 8.0 fold, at least about 9.0 fold, or at least about 10.0 fold compared to an unmodified phytochrome.

c. Altered Thermal Reversion Rate

The modified phytochrome polypeptide may have altered thermal reversion rate as compared to an unmodified phytochrome polypeptide. The modified phytochrome may have an increased Pfr to Pr thermal reversion rate compared to an unmodified phytochrome. The modified phytochrome may have an increased thermal reversion rate of at least about 0.001 fold to at least about 1000 fold, at least about 0.01 fold to at least about 1000 fold, at least about 0.1 fold to at least about 1000 fold, at least about 0.5 fold to at least about 1000 fold, at least about 1.0 fold to at least about 1000 fold, at least about 10 fold to at least about 1000 fold, at least about 50 fold to at least about 1000 fold, at least about 100 fold to at least about 1000 fold, at least about 200 fold to at least about 1000 fold, at least about 300 fold to at least about 1000 fold, at least about 400 fold to at least about 1000 fold, at least about 450 fold to at least about 1000 fold, at least about 500 fold to at least about 1000 fold, at least about 1.0 fold to at least about 750 fold, at least about 10 fold to at least about 750 fold, at least about 50 fold to at least about 750 fold, at least about 100 fold to at least about 750 fold, at least about 200 fold to at least about 750 fold, at least about 300 fold to at least about 750 fold, at least about 400 fold to at least about 750 fold, at least about 450 fold to at least about 750 fold, at least about 500 fold to at least about 750 fold, at least about 1.0 fold to at least about 500 fold, at least about 10 fold to at least about 500 fold, at least about 50 fold to at least about 500 fold, at least about 100 fold to at least about 500 fold, at least about 200 fold to at least about 500 fold, at least about 300 fold to at least about 500 fold, at least about 400 fold to at least about 500 fold, or at least about 450 fold to at least about 500 fold compared to an unmodified phytochrome. The modified phytochrome may have an increased thermal reversion rate of at least about 0.001 fold, at least about 0.01 fold, at least about 0.1 fold, at least about 0.5 fold, at least about 1.0 fold, at least about 2.0 fold, at least about 3.0 fold, at least about 4.0 fold, at least about 5.0 fold, at least about 6.0 fold, at least about 7.0 fold, at least about 8.0 fold, at least about 9.0 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 250 fold, at least about 300 fold, at least about 400 fold, at least about 500 fold, at least about 600 fold, at least about 700 fold, at least about 800 fold, at least about 900 fold, or at least about 1000 fold compared to an unmodified phytochrome.

The modified phytochrome may have a decreased Pfr to Pr thermal reversion rate compared to an unmodified phytochrome. The modified phytochrome may have a decreased thermal reversion rate of at least about 0.001 fold to at least about 1000 fold, at least about 0.01 fold to at least about 1000 fold, at least about 0.1 fold to at least about 1000 fold, at least about 0.5 fold to at least about 1000 fold, at least about 1.0 fold to at least about 1000 fold, at least about 10 fold to at least about 1000 fold, at least about 50 fold to at least about 1000 fold, at least about 100 fold to at least about 1000 fold, at least about 200 fold to at least about 1000 fold, at least about 300 fold to at least about 1000 fold, at least about 400 fold to at least about 1000 fold, at least about 450 fold to at least about 1000 fold, at least about 500 fold to at least about 1000 fold, at least about 1.0 fold to at least about 750 fold, at least about 10 fold to at least about 750 fold, at least about 50 fold to at least about 750 fold, at least about 100 fold to at least about 750 fold, at least about 200 fold to at least about 750 fold, at least about 300 fold to at least about 750 fold, at least about 400 fold to at least about 750 fold, at least about 450 fold to at least about 750 fold, at least about 500 fold to at least about 750 fold, at least about 1.0 fold to at least about 500 fold, at least about 10 fold to at least about 500 fold, at least about 50 fold to at least about 500 fold, at least about 100 fold to at least about 500 fold, at least about 200 fold to at least about 500 fold, at least about 300 fold to at least about 500 fold, at least about 400 fold to at least about 500 fold, or at least about 450 fold to at least about 500 fold compared to an unmodified phytochrome. The modified phytochrome may have a decreased thermal reversion rate of at least about 0.001 fold, at least about 0.01 fold, at least about 0.1 fold, at least about 0.5 fold, at least about 1.0 fold, at least about 2.0 fold, at least about 3.0 fold, at least about 4.0 fold, at least about 5.0 fold, at least about 6.0 fold, at least about 7.0 fold, at least about 8.0 fold, at least about 9.0 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 250 fold, at least about 300 fold, at least about 400 fold, at least about 500 fold, at least about 600 fold, at least about 700 fold, at least about 800 fold, at least about 900 fold, or at least about 1000 fold compared to an unmodified phytochrome. In some embodiments, the modified phytochrome polypeptide may fail to thermally revert from Pfr to Pr.

d. Altered Absorption Spectrum

The modified phytochrome polypeptide may have an altered absorption spectrum as compared to an unmodified phytochrome polypeptide. The modified phytochrome polypeptide may have a Pr absorption spectrum that is shifted to a longer wavelength compared to the unmodified phytochrome polypeptide. The modified phytochrome polypeptide may have a Pr absorption spectrum that is shifted to a shorter wavelength compared to the unmodified phytochrome polypeptide. The modified phytochrome polypeptide may have a Pfr absorption spectrum that is shifted to a longer wavelength compared to the unmodified phytochrome polypeptide. The modified phytochrome polypeptide may have a Pfr absorption spectrum that is shifted to a shorter wavelength compared to the unmodified phytochrome polypeptide. The altered absorption spectrum may be a shift in an absorption peak wavelength.

The modified phytochrome may have a hypsochromic (shorter) or bathochromic (longer) wavelength shift of at least about 0.5 nm to at least about 100 nm, at least about 1.0 nm to at least about 100 nm, at least about 2.0 nm to at least about 100 nm, at least about 3.0 nm to at least about 100 nm, at least about 4.0 nm to at least about 100 nm, at least about 6.0 nm to at least about 100 nm, at least about 7.0 nm to at least about 100 nm, at least about 8.0 nm to at least about 100 nm, at least about 9.0 nm to at least about 100 nm, at least about 10.0 nm to at least about 100 nm, at least about 0.5 nm to at least about 75 nm, at least about 1.0 nm to at least about 75 nm, at least about 2.0 nm to at least about 75 nm, at least about 3.0 nm to at least about 75 nm, at least about 4.0 nm to at least about 75 nm, at least about 6.0 nm to at least about 75 nm, at least about 7.0 nm to at least about 75 nm, at least about 8.0 nm to at least about 75 nm, at least about 9.0 nm to at least about 75 nm, at least about 10.0 nm to at least about 75 nm, at least about 0.5 nm to at least about 50 nm, at least about 1.0 nm to at least about 50 nm, at least about 2.0 nm to at least about 50 nm, at least about 3.0 nm to at least about 50 nm, at least about 4.0 nm to at least about 50 nm, at least about 6.0 nm to at least about 50 nm, at least about 7.0 nm to at least about 50 nm, at least about 8.0 nm to at least about 50 nm, at least about 9.0 nm to at least about 50 nm, at least about 10.0 nm to at least about 50 nm, at least about 0.5 nm to at least about 25 nm, at least about 1.0 nm to at least about 25 nm, at least about 2.0 nm to at least about 25 nm, at least about 3.0 nm to at least about 25 nm, at least about 4.0 nm to at least about 25 nm, at least about 6.0 nm to at least about 25 nm, at least about 7.0 nm to at least about 25 nm, at least about 8.0 nm to at least about 25 nm, at least about 9.0 nm to at least about 25 nm, at least about 10.0 nm to at least about 25 nm in the Pr or Pfr absorption spectrum compared to an unmodified phytochrome. The hypsochromic or bathochromic shift may be at least about 0.5 nm, at least about 1.0 nm, at least about 2.0 nm, at least about 3.0 nm, at least about 4.0 nm, at least about 1.0 nm, at least about 6.0 nm, at least about 7.0 nm, at least about 8.0 nm, at least about 9.0 nm, at least about 10.0 nm, at least about 15.0 nm, at least about 20.0 nm, at least about 25.0 nm, at least about 30.0 nm, at least about 35.0 nm, at least about 40.0 nm, at least about 45.0 nm, at least about 50.0 nm, at least about 55.0 nm, at least about 60.0 nm, at least about 65.0 nm, at least about 70.0 nm, at least about 75.0 nm, at least about 80.0 nm, at least about 85.0 nm, at least about 90.0 nm, at least about 95.0 nm, or at least about 100.0 nm compared to an unmodified phytochrome.

e. Altered Signal Output

The modified phytochrome polypeptide may have an altered signal output as compared to an unmodified phytochrome polypeptide. The modified phytochrome polypeptide may have a hyperactive or hypoactive signaling response. Examples where this modification would be beneficial include: making longer or shorter stems, making longer or shorter petioles, increasing or decreasing the angle of leaves/petioles relative to the stem, making the leaves more or less green due to increased/decreased number of chloroplasts or amount of chlorophyll, Making leaves larger or smaller, increased or decreased pigmentation of leaves or fruits, greater or less sensitivity of seed germination to light, earlier or delayed flowering time, and increased or decreased rates of leaf, flower or fruit senescence. It might also be possible to make plants grown similar to light grown plants without light. For example, plants having an amino acid substitution of G564 have a hyperactive signaling response.

### 3. METHODS OF PRODUCING TRANSGENIC PLANTS WITH MODIFIED PHYTOCHROMES

The present invention is directed to transgenic plants and plant cells having the modified phytochrome polypeptide or polynucleotide encoding said polypeptide and methods of generating said transgenic plants and plant cells. The transgenic plant cell may include the isolated polynucleotide encoding the modified phytochrome polypeptide described above. The isolated polynucleotide may be operably linked to a promoter not natively associated with said polynucleotide. A plant may comprise the transgenic plant cell.

The present invention is also directed to a method of producing a transgenic plant. The method includes (a) introducing into a plant cell an isolated polynucleotide encoding a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide has an amino acid sequence selected from SEQ ID NOs: 1-26 or 67-92; and (b) regenerating the transformed cell to produce a transgenic plant. The transgenic plant may be produced by introducing into a plant or plant cell a polynucleotide encoding a polypeptide comprising a sequence having at least about 80%, at least about 85%, at least about 90%, at least about 95%, at least about 98%, or at least about 99% identity to at least one amino acid sequence selected from SEQ ID NOs: 1-26 or 67-92, wherein the polypeptide has at least one amino acid substitution at a position corresponding to position 104, 108, 284, 358, 401, 403, 563, 565, 584, or a combination thereof, of SEQ ID NO:1. The polypeptide may have an amino acid other than tyrosine at the residue corresponding to position 104 of SEQ ID NO: 1, an amino acid other than isoleucine or methionine at the residue corresponding to position 108 of SEQ ID NO: 1, an amino acid other than glycine at the residue corresponding to position 284 of SEQ ID NO: 1, an amino acid other than histidine at the residue corresponding to position 358 of SEQ ID NO: 1, an amino acid other than valine at the residue corresponding to position 401 of SEQ ID NO: 1, an amino acid other than histidine H at the residue corresponding to position 403 of SEQ ID NO: 1, an amino acid other than tryptophan at the residue corresponding to position 563 of SEQ ID NO: 1, an amino acid other than glycine at the residue corresponding to position 565 of SEQ ID NO: 1, an amino acid other than serine at the residue corresponding to position 584 of SEQ ID NO: 1, or a combination thereof. The modified phytochrome polypeptide may comprise a substitution corresponding to at least one of Y104-A, I108-A, I108-Y, G284-V, H358-A, V401-S, H403-A, W563-S, G565-E, S584-A, S584-E, or a combination thereof, of SEQ ID NO:1. The modified phytochrome polypeptide may further comprise at least one amino acid substitution at a position corresponding to position 276, 307, 322, 352, 361, 564, 582, or a combination thereof, of SEQ ID NO:1. The modified phytochrome polypeptide may further comprise a substitution corresponding to at least one of Y276-H, D307-A, R322-A, R352-A, Y361-F, G564-E, R582-A, or a combination thereof, of SEQ ID NO:1. The modified phytochrome polypeptide may have at least one, at least two, at least three, at least four, at least five, at least six, at least seven, at least eight, at least nine or at least ten amino acid substitutions. In particular, the modified phytochrome may have an amino acid substitution of W563-S, G564-E, and G565-E. In certain embodiments, the polynucleotide is provided as a construct in which a promoter is operably linked to the polynucleotide.

The polynucleotide sequences may be introduced into plants which do not express the corresponding native form of unmodified phytochrome, such as plants lacking the native gene, or containing a mutated, truncated or downregulated version of the native gene, such that little or no phytochrome polypeptide is expressed, or a phytochrome polypeptide is expressed that is partially or substantially inactive. The modified phytochrome replaces or substitutes for the native gene function. The polynucleotides can also be expressed in wild-type plants containing the corresponding native phytochrome gene sequence. The modified phytochrome may over-ride the functions of the wild-type endogenous gene in a dominant fashion, since it is hyperactive.

The transgenic plant expressing the isolated polynucleotide encoding the modified phytochrome polypeptide may exhibit increased light sensitivity or altered photoresponses relative to a control plant lacking the isolated polynucleotide. The transgenic plant may exhibit decreased height, decreased diameter, or a combination thereof, relative to a control plant lacking the polynucleotide. The transgenic plant may exhibit at least one characteristic selected from decreased petiole length, decreased internode number, increased hyponasty, and decreased hypocotyl length under an R fluence rate of less than 1 μmole m⁻² sec⁻¹, relative to a control plant lacking the polynucleotide. The transgenic plant may exhibit enhanced germination relative to the control plant.

a. Altered Characteristics and Photoresponses

It is envisaged that a plant produced following the introduction of a polynucleotide disclosed herein exhibits altered or modified characteristics or photoresponses relative to the control plant. Altered photoresponses included: an improved or enhanced germination efficiency of seeds, such as in low light, altered light sensitivity, such as a hypersensitivity to white and red light with respect to hypocotyl and stem growth, larger leaf surface areas in white light, increased tolerance to shade, and a smaller plant size, such as decreased height, decreased diameter, or a combination thereof, relative to a control plant lacking the modified phytochrome or polynucleotide encoding said modified phytochrome.

The modified characteristics include, but are not limited to, increased hyponasty, decreased height, decreased diameter, decreased petiole length, decreased internode length, decreased stem length, decreased stem diameter, increased leaf chlorophyll concentration, decreased leaf length, increased root length, increased root branching, improved leaf unfolding, reduced leaf surface area, decreased hypocotyl length under an R fluence rate of less than 1 μmole m m⁻² sec⁻¹ (or less than 0.5 μmole m⁻² sec⁻¹, less than 0.6 μmole m⁻² sec⁻¹, less than 0.7 μmole m⁻² sec⁻¹, or less than 0.8 μmole m⁻² sec⁻¹), enhanced germination or any combination thereof. The altered characteristic may be decreased or enhanced by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 175%, at least about 200%, at least about 250%, at least about 300%, or at least about 400% relative to a control plant.

As a nonlimiting example, such modified plants may have a compact size, i.e., smaller mature plant size, and have a height or diameter that is at least about 20%, at least about 30%, at least about 50%, at least about 75%, or at least about 100% smaller than the height or diameter of a control plant. As another nonlimiting example, such modified plants may provide an increased yield of seed, grain, forage, fruit, root, leaf, or combination thereof, that is at least about 5%, at least about 10%, at least about 20%, at least about 25%, at least about 30%, at least about 40%, at least about 50%, at least about 60%, at least about 70%, at least about 75%, at least about 80%, at least about 90%, or at least about 100% increased over the yield from corresponding control plants. As used herein, “yield” refers to the maximum yield achievable per given planting area, and does not refer to the yield from an individual plant. Maximum or higher yields may be achieved by planting a higher number or density of plants in a given area.

The modified phytochrome polypeptide may generate a hyperactive photoreceptor that still requires light for activation. As such, plants expressing the modified phytochrome polypeptide may display accentuated phytochrome signaling, useful in agricultural settings with fewer side effects. The replacement of wild-type phytochrome with the modified phytochrome polypeptide in plants may increase the sensitivity of hypocotyls to R, generate seeds with a stronger germination response in white light, and further accentuate the end-of-day far-red light (EOD-FR) response of seedlings, substantially without altering flowering time, such as in short days. The phytochrome-mediated responses to R and EOD-FR are connected to the shade avoidance response. Without wishing to be bound to any theory, it is possible that increased signaling by the modified phytochrome polypeptide may attenuate shade avoidance response by enabling the small amounts of Pfr generated by low fluence R, or the residual Pfr remaining after EOD-FR (or presumably in high FR/R light environments) to more effectively promote normal photomorphogenesis.

### 4. CONSTRUCTS AND PLASMIDS

The genetic constructs may comprise a nucleic acid sequence that encodes the modified phytochrome polypeptide disclosed herein. The genetic construct, such as a plasmid, may comprise a nucleic acid that encodes the modified phytochrome polypeptide. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant cauliflower mosaic virus, recombinant tobacco mosaic virus, and recombinant potato virus X-based vectors. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer an initiation codon, a stop codon, or a polyadenylation signal.

In certain embodiments, the polynucleotides to be introduced into the plant are operably linked to a promoter sequence and may be provided as a construct. As used herein, a polynucleotide is “operably linked” when it is placed into a functional relationship with a second polynucleotide sequence. For instance, a promoter is operably linked to a coding sequence if the promoter is connected to the coding sequence such that it may effect transcription of the coding sequence. In various embodiments, the polynucleotides may be operably linked to at least one, at least two, at least three, at least four, at least five, or at least ten promoters.

The nucleic acid sequences may make up a genetic construct that may be a vector. The vector may be capable of expressing the modified phytochrome polypeptide in the cell of a plant. The vector may be recombinant. The vector may comprise heterologous nucleic acid encoding the modified phytochrome polypeptide. The vector may be a plasmid. The vector may be useful for transfecting cells with nucleic acid encoding the modified phytochrome polypeptide, after which the transformed host cell is cultured and maintained under conditions wherein expression of the modified phytochrome polypeptide takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the modified phytochrome polypeptide and may further comprise an initiation codon, which may be upstream of the modified phytochrome polypeptide coding sequence and a stop codon, which may be downstream of the modified phytochrome polypeptide coding sequence. The initiation and termination codon may be in frame with the modified phytochrome polypeptide coding sequence. The vector may also comprise a promoter that is operably linked to the modified phytochrome polypeptide coding sequence. The promoter that is operably linked to the modified phytochrome polypeptide coding sequence may be not natively associated with the polynucleotide encoding the modified phytochrome polypeptide. Promoters useful in the practice of the present invention include, but are not limited to, constitutive, inducible, temporally-regulated, developmentally regulated, chemically regulated, tissue-preferred and tissue-specific promoters. Suitably, the promoter causes sufficient expression in the plant to produce the phenotypes described herein. Suitable promoters include, without limitation, the 35S promoter of the cauliflower mosaic virus, ubiquitin, tCUP cryptic constitutive promoter, the Rsyn7 promoter, pathogen-inducible promoters, the maize In2-2 promoter, the tobacco PR-la promoter, glucocorticoid-inducible promoters, and tetracycline-inducible and tetracycline-repressible promoters.

The vector may also comprise a polyadenylation signal, which may be downstream of the modified phytochrome polypeptide coding sequence. The vector may also comprise an enhancer upstream of the modified phytochrome polypeptide coding sequence. The enhancer may be necessary for DNA expression. The vector may also comprise a plant origin of replication in order to maintain the vector extrachromosomally and produce multiple copies of the vector in a cell. The vector may also comprise a regulatory sequence, which may be well suited for gene expression in a plant cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., 1989, which is incorporated fully by reference. In some embodiments the vector may comprise the nucleic acid sequence encoding the modified phytochrome polypeptide.

### 5. PLANT TYPES

The plant to be transformed to produce the transgenic plant may be any plant species, including non-vascular plants and vascular plants. The non-vascular plant may include a bryophyte, such as Physcomitrella patens. The vascular plants may include pteridophyte, such as Selaginella martensii, angiosperms, and gymnosperms. The angiosperms may include a monocot plant or a dicot plant. The plant may be a crop plant, such as a cereal, a fruit, a legume, or a root crop, ornamental plants, or a non-food crop, such as cotton, hemp (Cannabis sativa), flax or linseed (Linum usitatissimum), oilseed rape or high erucic acid rape (Brassica napus), balsam poplar (Populus balsamifera), tobacco (Nicotiana tabacum), and switchgrass (e.g., Panicum virgatum).

Suitable plant species include, without limitation, corn (Zea mays), soybean (Glycine max), Brassica sp. (e.g., Arabidopsis thaliana, Brassica napus, B. rapa, and B. juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), tobacco (Nicotiana tabacum), potato (Solanum tuberosum), peanuts (Arachis hypogaea), pea (Pisum sativum), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), grape (Vitis vinifera), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals, and conifers.

Vegetables include, without limitation, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Ornamental plants are plants that are grown for decorative purposes in gardens and landscapes, as houseplants, and for cut flowers. Suitable ornamentals include, without limitation, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum (Chrysanthemum spp.).

### 6. PLANT TRANSFORMATION

The polynucleotides of the present invention may be introduced into a plant cell to produce a transgenic plant. As used herein, “introduced into a plant” with respect to polynucleotides encompasses the delivery of a polynucleotide into a plant, plant tissue, or plant cell using any suitable polynucleotide delivery method. Methods suitable for introducing polynucleotides into a plant useful in the practice of the present invention include, but are not limited to, freeze-thaw method, microparticle bombardment, direct DNA uptake, whisker-mediated transformation, electroporation, sonication, microinjection, plant virus-mediated, and Agrobacterium-mediated transfer to the plant. Any suitable Agrobacterium strain, vector, or vector system for transforming the plant may be employed according to the present invention. In certain embodiments, the polynucleotide is introduced using at least one of stable transformation methods, transient transformation methods, or virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al., Biotechniques 4:320-334 (1986)), electroporation (Riggs et al., Proc. Natl. Acad. Sci. USA 83:5602-5606 (1986)), Agrobacterium-mediated transformation (U.S. Pat. Nos. 5,981,840 and 5,563,055), direct gene transfer (Paszkowski et al., EMBO J. 3:2717-2722 (1984)), and ballistic particle acceleration (see, for example, U.S. Pat. Nos. 4,945,050; 5,879,918; 5,886,244; 5,932,782; Tomes et al., in Plant Cell, Tissue, and Organ Culture: Fundamental Methods, ed. Gamborg and Phillips (Springer-Verlag, Berlin) (1995); and McCabe et al., Biotechnology 6:923-926 (1988)). Also see Weissinger et al., Ann. Rev. Genet. 22:421-477 (1988); Sanford et al., Particulate Science and Technology 5:27-37 (1987) (onion); Christou et al., Plant Physiol. 87:671-674 (1988) (soybean); McCabe et al., Bio/Technology 6:923-926 (1988) (soybean); Finer and McMullen, In Vitro Cell Dev. Biol. 27P:175-182 (1991) (soybean); Singh et al., Theor. Appl. Genet. 96:319-324 (1998) (soybean); Datta et al., Biotechnology 8:736-740 (1990) (rice); Klein et al., Proc. Natl. Acad. Sci. USA 85:4305-4309 (1988) (maize); Klein et al., Biotechnology 6:559-563 (1988) (maize); U.S. Pat. Nos. 5,240,855; 5,322,783 and 5,324,646; Klein et al., Plant Physiol. 91:440-444 (1988) (maize); Fromm et al., Biotechnology 8:833-839 (1990) (maize); Hooykaas-Van Slogteren et al., Nature (London) 311:763-764 (1984); U.S. Pat. No. 5,736,369 (cereals); Bytebier et al., Proc. Natl. Acad. Sci. USA 84:5345-5349 (1987) (Liliaceae); De Wet et al., in The Experimental Manipulation of Ovule Tissues, ed. Chapman et al., (Longman, N.Y.), pp. 197-209 (1985) (pollen); Kaeppler et al., Plant Cell Reports 9:415-418 (1990) and Kaeppler et al., Theor. Appl. Genet. 84:560-566 (1992) (whisker-mediated transformation); D'Halluin et al., Plant Cell 4:1495-1505 (1992) (electroporation); Li et al., Plant Cell Reports 12:250-255 (1993) and Christou and Ford, Annals of Botany 75:407-413 (1995) (rice); Osjoda et al., Nature Biotechnology 14:745-750 (1996) (maize via Agrobacterium tumefaciens); all of which are herein incorporated by reference in their entireties.

In some embodiments, a plant may be regenerated or grown from the plant, plant tissue or plant cell. Any suitable methods for regenerating or growing a plant from a plant cell or plant tissue may be used, such as, without limitation, tissue culture or regeneration from protoplasts. Suitably, plants may be regenerated by growing transformed plant cells on callus induction media, shoot induction media and/or root induction media. See, for example, McCormick et al., Plant Cell Reports 5:81-84 (1986). These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. Thus as used herein, “transformed seeds” refers to seeds that contain the nucleotide construct stably integrated into the plant genome.

### 7. METHODS OF USING THE MODIFIED PLANT PHYTOCHROMES IN SPATIAL AND/OR TEMPORAL REGULATION OF PROTEINS IN CELLS BY LIGHT

The present invention is also directed to methods of using the modified plant phytochromes or fragments thereof to spatially and/or temporally regulate an interaction between cellular components using light. The method includes a genetically-encoded, light-switchable assay system comprising the modified plant phytochromes for modulating protein-protein interactions and regulating the association between proteins of interest in a cell using light. The method takes advantage of the ability of phytochromes to change conformation upon exposure to appropriate light conditions, and to bind in a conformation-dependent manner to phytochrome domain-interacting peptide (PIPs). As described above, phytochromes can efficiently and reversibly photointerconvert between red light absorbing Pr and far red light absorbing Pfr forms, a property conferred by covalent association of a linear tetrapyrrole (bilin or phytobilin) with a large apoprotein.

Binding between the modified plant phytochromes and the PIPs may result in a significant and detectable interaction within the cell, yet is reversible with fast association and dissociation rates. Photoreversibility of the modified plant phytochromes allow for the system to be turned on and off readily by changing the exposure of a cell to light. The methods allow spatial and temporal control of protein interactions. As described above, the modified plant phytochromes have altered photoconversion rates, altered thermal reversion rates, altered absorption spectra and altered signal outputs compared to a corresponding unmodified phytochrome polypeptide. These altered characteristics allow these modified plant phytochromes to be more versatile as optogenetic regeants compared to an unmodified phytochrome polypeptide.

The method may be used to regulate the interaction between a first protein of interest and second protein within a cell by light. The method may include (1) providing in the cell a first fusion protein which comprises the first protein and a modified plant phytochrome, and (2) providing in the cell a second fusion protein which comprises the second protein and a phytochrome domain-interacting peptide (PIP) that can bind selectively to the Pfr state, but not to the Pr state, of the phytothrome domain. The interaction between the first fusion protein and the second fusion protein can be regulated by controlling the exposure of the cell to red light and/or infra-red light. In some aspects, the first and second protein sequences of interest do not normally associate or interact with each other. In embodiments where the first and second protein can interact with each other in their naturally-occurring forms, either or both can be modified if desired in such a manner that they do not associate or interact with each other in the absence of association between the modified plant phytochrome and the PIP.

Association of the modified plant phytochrome and the PIP, and the resulting association between the first protein and/or the second proteins of interest, can result in a biologically significant effect upon the cell. In some embodiments, the first and second proteins interact when associated via the modified plant phytochrome and the PIP, and the interaction produces an effect on a cell structure or process. For example, the first protein can cause the second protein to be modified when both are brought into proximity by the association between the modified plant phytochrome and PIP, or vice versa. In another embodiment, the first and/or second protein can associate or interact with a third protein only when the first and second proteins are brought together through an association between the modified plant phytochrome and the PIP. In yet another example, the first protein can dissociate from a third protein (e.g., an inhibitory protein) only when brought together with the second protein through an association between the modified plant phytochrome and the PIP, or vice versa.

The association between the proteins of interest can modulate or have an effect on any biologically significant cellular process. In an aspect, the association for dissociation) between the proteins or protein fusions of interest can have an effect on a cellular signaling process (e.g., the first, and/or second proteins of interest are signaling proteins).

The modified plant phytochrome may be associated with a chromophore, such as phytobilin, that is associated with the phytochrome protein sequence of the modified plant phytochrome. Other chromophores include blue shifted tetrapyrroles, such as phycoviolobilin PVB, or synthetic tetrapyrrole derivatives of natural phytobilins. Chromophores can be obtained by purification from natural sources (e.g., A. thaliana cells spirulina cells, and the like). The chromophore may be introduced into a cell of interest by exogenous administration into the extracellular environment (e.g., the culture medium), such that the outer surface of the cell is placed in contact with the chromophore, and allowing the cell to internalize the chromophore. A cell of interest can optionally be engineered or modified to contain genes for enzymes that will generate the chromophore. In some embodiments, the method inch providing cells with chromophores (e.g., phytobilins) or precursors thereof that can form part of the PHD. Alternatively, chromophores can be isolated and purified, and added to the extracellular environment, whereupon the chromophore is naturally taken up by cells.

The PIP may comprise an APA (activated phyA-binding) or APB (activated PhyB-binding) domain from phytochrome-interacting factors (PIF), or any portion, variant or derivative thereof. The PIF may be one of PIF1 to PIF6 of Arabidopsis thaliana, such as PIF3 (Gel/bank ID. 837479), PIF6 (Genbank ID. 825382), PIF4 (Genbank ID. 818903), and PIL1 (Genbank ID. 819311).

Association can be visualized by adding appropriate labels or proteins to the first and/or second construct. For example, one fusion protein may contain a membrane localization sequence, while the other fusion protein may contain a detectable tag, e.g., GFP, wherein binding can be detected by localization of the GFP to the membrane. One or more proteins (or protein fusions) of the invention may be attached to a detectable label. A wide variety of detectable labels are known in the art. Such labels include molecules that can be attached to or form part of a protein or protein fusion of the invention and are capable of being detected (or are capable of reacting to form a chemical or physical entity (e.g., reaction product) that is detectable) in an assay according to the instant disclosure. Representative examples of detectable labels or reaction products include precipitates, fluorescent signals, compounds having a color, and the like. Representative labels include, fluorophores, bioluminescent and/or chemiluminescent compounds, radioisotopes, enzymes, binding proteins (e.g., biotin, avidin, streptavidin and the like), magnetic particles, chemically reactive compounds (e.g., colored stains), labeled-oligonucleotides; molecular probes (e.g., CY3, Research Organics, Inc.), and the like.

The interaction between the cellular components may be regulated by the wavelength of the light applied to the cell. For example, the light may be red light, far red light, or no light, i.e., darkness. Any light source may be used, such as a laser.

The fusion proteins and the polynucleotide sequences that encode said fusion proteins may be prepared using standard methods known to those skilled in the art.

A variety of cells can be used, such as eukaryotic cells, including yeast, algae, fungal, fish, insect, avian and mammalian cells, and prokaryotic cells, including bacteria. One or more proteins or protein fusions of the invention can be introduced into a host cell in a variety of ways. For example, a recombinant cell can be engineered that expresses one or more proteins or protein fusions. Alternatively, the proteins or protein fusions can be introduced by any known method, such as microinjection, transfection and/or transduction of nucleic acid and/or protein.

The methods, materials and systems of the invention can be used in a variety of ways. In an aspect, the invention can be used as a research tool to study the biological role of a protein or interest, or the role of an interaction between a first and second protein of interest. The invention can also be used to identify proteins that interact in a biologically significant manner with a protein of interest, in which a known protein of interest is attached to a PDF, and a variety of candidate proteins are attached in turn to a cognate PIP, or vice versa (similar to a two-hybrid assay). In another aspect, the invention can be used to identify mutants of a protein of interest that show different interaction from the wild-type protein with a second protein. In yet another application, the invention can be used to screen for potential modulators of a protein-protein interaction or a cellular pathway.

The invention can be used in a variety of settings. The disclosed method may be applied to control processes in living cells, such as a process that is dependent on a recruitment event. The modified plant phytochromes may be used as cell biological and/or optogenetic agents to use light to control precisely cellular behavior, such as to perturb directly neuronal networks. The method may be used in vitro with cultured cells, or in vivo using organisms into which cells containing or expressing protein fusions of the invention have been introduced.

The invention can be used to study a wide variety of proteins that are capable of interacting with other proteins. In an embodiment, interactions such as dimerization or multimerization can be studied, wherein the first and second protein fusion comprise the same protein of interest. In another variation, the first and second proteins are not involved in protein splicing.

The present invention has multiple aspects, illustrated by the following non-limiting examples.

### 8. EXAMPLES

The foregoing may be better understood by reference to the following examples, which are presented for purposes of illustration and are not intended to limit the scope of the invention.

### Example 1

### Biliprotein Expression and Purification

A. thaliana PhyB PSM constructions bearing N- or C-terminal 6His tags and assembled with PΦB were expressed in Escherichia coli BL21-AI cells using a dual plasmid expression system (Gambetta et al., Proc. Natl. Acad. Sci. USA 98:10566-10571 (2001); Zhang et al., Plant Physiol. 161:1445-1457 (2013)). For the PSM constructions with N-terminal 6His tags, the MGSSHHHHHHSSENLYFQGH (SEQ ID NO:27) sequence bearing a tobacco etch virus (TEV) protease cleavage site was appended that resulted in a Gly-His extension upon TEV protease cleavage. Cells were grown at 37° C. in terrific broth containing 0.4% glycerol and 1 mM MgCl₂, temperature was reduced to 16° C., and then the medium was made 100 μM in δ-aminolevulinic acid. After 1 hr, isopropyl β-D-1-thiogalactopyranoside was added to 1 mM, followed by the addition of arabinose to 0.2% after a second hr to induce PΦB and apoprotein synthesis, respectively. Cell growth and PhyB purification were performed in darkness or under green safelights.

PhyB expressing cells were sonicated, clarified, and the resulting extract was subjected to nickel (Ni)-nitriloacetic acid chromatography (Qiagen) as described (Burgie et al., Structure 21:88-97 (2013)). PhyB constructions containing TEV-protease sites were cleaved overnight with recombinant TEV-protease. The eluates were made 200 mM in NH₂SO₄, applied to a butyl Sepharose HP column, and eluted with a linear 200 to 0 mM NH₂SO₄ gradient in 10% glycerol, 10 mM 2ME, and 20 mM HEPES (pH 7.8). PhyB fractions were exchanged into 10% glycerol, 10 mM 2ME, 20 mM NaCl, and 20 mM HEPES (pH 7.8) and purified with a Q-Sepharose HP column (GE) using a 20 to 500 mM linear NaCl gradient. Samples were exchanged into crystallization buffer (CB) containing 50 mM NaCl, 0.3 mM Tris(2-carboxyethyl)phosphine (TCEP), and 5 mM HEPES (pH 7.8), or into standard assay buffer (SAB) containing 150 mM KCl, 0.3 mM TCEP, and 50 mM HEPES (pH 7.8 at 25° C.). Samples in SAB without TCEP were flash frozen as 30 μl drops and stored at −80° C.

### Example 2

### PhyB PSM Crystallography

PhyB(90-624) biliprotein bearing a C-terminal SLHHHHHH (SEQ ID NO: 28) tag was crystallized by sitting drop vapor diffusion using the Hampton Index screen and PhyB in CB supplemented with ethylene glycol or glycerol. Well ordered crystals were formed in solutions containing 15 mg/ml PhyB, 1.2 M MgSO₄, 4% glycerol, 1% poly(ethylene glycol) 3350, and 100 mM BisTris(HCl) (pH 5.5). Crystals were exchanged into 100 mM MgSO₄, 25% poly(ethylene glycol) 3350, 15% poly(ethylene glycol) 550 monomethyl ether, and 100 mM BisTris (pH 5.5), and flash cooled in liquid nitrogen. Final datasets were collected at the Life Sciences Collaborative Access Team at the Advanced Photon Source (Argonne, Ill.), and indexed, integrated, and scaled using HKL2000 (Otwinowski et al., Method Enzymol. 276-307-326 (1997)). Initial phases were calculated by PHASER (McCoy et al., J. Appl. Crystallogr 40:658-674 (2007)) using the PAS-GAF region of Syn-Cph1 as the search model (PDB 2VEA, (Essen et al., Proc. Natl. Acad. Sci. USA 105:14709-14714 (2008)). Manual model-building was conducted with COOT (Emlsey et al., Acta Crystallogr D Biol. Crystallogr 60:2126-2132 (2004)) and refined with PHENIX (McCoy et al., J. Appl. Crystallogr 40:658-674 (2007)) without real-space refinement and invoking non-crystallographic symmetry in torsion angle mode, and validated with MOLPROBITY (Chen et al., Acta Crystallogr D Biol. Crystallogr 66:12-21 (2010)). Superpositions were arranged with LSQKAB (Kabsch, Acta Crystallogr A 32:922-923 (1976)).

### Example 3

### Equilibrium Sedimentation and Size Exclusion Chromatography (SEC) of PhyB(PSM)

Equilibrium sedimentation was conducted at 20° C. in darkness with Beckmann XL-A analytical ultracentrifuge and PhyB dissolved as Pr in SAB. SEC was conducted at 20° C. by FPLC (0.2 ml/min) with a 0.5×20 cm analytical grade Superdex 200 column (GE) equilibrated with SAB and 50 μl of either Pr samples or samples continuously irradiated with red light (mostly Pfr).

### Example 4

### Spectroscopic Measurements

Absorption spectra, Pfr→Pr thermal reversion, and Pr/Pfr interconversion were measured at 25° C. in SAB (Zhang et al., Plant Physiol 161:1445-1457 (2013)). Red or far-red light was provided by 660 nm or 730 nm peak output LEDs filtered through 10-nm half-peak width 660 or 730 nm interference filters, respectively. Absorption spectra after denaturation were recorded after dissolution in 8 M urea (pH 2.0).

### Example 5

### Overall Structure of the Arabidopsis PhyB PSM

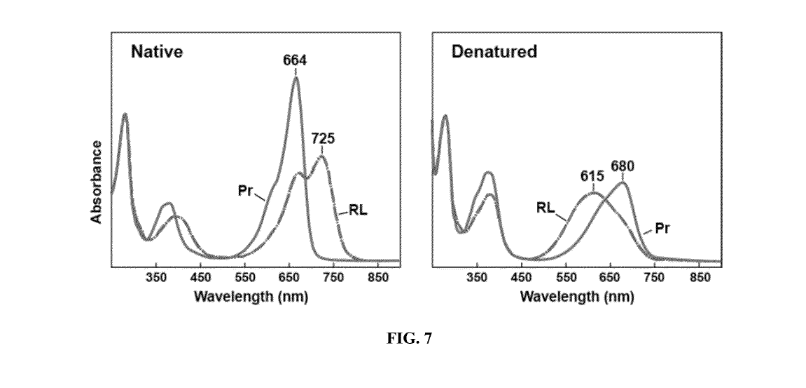

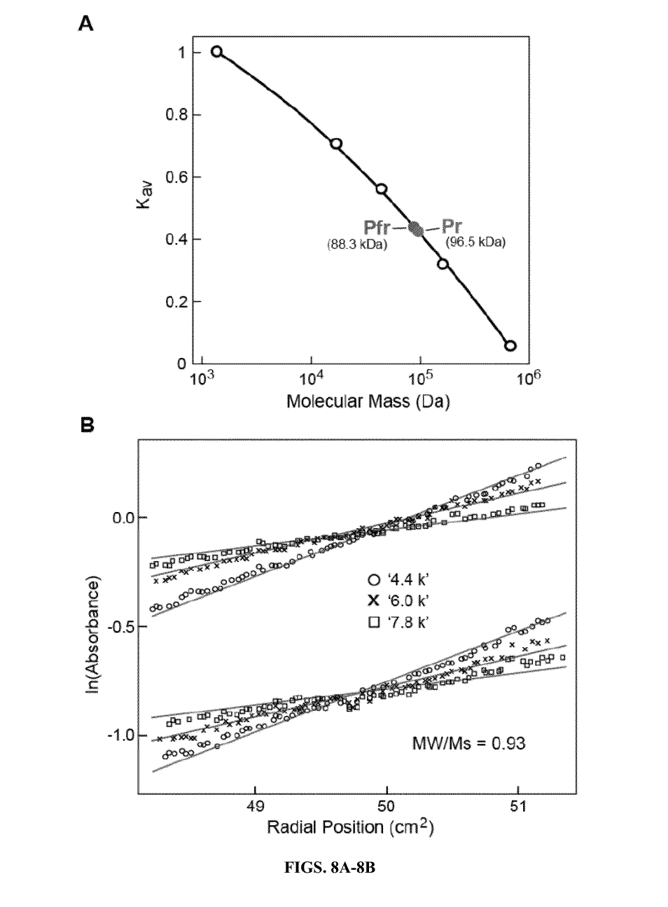

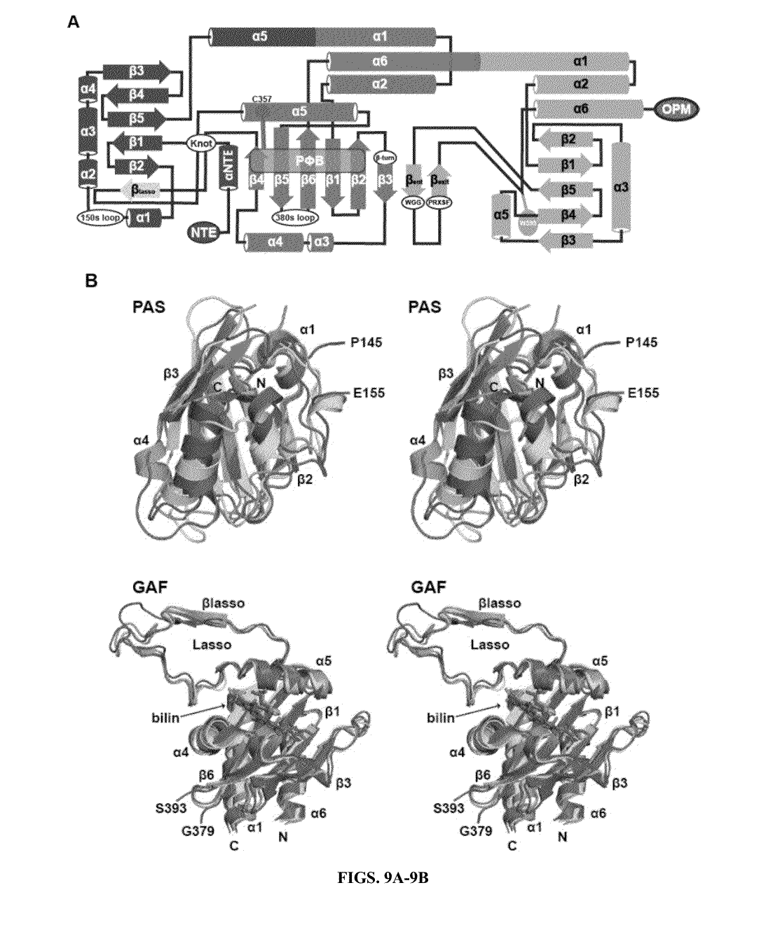

Among the seed plant Phy isoforms, PhyB (and its PhyD paralog in Arabidopsis thaliana) is distinguished by a long glycine/serine-rich NTE. The Arabidopsis PhyB PSM minus much of this possibly flexible NTE (“PhyB(90-624)”) was expressed recombinantly using a dual plasmid system that simultaneously synthesizes PΦB. PhyB(90-624) efficiently bound PΦB covalently, was stable in solution, displayed Pr and Pfr absorption spectra expected of a plant PhyB, and retained Pr→Pfr and Pfr→Pr photoconversion kinetics similar to the full-length PSM (residues 1-624) (FIG. 6). However, it showed a 7-nm hypsochromic shift in Pfr absorption and strongly accelerated Pfr→Pr thermal reversion, supporting the import of the NTE to Pfr stability. Absorption spectra of Pr and Pfr for the full PSM following acidic denaturation were consistent with the 15Z and 15E isomers of PΦB, respectively (FIG. 7).

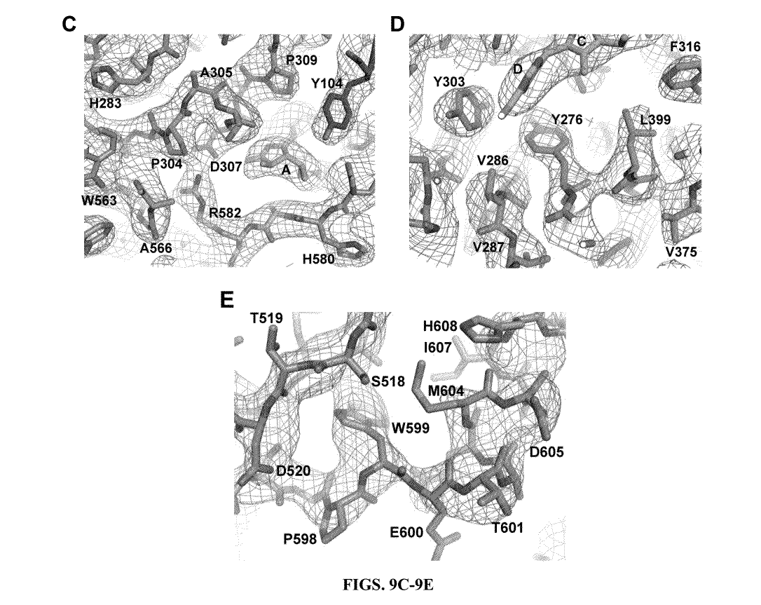

Crystallization screens of Pr identified several conditions for PhyB(90-624) crystal growth; although most crystals yielded anisotropic diffraction, the crystal studied here enabled collection of a complete X-ray dataset to 3.4-Å resolution. Molecular replacement utilizing the PAS/GAF domain from Synechocystis (Syn)-Cph1 allowed solution of initial phases. After molecular replacement, the calculated electron density was of sufficient quality to construct the remainder of the model. Statistical support for the model is presented in Table 2. See FIGS. 9C-9E for representative electron density maps.

TABLE 2
Arabidopsis PhyB(90-624) X-ray crystallographic
data collection and refinement statistics

| | |
|---|---|
| Data Collection^{a} | |
| Space group | P4₁2₁2 |
| Cell Dimensions | 127.5 Å × 127.5 Å × 300.8 Å |
| | 90° × 90° × 90° |
| Resolution (Å) | 50-3.4 (3.46-3.4) |
| R_{sym}^{b} | 0.175 (0.909) |
| CC1/2 | 0.836 (0.860) |
| I/σ(I) | 9.3 (2.0) |
| Completeness (%) | 100 (100) |
| Redundancy | 6.1 (6.3) |
| Refinement | |
| Resolution (Å) | 49.6-3.4 |
| No. reflections | 34999 |
| Rwork/Rfree^{c} | 0.243/0.270 |
| No. non-hydrogen atoms | 7100 |
| Protein | 6932 |
| Ligand | 163^{d} |
| Water | 5 |
| Average B-factors | 104.7 |
| Protein | 104.7 |
| Ligand | 105.6 |
| Water | 60.7 |
| R.m.s. deviations | |
| Bond lengths (Å) | 0.002 |
| Bond angles (°) | 0.57 |
| Steric clashes | |
| Molprobity clash score | 8.9 |
| Ramachandran (%) | |
| Favored | 94.0 |
| Outliers | 0.12 |
| PDB code | 4OUR |

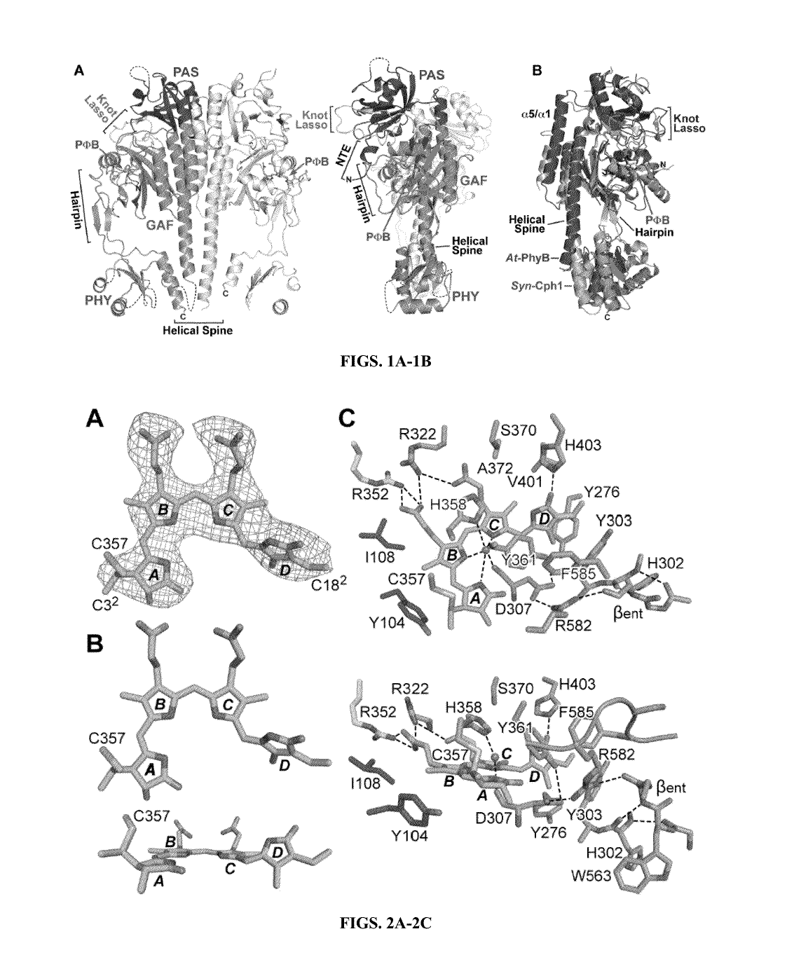

^{a}Outer shell values are in parentheses.
^{b}R_{sym} = ΣₕΣ_{I} |Iᵢ(h) − <I(h)>|/ΣₕΣᵢIᵢ(h), where Iᵢ(h) is the intensity of an individual measurement of the reflection and <I(h)> is the mean intensity of the reflection.
^{c}A 5.01% test set was selected at random for R_{free} calculation. Each R_{factor} = Σₕ ||F_{obs}| − |F_{calc}||/Σₕ |F_{obs}|, where F_{obs} and F_{calc} are the observed and calculated structure factor amplitudes, respectively.
^{d}Ligands comprised two phytochromobilin, one poly(ethylene)glycol, two glycerol, and eleven sulfate molecules.

Whereas PhyB(90-624) crystallized as a head-to-head dimer, both equilibrium sedimentation and SEC of the full PSM fragment revealed a monomeric size in solution (FIGS. 1A and 8). Equilibrium sedimentation and SEC of the full PSM fragment revealed a monomeric size in solution as both Pr and Pfr. SEC supported an elongated shape for the crystallographic subunits by estimating a Stokes radius ˜1.3 times predicted; similar values were obtained for Pr and Pfr, implying that photoconversion does not alter the oligomeric state of the PSM. The dimerization interface involved the GAF domain α1/α2/α6 helical bundle (FIGS. 1A and 9A), suggesting that these contacts are pertinent to PhyB homodimerization but insufficient without cooperation from the OPM. The sister PHY domains also contributed contacts at the dimer interface, primarily via helix α1 of subunit B and helix α6 of subunit A.

The Arabidopsis PhyB PSM structure (Protein Data Bank (PDB) ID code 4OUR) shared the same core PAS-GAF-PHY domain architecture with canonical bacterial phytochromes/cyanobacterial phytochromes (BphPs/Cphs) with the inclusion of unique features in each domain (FIG. 1A). β-sheet components of the PAS and GAF domains superposed reasonably well with those from Synechocystis (Syn)-Cph1, Deinococcus radiodurans (Dr) BphP, and Pseudomonas aeruginosa (Pa)-BphP (FIG. 9B and Table 3).

TABLE 3
Pairwise superposition statistics
At-PhyB subunit comparisons^{a}

| Structure | Residue range | Total Cα | RMSD (Å) |
|---|---|---|---|
| Overall | 101-610 | 403 | 1.6 |
| PAS | 116-222 | 95 | 0.81 |
| GAF | 234-432 | 185 | 0.73 |
| PHY | 480-610 | 80 | 1.8 |
| PHY w/out hairpin | 480-610 | 43 | 0.97 |
| Hairpin | 560-591 | 32 | 0.61 |

Superposition of PhyB with bacterial Phys^{b}

| | | | |
|---|---|---|---|
| Structure | Residue range | Cα excluded | RMSD (Å) |

PAS domain^{c}

| | | | |
|---|---|---|---|
| At-PhyB 0 | 115-218 | 57 | n/a |
| — | 27-124 | 51 | 1.2 |
| Dr-BphP | 36-128 | 45 | 1.2 |
| Pa-BphP | 24-112 | 42 | 1.3 |

GAF domain^{d}

| | | | |
|---|---|---|---|
| At-PhyB | 253-431 | 22 | n/a |
| Syn-Cph1 | 153-318 | 9 | 0.91 |
| Dr-BphP | 153-318 | 9 | 0.78 |
| Pa-BphP | 140-305 | 9 | 1.1 |

^{a}All residues found in both subunits were included in the PhyB/PhyB superposition analyses.
^{b}Synechocystis sp. PCC 6803 Cph1 (PDB- 2VEA), D. radiodurans BphP (PDB- 2O9C), and P. aeruginosa BphP (PDB- 3C2W, subunit A) were compared to PhyB subunit A.
^{c}Superpositions were against 47 Cα's from At-PhyB residues 115-124, 128-143, 189-194, 200-207, and 212-218.
^{d}Bacterial Phys were superposed with 157 Cα's from subunit A of PhyB, including residues 253-335, 341-377 and 395-431.

The PAS domains of sister PhyB subunits were highly congruent throughout, but in superpositions with bacterial Phys, the helical region between strands β2 and β varied substantially. PhyB also includes a large flexible loop comprising residues 145-155 (150 s loop) after PAS helix α1 (FIG. 9A). Although this loop is remote from the photoconversion machinery in PhyB, prior phenotypic analysis of a P149-L mutant implied functional significance. This loop is significant to PhyB signaling but innocuous to its photochemistry. At the C-terminal end of the 150 s loop, the hydrophobic side chain of M159 made a distinctive interaction with residues 335-337 of the GAF domain knot lasso. This interaction may stabilize the knot motif of PhyB, which has been implicated in PIF binding.

The PhyB GAF domain was highly congruent with bacterial Phys (Table 3). Significant differences in PhyB included an extended loop that includes residues 379-393 (380 s loop, FIG. 9A), and a distinctive lasso, whose novel shape generated a more extensive anti-parallel β-strand interaction with PAS strand β2. The α helix connecting the PAS and GAF domains (PAS helix α5/GAF helix α1) is longer than the analogous helix in bacterial Phys by 3 rotations. This addition substantially extends the helical spine to cover the full length of the PSM (FIG. 1B).

Electron density for the PHY domain was less resolved, especially in the loop regions (FIGS. 1 and 9E); presumably because the scarcity of crystal contacts in this region compromised resolution by enabling domain wobble within the crystal lattice. Due to a lack of connectivity and side chain features, this necessitated naming PHY domain residues in helices α3 and α5 as unknown in subunit B. By contrast, the signature PHY domain hairpin was well defined and consistent between sister subunits with a central feature being a stem formed by two anti-parallel β-strands designated βₑₙₜ and βₑₓᵢₜ (FIG. 9A, 9C). The sister subunits of PhyB(90-624) had different overall shapes that yielded a superposed rmsd of 1.6 Å over all matching α-carbons. Because the individual domains were highly congruent, this difference manifested itself at the domain transitions, especially at the junction between GAF and PHY domains.

### Example 6

### PΦB and the Bilin-Binding Pocket

The bilin and its GAF-domain pocket in PhyB shared strong homology with previously characterized bacterial Phys in the Pr state. PΦB was attached by a thioether linkage to Cys-357 through its C31 atom (FIGS. 2A, 2B). The electron density placed PΦB in the 5(Z)s, 10(Z)s, 15(Z)a configuration, with the A-C pyrrole rings mostly co-planar and the D ring rotated out of this plane by 58° (FIG. 2A). Dihedral angles about the C15=C16 bond averaged 35° at this low resolution versus 42° calculated previously from resonance Raman spectroscopy of Arabidopsis PhyA. A web of hydrogen bond and van der Waals interactions, involving a collection of conserved amino acids (e.g., Y104, I108, Y276, Y303, D307, R322, R352, H358, Y361, and H403 in PhyB) and the central pyrrole water, provided a comprehensive grasp of the bilin in the GAF pocket (FIG. 2C).

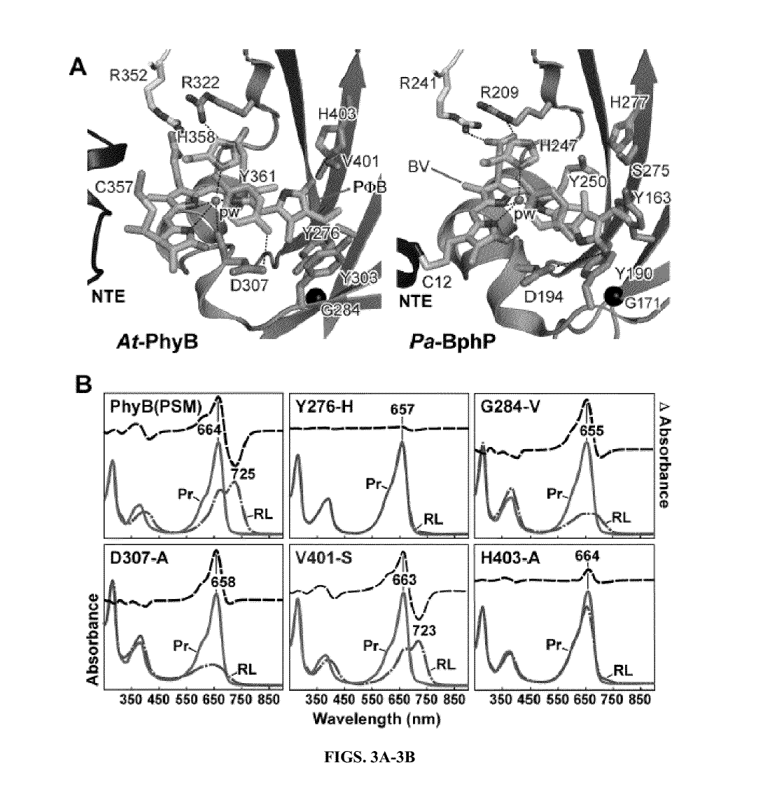

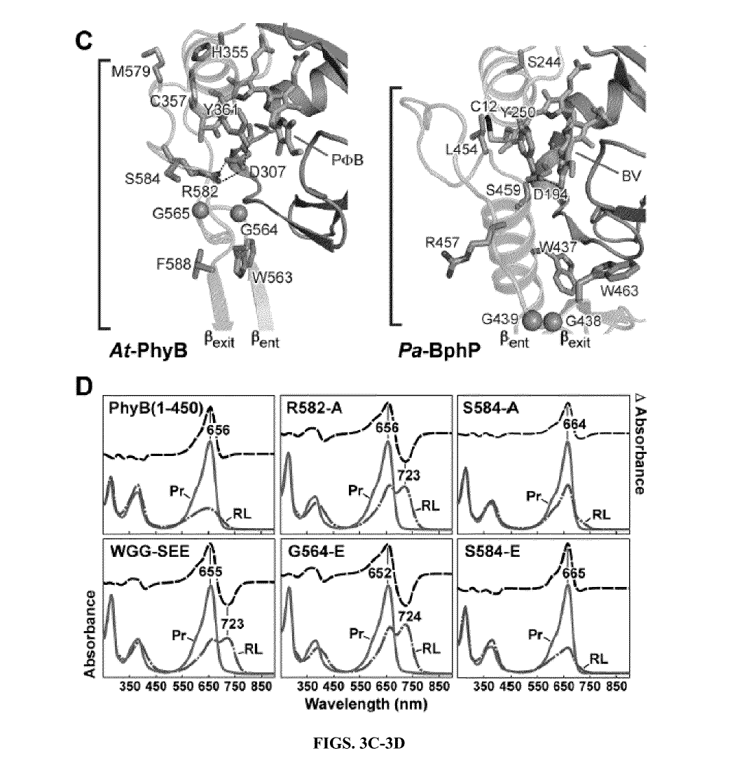

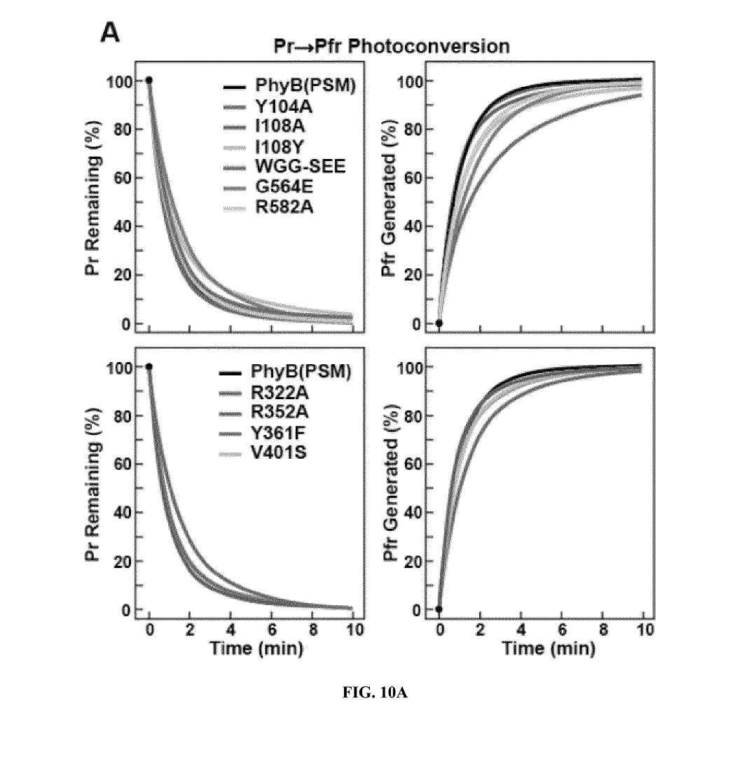

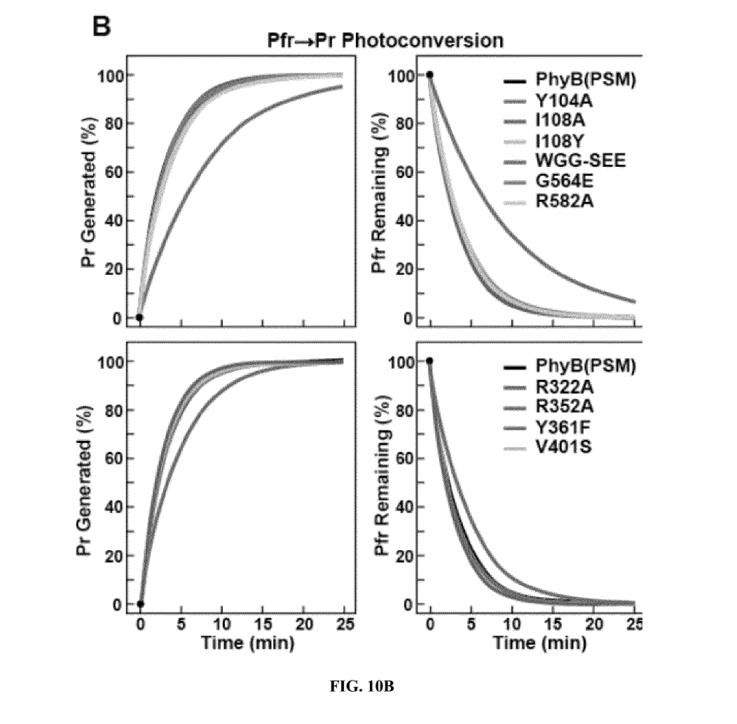

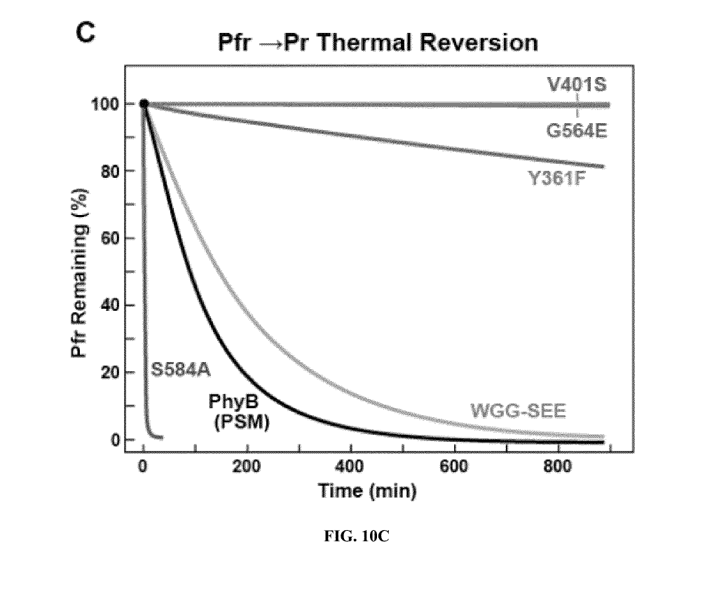

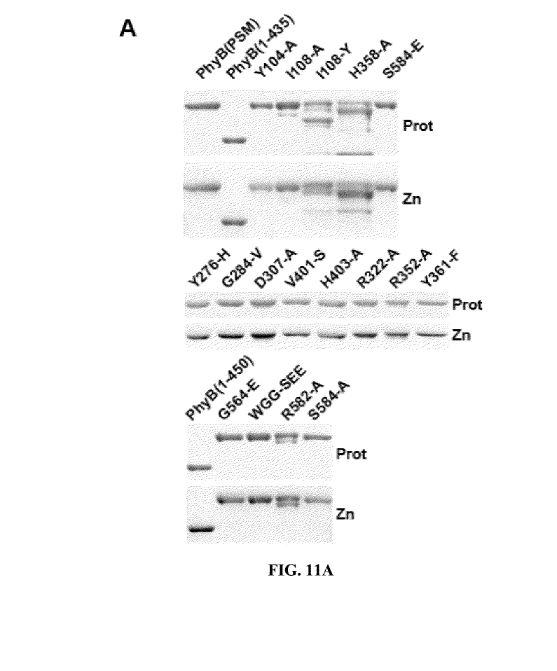

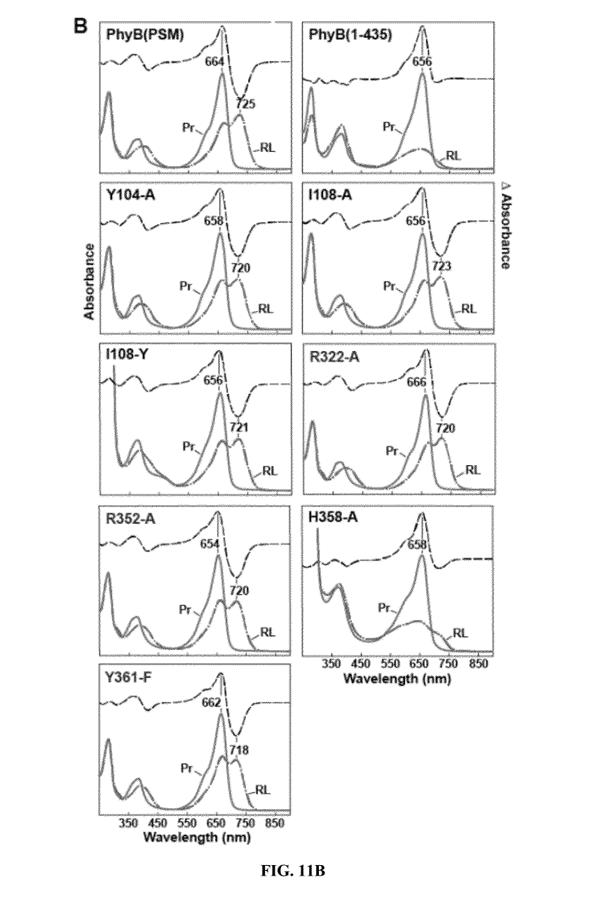

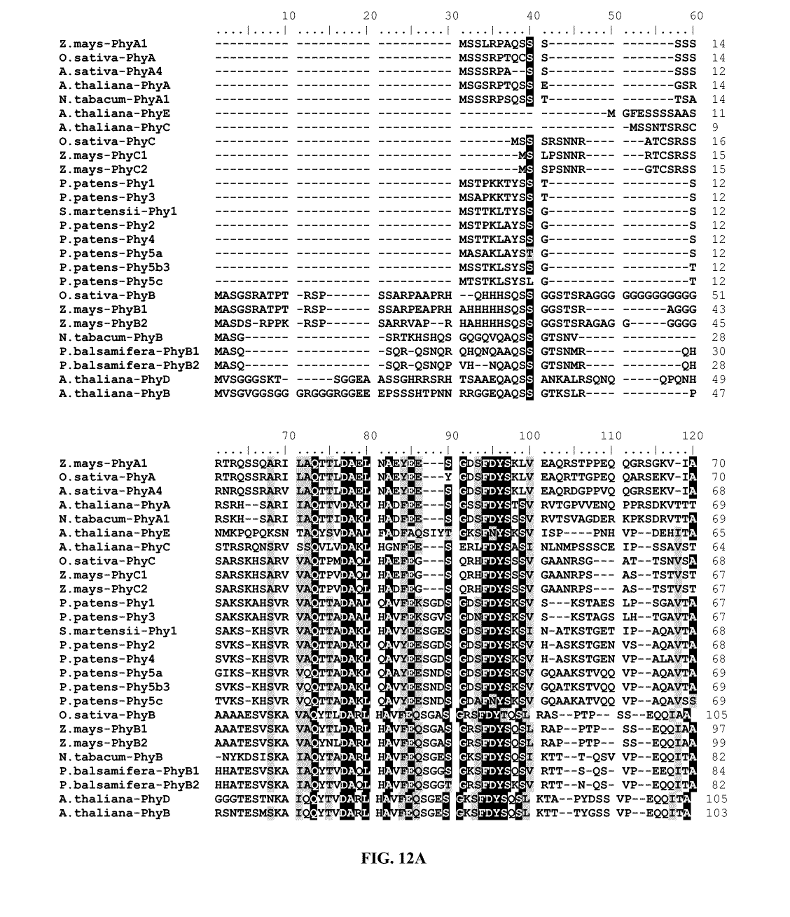

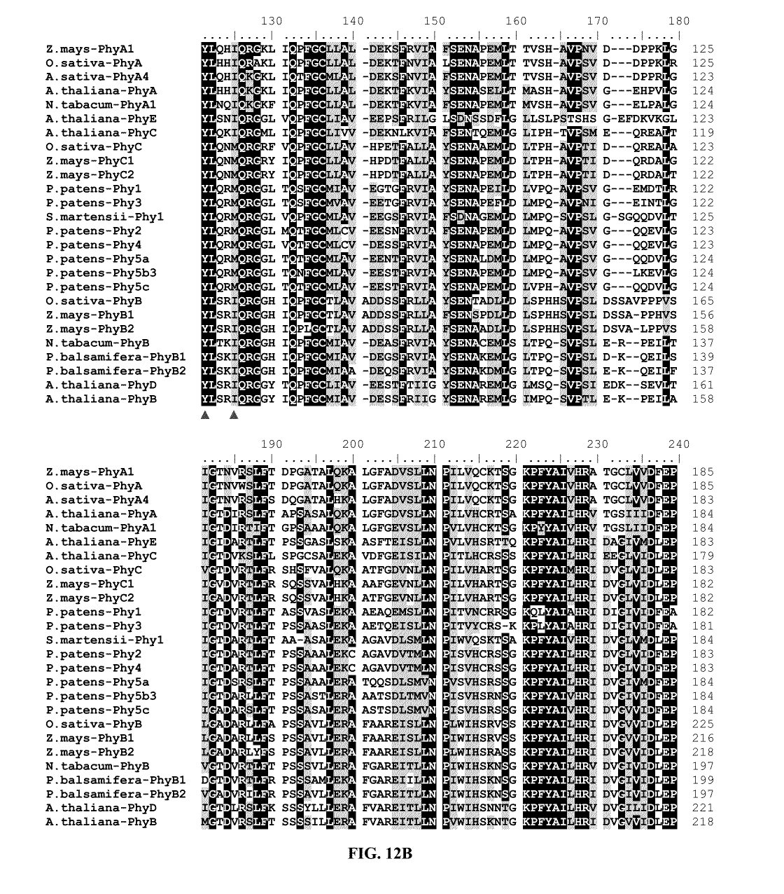

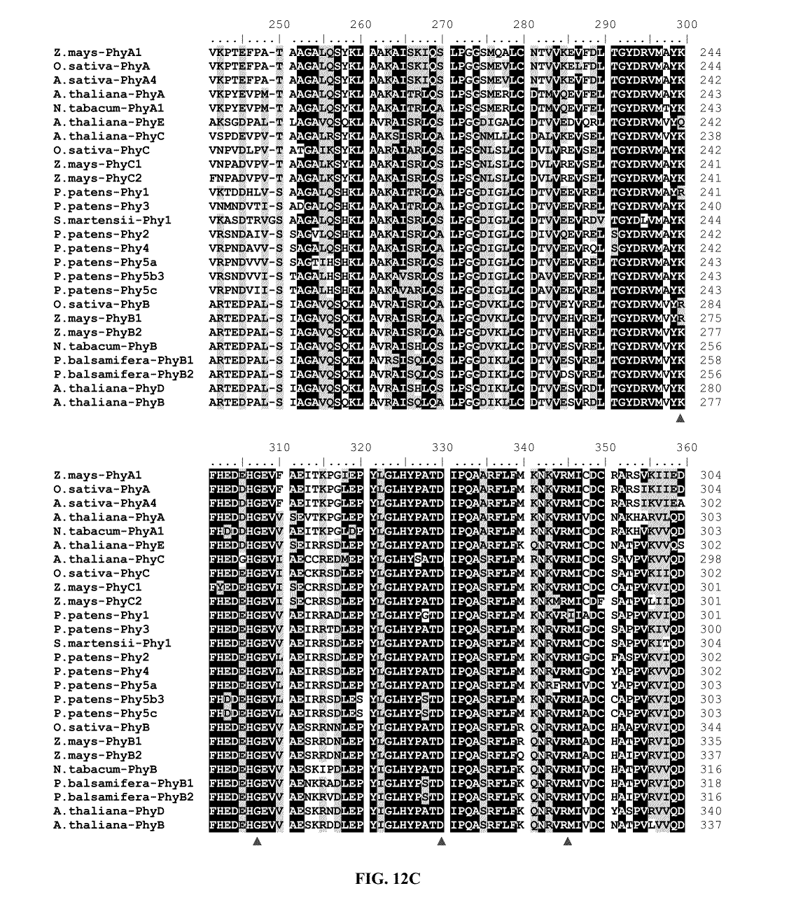

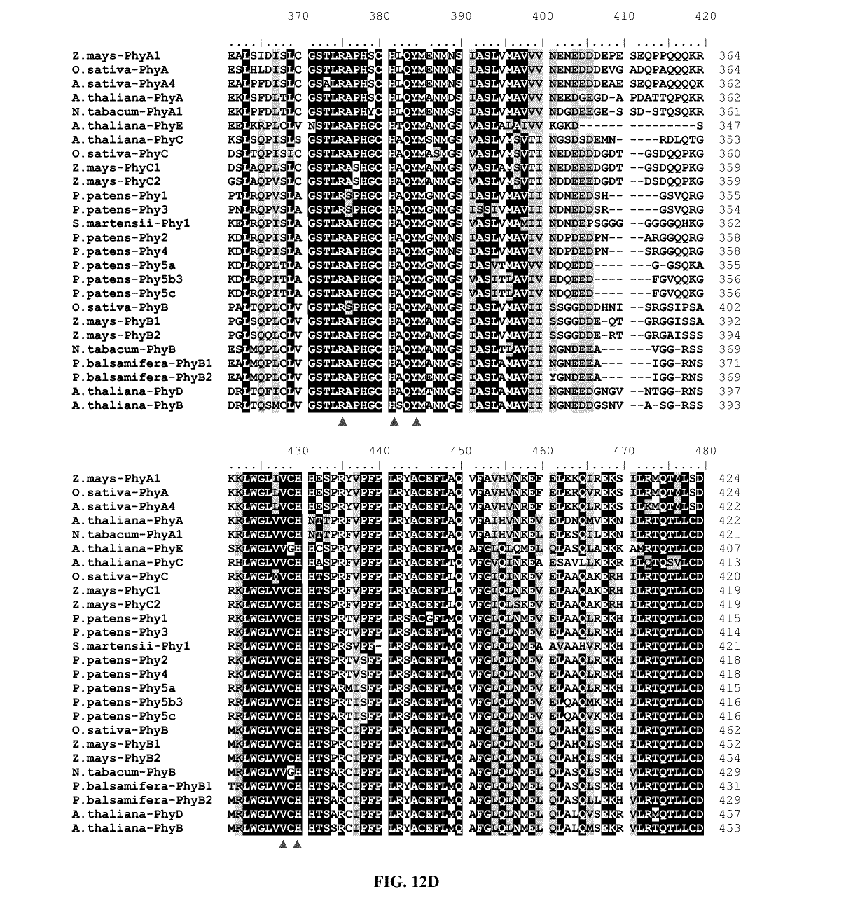

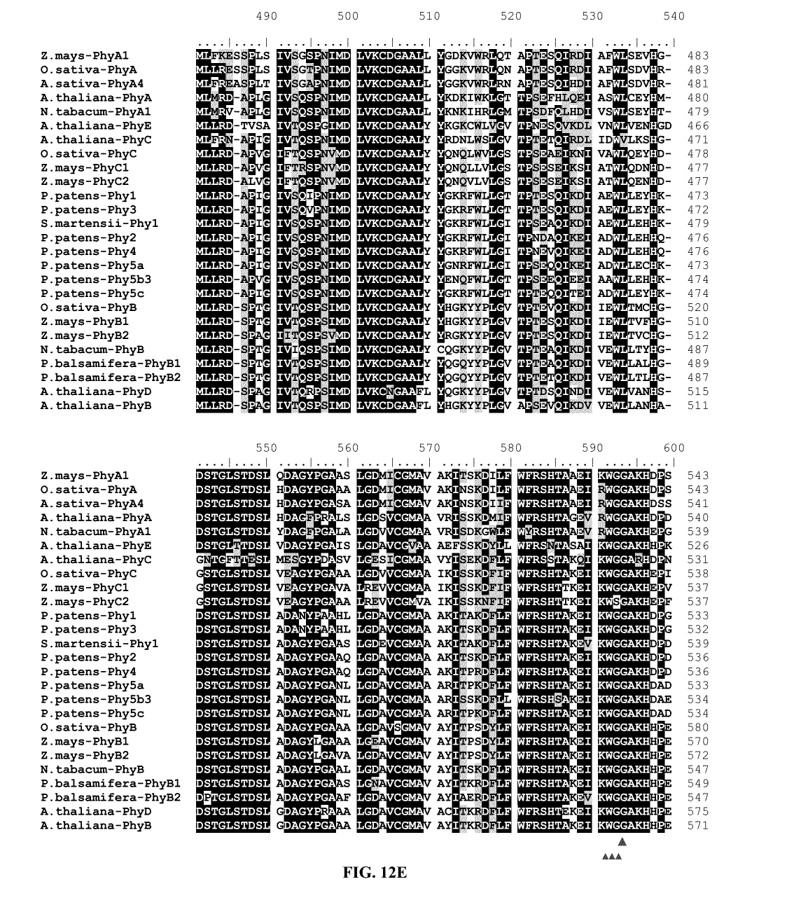

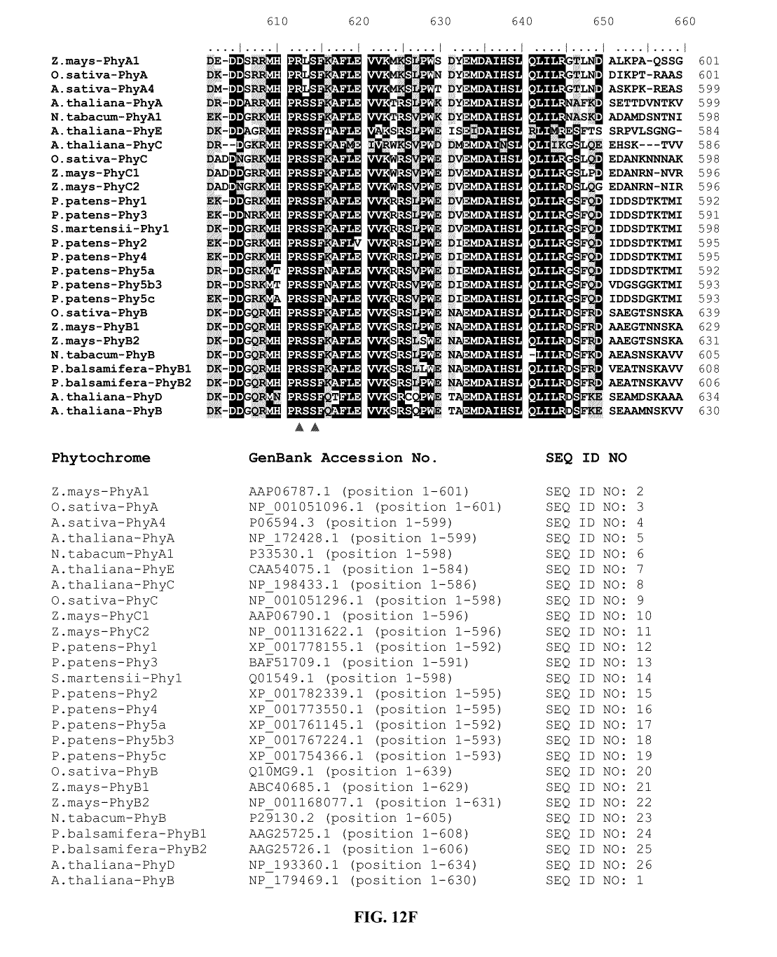

Mutational analyses with Arabidopsis PhyB confirmed the importance of several residues around PΦB. Although various point mutations altered the endstates of photoconversion, most mutants had little impact on initial photochemistry, implying that the first reaction step(s) are relatively insensitive to the protein environment. In contrast, a number of mutations greatly influenced Pfr→Pr thermal reversion (FIGS. 3, 4, 10, 11A, and Table 4). The mutants analyzed did not appreciably affect bilin ligation, and with the exception of WGG-SEE had relatively normal Pr/Pfr photointerconversion rates (FIGS. 10, 11 and Table 4).

TABLE 4
Arabidopsis PhyB photoconversion and thermal reversion rates

| | | | | | | PFR → PR | | | PFR → PR THERMAL | | |
|---|---|---|---|---|---|---|---|---|---|---|---|
| | PR → PFR PHOTOCONVERSION^{A} | | | | | PHOTOCONVERSION^{A} | | | REVERSION | | |
| | Initial | Fold- | Initial | Fold- | | Initial | Fold- | | | | Fold- |
| | Rate ± | Diff. (I₆₆₀ | Rate ± | Diff. (I₇₂₀ | Rate | Rate ± | Diff. (I₇₂₀ | Rate | Rate | | Diff. (t_{1/2} |
| | S.D. at | mut/I₆₆₀ | S.D. at | mut/I₇₂₀ | Constant | S.D. at | mut/I₇₂₀ | Constant | Constant | T_{1/2} | wt/t_{1/2} |
| Protein | 660 nm | wt)^{b} | 720 nm | wt)^{b} | (k) ± S.D. | 720 nm | wt)^{b} | (k) ± S.D. | (k) ± S.D. | (min) | mut)^{c} |
| PhyB | 0.144 ± | | 0.153 ± | | 1.2 ± | 0.045 ± | | 0.30 ± | 0.0085 ± | 81.6 | |
| (PSM) | 0.006 | | 0.002 | | 0.1 | 0.006 | | 0.04 | 0.0002 | | |
| PhyB | 0.150 ± | 1.04 | 0.140 ± | 0.91 | 1.5 ± | 0.060 ± | 1.344 | 0.42 ± | 0.16 ± | 4.2 | 19 |
| (90-624) | 0.005 | | 0.005 | | 0.2 | 0.002 | | 0.01 | 0.01 | | |
| PhyB | 0.132 ± | 0.91 | | | 1.1 ± | | | | 0.0142 ± | 48.7 | 1.7 |
| (1-435) | 0.002 | | | | 0.2 | | | | 0.0001 | | |
| PhyB | 0.117 ± | 0.81 | | | 0.8 ± | | | | 0.0155 ± | 44.8 | 1.8 |
| (1-450) | 0.009 | | | | 0.1 | | | | 0.0002 | | |
| Y104-A | 0.141 ± | 0.98 | 0.154 ± | 1.01 | 1.3 ± | 0.044 ± | 0.980 | 0.30 ± | 0.0162 ± | 42.7 | 1.9 |
| | 0.007 | | 0.008 | | 0.1 | 0.004 | | 0.03 | 0.0007 | | |
| I108-A | 0.142 ± | 0.97 | 0.144 ± | 0.94 | 1.4 ± | 0.046 ± | 1.017 | 0.31 ± | 0.0087 ± | 79.9 | 1.02 |
| | 0.008 | | 0.009 | | 0.1 | 0.003 | | 0.02 | 0.0002 | | |
| I108-Y | 0.117 ± | 0.81 | 0.12 ± | 0.78 | 1.0 ± | 0.037 ± | 0.832 | 0.25 ± | 0.0098 ± | 70.6 | 1.2 |
| | 0.004 | | 0.01 | | 0.1 | 0.003 | | 0.02 | 0.0005 | | |
| Y276-H | 0.010 ± | 0.07 | | | 2.4 ± | | | | | | |
| | 0.002 | | | | 0.3 | | | | | | |
| G284-V | 0.119 ± | 0.83 | | | 0.7 ± | | | | 0.000249 ± | 2786 | 0.03 |
| | 0.004 | | | | 0.1 | | | | 0.000005 | | |
| D307-A | 0.073 ± | 0.50 | | | 0.8 ± | | | | 0.0118 ± | 58.8 | 1.4 |
| | 0.005 | | | | 0.3 | | | | 0.0008 | | |
| R322-A | 0.146 ± | 1.01 | 0.156 ± | 1.02 | 1.6 ± | 0.053 ± | 1.185 | 0.36 ± | 0.0108 ± | 64.5 | 1.3 |
| | 0.005 | | 0.007 | | 0.1 | 0.003 | | 0.02 | 0.0002 | | |
| R352-A | 0.146 ± | 1.01 | 0.152 ± | 0.99 | 1.4 ± | 0.046 ± | 1.021 | 0.32 ± | 0.00044 ± | 1575 | 0.05 |
| | 0.009 | | 0.009 | | 0.1 | 0.001 | | 0.01 | 0.00004 | | |
| H358-A | 0.06 ± | 0.42 | | | 0.6 ± | | | | 0.0023 ± | 301 | 0.3 |
| | 0.01 | | | | 0.1 | | | | N/A | | |
| Y361-F | 0.085 ± | 0.59 | 0.100 ± | 0.66 | 1.0 ± | 0.031 ± | 0.694 | 0.22 ± | 0.000220 ± | 3151 | 0.03 |
| | 0.006 | | 0.007 | | 0.1 | 0.002 | | 0.01 | 0.000002 | | |
| V401-S | 0.122 ± | 0.85 | 0.137 ± | 0.9 | 1.4 ± | 0.048 ± | 1.074 | 0.33 ± | | | |
| | 0.008 | | 0.009 | | 0.2 | 0.003 | | 0.02 | | | |
| H403-A | 0.06 ± | 0.44 | | | 1.6 ± | | | | 0.231 ± | 3.0 | 27 |
| | 0.01 | | | | 0.2 | | | | 0.008 | | |
| G564-E | 0.098 ± | 0.68 | 0.093 ± | 0.61 | 0.8 ± | 0.042 ± | 0.929 | 0.27 ± | 0.00002 ± | 37068 | 0.002 |
| | 0.003 | | 0.003 | | 0.2 | 0.005 | | 0.03 | 0.00001 | | |
| WGG- | 0.139 ± | 0.96 | 0.057 ± | 0.37 | 1.0 ± | 0.018 ± | 0.407 | 0.12 ± | 0.0052 ± | 133 | 0.6 |
| SEE | 0.006 | | 0.003 | | 0.1 | 0.005 | | 0.03 | 0.0002 | | |
| R582-A | 0.134 ± | 0.93 | 0.123 ± | 0.81 | 1.1 ± | 0.039 ± | 0.864 | 0.27 ± | 0.0003 ± | 2068 | 0.04 |
| | 0.005 | | 0.008 | | 0.01 | 0.002 | | 0.01 | 0.0001 | | |
| S584-A | 0.09 ± | 0.64 | | | 7 ± | | | | 6.4 ± | 0.1 | 748 |
| | 0.02 | | | | 3 | | | | 0.1 | | |
| S584-E | 0.126 ± | 0.87 | | | 2.1 ± | | | | 1.92 ± | 0.4 | 226 |
| | 0.006 | | | | 0.3 | | | | 0.02 | | |

^{A}Photoconversions performed using 1 × 10⁻⁴ W light
^{b}Iₘᵤₜ/I_{wt}: >1 represents faster photoconversion than PhyB(PSM), <1 represents slower photoconversion than PhyB(PSM)
^{c}t_{1/2} wt/t_{1/2} mut: >1 represents faster thermal reversion than PhyB(PSM), <1 represents slower thermal reversion than PhyB(PSM)

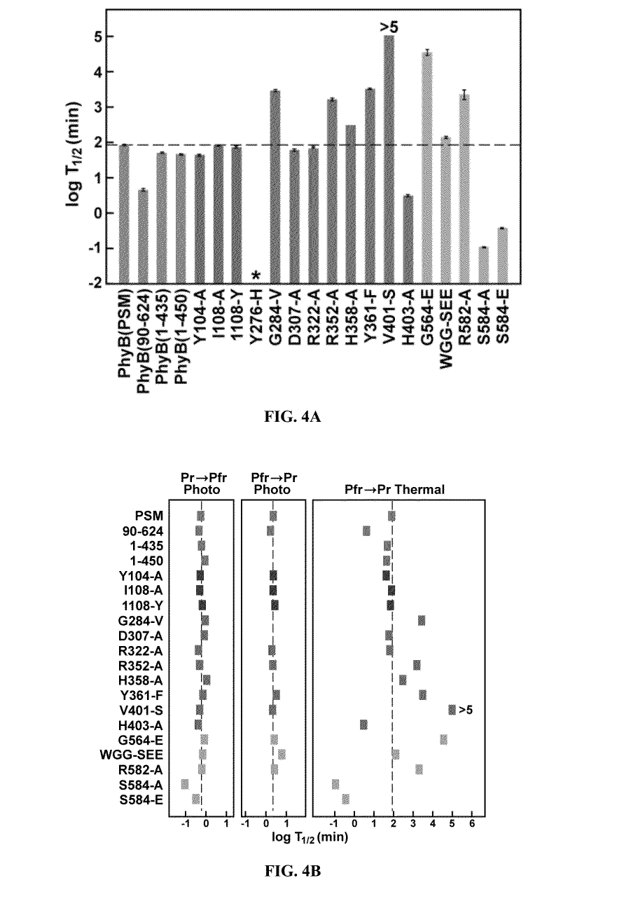

Substitutions of D307 within the invariant DIP motif and H358, which are focal points of the A-C pyrrole ring hydrogen-bond network and likely involved in the protonation/deprotonation cycle of the bilin, assembled hypsochromic-shifted Pr states (FIGS. 3B and 11B). The D307-A mutation precluded photoconversion to Pfr leading instead to a bleached species, and the H358-A biliprotein was partially photochromic but expressed poorly to suggest folding challenges. The D pyrrole ring is surrounded by H403 and a collection of bulky aromatic residues (Y276, Y303, and Y361) (FIG. 2C). The ε-nitrogen of H403 makes a crucial hydrogen bond with the carbonyl of ring D, thus anchoring the D ring in its Pr-state position. The Y276-H substitution created a photochemically inert, fluorescent variant, whereas the H403-A and Y361-F substitutions had strong opposite effects on thermal stability of Pfr (FIGS. 3B and 4).

The B-ring carboxylate bound a nearby arginine (R352). Accordingly, the R352-A substitution generated small hypsochromic-shifts in Pr and Pfr absorption with unaltered Pr/Pfr photointerconversion rates, but Pfr was markedly slower at thermal reversion (FIG. 11 and Table 4). The C-ring propionate was parallel with the B-ring propionate and contacted the adjacent R322 (FIG. 2C). This tether differs from bacterial Phys where the C-ring propionate points away from the B-ring propionate to associate with a conserved histidine (H358 in PhyB), a pair of positionally conserved serine residues (positions 370 and 372 in PhyB), and an ordered water (see FIG. 3A). Whereas Arabidopsis PhyB retained one of these serines (Ser370), the lack of a hydroxyl at position 372 might promote the B-ring propionate/R322 association. R322 is mechanistically relevant as the alanine substitution displayed a 5-nm hypsochromic shift in Pfr absorption and an increased rate of thermal reversion rate (FIGS. 4 and 11B and Table 4).

### Example 7

### Stabilization of Pfr by the PHY Domain Hairpin

The PHY domain in Arabidopsis PhyB is involved in photochemistry, as a PΦB-bound PSM fragment missing the entire PHY sequence (PhyB(1-450)) generated normal Pr but failed to photoconvert to Pfr (FIG. 3D). The hairpin protrudes from the PHY domain toward the GAF domain as an extended loop between strand β5 and helix α6 (FIGS. 1A, 3C, and 9A). Strikingly, the hairpin intimately connects the PHY domain to the bilin-binding cleft through several mechanisms. One is by extending the GAF domain β-sheet by the two β strands (βₑₙₜ and βₑₓᵢₜ) contributed from the hairpin stem, and another is by providing intimate contacts at the core of the bilin/GAF domain interface through a salt bridge between R582 and D307 and a hydrophobic interaction between F585 and GAF helix α5 (FIG. 3C). The 3-D structure of the hairpin differs from the Pr-state bacterial Phys as the loop connecting strands βₑₙₜ and βₑₓᵢₜ associates closely with the PhyB GAF domain throughout. The conserved WGG motif at the end of strand βₑₙₜ further reinforces the stem structure by creating a tightly curved motif that presses its tryptophan (W563) against a GAF domain pocket formed by H283, H302, and the main chain of Y303.

The mutational analyses of Arabidopsis PhyB revealed that the hairpin contacts are not essential to Pr but some residues are critical for proper Pr/Pfr interconversion and/or the thermal stability of Pfr (FIGS. 3D and 4). Substitutions affecting the arginine and serine residues within the PRXSF sequence profoundly affected thermal reversion, with the R582-A mutant slowing thermal reversion by 25-fold and the S584-A and S584-E mutants strikingly increasing it by up to 750-fold. The WGG motif also participates in Pfr stability. The G564E substitution, which should weaken or preclude the WGG motif GAF domain interaction in Pr, slowed thermal reversion by ˜450 fold (FIG. 4). A modest affect on thermal reversion was also seen by a whole-sale change of WGG to SEE, which should disrupt the sharp turn provided by the WGG sequence and increase solvent exposure, but this substitution likely has pleiotropic consequences given that it was one of the few mutations that appreciably affected both Pr→Pfr and Pfr→Pr photoconversion (FIG. 10). The hairpin loop is also likely important to PhyB based on prior analysis of the M579-T natural variant that greatly attenuates signaling in planta. The 3-D structure suggests that this threonine replacement stabilizes hairpin/GAF domain binding by an adventitious interaction with H355 (FIG. 3C).

### Example 8

### Role of the PhyB NTE

Unlike most bacterial Phys, plant Phys contain a long glycine/serine-rich NTE that is involved in the normal absorption of the photoreceptor, and stability of Pfr, as well as biological activity possibly via its light-dependent phosphorylation. For example, NTE deletion mutants such as PhyB(90-624) display hypsochromically shifted absorption maxima and more rapid thermal reversion (FIGS. 6D, 6F). The 3-D model of PhyB(90-624) revealed that part of the NTE contacts the GAF domain near the bilin crevice (FIG. 1A). In particular, an α-helix encompassing residues 104-110 forms a steric barrier for the A and B pyrrole rings with conserved residues Y104 and I108 directly abutting the bilin (FIG. 2C). Accordingly, both the Y104-A, I108-Y, and I108-A substitutions yielded PSMs with hypsochromically shifted Pr and Pfr absorption spectra (˜3-8 nm), with the Y104-A mutant also accelerating thermal reversion by 2 fold (FIGS. 4 and 11 and Table 4). Y104 was previously shown to be involved in PhyB nuclear localization and photoactivity in planta through its Pfr-dependent phosphorylation with evidence that neighboring NTE residues are also consequential. Y104 is buried in the Pr structure, suggesting that light-induced reorganization of the NTE/hairpin underpins its modification.

### Example 9

### A Comprehensive ‘Toggle’ Model for PhyB Photoconversion

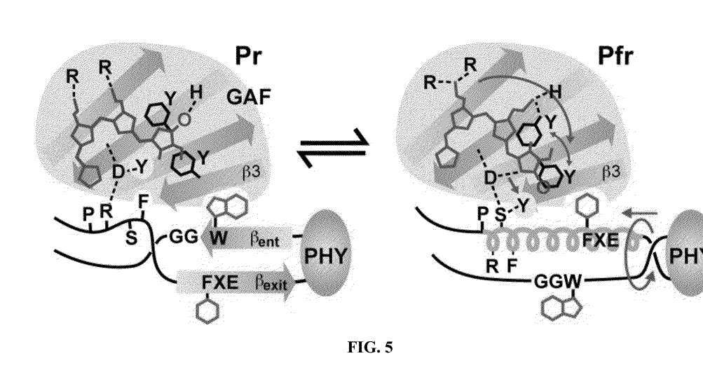

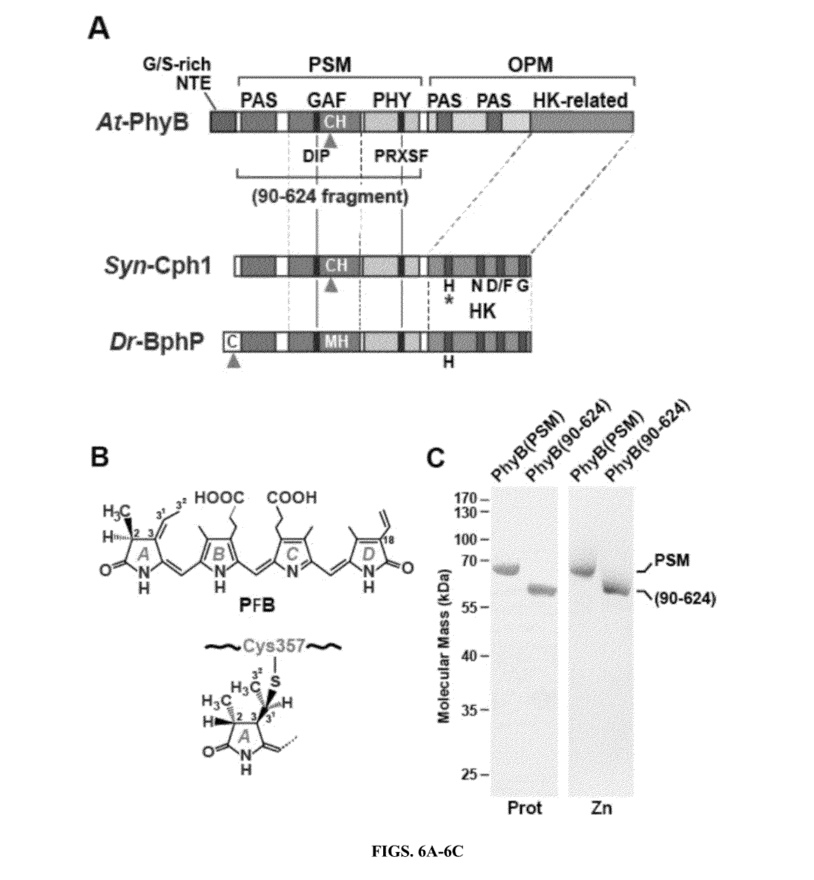

A feature of Phys among biological photoreceptors is their ability to reversibly photoconvert between two relatively stable endstates. By comparing the PSM structures from two Phys that use Pr as the dark-adapted state and two bathyphytochromes that assume a Pfr-like state without photoexcitation (Pa-BphP and Rhodopseudomonas plaustris (Rp)-BphP1), a “trytophan switch” model for Phy photoconversion has been proposed (see FIG. 5). Upon light-induced rotation of the D pyrrole ring, the bilin slides within the GAF domain crevice to break its D ring/H403 connection and assumes a new contact between the D-ring and D307 and the C ring propionate and H403. The tryptophan pair, Y276 and Y303 adjacent to the D ring, rotate in opposite directions as the D ring rotates. Together, the effects initiate a collision of Y361 with F588, weakening the hairpin interface, and leading to breakage of the D307/R582 contact. This release disconnects the hairpin stem from the GAF domain β-sheet, thus allowing the hairpin stem to become helical, swivel, reform a new contact between D307 and S584 in the PRXSF motif, and swap the βₑₙₜ for a βₑₓᵢₜ trytophan connection with the GAF surface. The rotation and helical conversion of strand βₑₓᵢₜ presumably reorients the PHY domain relative to the GAF domain and/or tugs on the helical spine connecting the PHY domain to OPM to eventually actuate signaling changes in the OPM.

The analyses of Arabidopsis PhyB supports this model for Phys with PAS/GAF/PHY domain architectures and extends it to other PSM features that promote the Pr/Pfr transitions and stabilize the two end states (FIG. 5). Isomerization driven rotation of the D pyrrole ring appears essential to the photointerconversion in canonical Phys. The D ring is chemically asymmetric; a hydrophobic face decorated with methyl and vinyl groups is on one side of the methine bridge and hydrophilic nitrogen and a carbonyl moieties are on the other. In Pr, the hydrophilic face binds the H403 imidazole via its carbonyl, and the pyrrole nitrogen likely interacts with solvent, whereas the hydrophobic face is surrounded by a compatible hydrophobic surface provided by M274, Y276, Y303, and M365 (FIG. 2C). A 180° rotation would then expose the D-ring functionalities to a non-ideal environment, which appears to recover by sliding the bilin within the GAF pocket, as illustrated in the paired endstate GAF domain models from the Phy relative PixJ from Thermosynechococcus elongatus (Te) and the PSM of Pa-BphP.

Bilin sliding would then induce a cascade of bilin/protein and protein/protein alterations that ultimately impinge on the hairpin (FIG. 5). From comparisons of Phy structures as Pr with that from Pa-BphP as Pfr, the tyrosine pair (Y276 and Y303) abutting the D-ring methyl and vinyl groups counter rotate upon D-ring flip. The photochemical necessity of these tyrosines and their rotations is vividly illustrated by the PhyB Y276-H and G284-V substitution. The Y276-H biliprotein may assume a Pfr-like signaling state without photoexcitation (FIG. 3B). The subtle G284-V substitution tested here generates a strongly stable but bleached species in red light, presumably by sterically hindering proper rotations of Y276 and Y303 (FIGS. 3B and 4). Bilin sliding would also reorient the B and C-ring propionates to attain positions similar to those found in the Pfr state of the bathyphytochromes Pa-BphP and Rp-BphP1. Whereas the B-ring propionate would move subtly to generate a new contact with R322, the C ring propionate would move dramatically to engage H403 after it breaks from the D-ring nitrogen. In Pa-BphP, an adjacent serine residue appears to stabilize its Pfr ground state, presumably by enforcing the position of ZZEssa configuration of the Pfr bilin through contact with the C ring propionate (FIG. 3A). Remarkably, serine replacement of the complementary residue in Arabidopsis PhyB (V401) with a serine also generated a strongly stable Pfr state. While this V401-S variant had relatively normal Pr and Pfr photochemistry, its thermal reversion was undetectable after 2 d at 25° C. (FIGS. 3B, 4 and 10C).

From the survey of Phy models, it appears that both bilin sliding and D-ring photo-isomerization impact the positions of D307 and Y361, in which D ring rotation repositions the Y361 hydroxyl group away from the carboxyl moiety of D307 and D307 forms a hydrogen bond with the Pfr-state D-ring nitrogen. Although the subtle movement of D307 between superposed Pr and Pfr structures would suggest preservation of the D307/R582 contact, the repositioned Y361 would collide with the conserved PRXSF residue, F585. Additionally, strand β3 of the GAF domain would move with Y276/Y303 repositioning, potentially compromising the GAF β3/hairpin βₑₙₜ interaction. Together, these effects presumably melt the GAF/hairpin interaction, including the D307/R582 salt bridge, to permit helical refolding of the stem. This refolding also would swivel the main chain positions of the melted strands βₑₙₜ and βₑₓᵢₜ with respect to the GAF domain, and swaps the positions of R582 and S584 to allow hydrogen bonding between S584 and D307 (FIG. 5). The stem/GAF domain contact could also be renewed in PhyB by association of the exit sequence and its FXE motif with the GAF domain β-sheet.

The importance of these R582, S584 and the WGG motif contacts is illustrated by the mutational studies with Syn-Cph1, Syn-Cph2, and Pa-BphP and our studies with Arabidopsis PhyB (FIGS. 3D and 4). For example, compromising the proposed Pfr contact in PhyB via the S584-A and S584-E substitutions generated a bleached species in red light with substantially accelerated thermal reversion (t_{1/2}˜7 and 22 sec, respectively, versus 83 min for wild-type) (Table 4). Loss of the NTE also exacerbated thermal reversion indicating a key role in Pfr stabilization (FIG. 4). While not essential to Arabidopsis PhyB photochemistry, effects of the G564-E and WGG-SEE mutations supports the proposed “tryptophan switch” that encourages Pr/Pfr photointerconversion and the thermal stability of Pfr (FIGS. 3D, 4 and Table 4). The importance of WGG to PhyB signaling is dramatically illustrated by observations that the G564-E mutation increases the red-light sensitivity of Arabidopsis seedlings by as much as 1000 fold.

The light-induced hairpin reconfiguration may strain the GAF/PHY domain interface as the stem swivels and impinges on the PHY/OPM interface as the stem contracts from its β strand to α helical configuration through a direct connection between the helical spine and strand βₑₓᵢₜ. Consequently, even a small torque and/or tug on the hairpin stem could have profound implications on OPM positioning and activity by toggling the endstate positions of sister OPMs relative to the PSM and to each other. For D. radiodurans BphP, this strain substantially splays the sister PHY domains that likely amplifies into nanometer-scale reorientations of the sister OPMs.

### Example 10

### Prophetic Examples

### Materials and Methods

Recombinant phyB protein expression, purification, and analysis. All the site-directed mutations in PHYB are introduced into the cDNA by the Quikchange method (Stratagene). cDNA fragments encoding the photosensory modules (residues 1-624) are appended in-frame corresponding to the N-terminus of the 6His tag (KLHHHHHH) (SEQ ID NO: 29) by introduction into the pBAD plasmid (Invitrogen), and then co-transformed into Escherichia coli BL21 (AI) cells (Invitrogen) with the pPL-PΦB plasmid expressing the Synechocystis PCC6803 HO1 heme oxygenase and A. thaliana HY2 PΦB synthase enzymes [40, 41] to direct apoprotein expression and chromophore assembly. Following sequential induction of the HO1/HY2 genes and PHYB genes with IPTG and arabinose, the cells are disrupted by sonication in extraction buffer (50 mM HEPES-NaOH (pH 7.8), 300 mM NaCl, 30 mM imidazole, 0.1% Tween-20, 10% glycerol, 1 mM 2-mercaptoethanol, and 1 mM PMSF) with the addition of 1 tablet of protease inhibitor cocktail (Roche) before use. The clarified supernatant is applied to a HisTrap HP column (GE) pre-equilibrated in extraction buffer, and the column is washed with extraction buffer followed by elution with a 30-300 mM imidazole gradient in extraction buffer. The phyB-containing fractions are pooled, dialyzed against 10 mM HEPES-NaOH (pH 7.8), 100 mM NaCl, 5 mM 2-mercaptoethanol, 5 mM Na₂EDTA, 50 mM imidazole, and 0.05% Tween-20 overnight, and subjected to size-exclusion chromatography using a 24-ml Superose 6 (GE) column pre-equilibrated with the same buffer. phyB-containing fractions are pooled and stored in 10 mM HEPES-NaOH (pH 7.8), 50 mM NaCl, 1 mM 2-mercaptoethanol, 0.05% Tween-20, and 10% glycerol.

Pr-Pfr photointerconversion and Pfr-Pr thermal-reversion of each phyB preparation are assayed by UV-vis absorption spectroscopy at 24° C., using white light filtered through 650- and 730-nm interference filters (Orion) to drive Pr→Pfr and Pfr→Pr phototransformation, respectively.

Plant Materials and Growth Conditions.

All the plant lines are derived from A. thaliana Col-0 ecotype. The phyB-9 and phyA-211 alleles are as described (Reed et al., Plant Cell 5:147-157 (1993); Reed et al., Plant Physiol. 104:1139-1149 (1994)). Seeds are surface-sterilized in chlorine gas, and stratified in water for 3 d at 4° C. before sowing. Unless otherwise noted, seedlings are grown at 22° C. under white light in LD (16-hr light/8-hr dark) on 0.7% (w/v) agar medium containing 1× Gamborg's (GM) salts, 2% (w/v) sucrose, 0.5 g/L MES (pH 5.7). After 10 d, seedlings are transferred to soil and grown at 22° C. under continuous white light in LD or SD (8-hr light/16-hr dark).

Plasmid Constructions for Plant Transformation.

The full coding regions of PHYA and PHYB (Sharrock and Quail, Genes Dev. 3: 1745-1757 (1989)) are inserted into the pDONR221 plasmid via BP reactions (Invitrogen), and appended the coding sequence in-frame for the FLAG-epitope (GGGDYKDDDDK) (SEQ ID NO: 30) to their 3′ ends. The PHYA/B promoter and 5′ UTRs (2634- and 1983-bp upstream beginning at the ATG translation initiation codon), and 3′ UTRs (242- and 279-bp downstream of the translation termination codon) are amplified by PCR from the Col-0 genomic DNA, and then sequentially inserted into the pDONR211 plasmids to appropriately flank the coding regions. The completed PHYB and PHYA transgenes are introduced into the pMDC123 plasmid (Invitrogen) via LR reactions. The PHYB yellow fluorescent protein (YFP) constructions are created by appending the UBQ10 promoter fragment (1986-bp fragment proximal to the ATG codon) and the cDNA encoding YFP, to the 5′ and 3′ ends of the PHYB cDNA in a pDONR211 plasmid, respectively. The complete transgenes are introduced into the pMDC123 plasmid via LR reactions.

Plant Transformation and Selection of Transgenic Lines.

The PHYA and PHYB transgenes are introduced into the homozygous Arabidopsis phyA-211 or phyB-9 mutants, respectively, via the Agrobacterium-mediated floral dip method using the pMDC123-derived plasmids. Transformed lines are selected by resistance to 10 μg/mL BASTA. T2 transgenic plants with a resistance segregation ratio of ˜3:1 are used to obtain isogenic lines in the T4 or T5 generation for all the biochemical, phenotypic, and localization assays.

Protein Extraction and Immunoblot Analysis.

Five-day-old, dark-grown Arabidopsis seedlings are frozen and pulverized at liquid nitrogen temperatures, and homogenized in 100 mM Tris-HCl (pH 8.5), 10 mM Na₂EDTA, 25% ethylene glycol, 2 mM PMSF, 10 mM N-ethylmaleimide, 5 μg/mL sodium metabisulfite, 2% (w/v) SDS, 10 μg/mL aprotinin, 10 μg/mL leupeptin and 0.5 μg/mL pepstatin. The extracts are heated to 100° C. for 10 min and clarified by centrifugation at 13,000×g for 10 min. The supernatants are subjected to SDS-PAGE and immunoblot analysis with a monoclonal antibody against phyA (073D, Shanklin et al., Biochemistry 28:6028-6034 (1989)), phyB (B1-B7, Hirschfeld et al., Genetics 149:523-535 (1998)), or green fluorescent protein (GFP) (Sigma). Anti-PBA1 antiserum (Book et al., J. Biol. Chem. 285:25554-25569 (2010)) or anti-histone H3 antibodies (Abcam) are used to confirm equal protein loading.

To measure phyB degradation in response to Rc, seeds are sown in liquid medium containing half-strength Murashige and Skoog (MS) salts, 0.5 g/L MES (pH 5.7), and 10 g/L sucrose, and irradiated with white light (24 hr for seeds carrying the modified phytochrome transgene and 12 hr for all others) to initiate germination before maintaining the seedlings in the dark for 4 d. Seedlings are collected after various exposure times to continuous 20 μmol·m⁻²·s⁻¹ R and subjected to immunoblot analysis as above. Seedlings are incubated for 12 hr in the dark with 100 μM MG132 or an equivalent volume of DMSO before R.

Phenotypic Assays.

Germination efficiency is measured according to Oh et al. (Plant Cell 19, 1192-1208). The parental plants (5 per genotype) are grown side by side at 22° C. in LDs, and the resulting seeds are harvested as separate seed pools. At least 60 seeds from each pool are sown on 0.7% (w/v) water agar after 20-min FR irradiation (4 μmol m⁻² s⁻¹). The seeds are then exposed to white light for 2 hr, and either kept in dark or irradiated with 4 μmol m⁻² s⁻¹ FR for 5 min. The plates are kept in darkness for an additional 5 d before measurement of germination, which is scored as emergence of the radical from the seed coat. For hypocotyl elongation, seeds are sown on solid half-strength MS salts, 0.5 g/L MES (pH 5.7), and 0.7% (w/v) agar, and irradiated with 12-hr white light. The plates are exposed to either R or FR for 3.5 d using a bank of diodes (E-30LED-controlled environment chamber, Percival), before measurement of hypocotyl length. For measurement of the EOD-FR response, seedlings are irradiated over a 4-d cycle with 90 μmol·m⁻²·s⁻¹ R for 8 hr followed by either darkness or by a 10-min pulse of 100 μmol·m⁻²·s⁻¹ FR and then darkness for 16 hr. Effect on flowering time is measured for plants grown under white light in SD.

Confocal Microscopic Analysis.

Transgenic seeds expressing wild-type and mutant versions of phyB-YFP are sown on solid medium containing half-strength MS salts, 0.5 g/L MES (pH 5.7), 2% (w/v) sucrose, and 0.7% (w/v) agar and irradiated for 12 hr at 22° C. with white light before incubation in the dark for 5 d. Fluorescence of hypocotyl cells, either kept in the dark or irradiated with 90 μmol·m⁻²·s⁻¹ R for 12 hr, is imaged using a Zeiss 510-Meta laser scanning confocal microscope. YFP fluorescence is visualized in the single-track mode by excitation with 488-nm light using the BP 500-530 IR filter. Images are processed with the LSM510 image browser.

### Example 11

### Prophetic Example

### Rational Design of phyB Variants to Alter Light Signaling

Site-directed substitutions of certain amino acids based on the microbial scaffolds are introduced into the Arabidopsis phyB isoform. The photochemistry of the mutant photosensory modules are examined after recombinant assembly with the native chromophore phytochromobilin (PΦB), and the full-length versions are assessed for their phenotypic rescue of the phyB-9 null mutant using the native PHYB promoter to drive expression. The results will collectively demonstrate that various aspects of phy dynamics and signaling can be adjusted, which in some cases will generate plants with unique photobehavioral properties.

Mutations are predicted to compromise Pr to Pfr photoconversion, interaction of the bilin with its binding pocket, and/or possible signal transmission from the cGMP phosphodiesterase/adenylyl cyclase/Fh1A (GAF) domain to the downstream phytochrome (PHY) domain in the photosensory module.

To examine the ability of the mutants to concentrate in nuclear bodies/speckles as Pfr, a parallel set of transgenic lines expressing the phyB mutants is created as N-terminal fusions to yellow fluorescent protein (YFP). These bodies are easily seen by confocal fluorescence microscopy as numerous intense punctum that accumulate in the nucleus upon prolonged R irradiation.

### Example 12

### Prophetic Example

### Transgenic Maize

The promoter and coding regions of Zea maize (Zm)PHYB1 are cloned from maize genomic DNA and total mRNA, respectively, according to the publically available Zea mays genome sequence data (see Nucleic Acids Res. 40 (Database issue):D1178-86), and are built into a construction containing a Bar gene for Basta resistance and the nopaline synthase transcription terminator directly after the PHYB1 coding region. The corresponding mutation(s), as described above, is further introduced into the coding region of ZmPHYB1 in the construction via Quikchange method (Stratagene). Transgenic maize is made by Agrobacterium tumefaciens-mediated transformation (Nat. Protoc. 2: 1614-1621), and selected for Basta resistance. A total of eight transgenic lines at T1 generation are chosen for further screening based on transgene number, phyB protein level and genetic stability from a large pool of transgenic plants (>100 plants), and are grown, self-pollinated to T4 generation to produce isogenic lines for phenotypic assays.

The selected homogeneous transgenic maize containing the corresponding mutation(s) are grown in green house for phenotypic characterization. After 30 days, the plant height, size of both the transgenic and wild-type maize will be measured, and the flowering time and seed yield will also be recorded in mature plants. These phenotypic data will also be statistically analyzed, and compared to wild-type plants. The transgenic lines are expected to have much reduced height and size with unaltered flowering time and seed yield. These dwarf maize are expected to require much less growth space and therefore increase the maize yield per acre.

### Example 13

### Prophetic Example

### Transgenic Rice

The promoter and coding regions of Oryza sativa L. (Os) PHYB are cloned from rice genomic DNA and total mRNA, respectively, according to the OsPHYB coding sequence data from National Center for Biotechnology Information, and are built into a construction containing a Neomycin Phosphotransferase II (NPTII) gene for kanamycin resistance and the nopaline synthase transcription terminator directly after the PHYB coding region. The corresponding mutation(s), as described above, is further introduced into the coding region of OsPHYB in the construction via Quikchange method (Stratagene). Transgenic rice is made by Agrobacterium tumefaciens-mediated transformation (Plant J. 1994 (2):271-82), and selected for kanamycin resistance. A total of eight transgenic lines at T1 generation are chosen for further screening based on transgene number, phyB protein level and genetic stability from a pool of over 20 transgenic plants, and are grown and self-pollinated to T4 generation to produce isogenic lines for phenotypic assays.

The selected homogeneous transgenic rice containing the corresponding mutation(s) are grown in green house for phenotypic characterization. After 30 days, the plant height and size of both the transgenic and wild-type rice will be measured, and the flowering time and seed yield will also be recorded in mature plants. These phenotypic data will also be statistically analyzed. Compared to the wild-type plant, the transgenic lines are expected to have much reduced height and size with unaltered flowering time and seed yield. These dwarf rice are expected to require much less growth space and therefore increase the rice yield per acre.

### Example 14

### Prophetic Example

### Transgenic Soybean

The promoter and coding regions of Glycine max (Gm) PHYB1 are cloned from soybean genomic DNA and total mRNA, respectively, according to the GmPHYB1 coding sequence data from National Center for Biotechnology Information, and are built into a construction containing a Bar gene for Basta resistance and the nopaline synthase transcription terminator directly after the GmPHYB1 coding region. The corresponding mutation(s), as described above, is further introduced into the coding region of GmPHYB1 in the construction via Quikchange method (Stratagene). Transgenic soybean is made by Agrobacterium tumefaciens-mediated transformation (Plant Biotechnol. 2007, (24): 533-536), and selected for Basta resistance. A total of eight transgenic lines at T1 generation are chosen for further screening based on transgene number, phyB protein level and genetic stability from a large pool of over 100 transgenic plants, and are grown and self-pollinated to T4 generation to produce isogenic lines for phenotypic assays.

The selected homogeneous transgenic soybean containing the corresponding mutation(s) are grown in the green house for phenotypic characterization. After 30 days, the plant height, size of both the transgenic and wild-type soybean will be measured, and the flowering time and seed yield will also be recorded in mature plants. These phenotypic data will also be statistically analyzed. Compared to the wild-type plant, the transgenic lines are expected to have much reduced height and size with unaltered flowering time and seed yield. These resulting dwarf soybean should require much less growth space and therefore increase the soybean yield per acre.

### Example 15

### Prophetic Example

### Spectroscopy Analyses of Maize phyB Mutants

A library of structure-guided variants has the potential to alter phy signaling in a number of ways, which in turn offers a host of opportunities to manipulate light perception in maize. To test this notion, we will examine how the mutations corresponding to Y104-A, I108-A, I108-Y, G284-V, H358-A, V401-S, H403-A, W563-S, G565-E, S584-A, and/or S584-E of the Arabidopsis sequence affect maize phyB photochemistry and/or phyB-directed photomorphogenesis.

Using the protocols described herein, the photochemical effects of these amino acid substitutions on the recombinant 6His-tagged PSM of maize phyB1 (amino acids 1-623), the dominance of the two maize phyB paralogs with respect to phenotypes, will be examined. These mutations will be introduced by the Quikchange method (Stratagene) into the full-length ZmPHYB1 cDNA modified to also contain a C-terminal 6His sequence. They will be expressed in E. coli by well defined, two-plasmid pBAD (Invitrogen) system; one LacZ-controlled plasmid encodes the HO (heme oxygenase) from Synechocystis PCC6803 and the PΦB synthase from Arabidopsis (HY2 locus) needed to synthesize the PΦB chromophore from heme, and the second arabinose-controlled plasmid encodes the ZmphyB1 polypeptide. By sequential induction with IPTG and arabinose, high level accumulation of fully assembled and photochemically active ZmphyB1 PSMs will be possible. The recombinant biliproteins will then be purified by nickel-nitrilotriacetic acid (NiNTA) affinity (Qiagen) chromatography based on the 6His tag, followed by Phenyl Sepharose chromatography. Bilin occupancy of the purified photoreceptors will be assessed by zinc-induced fluorescence of the bound chromophore following SDS-PAGE of the preparation. These samples will be examined for atypical absorption spectra, photoconversion rates, and Pfr stability by spectrometric techniques.

### Example 16

### Prophetic Example

### Assessment of Signaling Strength for the ZmphyB1 Mutants in Maize

The ZmphyB1 mutations generated in prophetic example 15 will be introduced into maize plants and tested for their ability to direct various processes under ZmphyB control. The amino acid substitutions will be introduced into the full-length ZmPHYB1cDNA, also appended to a DNA sequence encoding a short C-terminal FLAG epitope tag (GGDYKDDDDK) (SEQ ID NO: 31), and expressed under the control of the native ZmPHYB1 promoter (2-kbp region upstream of the initiation codon). Use of the native promoter will help avoid artifactual responses generated by ectopic expression of the mutant chromoproteins. These transgenes along with a transgene encoding wild-type ZmphyB-FLAG will be stably introduced into maize using a Maize Transformation protocol which exploits the Hi Type-II background for most transformations, generated from a cross between the B73 and A188 hybrids followed by selection for efficient regeneration of plantlets from cultured embryos. The transgenic plants expressing a range of ZmphyB1 polypeptide levels will be identified by immunoblot analysis with available FLAG and phyB-specific monoclonal antibodies. Independent transformants that express the mutant phyB proteins at a level near to that in wild-type plants will be identified since artificially increased or decreased levels of ZmphyB might significantly influence photomorphogenesis by themselves. Those lines deemed useful will then be backcrossed at least three times to the B73 inbred to generate lines suitable to phenotypic testing. A library of suitable independent lines for each mutation will be generated to avoid potential artifacts generated by insertion position of the transgene and/or differing accumulation of the ZmphyB1 biliprotein.

Some mutants are expected to work dominantly even in the presence of wild-type ZmphyB1/2. However, others will likely confer more subtle phenotypes that will require eliminating the wild-type photoreceptor for observation. This situation will be accomplished through crosses with the ZmphyB1 and ZmphyB2 mutants developed by Sheehan et al., Plant J 49:338-353 (2007) using Mu insertional mutagenesis, followed by selfing to identify triple homozygous progeny. Single and double mutant combinations will be generated for the strongest ZmphyB1-Mu563 and ZmphyB2-Mu12053 alleles, which have been backcrossed 4 times into both the B73 and W22 backgrounds.

Plants containing unmodified ZmphyB1-FLAG or the modified phytochromes in either the wild-type B73 or the ZmphyB1-Mu563 and ZmphyB2-Mu12053 B73-introgressed backgrounds will be examined by various phenotypic assays that specifically measure phyB activity. The germplasm will be tested alongside several controls including, near isogenic wild-type B73, B73 expressing unmodified ZmphyB1, and the ZmphyB1-Mu563 and ZmphyB2-Mu12053 B73-introgressed lines either singly or as double mutants. To reduce environmental variability, the plants will be grown in controlled environment cabinets equipped with monochromatic R and FR LED light sources and growth chambers illuminated with white light within the lab and greenhouses supplemented with artificial lighting if needed. Randomized block design will be used to avoid biases based on positions of the plants within the group. Testing of plants in outdoor agricultural plots under natural lighting conditions will be carried out to assess their impact on maize seed yield and plant stature in more representative field settings.

The phenotypes to be tested have been well established in maize and include:

(1) Architecture of seedling grown in the dark (etiolated), which is expected to be unaffected by the mutations.
(2) Effect of R, FR. R-FR. and white light pulses on coleoptile, mesocotyl, and leaf sheath and blade elongation for young seedlings.
(3) Effect of EOD-FR on mesocotyl, and leaf blade elongation for young seedlings grown in light/dark cycles.
(4) Chlorophyll and anthocyanin accumulation in seedlings grown in light/dark cycles.
(5) Effect on internode length, stem diameter, and overall plant height on plants grown in long-day photoperiods.
(6) Effect on flowering time for plants grown in long- and short-day photoperiods.
(7) Number of tillers, cobs, and kernels produced in long-days.

Examining a range of R and FR fluence rates on the photomorphogenic responses of young seedlings will facilitate the quantification of the degree of hypo- or hyperactivity for each mutant. It is expected that at least some of the ZmphyB mutants will confer useful new traits such as altered flowering time or reduced SAR (shade avoidance response) to maize grown in field situations.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. An isolated polynucleotide encoding a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92.

Clause 2. The isolated polynucleotide of clause 1, wherein the modified phytochrome polypeptide comprises an amino acid other than tyrosine at the residue corresponding to position 104 of SEQ ID NO: 1, an amino acid other than isoleucine or methionine at the residue corresponding to position 108 of SEQ ID NO: 1, an amino acid other than glycine at the residue corresponding to position 284 of SEQ ID NO: 1, an amino acid other than histidine at the residue corresponding to position 358 of SEQ ID NO: 1, an amino acid other than valine at the residue corresponding to position 401 of SEQ ID NO: 1, an amino acid other than histidine at the residue corresponding to position 403 of SEQ ID NO: 1, an amino acid other than tryptophan at the residue corresponding to position 563 of SEQ ID NO: 1, an amino acid other than glycine at the residue corresponding to position 565 of SEQ ID NO: 1, an amino acid other than serine at the residue corresponding to position 584 of SEQ ID NO: 1, or combinations thereof.

Clause 3. The isolated polynucleotide of clause 1 or 2, wherein the modified phytochrome polypeptide comprises a substitution corresponding to at least one of Y104-A, I108-A, I108-Y, G284-V, H358-A, V401-S, H403-A, W563-S, G565-E, S584-A, S584-E, or a combination thereof, of SEQ ID NO:1.

Clause 4. The isolated polynucleotide of any one of clauses 1-3, wherein the modified phytochrome polypeptide has at least one of an altered thermal reversion rate, an altered photoconversion rate, an altered absorption spectrum, an altered signal output compared to the unmodified phytochrome polypeptide, or combinations thereof.

Clause 5. The isolated polynucleotide of any one of clauses 1-4, wherein the modified phytochrome polypeptide has an altered thermal reversion rate compared to the unmodified phytochrome polypeptide.

Clause 6. The isolated polynucleotide of clause 5, wherein the rate of thermal reversion of the modified phytochrome polypeptide is decreased compared to the unmodified phytochrome polypeptide.

Clause 7. The isolated polynucleotide of clause 5, wherein the rate of thermal reversion of the modified phytochrome polypeptide is decreased at least 0.5 fold compared to the unmodified phytochrome polypeptide.

Clause 8. The isolated polynucleotide of clause 5, wherein the rate of thermal reversion of the modified phytochrome polypeptide is increased compared to the unmodified phytochrome polypeptide.

Clause 9. The isolated polynucleotide of clause 5, wherein the rate of thermal reversion of the modified phytochrome polypeptide is increased at least 0.5 fold compared to the unmodified phytochrome polypeptide.

Clause 10. The isolated polynucleotide any one of clauses 1-4, wherein the modified phytochrome polypeptide has an altered photoconversion rate compared to the unmodified phytochrome polypeptide.

Clause 11. The isolated polynucleotide of clause 10, wherein the photoconversion rate from the Pfr form to the Pr form of the modified phytochrome polypeptide is increased compared to the unmodified phytochrome polypeptide.

Clause 12. The isolated polynucleotide of clause 10, wherein the photoconversion rate from the Pfr form to the Pr form of the modified phytochrome polypeptide is decreased compared to the unmodified phytochrome polypeptide.

Clause 13. The isolated polynucleotide of clause 11 or 12, wherein the photoconversion rate is determined at a wavelength of about 720 nm.

Clause 14. The isolated polynucleotide of clause 10, wherein the photoconversion rate from the Pr form to the Pfr form of the modified phytochrome polypeptide is increased compared to the unmodified phytochrome polypeptide.

Clause 15. The isolated polynucleotide of clause 10, wherein the photoconversion rate from the Pr form to the Pfr form of the modified phytochrome polypeptide is decreased compared to the unmodified phytochrome polypeptide.

Clause 16. The isolated polynucleotide of clause 14 or 15, wherein the photoconversion rate is determined at a wavelength of about 660 nm or about 720 nm.

Clause 17. The isolated polynucleotide of clause 11 or 14, wherein the photoconversion rate of the modified phytochrome polypeptide is increased at least 0.5 fold compared to the unmodified phytochrome polypeptide.

Clause 18. The isolated polynucleotide of clause 12 or 15, wherein the photoconversion rate of the modified phytochrome polypeptide is decreased at least 0.5 fold compared to the unmodified phytochrome polypeptide.

Clause 19. The isolated polynucleotide of any one of clauses 1-4, wherein the modified phytochrome polypeptide has an altered absorption spectrum compared to the unmodified phytochrome polypeptide.

Clause 20. The isolated polynucleotide of any one of clauses 1-3, wherein the altered absorption spectrum is a shift in an absorption peak wavelength.

Clause 21. The isolated polynucleotide of clause 19 or 20, wherein the modified phytochrome polypeptide has a Pr absorption spectrum that is shifted to a longer wavelength compared to the unmodified phytochrome polypeptide.

Clause 22. The isolated polynucleotide of clause 19 or 20, wherein the modified phytochrome polypeptide has a Pr absorption spectrum that is shifted to a shorter wavelength compared to the unmodified phytochrome polypeptide.

Clause 23. The isolated polynucleotide of clause 19 or 20, wherein the modified phytochrome polypeptide has a Pfr absorption spectrum that is shifted to a longer wavelength compared to the unmodified phytochrome polypeptide.

Clause 24. The isolated polynucleotide of clause 19 or 20, wherein the modified phytochrome polypeptide has a Pfr absorption spectrum that is shifted to a shorter wavelength compared to the unmodified phytochrome polypeptide.

Clause 25. The isolated polynucleotide of any one of clauses 1-4, wherein the modified phytochrome polypeptide has an altered signal output compared to the unmodified phytochrome polypeptide.

Clause 26. The isolated polynucleotide of any one of the preceding clauses, wherein the modified phytochrome polypeptide further comprises at least one amino acid substitution at a position corresponding to position 276, 307, 322, 352, 361, 564, 582, or a combination thereof, of SEQ ID NO:1.

Clause 27. The isolated polynucleotide of clause 26, wherein the modified phytochrome polypeptide further comprises a substitution corresponding to at least one of Y276-H, D307-A, R322-A, R352-A, Y361-F, G564-E, R582-A, or a combination thereof, of SEQ ID NO:1.

Clause 28. A vector comprising the isolated polynucleotide of any one of the preceding clauses.

Clause 29. An isolated polynucleotide construct comprising a promoter not natively associated with the polynucleotide of clause 1 operably linked to the polynucleotide of any one of clauses 1-27.

Clause 30. A plant cell comprising the isolated polynucleotide of any one of clauses 1-27 operably linked to a promoter not natively associated with the polynucleotide of clause 1.

Clause 31. A plant comprising the plant cell of clause 30.

Clause 32. The plant of clause 31, wherein the plant exhibits increased light sensitivity relative to a control plant lacking the polynucleotide.

Clause 33. The plant of clause 31 or 32, wherein the plant exhibits a decreased height, decreased diameter or a combination thereof, relative to a control plant lacking the polynucleotide.

Clause 34. The plant of any one of clauses 31-33, wherein the plant exhibits at least one characteristic selected from, increased hyponasty, decreased petiole length, decreased internode length, and decreased hypocotyl length under an R fluence rate of less than 1 μmole m⁻² sec⁻¹, relative to a control plant lacking the polynucleotide.

Clause 35. The plant of any one of clauses 31-34, wherein the plant exhibits enhanced germination relative to the control plant.

Clause 36. The plant of clause 35, wherein the plant is corn, soybean or rice.

Clause 37. The plant of clause 35, wherein the plant is an ornamental plant.

Clause 38. A method of producing a transgenic plant, the method comprising:
(a) introducing into a plant cell an isolated polynucleotide encoding a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92; and
(b) regenerating the transformed cell to produce a transgenic plant.

Clause 39. The method of clause 38, wherein the transgenic plant exhibits increased light sensitivity relative to a control plant lacking the isolated polynucleotide.

Clause 40. The method of clause 38 or 39, wherein the transgenic plant exhibits decreased height, decreased diameter, or a combination thereof, relative to a control plant lacking the polynucleotide.

Clause 41. The method of any one of clauses 38-40, wherein the transgenic plant exhibits at least one characteristic selected from decreased petiole length, decreased internode number, increased hyponasty, and decreased hypocotyl length under an R fluence rate of less than 1 μmole m⁻² sec⁻¹, relative to a control plant lacking the polynucleotide.

Clause 42. The method of any one of clauses 38-41, wherein the transgenic plant exhibits enhanced germination relative to the control plant.

Clause 43. The method of clause 42, wherein the transgenic plant is a corn, soybean or rice plant.

Clause 44. The method of clause 42, wherein the transgenic plant is an ornamental plant.

Clause 45. A transgenic plant produced by the method of any one of clauses 38-44.

Clause 46. An isolated polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92.

1. An isolated polynucleotide encoding a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92.

2. The isolated polynucleotide of claim 1, wherein the modified phytochrome polypeptide comprises an amino acid other than tyrosine at the residue corresponding to position 104 of SEQ ID NO:1, an amino acid other than isoleucine or methionine at the residue corresponding to position 108 of SEQ ID NO:1, an amino acid other than glycine at the residue corresponding to position 284 of SEQ ID NO:1, an amino acid other than histidine at the residue corresponding to position 358 of SEQ ID NO: 1, an amino acid other than valine at the residue corresponding to position 401 of SEQ ID NO:1, an amino acid other than histidine at the residue corresponding to position 403 of SEQ ID NO:1, an amino acid other than tryptophan at the residue corresponding to position 563 of SEQ ID NO:1, an amino acid other than glycine at the residue corresponding to position 565 of SEQ ID NO:1, an amino acid other than serine at the residue corresponding to position 584 of SEQ ID NO:1, or combinations thereof.

3. The isolated polynucleotide of claim 1, wherein the modified phytochrome polypeptide comprises a substitution corresponding to at least one of Y104-A, I108-A, I108-Y, G284-V, H358-A, V401-S, H403-A, W563-S, G565-E, S584-A, S584-E, or a combination thereof, of SEQ ID NO:1.

4. The isolated polynucleotide of claim 1, wherein the modified phytochrome polypeptide has at least one of an altered thermal reversion rate, an altered photoconversion rate, an altered absorption spectrum, an altered signal output compared to the unmodified phytochrome polypeptide, or combinations thereof.

5.-25. (canceled)

26. The isolated polynucleotide of claim 1, wherein the modified phytochrome polypeptide further comprises at least one amino acid substitution at a position corresponding to position 276, 307, 322, 352, 361, 564, 582, or a combination thereof, of SEQ ID NO:1.

27. The isolated polynucleotide of claim 26, wherein the modified phytochrome polypeptide further comprises a substitution corresponding to at least one of Y276-H, D307-A, R322-A, R352-A, Y361-F, G564-E, R582-A, or a combination thereof, of SEQ ID NO:1.

28. A vector comprising the isolated polynucleotide of claim 1.

29. An isolated polynucleotide construct comprising a promoter not natively associated with the polynucleotide of claim 1 operably linked to the polynucleotide of claim 1.

30. A plant cell comprising the isolated polynucleotide of claim 1 operably linked to a promoter not natively associated with the polynucleotide of claim 1.

31. A plant comprising the plant cell of claim 30.

32. The plant of claim 31, wherein the plant exhibits:
(a) increased light sensitivity;
(b) a decreased height, decreased diameter or a combination thereof;
(c) at least one characteristic selected from, increased hyponasty, decreased petiole length, decreased internode length, and decreased hypocotyl length under an R fluence rate of less than 1 μmole m⁻² sec⁻¹; or
(d) enhanced germination,
relative to a control plant lacking the polynucleotide.

33.-35. (canceled)

36. The plant of claim 35, wherein the plant is corn, soybean or rice.

37. The plant of claim 35, wherein the plant is an ornamental plant.

38. A method of producing a transgenic plant, the method comprising:
(a) introducing into a plant cell an isolated polynucleotide encoding a modified phytochrome polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92; and
(b) regenerating the transformed cell to produce a transgenic plant.

39. The method of claim 38, wherein the transgenic plant exhibits
(a) increased light sensitivity;
(b) decreased height, decreased diameter, or a combination thereof;
(c) at least one characteristic selected from decreased petiole length, decreased internode number, increased hyponasty, and decreased hypocotyl length under an R fluence rate of less than 1 μmole m⁻² sec⁻¹; or
(d) enhanced germination,
relative to a control plant lacking the isolated polynucleotide.

40.-42. (canceled)

43. The method of claim 42, wherein the transgenic plant is a corn, soybean or rice plant.

44. The method of claim 42, wherein the transgenic plant is an ornamental plant.

45. A transgenic plant produced by the method of claim 38.

46. An isolated polypeptide comprising an amino acid sequence that is at least 80% identical to an unmodified phytochrome polypeptide and having at least one amino acid substitution at a position corresponding to position 104, 108, 284, 358, 401, 403, 563, 565, 584, or a combination thereof, of SEQ ID NO:1, the unmodified phytochrome polypeptide having an amino acid sequence selected from SEQ ID NOs: 1-26 or 67-92.
